(19) United States
(12) Patent Application Publication
(10) Pub. No.: US 2023/0174975 A1
(43) Pub. Date: Jun. 8, 2023

(54) SYSTEMS AND METHODS FOR VIRAL GENOME TARGETING

(71) Applicant: THE BOARD OF TRUSTEES OF THE LELAND STANFORD JUNIOR UNIVERSITY, Stanford, CA (US)

(72) Inventors: Lei S. Qi, Stanford, CA (US); Marie F. LA RUSSA, Stanford, CA (US); Timothy R. ABBOTT, Stanford, CA (US); David B. LEWIS, Stanford, CA (US)

(73) Assignee: THE BOARD OF TRUSTEES OF THE LELAND STANFORD JUNIOR UNIVERSITY, Stanford, CA (US)

(21) Appl. No.: 17/911,079

(22) PCT Filed: Mar. 12, 2021
(86) PCT No.: PCT/US2021/022107

§ 371 Date: Sep. 12, 2022

Related U.S. Application Data
(60) Provisional application No. 62/989,087, filed on Mar. 13, 2020.
(60) Provisional application No. 62/989,181, filed on Mar. 13, 2020.

(51) Int. Cl. C12N 15/11; C12N 9/22; C12N 15/86; A61K 38/46; A61K 31/7088; A61P 31/14; A61K 45/06

(52) U.S. Cl. CPC C12N 15/11; C12N 9/22; C12N 15/86; A61K 38/465; A61K 31/7088; A61P 31/14; A61K 45/06; C12N 2310/20; C12N 2800/80; C12N 2320/31; C12N 2740/15043

(57) ABSTRACT

Described herein are systems for targeting viral genomes. Also described herein are methods for targeting viral genomes utilizing the systems described in the instant disclosure. In some cases, systems and methods disclosed herein can be used to treat viral infections. In preferred embodiments, the systems utilise a CRISPR/Cas13d complex to target the genome of an RNA virus such as coronavirus or influenza virus.

### CROSS-REFERENCE

This application claims the benefit of U.S. Provisional Application Ser. No. 62/989,087, filed on Mar. 13, 2020, and U.S. Provisional Application Ser. No. 62/989,181, filed on Mar. 13, 2020, each of which is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under contract HR001119C0060 awarded by DARPA. The Government has certain rights in the invention.

### SEQUENCE LISTING

The instant application contains a Sequence Listing which has been submitted electronically in ASCII format and is hereby incorporated by reference in its entirety. Said ASCII copy, created on Mar. 9, 2021, is named 55176-709_601_SL.txt and is 2,494,464 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

A number of viruses (e.g., coronaviruses, influenza viruses, etc.) can cause severe diseases and even deaths in mammals, including humans. Examples of the coronaviruses can include Severe Acute Respiratory Syndrome (SARS-CoV), SARS-CoV-2 (i.e., COVID-19), and Middle East Respiratory Syndrome (MERS-CoV). In some cases, certain virus strains can evolve and transfer to humans from diverse animal hosts that act as viral reservoirs, thereby exacerbating the threat of viral infections in humans. For example, two strains (L and S) of a virus (e.g., SARS-CoV-2) with different genome sequences can evolve and circulate, thus rendering existing vaccination or treatment strategies ineffective. In some cases, vaccines or treatment options often do not exist for some existing or new viruses.

### SUMMARY

The outbreak of unpredictable and highly contagious viral infections presents ongoing challenges. With a rapidly growing number of diseases and deaths around the world caused by viruses (e.g., coronaviruses, influenza viruses, etc.), there remains a pressing need to effectively detect, mutate, and/or degrade viral genomes to treat or prevent viral infections. There also remains a need for systems and methods to treat viral infections or diseases by targeting the transcripts of viral genomes or viral genes for degradation.

Described herein are systems and methods for targeting viral genomes or viral genes. In some cases, the viral genomes or viral genes are targeted for reducing expression or activity thereof.

Described herein, in some embodiments, is a system for reducing viral infection in a cell, comprising: a heterologous polypeptide comprising a gene regulating moiety; or one or more heterologous nucleic acid molecules, wherein at least the gene regulating moiety or the one or more heterologous nucleic acid molecules are configured to specifically bind one or more target viral genes of a coronavirus in the cell, to reduce expression or activity of the one or more target viral genes of the coronavirus in the cell, and wherein the one or more target viral genes encode: a viral RNA-dependent RNA polymerase (RdRP); or a viral nucleocapsid protein (N-protein).

In some embodiments, the one or more heterologous nucleic acid molecules comprises a plurality of different heterologous nucleic acid molecules that is collectively configured to: bind different portions of a same target viral gene of the coronavirus; or different target viral genes of the coronavirus. In some embodiments, the plurality of different heterologous nucleic acid molecules comprises at least 3, 4, 5, or 6 different heterologous nucleic acid molecules.

In some embodiments of any of the system disclosed herein, a heterologous nucleic acid molecule of the one or more heterologous nucleic acid molecules exhibits at most 3, 2, or 1 mismatch(es) when optimally aligned with a MERS-CoV genome or a SARS-CoV genome.

In some embodiments of any of the system disclosed herein, a heterologous nucleic acid molecule of the one or more heterologous nucleic acid molecules has at least about 80%, 85%, 90%, 95%, or 99% sequence identity or complementarity to a sequence selected from the group consisting of SEQ ID NOs: 4001-7203, 12101-12140, and 13101-13122.

In some embodiments of any of the system disclosed herein, the one or more target viral genes comprise a plurality of target viral genes encoding the viral RdRP and the N-protein.

In some embodiments of any of the system disclosed herein, the system is sufficient to reduce expression or activity level of the RdRP in the cell by at least about 70% or 75%, as compared to a corresponding control.

In some embodiments of any of the system disclosed herein, the system is sufficient to reduce expression or activity level of the N-protein in the cell by at least about 70%, 75%, 80%, or 85%, as compared to a corresponding control.

In some embodiments of any of the system disclosed herein, the system is sufficient to reduce infection of at least about 30%, 35%, 40%, 50%, 60%, 70%, 80%, or 85% of a plurality of different strains of the coronavirus, as compared to a corresponding control. In some embodiments, the plurality of different strains of the coronavirus comprises at least one strain from a member selected from the group consisting of alphacoronavirus, betacoronavirus, deltacoronavirus, and gammacoronavirus. In some embodiments, the plurality of different strains of the coronavirus comprises at least one strain from each of two, three, or four members selected from the group consisting of alphacoronavirus, betacoronavirus, deltacoronavirus, and gammacoronavirus. In some embodiments of any of the system disclosed herein, the plurality of different strains of the coronavirus comprises at least about 100, 500, 1000, 1500, 2000, or 2500 different strains.

In some embodiments of any of the system disclosed herein, at least the gene regulating moiety is configured to specifically bind the one or more target viral genes of the coronavirus in the cell. In some embodiments of any of the system disclosed herein, at least the one or more heterologous nucleic acid molecules are configured to specifically bind the one or more target viral genes of the coronavirus in the cell. In some embodiments of any of the system disclosed herein, the gene regulating moiety and the one or more heterologous nucleic acid molecules are collectively configured to specifically bind the one or more target viral genes of the coronavirus in the cell. In some embodiments, the gene regulating moiety is configured to form a complex with the one or more heterologous nucleic acid molecules, the complex being configured to bind the one or more target viral genes.

Described herein, in some embodiments, is a system for reducing viral infection in a cell, comprising: a heterologous polypeptide comprising a gene regulating moiety; or one or more heterologous nucleic acid molecules, wherein at least the gene regulating moiety or the one or more heterologous nucleic acid molecules are configured to specifically bind one or more non-coding regions of at least one target viral gene in the cell, to reduce expression or activity of the at least one target viral gene in the cell.

In some embodiments, the one or more heterologous nucleic acid molecules comprises a plurality of different heterologous nucleic acid molecules that is collectively configured to bind a plurality of different non-coding regions of the at least one target viral gene.

In some embodiments of any of the system disclosed herein, the plurality of different non-coding regions is operatively coupled to a same target viral gene. In some embodiments of any of the system disclosed herein, a viral sequence encoding a viral protein is flanked by the plurality of different non-coding regions. In some embodiments, the same target viral gene encodes a neuraminidase. In some embodiments, the same target viral gene encodes a hemagglutinin.

In some embodiments of any of the system disclosed herein, the plurality of different non-coding regions is operatively coupled to different target viral genes. In some embodiments, the system comprises a first target viral gene of the different target viral genes encodes a neuraminidase and a second target viral gene of the different target viral genes encodes a viral protein selected from the group consisting of a polymerase, a hemagglutinin, a nucleoprotein, a matrix protein, a non-structural protein, and a nuclear export protein. In some embodiments, the system targets a first target viral gene of the different target viral genes encodes a hemagglutinin and a second target viral gene of the different target viral genes encodes a viral protein selected from the group consisting of a polymerase, a nucleoprotein, a matrix protein, a non-structural protein, and a nuclear export protein.

In some embodiments of any of the system disclosed herein, the plurality of different heterologous nucleic acid molecules comprises at least 3, 4, 5, or 6 different heterologous nucleic acid molecules.

In some embodiments of any of the system disclosed herein, at least one target viral gene is from an influenza virus. In some embodiments of any of the system disclosed herein, one or more non-coding regions are conserved across a plurality of different influenza virus strains. In some embodiments, the plurality of different influenza virus strains comprises at least one strain from a member selected from the group consisting of influenza A, influenza B, influenza C, and influenza D. In some embodiments, the plurality of different influenza virus strains comprises subtypes of influenza A. In some embodiments, the plurality of different influenza virus strains comprises at least one strain from each of two, three, or four members selected from the group consisting of influenza A, influenza B, influenza C, and influenza D. In some embodiments, the plurality of different influenza virus strains comprises at least about 10, 20, 50, 100, or 120 different influenza virus strains.

In some embodiments of any of the system disclosed herein, the one or more non-coding regions comprise a sequence having at least about 80%, 85%, 90%, 95%, or 99% sequence identity or complementarity to a sequence selected from the group consisting of SEQ ID NOs: 13501-13548.

In some embodiments of any of the system disclosed herein, the system is sufficient to reduce infection of an influenza virus in the cell by at least about 50%, 55%, 60%, 65%, or 70%, as compared to a corresponding control.

In some embodiments of any of the system disclosed herein, at least the gene regulating moiety is configured to specifically bind the one or more non-coding regions of the at least one target viral gene. In some embodiments of any of the system disclosed herein, at least the one or more heterologous nucleic acid molecules are configured to specifically bind the one or more non-coding regions of the at least one target viral gene. In some embodiments of any of the system disclosed herein, the gene regulating moiety and the one or more heterologous nucleic acid molecules are collectively configured to specifically bind the one or more non-coding regions of the at least one target viral gene. In some embodiments, the gene regulating moiety is configured to form a complex with the one or more heterologous nucleic acid molecules, the complex being configured to bind the one or more non-coding regions of the at least one target viral gene.

Described herein, in some embodiments, is a system for reducing viral infection in a cell, comprising: a plurality of heterologous gene regulating moieties that comprises no more than 20 members; or a plurality of heterologous nucleic acid molecules that comprises no more than 20 members; wherein the plurality of heterologous gene regulating moieties or the plurality of heterologous nucleic acid molecules is configured to specifically bind at least about 70% of a plurality of strains from a plurality of RNA virus families.

In some embodiments, the plurality of strains comprises at least about 1000, 5000, 10000, 15000, 20000, or 22000 different strains. In some embodiments of any of the system disclosed herein, the specific binding is to at least about 75%, 80%, 85%, or 90% of the plurality of strains from the plurality of RNA virus families. In some embodiments of any of the system disclosed herein, the plurality of RNA virus families comprises two or members selected from the group consisting of Coronaviridae, Pneumoviridae, Paramyxoviridae, Caliciviridae, Hepeviridae, Picomaviridae, Filoviridae, Flaviviridae, Rhabdoviridae, Togaviridae, Orthomyxoviridae, and Retroviridae. In some embodiments of any of the system disclosed herein, the plurality of RNA virus families comprises two or more members selected from the group consisting of Coronaviridae, Pneumoviridae, Paramyxoviridae, Caliciviridae, Hepeviridae, Picomaviridae, Filoviridae, Flaviviridae, Rhabdoviridae, and Togaviridae.

In some embodiments of any of the system disclosed herein, at least the plurality of heterologous gene regulating moieties is configured to specifically bind at least about 70% of the plurality of strains. In some embodiments of any of the system disclosed herein, at least the plurality of heterologous nucleic acid molecules is configured to specifically bind at least about 70% of the plurality of strains. In some embodiments of any of the system disclosed herein, at least the plurality of heterologous gene regulating moieties and the plurality of heterologous nucleic acid molecules are collectively configured to specifically bind at least about 70% of the plurality of strains. In some embodiments, the system comprises a gene regulating moiety of the plurality of heterologous gene regulating moieties is configured to form a complex with a heterologous nucleic acid molecule of the plurality of plurality of heterologous nucleic acid molecules, the complex being configured to bind a target viral gene from the plurality of strains.

Described herein, in some embodiments, is a system for reducing viral infection in a cell, comprising: a plurality of heterologous gene regulating moieties that comprises no more than 20 members; or a plurality of heterologous nucleic acid molecules that comprises no more than 20 members; wherein the plurality of heterologous gene regulating moieties or the plurality of heterologous nucleic acid molecules is configured to specifically bind at least about 30% of a plurality of strains from each RNA virus family of a plurality of RNA virus families.

In some embodiments, the plurality of strains from said each RNA virus family comprises at least about 100, 200, 300, 400, 500, 1,000, 2,000, or more different strains. In some embodiments of any of the system disclosed herein, the specific binding is to at least about 35%, 40%, 45%, or 50% of the plurality of strains from said each RNA virus family. In some embodiments of any of the system disclosed herein, the specific binding is to at least about 55%, 60%, 65%, 70%, 75%, or 80% of the plurality of strains of one or more RNA virus families selected from the group consisting of Coronaviridae, Pneumoviridae, Caliciviridae, Hepeviridae, Picomaviridae, Filoviridae, Flaviviridae, and Togaviridae. In some embodiments of any of the system disclosed herein, the specific binding is to at least about 85% of the plurality of strains of one or more RNA virus families selected from the group consisting of Coronaviridae, Pneumoviridae, Hepeviridae. Picomaviridae, Filoviridae. Flaviviridae, and Togaviridae.

In some embodiments of any of the system disclosed herein, at least the plurality of heterologous gene regulating moieties is configured to specifically bind at least about 30% of the plurality of strains. In some embodiments of any of the system disclosed herein, at least the plurality of heterologous nucleic acid molecules is configured to specifically bind at least about 30% of the plurality of strains. In some embodiments of any of the system disclosed herein, at least the plurality of heterologous gene regulating moieties and the plurality of heterologous nucleic acid molecules are collectively configured to specifically bind at least about 30% of the plurality of strains. In some embodiments, the system comprises a gene regulating moiety of the plurality of heterologous gene regulating moieties is configured to form a complex with a heterologous nucleic acid molecule of the plurality of plurality of heterologous nucleic acid molecules, the complex being configured to bind a target viral gene from the plurality of strains.

In some embodiments of any of the system disclosed herein, the plurality of RNA virus families is a plurality of human-infectious RNA virus families. In some embodiments, the plurality of heterologous gene regulating moieties comprises between about 5 and about 20 different members. In some embodiments, the plurality of heterologous gene regulating moieties comprises between about 8 and about 20 different members. In some embodiments, the plurality of heterologous gene regulating moieties comprises between about 10 and about 18 different members. In some embodiments, the plurality of heterologous gene regulating moieties comprises between about 12 and about 16 different members. In some embodiments, the plurality of heterologous gene regulating moieties comprises between about 13 and about 15 different members. In some embodiments, the plurality of heterologous gene regulating moieties comprises about 14 different members.

In some embodiments of any of the system disclosed herein, a heterologous gene regulating moiety of the plurality of gene regulating moieties exhibits a specific binding to a target polynucleotide sequence, wherein the target polynucleotide sequence has at least about 50%, 60%, 70%, 80%, 85%, 90%, 95%, or 99% sequence identity or complementarity to a sequence selected from Table 11.

In some embodiments of any of the system disclosed herein, the plurality of heterologous nucleic acid molecules comprises between about 5 and about 20 different members. In some embodiments, the plurality of heterologous nucleic acid molecules comprises between about 8 and about 20 different members. In some embodiments, the plurality of heterologous nucleic acid molecules comprises between about 10 and about 18 different members. In some embodiments, the plurality of heterologous nucleic acid molecules comprises between about 12 and about 16 different members. In some embodiments, the plurality of heterologous nucleic acid molecules comprises between about 13 and about 15 different members. In some embodiments, the plurality of heterologous nucleic acid molecules comprises about 14 different members. In some embodiments of any of the system disclosed herein, a heterologous nucleic acid molecule of the plurality of heterologous nucleic acid molecules has at least about 50%, 60%, 70%, 80%, 85%, 90%, 95%, or 99% sequence identity or complementarity to a sequence selected from Table 11.

In some embodiments of any of the system disclosed herein, the heterologous nucleic acid molecule(s) has a length ranging from about 10 to about 30 nucleotides.

In some embodiments of any of the system disclosed herein, the heterologous nucleic acid molecule(s) is a heterologous guide RNA molecule.

In some embodiments of any of the system disclosed herein, the gene regulating moiety is a heterologous endonuclease. In some embodiments, the heterologous endonuclease is an RNA-targeting endonuclease. In some embodiments, the heterologous endonuclease exhibits RNA cleavage activity. In some embodiments, the heterologous endonuclease comprises a CRISPR/Cas protein or a modification thereof. In some embodiments, the CRISPR/Cas protein is Cas13. In some embodiments, the CRISPR/Cas protein is Cas13d.

In some embodiments of any of the system disclosed herein, the system comprises the heterologous polypeptide or a gene encoding thereof or the heterologous nucleic acid molecule(s) or a gene encoding thereof is packaged in a delivery vehicle selected from the group consisting of a liposome, a nanoparticle, and a viral capsule.

Described herein, in some embodiments, is a system for reducing viral infection in a cell, comprising: a first therapeutic agent configured to specifically bind a target viral gene, the first therapeutic agent comprising: a heterologous polypeptide comprising a gene regulating moiety; or a heterologous nucleic acid molecule; and a second therapeutic agent that does not specifically bind a viral gene, wherein a combination of the first and second therapeutic agents reduces viral infection in the cell.

In some embodiments, the combination effects enhanced reduction in the viral infection in the cell by at least about 10%, 20%, 30%, 40%, or 45% as compared to a corresponding control. In some embodiments, the corresponding control comprises a cell lacking the first therapeutic agent or the second therapeutic agent. In some embodiments, the corresponding control comprises a cell lacking the second therapeutic agent.

In some embodiments of any of the system disclosed herein, the first therapeutic agent or a gene encoding thereof and the second therapeutic agent are in a same composition or in different compositions.

In some embodiments of any of the system herein, a synergistic effect of the combination on the reduction of the viral replication in the cell is greater than an additive effect of the combination when performed separately. In some embodiments, a fold decrease in the viral replication from the synergistic effect is greater than that from the additive effect by at least about 1-fold, 2-fold, 3-fold, 4-fold, 6-fold, 8-fold, or 10-fold.

In some embodiments of any of the system disclosed herein, the second therapeutic agent is an immune regulator. In some embodiments, the immune regulator is a cytokine. In some embodiments, the cytokine is interferon-gamma.

In some embodiments of any of the system disclosed herein, the second therapeutic agent is an antiviral agent. In some embodiments, the antiviral agent is a small molecule. In some embodiments, the antiviral agent is Camostat.

In some embodiments of any of the system disclosed herein, the second therapeutic agent is configured to specifically bind an target endogenous gene of the cell, the target endogenous gene encoding a protein involved in viral infection or replication. In some embodiments, the protein is a transmembrane protease. In some embodiments, the transmembrane protease is TMPRSS2. In some embodiments, the protein is a MTHFD1. In some embodiments, the protein is a peptidase. In some embodiments, the protein is an aminopeptidase. In some embodiments, the protein is ANPEP.

In some embodiments of any of the system disclosed herein, the second therapeutic agent comprises: an additional heterologous polypeptide comprising an additional gene regulating moiety; or an additional heterologous nucleic acid molecule. In some embodiments, the additional gene regulating moiety is configured to specifically bind the target endogenous gene. In some embodiments, the additional heterologous nucleic acid molecule is configured to specifically bind the target endogenous gene. In some embodiments, the additional gene regulating moiety is configured to form a complex with the additional heterologous nucleic acid molecule, the complex being configured to bind the target endogenous gene. In some embodiments, the additional heterologous nucleic acid molecule is a guide nucleic acid molecule.

In some embodiments of any of the system disclosed herein, the gene regulating moiety is configured to specifically bind the target viral gene. In some embodiments of any of the system disclosed herein, the heterologous nucleic acid molecule is configured to specifically bind the target viral gene. In some embodiments of any of the system disclosed herein, the gene regulating moiety is configured to form a complex with the heterologous nucleic acid molecule, the complex being configured to bind the target viral gene. In some embodiments of any of the system disclosed herein, the heterologous nucleic acid molecule is a guide nucleic acid molecule.

In some embodiments of any of the system disclosed herein, the guide nucleic acid molecule has a length ranging from about 10 to about 30 nucleotides. In some embodiments of any of the system disclosed herein, the gene regulating moiety is a heterologous endonuclease. In some embodiments, the heterologous endonuclease is an RNA-targeting endonuclease. In some embodiments, the heterologous endonuclease exhibits RNA cleavage activity. In some embodiments, the heterologous endonuclease comprises a CRISPR/Cas protein or a modification thereof. In some embodiments, the CRISPR/Cas protein is Cas13. In some embodiments, the CRISPR/Cas protein is Cas13d.

Described herein, in some embodiments, is a system for reducing viral infection in a cell, comprising: a heterologous polypeptide comprising a gene regulating moiety that is operatively coupled to a nuclear localization signal (NLS) (gene regulating moiety-NLS); and a heterologous polynucleotide sequence encoding a guide nucleic acid molecule, wherein, upon expression of the guide nucleic acid molecule from the heterologous polynucleotide sequence in the cell, the gene regulating moiety-NLS forms a complex with the guide nucleic acid molecule, the complex being configured to bind a target viral gene to reduce the viral infection in the cell.

In some embodiments, the heterologous polynucleotide sequence is not integrated into a genome of the cell.

In some embodiments of any of the system disclosed herein, the binding effects reduction of the viral infection in the cell by at least about 10%, 20%, 30%, 40%, or 50%, as compared to a corresponding control. In some embodiments, the corresponding control comprises a cell that comprises a gene regulating moiety in absence of the NLS and the heterologous polynucleotide sequence encoding the guide nucleic acid molecule. In some embodiments, in the corresponding control, the gene regulating moiety is operatively coupled to a nuclear export signal (NES).

In some embodiments of any of the system disclosed herein, the viral infection is viral replication in the cell. In some embodiments of any of the system disclosed herein, the guide nucleic acid molecule is a guide RNA molecule. In some embodiments of any of the system disclosed herein, the guide nucleic acid molecule has a length ranging from about 10 to about 30 nucleotides. In some embodiments of any of the system disclosed herein, the gene regulating moiety is a heterologous endonuclease. In some embodiments of any of the system disclosed herein, the heterologous endonuclease is an RNA-targeting endonuclease. In some embodiments of any of the system disclosed herein, the heterologous endonuclease exhibits RNA cleavage activity. In some embodiments of any of the system disclosed herein, the heterologous endonuclease comprises a CRISPR/Cas protein or a modification thereof. In some embodiments of any of the system disclosed herein, the CRISPR/Cas protein is Cas13. In some embodiments of any of the system disclosed herein, the CRISPR/Cas protein is Cas13d.

In some embodiments of any of the system disclosed herein, heterologous polynucleotide sequence is part of a non-replicating viral vector.

Described herein, in some embodiments, is a composition comprising any of the system disclosed herein or one or more polynucleotide sequences encoding thereof. Described herein, in some embodiments, is a kit comprising the composition.

Described herein, in some embodiments, is a reaction mixture comprising: any of the system disclosed herein; and a biological sample. In some embodiments, the biological sample is selected from the group consisting of urine, blood, cell, tissue, organ, organism, and a combination thereof.

Described herein, in some embodiments, is a method of reducing viral infection in a population of cells, the method comprising contacting a cell or a population of cells with any of the system disclosed herein, wherein upon the contacting, viral infection in the cell or the population of cells is reduced. In some embodiments, the contacting occurs in vivo. In some embodiments, the contacting occurs ex vivo. In some embodiments, the contacting occurs in vitro. In some embodiments of any of the method disclosed herein, the method further comprises expressing, in the population of cells, the system or one or more polynucleotide sequences encoding thereof. In some embodiments of any of the method disclosed herein, the method further comprises administering the system or the one or more polynucleotide sequences to a subject in need thereof. In some embodiments of any of the method disclosed herein, the method comprises, subsequent to the contacting, administering one or more cells of the population of cells to a subject in need thereof.

Described herein, in some embodiments, is a method for reducing viral infection in a cell, comprising: (a) contacting the cell with a first therapeutic agent of the system disclosed herein, the first therapeutic agent comprising: (i) a heterologous polypeptide comprising a gene regulating moiety; or (ii) a heterologous nucleic acid molecule; and (b) contacting the cell with a second therapeutic agent of the system as disclosed herein, wherein a second therapeutic agent does not specifically bind a viral gene, and wherein contacting the cell with the first and second therapeutic agents reduces viral infection in the cell.

In some embodiments, the contacting the cell with the first therapeutic agent is performed prior to, simultaneously with, or subsequent to contacting the cell with the second therapeutic agent.

In some embodiments of any of the method disclosed herein, the method further comprises administering the system disclosed herein or one or more polynucleotide sequences encoding the system to a subject in need thereof, wherein the subject comprises the cell.

In some embodiments of any of the method disclosed herein, the method further comprises, subsequent to the contacting the cell with the second therapeutic agent, administering the cell to a subject in need thereof.

In some embodiments of any of the method disclosed herein, the first therapeutic agent and the second therapeutic agent are in a same composition or separate compositions.

Disclosed herein, in some embodiments, is a method for reducing viral infection in a cell, comprising: (a) contacting the cell with any of the system disclosed herein, the system comprising: (1) a heterologous polypeptide comprising a gene regulating moiety that is operatively coupled to a nuclear localization signal (NLS) (gene regulating moiety-NLS); and (2) a heterologous polynucleotide sequence encoding a guide nucleic acid molecule; and (b) expressing the guide nucleic acid molecule in the cell from the heterologous polynucleotide sequence, wherein, upon the expressing, the gene regulating moiety-NLS forms a complex with the guide nucleic acid molecule, and the complex binds a target viral gene to reduce the viral infection in the cell.

In some embodiments, the method further comprises administering the system or one or more polynucleotide sequences encoding the system to a subject in need thereof, wherein the subject comprises the cell. In some embodiments of any of the method disclosed herein, the method further comprises, subsequent to contacting the cell with the system and expressing the guide nucleic acid molecule, administering the cell to a subject in need thereof.

Described herein, in some embodiments, a nucleic acid composition comprising a RNA-binding guide RNA, wherein the RNA-binding guide RNA comprises a targeting sequence that has a length ranging between about 10 to about 30 nucleotides and is characterized by one or more members selected from the group consisting of: (i) the targeting sequence exhibits at least about 80% sequence identity or complementarity to any one of SEQ ID NOs: 4001-7203, 12101-12140, and 13101-13122; (ii) the targeting sequence is configured to bind a polynucleotide fragment within a coronavirus genome, wherein the polynucleotide fragment has a length of between about 10 and about 30 nucleotides and exhibits at least about 80% sequence identity or complementarity to a conserved region of the coronavirus genome that encodes a RNA-dependent RNA polymerase (RdRP) a nucleocapsid protein (N-protein); and (iii) the targeting sequence comprises at most 3 mismatches when optimally aligned with a MERS-CoV genome or a SARS-CoV genome.

In some embodiments, the targeting sequence exhibits at least about 80%, 85%, 90%, 95%, or 99% sequence identity or complementarity to any one of SEQ ID NOs: 4001-7203, 12101-12140, and 13101-13122. In some embodiments, the targeting sequence exhibits at least about 80%, 85%, 90%, 95%, or 99% sequence identity or complementarity to any one of SEQ ID NOs: 4001-7203. In some embodiments, the targeting sequence exhibits at least about 80%, 85%, 90%, 95%, or 99% sequence identity or complementarity to any one of SEQ ID NOs: 12101-12140. In some embodiments, the targeting sequence exhibits at least about 80%, 85%, 90%, 95%, or 99% sequence identity or complementarity to any one of SEQ ID NOs: 13101-13122. In some embodiments, the targeting sequence exhibits at least about 80%, 85%, 90%, 95%, or 99% sequence identity or complementarity to any one of SEQ ID NO: 13101-13106. In some embodiments, targeting sequence is configured to bind the polynucleotide fragment within the coronavirus genome, wherein the polynucleotide fragment has a length of between about 10 and about 30 nucleotides and exhibits at least about 80%, 85%, 90%, 95%, or 99% sequence identity or complementarity to the conserved region of the coronavirus genome that encodes the RdRP or the N-protein. In some embodiments, the targeting sequence comprises at most 3, 2, or 1 mismatch(es) when optimally aligned with the MERS-CoV genome or the SARS-CoV genome.

In some embodiments of any of the nucleic acid composition disclosed herein, a mismatch is an insertion or a deletion. In some embodiments of any of the nucleic acid composition disclosed herein, the nucleic acid composition is characterized by two or more members selected from the group consisting of (i), (ii), and (iii). In some embodiments of any of the nucleic acid composition disclosed herein, the nucleic acid composition is characterized by all of (i), (ii), and (iii). In some embodiments of any of the nucleic acid composition disclosed herein, the nucleic acid composition comprises a plurality of different RNA-binding guide RNAs comprising the RNA-binding guide RNA, wherein each of the plurality of different RNA-binding guide RNAs is characterized by one or more members selected from the group consisting of (i), (ii), and (iii). In some embodiments of any of the nucleic acid composition disclosed herein, each of the plurality of different RNA-binding guide RNAs is characterized by two or more members selected from the group consisting of (i), (ii), and (iii). In some embodiments of any of the nucleic acid composition disclosed herein, each of the plurality of different RNA-binding guide RNAs is characterized by all of (i), (ii), and (iii). In some embodiments of any of the nucleic acid composition disclosed herein, the plurality of different RNA-binding guide RNAs comprises at least 3, 4, 5, or 6 different RNA-binding guide RNAs.

Described herein, in some embodiments, is a nucleic acid composition comprising a plurality of different RNA-binding guide RNAs, wherein no more than six members of the plurality collectively exhibits specific binding to at least about 50% of a plurality of different strains of coronaviruses selected from the group consisting of alphacoronavirus, betacoronavirus, deltacoronavirus, and gammacoronavirus.

Described herein, in some embodiments, is a nucleic acid composition comprising a plurality of different RNA-binding guide RNAs, wherein no more than six members of the plurality collectively exhibits specific binding to at least about 50% of a plurality of different strains of coronaviruses selected from the group consisting of coronaviruses as identified by GenBank Access ID.

In some embodiments of any of the nucleic acid composition disclosed herein, the specific binding is to at least about 50%, 60%, 70%, 80%, 85%, 90%, 95%, or 99% of the plurality of different strains. In some embodiments of any of the nucleic acid composition disclosed herein, the plurality of different strains of the coronavirus comprises at least about 100, 500, 1000, 1500, 2000, or 2500 different strains. In some embodiments of any of the nucleic acid composition disclosed herein, the plurality of different strains of the coronavirus comprises at least one strain from each of two, three, or four members selected from the group consisting of alphacoronavirus, betacoronavirus, deltacoronavirus, and gammacoronavirus. In some embodiments of any of the nucleic acid composition disclosed herein, the plurality of different RNA-binding guide RNAs is selected from the group consisting of the sequences in Table 4.

Described herein, in some embodiments, is a nucleic acid composition comprising one or more RNA-binding guide RNAs, wherein a RNA-binding guide RNA of the one or more RNA-binding guide RNAs comprises a targeting sequence that has a length ranging between about 10 to about 30, and wherein the RNA-binding guide RNA has at least about 80% sequence identity or complementarity to any one of SEQ ID NOs: 13301-13348.

In some embodiments, the RNA-binding guide RNA has at least about 85%, 90%, 95%, or 99% sequence identity or complementarity to any one of SEQ ID NOs: 13301-13348. In some embodiments, the RNA-binding guide RNA has at least about 80%, 85%, 90%, 95%, or 99% sequence identity or complementarity to any one of SEQ ID NOs: 13319-13324 and 13331-13336. In some embodiments, the one or more RNA-binding guide RNAs comprise a plurality of different RNA-binding guide RNAs. In some embodiments, the plurality of different RNA-binding guide RNAs comprises between about two and about eight different RNA-binding guide RNAs. In some embodiments, the plurality of different RNA-binding guide RNAs comprises between about four and about eight different RNA-binding guide RNAs. In some embodiments, the plurality of different RNA-binding guide RNAs comprises between about five and about seven different RNA-binding guide RNAs. In some embodiments, the plurality of different RNA-binding guide RNAs comprises about six different RNA-binding guide RNAs.

In some embodiments of any of the nucleic acid composition disclosed herein, the plurality of different RNA-binding guide RNAs comprises (i) a first RNA-binding guide RNA having at least about 80%, 85%, 90%, 95%, or 99% sequence identity or complementarity to any one of SEQ ID NOs: 13319-13324 or (ii) a second RNA-binding guide RNA having at least about 80%, 85%, 90%, 95%, or 99% sequence identity or complementarity to any one of SEQ ID NOs: 13331-13336.

In some embodiments of any of the nucleic acid composition disclosed herein, the plurality of different RNA-binding guide RNAs comprises the first RNA-binding guide RNA and the second RNA-binding guide RNA.

Described herein, in some embodiments, is a nucleic acid composition comprising one or more RNA-binding guide RNAs, wherein a RNA-binding guide RNA of the one or more RNA-binding guide RNAs comprises a targeting sequence that has a length ranging between about 10 to about 30, and wherein the RNA-binding guide RNA has at least about 80% sequence identity or complementarity to a sequence selected from Table 11.

In some embodiments, the RNA-binding guide RNA has at least about 85%, 90%, 95%, or 99% sequence identity or complementarity to a sequence selected from Table 11.

In some embodiments of any of the nucleic acid composition disclosed herein, the one or more RNA-binding guide RNAs comprise a plurality of different RNA-binding guide RNAs. In some embodiments, the plurality of different RNA-binding guide RNAs comprises between about 5 and about 20 different RNA-binding guide RNAs. In some embodiments, the plurality of different RNA-binding guide RNAs comprises between about 8 and about 20 different RNA-binding guide RNAs. In some embodiments, the plurality of different RNA-binding guide RNAs comprises between about 10 and about 18 different RNA-binding guide RNAs. In some embodiments, the plurality of different RNA-binding guide RNAs comprises between about 12 and about 16 different RNA-binding guide RNAs. In some embodiments, the plurality of different RNA-binding guide RNAs comprises about 14 different RNA-binding guide RNAs.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the hypothetical life cycle of SARS-CoV-2 and the approach for using CRISPR-Cas13 for preventing viral gene expression and degrading viral genome. FIG. 1A illustrates a hypothetical life cycle of SARS-CoV-2 based on information about other coronavirus life cycles. SARS-CoV-2 virions bind to the ACE2 receptor on the surface of cells via interactions with the Spike protein. Upon viral release, the positive strand RNA genome serves as a template to make negative strand genomic and subgenomic templates, which are used to produce more copies of the positive strand viral genome and viral mRNAs.

FIG. 1B illustrates how Cas13d can inhibit viral function and replication by directly targeting and cleaving all viral positive-sense RNA.

FIG. 2 illustrates bioinformatic analysis of Cas13d target sites for SARS-CoV-2 and construction of a prophylactic antiviral CRISPR in human cells (PAC-MAN) system. FIG. 2A depicts alignment of sequences from 47 patient-derived SARS-CoV-2 strains along with SARS-CoV and MERS-CoV. Top panel: predicted abundance of crRNAs that are able to target SARS-CoV-2 genomes and SARS or MERS; Middle panel: annotation of genes in the SARS-CoV-2 genomes, along with conserved regions chosen to be synthesized into the SARS-CoV-2 reporters (in light gray and dark gray). These regions indicate the synthesized SARS-CoV-2 fragments; Bottom panel: the percent of conservation between aligned viral genomes. See SEQ ID NOs: 12001-12040: 12101-12140; or 12201-12240 for the designed crRNA sequences and Table 2 (SEQ ID NOs: 12251 and 12252) for synthesized SARS-CoV-2 fragments. FIG. 2B depicts schematic of the two reporters (SARS-CoV-2-F1/F2) created with synthesized viral sequences. SARS-CoV-2-F1 contains GFP fused to a portion of RdRP (RdRP-F1) and SARS-CoV-2-F2 contains GFP fused to portions of both RdRP (RDRP-F2) and N. FIG. 2C shows schematics for the constructs used to express Cas13d or its crRNAs. FIG. 2D shows the workflow used to challenge Cas13d A549 lung epithelial cells with SARS-CoV-2 reporters.

FIG. 3 illustrates how Cas13d PAC-MAN can effectively repress SARS-CoV-2 reporters. FIG. 3A, left panel: schematics of pools of crRNAs targeting the SARS-CoV-2-F1 reporter for transfection; middle penal: GFP expression as measured by flow cytometry; and right panel: mRNA abundance as measured by qRT-PCR. P values for each group are shown in Table 3. FIG. 3B, left panel: schematics of pools of crRNAs targeting the SARS-CoV-2-F2 reporter for transfection; middle panel: GFP expression as measured by flow cytometry; and right panel: mRNA abundance as measured by qRT-PCR. P values for each group are shown in Table 3. FIG. 3C illustrates GFP expression as measured by flow cytometry when SARS-CoV-2-F1 (left) or SARS-CoV-2-F2 (right) was delivered via lentiviral transduction. P values are labeled for each group; MOI=0.5. FIG. 3D illustrates RNA abundance measured by qRT-PCR when SARS-CoV-2-F1 (left) or SARS-CoV-F2 (right) is delivered via lentiviral transduction. P values are labeled for each group; MOI=0.5. FIG. 3E, top panel: schematic of pools of crRNAs tiling across SARS-CoV-2-F1 and SARS-CoV-2-F2 reporters; and bottom panel: GFP expression levels as measured by flow cytometry. Dark gray, SARS-CoV-2-F1 reporter; light gray, SARS-CoV-2-F2 reporter. NT, non-targeting crRNA controls.

FIG. 4 illustrates the minimum numbers of crRNA necessary to target certain percentages of all known coronavirus genomes. FIG. 4A illustrates a phylogenetic tree of all sequenced coronaviruses, organized by genus. The inner ring depicts coverage by each of the top 6 PAC-MAN-T6 pan-coronavirus crRNAs. The outer ring shows current species of coronaviruses that are infectious to humans, including SARS-COV-2 (444). FIG. 4B illustrates a histogram showing the minimum number of predicted crRNAs it would take to cover a maximum number of coronavirus genomes.

FIG. 5 illustrates a flowchart to design crRNA by analyzing all possible crRNAs that target regions conserved between reported SARS-CoV-2. SARS-CoV, and MERS-CoV.

FIG. 6 illustrates flow cytometry plots demonstrating SARS-CoV-2-19 F1 and F2 transfection and transduction. FIG. 6A illustrates examination of SARS-CoV-2-19 reporter levels in A549 cells 48 hours after transfection or transduction. While transfection led to a much lower percentage of GFP positive cells compared to transduction, transfection also led to much higher levels of GFP expression, giving a better dynamic range of repression. FIG. 6B shows representative flow histograms of GFP reporter levels after SARS-CoV-2 reporter challenge. Cells shown were gated for GFP+ cells only.

FIG. 7 illustrates Flow cytometry plots demonstrating tiled crRNAs with a strong repression of the SARS-CoV-2 F1 and F2 reporters. FIG. 7A shows flow cytometry histograms of GFP expression after SARS-CoV-2-F1 (left) and SARS-CoV-2-F2 (right) reporter challenge. FIG. 7B shows analysis of different expression levels of Cas13d (marker: mCherry) and crRNA pools (marker: BFP) and the resulting effects on SARS-CoV-2-F1/F2 reporter inhibition. Percent reduction of GFP expression is labeled on the top.

FIG. 8 illustrates a bioinformatic pipeline to predict minimum sets of pan-coronavirus crRNA designs. The bioinformatic pipeline analyzed and predicted pan-coronavirus targeting crRNAs. The number of crRNAs at each step of the workflow is denoted in parentheses.

FIGS. 9A-9D illustrate the crRNA-N18f can target all 202 sequenced SARS-CoV-2 genomes, including S and L strains. Different strains of SARS-CoV-2 are shown in labels on the right: S, L, or unknown (U). * indicates the SARS-CoV-2 sequence that carries a single mutation in the crRNA-N18f targeting region. FIGS. 9A-9D disclose

″UGAACCAAGACGCAGUAUUAUU″ as SEQ ID NO: 5590,
″AGGTTTACCCAATAATACTGCGTCTTGGTTCACCGCTCTCAC″ as SEQ ID NO: 13737,
″GGGATTACCCAATAATACTGCGTCTTGGTTCACCGCTCTCAC″ as SEQ ID NO: 13738,
and
″GGGATTACCCAATAGTACTGCGTCTTGGTTCACCGCTCTCAC″ as SEQ ID NO: 13739.

FIG. 10 illustrates crRNAs (guide RNA) design targeting the human 229E coronavirus genome.

FIG. 11 illustrates that the 40 crRNAs (SEQ ID NO: 13607-SEQ ID NO: 13646) targeting 229E were tested in MRC-5 cells. The crRNAs which helped cell survive better than control were selected. The viral titer in the supernatant were determined by RT-qPCR. One or more guides based on one or more crRNAs provided herein inhibited the virus replication to >60%, e.g., the best one N20 reached to 95%.

FIG. 12 illustrates repeat testing of crRNAs targeting 229E. FIG. 12A illustrates crRNAs from the screen assay for repeat testing. While crR8 (SEQ ID NO: 13614), crR19 (SEQ ID NO: 13625), crN1 (SEQ ID NO: 13627), and crN2 (SEQ ID NO: 13628) inhibited about 80% of virus replication, crN4 (SEQ ID NO: 13630), crN9 (SEQ ID NO: 13635), and crN20 (SEQ ID NO: 13646) reached to more than 90%. NLS CasRx were compared with NES version using the crN1 guide. The NLS version (92.5% inhibition) worked better than the NLS and NES mixture (81%). The NES version worked the worst (10%). The result from cellular RNA was in consistence with the supernatant. PAC-MAN may decrease the viral RNA in cellular and the released virus in supernatant. FIG. 12B illustrates repeat testing with cellular RNA. FIG. 12C illustrates repeat testing with cellular RNA and with ActB as inner control.

FIG. 13 illustrates testing of CasRx with no tags or tagged with nuclear localization signal (NLS), nuclear export signal (NES), or endoplasmic reticulum (ER) signal. Since the NLS Cas13d worked better than NES, Cas13d without a tag or was fused with ER location signal. From the result, Cas13d-NLS still worked the best, with a 83% inhibition effect, while Cas13d with no tag had 56% inhibition effect, Cas13d-ER had no effect, and Cas13d-NES had only 16% inhibition effect.

FIG. 14 illustrates how NLS-version of the PAC-MAN worked better than NES-version of the PAC-MAN. MRC5 cells were treated with NLS- or NES-version of PAC-MAN followed by infection of 0.1 MOI 229E. Then cells were stained with 229E RNA FISH N-probe and imaged by confocal. FIG. 14A. RNA FISH probe set-1: targeting N gene (detecting all viral RNA). Same contrast adjustment was used for “Normal contrast” or “High contrast” lane of each group; thus, the intensity was comparable between each group. The signal of 229E FISH in N20 group was significantly decreased compared with non-target guide (NT6) control group, supporting the high efficiency of PAC-MAN. FIG. 14B. The cell without detectable CasRx in cytosol (pointed by white arrow) had intensive signals of 229E FISH, while the cell with CasRx in cytosol (pointed by grey arrow) did not have detectable signals of 229E FISH, indicating that the cytosolic CasRx may be important for the high efficiency of PAC-MAN. FIG. 14C. The signals of 229E FISH were much more intensive in NES-CasRx group compared with that in NLS-CasRx group, though the signals of CasRx in cytosol of the two groups did not have significant difference as shown by the cells pointed by the white or grey arrows. The high efficiency of the NLS-version PAC-MAN compared with the NES-version PAC-MAN was due to the low efficient transport of gRNA to the cytosol and the disability of gRNA in cytosol.

FIG. 15 illustrates another representative image of cells with/without cytosolic CasRx. High expression of Cas13d (CasRx) may leak some Cas13d to cytosol, these Cas13d-crRNA may repress virus replication. The merge picture showed that the cell with Cas13d cytoplasm expression delete the virus. Some cells may not delete virus, because of lack of Cas13d cytoplasm expression.

FIG. 16 illustrates analysis of captured images of FIG. 14 and FIG. 15. Referring to FIG. 16A, for cells expressing NLS-CasRx and gRNA in N20 (SEQ ID NO: 13646) and NT6 groups, the number of cells with/without 229E FISH signal was counted. Then, the ratio of cells without 229E FISH signal was calculated. The number of cells with or without 229E viral RNAs (top) and the ratio of cells without 229E viral RNAs (bottom) in MRC5 cells expressed NLS-dCasRx-mCherry and gRNA-N20 or gRNA-NT6. FIG. 16B. For cells with or without NLS-CasRx expressed in cytosol in N20 group, the number of cells with/without 229E FISH signal was counted. Then, the ratio of cells without 229E FISH signal was calculated. The expression level of CasRx in most cells with NLS-CasRx in cytosol was higher than that of cells without NLS-CasRx in cytosol. The number of cells with or without 229E viral RNAs (top) and the ratio of cells without 229E viral RNAs (bottom) in gRNA-N20-transduced MRC5 cells with NLS-dCasRx-mCherry expressed in both cytoplasm and nucleus or only in nucleus. The MRC5 cells also expressed ER-GFP and were infected with 0.1 MOI 229E.

FIG. 17 illustrates that the lentivirus transduced gRNA stayed in nucleus, thus NLS-Cas13d may enter into the nucleus to complex with the gRNA and leached out of nucleus. In contrast, NES-Cas13d may not enter the nucleus, thus may not form a complex with the gRNA in the nucleus to the same extent, thus not as effective in reducing coronavirus replication. The MRC-5 cells in “Lentivirus-Guide” were co-transduced with NES- or NLS-CasRx and guide. 6 h post transfection, the cells were infected with 229E at an MOI of 0.01. The virus titer in media was determined by RT-qPCR at 24 and 48 hpi.

FIG. 18 illustrates how PAC-MAN helped cell surviving 229E infection. The MRC-5 cells were co-transduced with NLS-CasRx and guide, then infected with 229E at an MOI of 0.01 at 2 days after transduction. The culture media was added with 100-250 nM of incucyte cytotox green dye for real-time quantification of cell death. The cells were imaged for 3 days using IncuCyte live-cell imaging system.

FIG. 19 illustrates repeat testing PAC-MAN with mock control. FIG. 19A illustrates 229E targeting guide N20 protected the cells from death during a 5 day infection, while most cells transduced with a non-targeting guide (NT6) or mock control were dead. FIG. 19B illustrates integrated intensity of the cells transuded with NT6, N20, or mock control.

FIG. 20 illustrates testing with PAC-MAN with 229E fused with GFP (229E-GFP). The 229E-GFP virus was used to validate the effect of PAC-MAN. The virus may infect and replicate in the cells transduced with non-targeting guide with many GFP positive cells showed up, while not in the cells transduced with 229E targeting guides.

FIG. 21 illustrates testing of PAC-MAN against 229E infection at different multiplicity of infections (MOIs). The 229E targeting guide N20 protected the cells from death, while most cells transduced with a non-targeting guide NT6 were dead. FIG. 21A illustrates that the PAC-MAN inhibited virus replication and protected cells from death even when infected at an MOI as high as 1.0, 100-fold higher than the frequently used 0.01 in the in vitro assays. FIG. 21B illustrates a lentiviral dose test.

FIG. 22 illustrates PAC-MAN dose effect. In consistent with the dose effect assay in FIG. 21, FIG. 22 illustrates that the higher dose of PAC-MAN components were transduced, the higher number of cells survived the 229E infection and the longer the cells kept alive.

FIG. 23 illustrates synergistic effect of treating the infected cells with PAC-MAN and IFN alpha (IFNα) 4b and Camostat mesylate. FIG. 23A illustrates the cytotoxicity of the IFNα 4b and Camostat mesylate and their effect on inhibition of 229E virus. These two drug showed no obvious cytotoxicity at the tested concentrations. The EC50 of IFNα 4b is 1 U/ml. The EC50 of Camostat mesylate was about 500 μM. When combining these two drugs with PAC-MAN, an enhance effect was observed as the combination further decreased virus replication. FIG. 23B illustrates the synergistic effect at 24 hour post-infection (hpi). FIG. 23C illustrates IFNα showing redundant effect with PAC-MAN and Camostat mesylate showing synergistic effects with PAC-MAN.

FIG. 24 illustrates RNAseq for determining the off-target effect of PAC-MAN targeting 229E. The top graphs are replicates of every sample. The graphs show that the replicates are consistent. The bottom graphs show Cas13d+crRNA compared to Cas13d only sample, which doesn't show any off-target effect. The axes plot FPKM (Fragments Per Kilobase Per Million Reads) for each gene. FPKM was calculated by dividing the number of reads for each gene by the gene's length in kilobases and by the total number of reads in millions.

FIG. 25 illustrates screening of crRNAs targeting SARS-CoV-2. FIG. 25A illustrates supernatant test showing that many crRNAs may decrease viral RNA load. Cas13d expressing VeroE6 cells were transduced with different crRNAs targeting SARS-CoV-2. After two days, cells were infected with SARS-CoV-2 (MOI=0.01). At 48 hpi, qPCR was performed for RdRP copy number of the supernatant. Lines indicate means, error bars indicate s.d. (n=3 independent biological replicates). FIG. 25B illustrates supernatant test at 24 hpi. Cas13d expressing clonal VeroE6 cells were transduced with different crRNAs or crRNA combinations which target SARS-CoV-2. After two days, cells were infected with SARS-CoV-2 (MOI=0.01). (B) At 24 hpi, (D) At 48 hpi, (E) At 72 hpi, qPCR was performed for RdRP copy number of the supernatant. Lines indicate means; error bars indicate s.d. The data represent three independent biological experiments performed in technical replicates (n=9). FIG. 25C illustrates cell sample test at 24 hpi. Cas13d expressing clonal VeroE6 cells were transduced with different crRNAs or crRNA combinations which target SARS-CoV-2. After two days, cells were infected with SARS-CoV-2 (MOI=0.01). At 24 hpi, qPCR was performed for intracellular RdRP copy number of the cell samples. Copy numbers of each condition were normalized against NT6 control condition. Lines indicate means; error bars indicate s.d. The data represent three independent biological experiments performed in technical replicates (n=9). FIG. 25D illustrates supernatant test at 48 hpi. FIG. 25D illustrates cell sample test at 48 hpi.

FIG. 26 illustrates screening of crRNAs targeting SARS-CoV-2 at 72 hpi. FIG. 26A illustrates supernatant test showing that crRNAs targeting SARS-CoV-2 may dramatically decrease viral RNA load. FIG. 26B and FIG. 26C illustrate summary of repeat testing of protecting cells from SARS-CoV-2 infection at 72 hpi. At 24 hpi, 48 hpi, and 72 hpi, qPCR was performed for RdRP copy number of the supernatant. Lines indicate means; error bars indicate s.d. The data represented three independent biological experiments performed in technical replicates (n=9).

FIG. 27A illustrates comparison of gene expression in A549 cells transfected with Cas13d and A549 cells transfected Cas13d plus crRNAs targeting SARS-CoV-2. The axes plot FPKM (Fragments Per Kilobase Per Million Reads) for each gene. FPKM was calculated by dividing the number of reads for each gene by the gene's length in kilobases and by the total number of reads in millions.

FIG. 27B illustrates how the PAC-MAN with designed crRNAs efficiently knocked down endogenous genes: alanine aminopeptidase (APN, SEQ ID NOs: 13601-13064), transmembrane protease, serine 2 (TMPRSS2, SEQ ID NO: 13605), methylenetetrahydrofolate dehydrogenase 1 (MTHFD1, SEQ ID NO: 13606) and also helped blocking virus replication or propagation. A549 cells were transduced with Cas13d and crRNAs targeting to endogenous genes: APN (left), TMPRSS2 (middle), and MTHFD1 (right). After 3 days, qPCR was performed for targeting gene expression. Relative gene mRNA expression was normalized against wild type cell condition. Lines indicate means; error bars indicate s.d. The data represent three independent biological experiments performed in technical replicates (n=9).

FIG. 28 illustrates inhibition of IAV infection using Cas13d PAC-MAN. FIG. 28A is the workflow used to challenge Cas13d A549 lung epithelial cells with PR8 mNeon IAV. FIG. 28B shows the screening of pools of 6 crRNAs targeting each of the eight IAV genome segments. The percentage of mNeon+ cells in each of the eight crRNA conditions compared to a pool of non-targeting control (NT) crRNAs at an MOI of 2.5 (left) or 5 (right). FIG. 28C shows microscopy quantification of the percentage of mNeon+ cells per field of view (FOV) at an MOI of 2.5 (p=1×10⁻⁵, left) and 5 (p=5×10⁻⁹, right). Each dot represents the percentage for a single microscopy FOV. N=48 FOV. FIG. 28D shows flow cytometry evaluation of the percentage of mNeon+ cells at an MOI of 2.5 (p=0.039, left) and 5 (p=0.0058, right). N=6.

FIG. 29A illustrates screening of pools of 6 crRNAs targeting each of the eight IAV genome segments. Each panel shows the percentage of quantified mNeon+ cells under two infection conditions (MOI=2.5 or 5). Each dot represents the percentage for a single microscopy field of view (FOV). Light gray, non-targeting (NT) crRNAs; dark gray, targeting crRNAs.

FIG. 29B illustrates testing of pools of 6 crRNAs targeting IAV S4 and S6 using a lower MOI of 0.5. Each dot represents the percentage for a single microscopy field of view (FOV). Light gray, non-targeting (NT) crRNAs; dark gray, targeting crRNAs.

FIG. 30 illustrates analysis of PAC-MAN for targeting broad-spectrum RNA viruses.

FIG. 30A. An integrated pipeline for Cas13 crRNA analysis for virus-targeting coverage, efficiency, and specificity. FIG. 30B. Targeting pandemic/epidemic-causing pathogenic RNA virus species using PAC-MAN. FIG. 30C. Schematic of 10 RNA virus families that target different organs and systems in the human host. FIG. 30D. A diagram showing the minimal number of crRNAs needed to target all sequenced human-infectious strains in each of 10 RNA virus families and HIV and IAV. FIG. 30E. A diagram showing the minimal number of crRNAs needed to target 90% of sequenced human-infectious strains in each of 10 RNA virus families and HIV and IAV. FIG. 30F. A cumulative curve showing the predicted minimum number of crRNAs to target all human-infectious strains in 10 RNA virus families in FIG. 30C. FIG. 30G. Coverage of pathogenic RNA virus using the 14 crRNA minipool (SEQ ID NOs: 13651-13664). FIG. 30H. A cumulative curve showing the predicted minimum number of crRNAs to target livestock-infectious strains in 10 RNA virus families in FIG. 30C. Bovine, ovine, swine, feline, and canine viruses are included in the analysis.

FIG. 31 illustrates determinants of required crRNAs targeting broad-spectrum RNA virus families. FIG. 31A. Correlation between the number of strains in the RNA virus family and the number of crRNAs needed for targeting all human-infectious strains in that family. FIG. 31B. Correlation between the number of strains in the RNA virus family and the number of crRNAs needed for targeting all human- and animal-infectious strains in that family. FIG. 31C. A diagram showing the minimal number of crRNAs needed to target all sequenced human-infectious strains in each of 6 RNA virus families with segmented genomes. FIG. 31D. A diagram showing the minimal number of crRNAs needed to target 90% of sequenced human-infectious strains in each of 6 RNA virus families with segmented genomes. FIG. 31E. Correlation between the number of strains in the RNA virus family and the number of crRNAs needed for targeting all human viruses in 6 segment RNA virus families.

FIG. 32 illustrates COMBAT (COMputer-aided Broad-spectrum AnTiviral) as an online resource of using CRISPR-Cas13d for broad-spectrum antiviral targeting. FIG. 32A-C. An exemplary diagram showing the crRNAs designed to target SARS-CoV-2 and its alignment to the genes of SARS-CoV-2. These crRNAs are used for testing in FIG. 33. The intensity of shading for each crRNA represent its predicted crRNA score. Referring to FIG. 32D, an exemplary diagram showing the analysis of family-covering crRNAs targeting the Filoviridae family including Ebola virus.

FIG. 33 illustrates validation of predicted crRNAs for targeting SARS-CoV-2 sequences. FIG. 33A. Repression on SARS-CoV-2 GFP reporter using the first batch of crRNAs measured by flow cytometry. FIG. 33B. Correlation between predicted efficiency score and SARS-CoV-2 GFP repression efficiency for the first batch of crRNAs. FIG. 33C. Repression on SARS-CoV-2 reporter RNA abundance using the first batch of crRNAs measured by qPCR. FIG. 33D. Correlation between predicted efficiency score and RNA abundance repression efficiency for the first batch of crRNAs. FIG. 33E. Comparison of repression on SARS-CoV-2 GFP reporter (red) and predicted efficiency scores (pink) using the second batch of crRNAs. FIG. 33F. Correlation between predicted efficiency score and SARS-CoV-2 GFP repression efficiency for the second batch of crRNAs. FIG. 33G. Correlation between predicted efficiency score and SARS-CoV-2 GFP repression efficiency for combined first and second batches. FIG. 33H-I. Repression effects of the optimized minipool of crRNAs (crRNA-MP5) on SARS-CoV-2 GFP reporter (FIG. 33H) and RNA abundance (FIG. 33I). Referring to FIG. 33J, bar graphs show the percentage of genomes of SARS-CoV-2 isolates targeted by individual or multiple crRNAs in the optimized crRNA minipool.

FIG. 34 illustrates analysis of crRNAs targeting broad-spectrum RNA viruses relating to FIG. 30. Referring to FIG. 34A, coverage of each of 10 RNA virus families using a minipool of 14 crRNAs (SEQ ID NOs: 13651-13664). FIG. 34B. Cumulative curves showing the predicted minimum number of crRNAs to target livestock-infectious strains in 10 RNA virus families in.

FIG. 35 illustrates schematics of experimental systems and workflow used to validate crRNAs related to FIG. 33. Referring to FIG. 35A, the SARS-CoV-2 reporters used in this study. FIG. 35B. PAC-MAN expression system used in this study. FIG. 35C. Workflow for quantifying all individual or combinatorial crRNAs for targeting SARS-CoV-2 sequences.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Any systems, methods, software, systems, systems, and platforms described herein are modular and not limited to sequential steps. Accordingly, terms such as “first” and “second” do not necessarily imply priority, order of importance, or order of acts.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10%-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10%-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “patient” or “subject” are used interchangeably herein, and encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse.

As used herein, a “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

As used herein, the term “influenza virus” generally refers to an RNA virus that is a member of the Orthomyxoviruses family. In some embodiments, the influenza virus is selected from the genera consisting of Influenza virus A, Influenza virus B, Influenza virus C and Influenza virus D. In further embodiments, the influenza A virus is of the subtype H1N1, H2N2, H3N2, H5N1, H7N7, H1N2, H9N2, H7N2, H7N3, H10N7, H7N9, or H6N1. In further embodiments, the influenza B virus of the B/Yamagata/16/88-like lineage or the B/Victoria/2/87-like lineage.” In some embodiments, the influenza can be any strain of the influenza virus or any serotypes within a stain of influenza virus. In some cases, the influenza virus can comprise any combination viral surface glycoproteins haemagglutinin (H or HA) and neuraminidase (N or NA).

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddiTP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif: FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP. BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP. Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodanine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), antisense oligonucleotide (ODN), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The terms “transfection” or “transfected” generally refers to introduction of a nucleic acid into a cell by non-viral or viral-based methods. The nucleic acid molecules can be gene sequences encoding complete proteins or functional portions thereof.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which can be located upstream or downstream of the coding sequence. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” can encompass mRNA, cDNA and genomic forms of a gene.

The term “mutation,” as used herein, can refer to a substitution of a residue within a sequence, e.g., a nucleic acid or amino acid sequence, with another residue, or a deletion or insertion of one or more residues within a sequence. One or more mutations can be described by identifying the original residue followed by the position of the residue within the sequence and by the identity of the newly substituted residue. Mutation can be a change or alteration in a sequence (e.g., nucleic acid sequence, genomic sequence, genetic sequence such as DNA. RNA, or protein sequence) relative to a reference sequence. The reference sequence can be a wild-type sequence, a sequence of a healthy or normal cell, or a sequence that is not associated with a disease or a disorder. A reference sequence can be a sequence not associated with a cancer. Non-limiting examples of mutations include point mutations, substitution of one or more nucleotides, deletion of one or more nucleotides, insertion of one or more nucleotides, fusion of one or more nucleotides, frame shift mutation, aberration, alternative splicing, abnormal methylation, missense mutation, conservative mutation, non-conservative mutation, nonsense mutation, splice variant, alternative splice variant, transition, transversion, de novo mutation, deleterious mutation, disease-causing mutation, epimutation, founder mutation, germline mutation, somatic mutation, predisposing mutation, splice-site mutation, or susceptibility gene mutation. The mutation can be a pathogenic variant or mutation that increases an individual's susceptibility or predisposition to a certain disease or disorder. The mutation can be a driver mutation (e.g., a mutation that can confer a fitness advantage to cells in their microenvironment, thereby driving the cell lineage to cancer). The driver mutation can be a lost function mutation. The mutation can be a lost function mutation. The mutation can be a passenger mutation (e.g., a mutation that occurs in a genome with the driver mutation and can be associated with clonal expansion). As used herein, the term “gene” can refer to a combination of polynucleotide elements, that when operatively linked in either a native or recombinant manner, provide some product or function.

As used herein, the terms “polypeptide,” “peptide” and “protein” can be used interchangeably herein in reference to a polymer of amino acid residues. A protein can refer to a full-length polypeptide as translated from a coding open reading frame, or as processed to its mature form, while a polypeptide or peptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the terms “fragment,” or equivalent terms can refer to a portion of a protein that has less than the full length of the protein and optionally maintains the function of the protein. Further, when the portion of the protein is blasted against the protein, the portion of the protein sequence can align, for example, at least with 80% identity to a part of the protein sequence.

The term “target polynucleotide”, “target viral genome”, or “target viral gene” as used herein can refer to a nucleic acid or polynucleotide which is targeted by the heterologous RNA polynucleotide and the gene regulating moiety of the present disclosure. A target polynucleotide can be DNA (e.g., endogenous or exogenous), for example, a DNA that can serve as a template to generate mRNA transcripts and/or the various regulatory regions which regulate transcription of an mRNA from a DNA template. A target polynucleotide can be a portion of a larger polynucleotide, for example a chromosome or a region of a chromosome. A target polynucleotide can be RNA. RNA can be, for example, mRNA which can serve as template encoding for a protein. A target polynucleotide comprising RNA can include or be within the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression of a gene product. Target polynucleotide can refer to a nucleic acid sequence on a single strand of a target nucleic acid. The target polynucleotide can be a portion of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by a gene regulating moiety, can result in altered gene expression (e.g., increased transcription or translation of a mutated gene) and/or activity. A target polynucleotide can comprise a nucleic acid sequence that cannot be related to any other sequence in a nucleic acid sample by a single nucleotide substitution. A target polynucleotide can comprise or can be a portion of a gene sequence or a regulatory element thereof. A target polynucleotide can comprise or can be a portion of an exon sequence, an intron sequence, an exon-intron junction, splice acceptor-splice donor site, a start codon sequence, a stop codon sequence, a promoter site, an alternative promoter site, 5′ regulatory element, enhancer, 5′ UTR region, 3′ UTR region, poly adenylation site, or binding site of a polymerase, nuclease, gyrase, topoisomerase, methylase or methyl transferase, transcription factors, enhancer, or zinc finger. A target polynucleotide can comprise or be a portion of a splice variant or an alternative splice variant. A target polynucleotide can be present only in a cell to be targeted (e.g., a cancer cell, a diseased cell, cell infected with a microbe such as a virus or bacteria) and can be absent from normal or healthy cells. A target polynucleotide can comprise or be a portion of a microorganism or a microbe, such as a virus or a bacteria. A target polynucleotide can comprise or be a portion of a variant polynucleotide, for example, splice-site variant, point variant, pathogenic variant, unclassified variant, copy number variant, de novo variant, epigenetic variant, founder variant, frameshift variant, germline variant, somatic variant, missense variant, nonsense variant, or a pathogenic variant. A target polynucleotide can comprise or be a portion of an alternative splice variant resulting from a driver mutation.

The term “non-coding region” as used herein generally refers to a polynucleotide sequence (e.g., DNA sequence, RNA sequence) that is ultimately not translated into an amino acid sequence (e.g., a polypeptide). The non-coding region may not encode a polypeptide (e.g., a protein). For example, the non-coding region may be an intergenic region or an intronic region of a genetic sequence. In some cases, the target viral genome segment as disclosed herein may comprise at least one non-coding region (e.g., at least 1, 2, 3, 4, 5, or more non-coding regions) of a viral genome segment (e.g., influenza viral genome segment) that encodes one or more members selected from the group consisting of neuraminidase (NA), hemagglutinin (HA), and hemagglutinin-esterase-fusion (HEF). In some cases, the target viral genome segment as disclosed herein may comprise at least one non-coding region (e.g., at least 1, 2, 3, 4, 5, or more non-coding regions) of genome segments IV or VI, of influenza A or B. In some cases, the target viral genome segment as disclosed herein may comprise at least one non-coding region (e.g., at least 1, 2, 3, 4, 5, or more non-coding regions) of genome segments IV, of influenza C or D. In some cases, the target viral genome segment as disclosed herein may comprise at least one non-coding region (e.g., at least 1, 2, 3, 4, 5, or more non-coding regions) of a viral genome segment (e.g., influenza viral genome segment) that does not encode one or more members selected from the group consisting of polymerase basic protein 1 (PB1), polymerase basic protein 2 (PB2), polymerase acidic protein (PA), nucleocapsid protein (NP), matrix protein (M), and a non-structural protein (NS).

The terms “complement,” “complements,” “complementary,” and “complementarity.” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “mismatch” generally refers to lack of complementarity between two nucleotides when aligned. Complementary bases in DNA are A-T and G-C. Complementary bases in RNA are A-U and G-C. Thus a mismatch occurs when two oligonucleotide sequences are aligned and at one or more nucleotide positions that an A is not paired with T or a G is not paired with C in DNA or an A is not paired with U or a G is not paired with C in RNA.

As used herein, the term “in vivo” can be used to describe an event that takes place in a subject's body.

As used herein, the term “ex vivo” can be used to describe an event that takes place outside of a subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

“Treating” or “treatment” can refer to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease cannot have been made.

The term “effective amount” and “therapeutically effective amount,” as used interchangeably herein, generally refer to the quantity of a system, for example a system comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a system that is sufficient to delay the manifestation, arrest the progression, relieve or alleviate at least one symptom of a disorder treated by the methods of the present disclosure.

The term “viral infection” generally refers to any stage of an infection by a virus in a host (e.g., a cell, an animal, a human, etc.), including, but not limited to, attachment of the virus to a cell, penetration of the virus to the cell (e.g., fusion of the virus to the cell membrane, uncoating of the virus), uncoating of capsid of the virus, replication (e.g., transcription or reverse transcription of the viral genome, translation of the viral genome or a derivative thereof, viral particle assembly), and lysis (e.g., release of new viral particles from the cell). The term viral infection can also refer to any period of viral infection, including, but not limited to incubation phase, latent phase, dormant phase, acute phase, and development and maintenance of immunity towards a virus.

A change (e.g., reduction) in viral infection in a cell or an organism cam be determined by one or more assays including, but not limited to, microscopy (e.g., transmission electron microscopy (TEM), light microscopy, fluorescence microscopy using one or more antibodies against a viral antigen), detection of viral genetic material or a derivative thereof (e.g., via polymerase chain reaction (PCR), quantitative PCR, loop-mediated amplification (LAMP), next generation sequencing (NGS), western blotting, flow cytometry), quantitative virus assays (e.g., plaque assay, focus forming assay (FFA), 50% Tissue Culture Infective Dose (TCDi50) assay, hemagglutination assay and hemagglutination inhibition Assay (HIA), enzyme-linked immunosorbent assay (ELISA), homogeneous assay), etc.

The term “pharmaceutically acceptable carrier,” “pharmaceutically acceptable excipient,” “physiologically acceptable carrier,” or “physiologically acceptable excipient” refers to a pharmaceutically-acceptable material, system, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio.

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

### 1. Overview

Aspects of the present disclosure provides a system for reducing viral infection in a cell. In some embodiments, the system can comprise one or more therapeutic agents (e.g., at least 1, 2, 3, 4, 5, or more different therapeutic agents). In some embodiments, the system can comprise a therapeutic agent comprising (i) a heterologous polypeptide comprising a gene regulating moiety or (ii) a heterologous nucleic acid molecule. In some embodiments, at least the gene regulating moiety or the one or more heterologous nucleic acid molecules can be configured to specifically bind at least a portion of one or more target viral genes of one or more viruses in the cell, to reduce expression or activity of the one or more target viral genes of the one or more viruses in the cell. Alternatively or in addition to, the system can comprise a therapeutic agent comprising an agent that reduces viral infection in the cell by one or more mechanisms other than binding a target viral gene.

In some embodiments, the system as disclosed herein can comprise only one of (i) heterologous polypeptide comprising the gene regulating moiety and (ii) the heterologous nucleic acid molecule. In alternative embodiments, the system can comprise both (i) the heterologous polypeptide comprising the gene regulating moiety and (ii) the heterologous nucleic acid molecule. In some cases, (i) the heterologous polypeptide comprising the gene regulating moiety (e.g., non-CRISPR/Cas endonucleases, such as TALEN, ZFN, etc.) and (ii) the heterologous nucleic acid molecule (e.g., silencing nucleic acid molecules) can function independently of one another. In some cases, (i) the heterologous polypeptide comprising the gene regulating moiety (e.g., a CRISPR/Cas endonuclease or a variant thereof) and (ii) the heterologous nucleic acid molecule (e.g., a guide nucleic acid molecule, such as a small guide RNA) can exhibit functions that dependent on each other, e.g., by forming a complex (e.g., CRISPR/Cas-guide RNA complex).

Additional aspects of the present disclosure provides compositions, kits, or reaction mixtures that comprise or are derived from any one of the systems provided herein. Additional aspects of the present disclosure provides methods for reducing viral infection in a cell by utilizing any one of the systems, compositions, kits, or reaction mixtures provided herein.

In some embodiments, the reducing expression or activity of the one or more target viral genes in the cell contacted with the system described herein is determined by comparing with a corresponding control cell infected with the same strain or strains of virus infecting the cell contacted with the system. In some embodiments, the corresponding control comprises a corresponding control cell. In some embodiments, the corresponding control cell and the cell being treated are infected by the same strain or strains of virus. In some embodiments, the corresponding control cell is a cell comprising a non-functional gene regulating moiety; a cell lacking a heterologous nucleic acid molecule (e.g., no sgRNA); a cell comprising a control heterologous nucleic acid molecule (e.g., a control sgRNA that does not bind a target polynucleotide sequence of interest); or a cell comprising a reduced number of heterologous nucleic acid molecules (e.g., a fewer number of sgRNAs), in cases where a pool of different guide nucleic acid molecules was used to reduce viral infection.

### II. Systems, Compositions, and Methods for Reducing Viral Infection

In an aspect, the present disclosure provides a system for reducing viral infection in a cell. The system can regulate (e.g., reduce) expression or activity of one or more target viral genes in a cell. The system can comprise a heterologous polypeptide comprising a gene regulating moiety, wherein the gene regulating moiety can be configured to bind one or more target viral genes in the cell, to reduce the expression or activity of the one or more target viral genes. Alternatively or in addition to, the system can comprise at least one heterologous nucleic acid molecule (e.g., at least one heterologous RNA polynucleotide) configured to bind the one or more target viral genes in the cell, to reduce the expression or activity of the one or more target viral genes. In some cases, the one or more target viral genes can encode (i) a viral RNA-dependent RNA polymerase (RdRP) or a fragment thereof or (ii) a viral nucleocapsid protein (N-protein) or a fragment thereof. In some examples, the one or more target viral genes can encode (i) the viral RdRp or a fragment thereof and (ii) the viral N-protein or a fragment thereof.

In some embodiments of any one of the systems disclosed herein, a plurality of heterologous nucleic acid molecules (e.g., a plurality of different heterologous RNA molecules) can collectively exhibit specific binding to a threshold percentage (e.g., at least about 50%, 60%, 70%, 80%, 90%, or more) of different types of viruses (e.g., coronaviruses). In some cases, no more than six members of the heterologous RNA polynucleotide (e.g., crRNA or guide RNA) can collectively exhibit specific binding to at least 50% of different types of coronaviruses selected from the group consisting of: alphacoronavirus, betacoronavirus, deltacoronavirus, and gammacoronavirus.

In another aspect, the present disclosure provides a system for reducing viral infection in a cell (e.g., a population of cell. The system can comprise a heterologous polypeptide comprising a gene regulating moiety, wherein the gene regulating moiety can be configured to regulate (e.g., reduce) expression or activity of a target viral genome segment (e.g., one or more target viral genome segments) of a virus in the cell. Alternatively or in addition to, the system can comprise at least one heterologous nucleic acid molecule (e.g., at least one heterologous RNA molecule) configured to regulate (e.g., reduce) expression or activity of the target viral gene segment of the virus in the cell. In some embodiments, the target viral genome segment can comprise at least one non-coding region or a fragment thereof (e.g., at least 1, 2, 3, 4, 5, or more non-coding regions of fragments thereof).

In some embodiments of any one of the systems disclosed herein, the at least one heterologous nucleic acid molecule can comprise a plurality of heterologous nucleic acid molecule (e.g., a plurality of heterologous RNA polynucleotides) configured to bind a plurality of different portions of the target viral genome segment. In some cases, the plurality of nucleic acid molecule (e.g., the plurality of heterologous RNA polynucleotides) can comprises at least two, three, four, five, or six different RNA polynucleotides.

In some embodiments of any one of the systems disclosed herein, the target viral genome segment can be from an influenza virus, and the system can effect reduced transfection of the influenza virus in the cell (e.g., in the population of cells). In some embodiments, the at least one non-coding region is conserved across a plurality of influenza virus types selected from the group consisting of: influenza A, influenza B, influenza C, and influenza D. In some embodiments, the at least one non-coding region is conserved across a plurality of influenza subtypes.

In some embodiments one of the systems disclosed herein, the target viral genome segment can comprise a first non-coding region and a second non-coding region that is different from the first non-coding region. In some embodiments, the first and second non-coding regions can be operatively coupled to a same viral polypeptide. In some cases, a polynucleotide sequence encoding the same viral polypeptide or a fragment thereof can be flanked by the first and second non-coding regions. In some cases, the same viral polypeptide can be selected from the group consisting of: a neuraminidase and a hemagglutinin. In some cases, the same viral polypeptide can be the neuraminidase. In alternative embodiments, the first and second non-coding regions can be operatively coupled to different viral polypeptides. In some cases, the first viral polypeptide can be a neuraminidase and the second viral polypeptide can be selected from the group consisting of: a polymerase, a hemagglutinin, a nucleoprotein, a matrix protein, a non-structural protein, and a nuclear export protein.

In another aspect, the present disclosure provides a system for reducing viral infection in a cell. The system can comprise (1) a plurality of heterologous gene regulating moieties; or (2) a plurality of heterologous nucleic acid molecules. In some cases, the plurality of heterologous gene regulating moieties or the plurality of heterologous nucleic acid molecules can be configured to specifically bind a plurality of virus strains (e.g., a plurality of target viral genes or proteins of the plurality of virus strains, such as RNA virus strains). The term “strain” or “viral strain” can be used interchangeably throughout the present disclosure.

In some embodiments, the system can comprise only the plurality of heterologous gene regulating moieties (e.g., TALEN, ZFN, etc.). In some embodiments, the system can comprise only the plurality of heterologous nucleic acid molecules (e.g., silencing nucleic acids or inhibitory RNAs such as short interfering RNA (siRNA), short-hairpin RNA (shRNA), micro-RNA (miRNA), antisense oligonucleotide (ODN), etc). In some embodiments, the system can comprise both the plurality of heterologous gene regulating moieties and the plurality of heterologous nucleic acid molecules. In some cases, a first number of the plurality of heterologous gene regulating moieties and a second number of the plurality of heterologous nucleic acid molecules can be the same. Alternatively, the first number of the plurality of heterologous gene regulating moieties and the second number of the plurality of heterologous nucleic acid molecules can be different. For example, the first number can be greater than the second number. Alternatively, the first number can be less than the first number.

In some embodiments of any one of the systems disclosed herein, (i) a gene regulating moiety of the plurality of heterologous gene regulating moieties can be configured to form a complex (e.g., non-covalently complex) with (ii) a heterologous nucleic acid molecule of the plurality of plurality of heterologous nucleic acid molecules, and such complex can be configured to bind a virus strain (e.g., bind a target viral gene of the virus strain) of the plurality of virus strains. For example, the complex can be an endonuclease-guide nucleic acid complex, such as a CRISPR-Cas nuclease-guide RNA complex (e.g., Cas13d-sgRNA complex). As such, the plurality of heterologous gene regulating moieties and the plurality of plurality of heterologous nucleic acid molecules can form a plurality of complexes. The plurality of complexes collectively can be configured to specifically bind the plurality of virus strains (e.g., bind a plurality of target viral genes from the plurality of virus strains).

In some embodiments of any one of the systems disclosed herein, the plurality of heterologous gene regulating moieties of the system can comprise no more than 100 members, 95 members, 90 members, 85 members, 80 members, 75 members, 70 members, 65 members, 60 members, 55 members, 50 members, 45 members, 40 members, 35 members, 34 members, 33 members, 32 members, 31 members, 30 members, 29 members, 28 members, 27 members, 26 members, 25 members, 24 members, 23 members, 22 members, 21 members, 20 members, 19 members, 18 members, 17 members, 16 members, 15 members, 14 members, 13 members, 12 members, 11 members, 10 members, 9 members, 8 members, 7 members, 6 members, 5 members, 4 members, 3 members, or 2 members. In some cases, the plurality of heterologous gene regulating moieties can comprise between about 2 members and about 50 members, between about 2 members and about 45 members, between about 2 members and about 40 members, between about 2 members and about 35 members, between about 2 members and about 30 members, between about 5 members and about 30 members, between about 5 members and about 25 members, between about 10 members and about 20 members, between about 10 members and about 18 members, or between about 12 members and about 16 members. For example, the plurality of heterologous gene regulating moieties can comprise no more than about 14 members. The members of the plurality of heterologous gene regulating moieties can be different members (e.g., configured to bind to different target viral gene sequences or viral proteins).

In some embodiments of any one of the systems disclosed herein, a heterologous gene regulating moiety of the plurality of gene regulating moieties can exhibit a specific binding to a target polynucleotide sequence. The target polynucleotide sequence can have at least about 50%, 55%, 60%, 65%, 70%, 75%, 80%, 85%, 90%, 95%, 96%, 97%, 98%, 99%, or 100% sequence identity or complementarity to a sequence selected from SEQ ID NOs: 13651-13664 or SEQ ID NOs: 13671-13684.

In some embodiments of any one of the systems disclosed herein, the plurality of heterologous nucleic acid molecules can comprise no more than 100 members, 95 members, 90 members, 85 members, 80 members, 75 members, 70 members, 65 members, 60 members, 55 members, 50 members, 45 members, 40 members, 35 members, 34 members, 33 members, 32 members, 31 members, 30 members, 29 members, 28 members, 27 members, 26 members, 25 members, 24 members, 23 members, 22 members, 21 members, 20 members, 19 members, 18 members, 17 members, 16 members, 15 members, 14 members, 13 members, 12 members, 11 members, 10 members, 9 members, 8 members, 7 members, 6 members, 5 members, 4 members, 3 members, or 2 members. In some cases, the plurality of heterologous nucleic acid molecules can comprise between about 2 members and about 50 members, between about 2 members and about 45 members, between about 2 members and about 40 members, between about 2 members and about 35 members, between about 2 members and about 30 members, between about 5 members and about 30 members, between about 5 members and about 25 members, between about 10 members and about 20 members, between about 10 members and about 18 members, or between about 12 members and about 16 members. For example, the plurality of heterologous nucleic acid molecules can comprise no more than about 14 members. The members of the plurality of heterologous nucleic acid molecules can be different members (e.g., configured to bind to different target viral gene sequences or viral proteins).

In some embodiments of any one of the systems disclosed herein, a heterologous nucleic acid molecule of the plurality of nucleic acid molecules can exhibit a specific binding to a target polynucleotide sequence. The target polynucleotide sequence can have at least about 50%, 55%, 60%, 65%, 70%, 75%, 80%, 85%, 90%, 95%, 96%, 97%, 98%, 99%, or 100% sequence identity or complementarity to a sequence selected from SEQ ID NOs: 13651-13664 or SEQ ID NOs: 13671-13684.

In some embodiments of any one of the systems disclosed herein, the plurality of strains can comprise at least about 1,000 different strains, 2,000 different strains, 3,000 different strains, 4,000 different strains, 5,000 different strains, 6,000 different strains, 7,000 different strains, 8,000 different strains, 9,000 different strains, 10,000 different strains, 11,000 different strains, 12,000 different strains, 13,000 different strains, 14,000 different strains, 15,000 different strains, 16,000 different strains, 17,000 different strains, 18,000 different strains, 19,000 different strains, 20,000 different strains, 21,000 different strains, 22,000 different strains, 23,000 different strains, 24,000 different strains, 25,000 different strains, 30,000 different strains, 40,000 different strains, 50,000 different strains, or more. The plurality of strains can comprise at most about 50,000 different strains, 45,000 different strains, 40,000 different strains, 35,000 different strains, 30,000 different strains, 25,000 different strains, 24,000 different strains, 23,000 different strains, 22,000 different strains, 21,000 different strains, 20,000 different strains, 19,000 different strains, 18,000 different strains, 17,000 different strains, 16,000 different strains, 15,000 different strains, 14,000 different strains, 13,000 different strains, 12,000 different strains, 11,000 different strains, 10,000 different strains, 9,000 different strains, 8,000 different strains, 7,000 different strains, 6,000 different strains, 5,000 different strains, 4,000 different strains, 3,000 different strains, 2,000 different strains, 1,000 different strains, or less.

In some embodiments of any one of the systems disclosed herein, a plurality of viral genes from the plurality of strains can comprise at least about 1,000 viral genes, 2,000 viral genes, 3,000 viral genes, 4,000 viral genes, 5,000 viral genes, 6,000 viral genes, 7,000 viral genes, 8,000 viral genes, 9,000 viral genes, 10,000 viral genes, 11,000 viral genes, 12,000 viral genes, 13,000 viral genes, 14,000 viral genes, 15,000 viral genes, 16,000 viral genes, 17,000 viral genes, 18,000 viral genes, 19,000 viral genes, 20,000 viral genes, 21,000 viral genes, 22,000 viral genes, 23,000 viral genes, 24,000 viral genes, 25,000 viral genes, 30,000 viral genes, 40,000 viral genes, 50,000 viral genes, or more. The plurality of strains can comprise at most about 50,000 viral genes, 45,000 viral genes, 40,000 viral genes, 35,000 viral genes, 30,000 viral genes, 25,000 viral genes, 24,000 viral genes, 23,000 viral genes, 22,000 viral genes, 21,000 viral genes, 20,000 viral genes, 19,000 viral genes, 18,000 viral genes, 17,000 viral genes, 16,000 viral genes, 15,000 viral genes, 14,000 viral genes, 13,000 viral genes, 12,000 viral genes, 11,000 viral genes, 10,000 viral genes, 9,000 viral genes, 8,000 viral genes, 7,000 viral genes, 6,000 viral genes, 5,000 viral genes, 4,000 viral genes, 3,000 viral genes, 2,000 viral genes, 1,000 viral genes, or less.

In some embodiments of any one of the systems disclosed herein, a plurality of viral genes from the plurality of strains can comprise at least about 1,000 viral polynucleotide sequences, 2,000 viral polynucleotide sequences, 3,000 viral polynucleotide sequences, 4,000 viral polynucleotide sequences, 5,000 viral polynucleotide sequences, 6,000 viral polynucleotide sequences, 7,000 viral polynucleotide sequences, 8,000 viral polynucleotide sequences, 9,000 viral polynucleotide sequences, 10,000 viral polynucleotide sequences, 11,000 viral polynucleotide sequences, 12,000 viral polynucleotide sequences, 13,000 viral polynucleotide sequences, 14,000 viral polynucleotide sequences, 15,000 viral polynucleotide sequences, 16,000 viral polynucleotide sequences, 17,000 viral polynucleotide sequences, 18,000 viral polynucleotide sequences, 19,000 viral polynucleotide sequences, 20,000 viral polynucleotide sequences, 21,000 viral polynucleotide sequences, 22,000 viral polynucleotide sequences, 23,000 viral polynucleotide sequences, 24,000 viral polynucleotide sequences, 25,000 viral polynucleotide sequences, 30,000 viral polynucleotide sequences, 40,000 viral polynucleotide sequences, 50,000 viral polynucleotide sequences, or more. The plurality of strains can comprise at most about 50,000 viral polynucleotide sequences, 45,000 viral polynucleotide sequences, 40,000 viral polynucleotide sequences, 35,000 viral polynucleotide sequences, 30,000 viral polynucleotide sequences, 25,000 viral polynucleotide sequences, 24,000 viral polynucleotide sequences, 23,000 viral polynucleotide sequences, 22,000 viral polynucleotide sequences, 21,000 viral polynucleotide sequences, 20,000 viral polynucleotide sequences, 19,000 viral polynucleotide sequences, 18,000 viral polynucleotide sequences, 17,000 viral polynucleotide sequences, 16,000 viral polynucleotide sequences, 15,000 viral polynucleotide sequences, 14,000 viral polynucleotide sequences, 13,000 viral polynucleotide sequences, 12,000 viral polynucleotide sequences, 11,000 viral polynucleotide sequences, 10,000 viral polynucleotide sequences, 9,000 viral polynucleotide sequences, 8,000 viral polynucleotide sequences, 7,000 viral polynucleotide sequences, 6,000 viral polynucleotide sequences, 5,000 viral polynucleotide sequences, 4,000 viral polynucleotide sequences, 3,000 viral polynucleotide sequences, 2,000 viral polynucleotide sequences, 1,000 viral polynucleotide sequences, or less.

In some embodiments of any one of the systems disclosed herein, the plurality of virus strains can be from a plurality of virus families (e.g., a plurality of different reference viral strains from a plurality of different reference virus families, such as RNA virus families).

In some embodiments of any one of the systems disclosed herein, the plurality of heterologous gene regulating moieties or the plurality of heterologous nucleic acid molecules can be configured to specifically bind at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% of the plurality of strains (e.g., a plurality of viral genes thereof) from the plurality of virus families (e.g., RNA virus families). The plurality of heterologous gene regulating moieties or the plurality of heterologous nucleic acid molecules can be configured to specifically bind between about 50% and about 99% of the plurality of strains from the plurality of virus families, between about 55% and about 99% of the plurality of strains from the plurality of virus families, between about 60% and about 99% of the plurality of strains from the plurality of virus families, between about 60% and about 95% of the plurality of strains from the plurality of virus families, between about 60% and about 95% of the plurality of strains from the plurality of virus families, between about 65% and about 95% of the plurality of strains from the plurality of virus families, between about 60% and about 95% of the plurality of strains from the plurality of virus families, between about 65% and about 95% of the plurality of strains from the plurality of virus families, between about 70% and about 95% of the plurality of strains from the plurality of virus families, between about 75% and about 95% of the plurality of strains from the plurality of virus families, or between about 80% and about 95% of the plurality of strains from the plurality of virus families.

In some embodiments of any one of the systems disclosed herein, the plurality of virus families can comprise a plurality of RNA virus families. In some cases, the plurality of RNA virus families can comprise two or more members (e.g., 2, 3, 4, 5, 6, 7, 8, 9, 10, 11, or 12 members) selected from the group consisting of Coronaviridae, Pneumoviridae, Paramyxoviridae, Caliciviridae, Hepeviridae, Picomaviridae, Filoviridae, Flaviviridae, Rhabdoviridae. Togaviridae, Orthomyxoviridae, and Retroviridae. In some cases, the plurality of RNA virus families can comprise two or more members (e.g., 2, 3, 4, 5, 6, 7, 8, 9, or 10 members) selected from the group consisting of Coronaviridae, Pneumoviridae, Paramyxoviridae, Caliciviridae, Hepeviridae, Picomaviridae, Filoviridae, Flaviviridae, Rhabdoviridae, and Togaviridae.

In some embodiments of any one of the systems disclosed herein, the plurality of virus strains can be from a single virus family of a plurality of virus families (e.g., from a single RNA virus family of a plurality of RNA virus families). In some embodiments, the plurality of heterologous gene regulating moieties or the plurality of heterologous nucleic acid molecules can be configured to specifically bind a plurality of strains from a single RNA virus family of a plurality of RNA virus families. In some embodiments, the plurality of heterologous gene regulating moieties or the plurality of heterologous nucleic acid molecules can be configured to specifically bind at least a threshold portion of a plurality of strains from each RNA virus family of a plurality of RNA virus families.

In some embodiments of any one of the systems disclosed herein, the plurality of strains from the each RNA virus family can comprise at least about 500 different strains, 1,000 different strains, 1,500 different strains, 2,000 different strains, 2,500 different strains, 3,000 different strains, 3,500 different strains, 4,000 different strains, 4,500 different strains, 5,000 different strains, 5,500 different strains, 6,000 different strains, 6,500 different strains, 7,000 different strains, 7,500 different strains, 8,000 different strains, 8,500 different strains, 9,000 different strains, 9,500 different strains, 10,000 different strains, 10,500 different strains, 1,000 different strains, 11,500 different strains, 12,000 different strains, 12,500 different strains, 13,000 different strains, 13,500 different strains, 14,000 different strains, 14,500 different strains, 15,000 different strains, or more. The plurality of strains from the each RNA virus family can comprise at most about 15,000 different strains, 14,500 different strains, 14,000 different strains, 13,500 different strains, 13,000 different strains, 12,500 different strains, 12,000 different strains, 11,500 different strains, 11,000 different strains, 10,500 different strains, 10,000 different strains, 9,500 different strains, 9,000 different strains, 8,500 different strains, 8,000 different strains, 7,500 different strains, 7,000 different strains, 6,500 different strains, 6,000 different strains, 5,500 different strains, 5,000 different strains, 4.500 different strains, 4,000 different strains, 3,500 different strains, 3,000 different strains, 2,500 different strains, 2,000 different strains, 1,500 different strains, 1,000 different strains, 500 different strains, or less.

In some embodiments of any one of the systems disclosed herein, a first number of a first plurality of strains from a first RNA virus family and a second number of a second plurality of strains from a second RNA virus family can be the same. Alternatively, the first number of the first plurality of strains from the first RNA virus family and the second number of the second plurality of strains from the second RNA virus family can be different.

In some embodiments of any one of the systems disclosed herein, the threshold portion of the plurality of strains from the each RNA virus family of the plurality of RNA virus families can be at least about 20%, 25%, 30%, 35%, 40%, 45%, 50%, 55%, 60%, 65%, 70%, 75%, 80%, 85%, 90%, or more. The threshold portion of the plurality of strains from the each RNA virus family of the plurality of RNA virus families can be at most about 90%, 85%, 80%, 75%, 70%, 65%, 60%, 55%, 50%, 45%, 40%, 35%, 30%, 25%, 20%, or less. In some cases, the threshold portion of the plurality of strains from the each RNA virus family of the plurality of RNA virus families can be between about 20% to about 60%, between about 25% to about 60%, between about 30% to about 60%, between about 35% to about 60%, between about 40% to about 60%, between about 45% to about 60%, or between about 50% to about 60%.

In some embodiments, the threshold portion of the plurality of strains from the each RNA virus family of the plurality of RNA virus families can be at least about 55%, 60%, 65%, 70%, 75%, 80%, 85%, 90%, or more. In some cases, the plurality of RNA virus families can comprise two or more members (e.g., 2, 3, 4, 5, 6, 7, or 8 members) selected from the group consisting of Coronaviridae, Pneumoviridae, Caliciviridae, Hepeviridae, Picornaviridae, Filoviridae, Flaviviridae, and Togaviridae.

In some embodiments, the threshold portion of the plurality of strains from the each RNA virus family of the plurality of RNA virus families can be at least about 70%, 75%, 80%, 85%, or more. In some cases, the plurality of RNA virus families can comprise two or more members (e.g., 2, 3, 4, 5, 6, or 7 members) selected from the group consisting of Coronaviridae, Pneumoviridae, Hepeviridae, Picomaviridae, Filoviridae, Flaviviridae, and Togaviridae.

In some embodiments of any one of the systems disclosed herein, the plurality of RNA virus families can be a plurality of human-infectious RNA virus families.

In some embodiments of any one of the systems disclosed herein, the heterologous polypeptide can comprise the gene regulating moiety that is operatively coupled to a nuclear localization signal (NLS) (gene regulating moiety-NLS). The term “NLS” or “nuclear localization domain (NLD)” can be used interchangeably throughout the present disclosure. The NLS can be heterologous to the gene regulating moiety. The gene regulating moiety can be coupled to the NLS via covalent or non-covalent interactions (e.g., via an additional molecule configured to bind the gene regulating moiety in one arm and the NLS in another arm). The NLS can be a polypeptide sequence or a functional variant thereof. The NLS can be capable of traversing a nuclear membrane, thereby entering the nucleus of a cell. Thus, the NLS can be capable of traversing the nuclear membrane with the gene regulating moiety, thereby assisting the gene regulating moiety to enter the nuclease of the cell.

In some embodiments of any one of the systems disclosed herein, the system for reducing viral infection in the cell can comprise both (i) the heterologous polypeptide comprising the gene regulating moiety that is operatively coupled to a nuclear localization signal (NLS) (gene regulating moiety-NLS) and (ii) a heterologous polynucleotide sequence encoding a guide nucleic acid molecule. Upon expression of the guide nucleic acid molecule from the heterologous polynucleotide sequence in the cell, the gene regulating moiety-NLS can form a complex with the guide nucleic acid molecule. The complex can be configured to bind a target viral gene, to reduce the viral infection in the cell. In some cases, the heterologous polypeptide or the heterologous polynucleotide sequence encoding the guide nucleic acid may not be integrated into a genome of the host cell. In some cases, the heterologous polypeptide and the heterologous polynucleotide sequence encoding the guide nucleic acid may not be integrated into a genome of the host cell. Without wishing to be bound by theory, the heterologous polynucleotide sequence may be transcribed to the guide nucleic acid molecule in the nuclease of the cell. For example, the heterologous polynucleotide sequence may be a viral vector (e.g., lentiviral vector) that is ultimately transcribed to the guide nucleic acid molecule in the nucleus of the cell. In another example, the heterologous polynucleotide sequence may not be a guide RNA nucleic acid molecule that is directly transfected into the cell.

In some embodiments of any one of the systems disclosed herein, the complex comprising the gene regulating moiety-NLS and the corresponding guide nucleic acid sequence can specifically bind a target viral gene, and the specific binding can effect reduction of the viral infection in the cell by at least about 5%, 10%, 15%, 20%, 25%, 30%, 35%, 40%, 45%, 50%, 55%, 60%, 65%, 70%, 80%, 90%, or more, as compared to a corresponding control. For example, the corresponding control can comprise a gene regulating moiety that is not operatively coupled to the NLS (e.g., a gene regulating moiety operatively coupled to a nuclear export signal (NES), a gene regulating moiety that is not operatively coupled to NLS or NES). In another example, the corresponding control can be the gene regulating moiety-NLS in absence of the guide nucleic acid sequence or in the presence of a control guide nucleic acid sequence that does not bind the target viral gene.

In some embodiments of any one of the systems disclosed herein, the heterologous polypeptide comprising the gene regulating moiety-NLS can be expressed in the cell (e.g., the host cell) via viral transduction of a transgene encoding at least the gene-regulating moiety-NLS. In some cases, a gene encoding the heterologous polypeptide comprising the gene regulating moiety-NLS may not be transfected into the cell (e.g., via cationic transfection agents).

In some embodiments of any one of the systems disclosed herein, the system can comprise (1) a first therapeutic agent configured to specifically bind a target viral gene and (2) a second therapeutic agent that does not specifically bind a viral gene. The first therapeutic agent can comprise (i) a heterologous polypeptide comprising a gene regulating moiety; or (ii) a heterologous nucleic acid molecule. In some cases, the first therapeutic agent and the second therapeutic agent can be different. In some cases, a combination of the first and second therapeutic agents can reduce viral infection in the cell, as compared to a corresponding control. In some embodiments, the corresponding control comprises a corresponding control cell. In some embodiments, the corresponding control cell and the cell being treated are infected by the same strain or strains of virus. In some embodiments, the corresponding control cell is a cell comprising a non-functional gene regulating moiety; a cell lacking a heterologous nucleic acid molecule (e.g., no sgRNA); a cell comprising a control heterologous nucleic acid molecule (e.g., a control sgRNA that does not bind a target polynucleotide sequence of interest); or a cell comprising a reduced number of heterologous nucleic acid molecules (e.g., a fewer number of sgRNAs), in cases where a pool of different guide nucleic acid molecules was used to reduce viral infection,

In some embodiments of any one of the systems disclosed herein, the corresponding control can comprise a cell lacking the first therapeutic agent or the second therapeutic agent. For example, the control can comprise a cell having the first therapeutic agent but lacking the second therapeutic agent. In another example, the control can comprise a cell lacking the first therapeutic agent but having the second therapeutic agent. In a different example, the control can comprise a cell lacking both the first therapeutic agent and the second therapeutic agent.

In some embodiments of any one of the systems disclosed herein, the combination of the first therapeutic agent and the second therapeutic agent can effect enhanced reduction in the viral infection in the cell by at least about 10%, 15%, 20%, 25%, 30%, 35%, 40%, 45%, 50%, 55%, 60%, 65%, 70%, 75%, 80%, 85%, 90%, 95%, 100%, 150%, 200%, or more as compared to the corresponding control. In some embodiments of any one of the systems disclosed herein, the combination of the first therapeutic agent and the second therapeutic agent can effect enhanced reduction in the viral infection in the cell by at most about 100%, 95%, 90%, 85%, 80%, 75%, 70%, 65%, 60%, 55%, 50%, 45%, 40%, 35%, 30%, 25%, 20%, 15%, 10%, or less as compared to the corresponding control.

In some embodiments of any one of the systems disclosed herein, the combination of the first therapeutic agent and the second therapeutic agent can effect enhanced reduction in the viral infection in the cell by at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold, at least about 1 fold, at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 30-fold, at least about 40-fold, at least about 50-fold, at least about 60-fold, at least about 70-fold, at least about 80-fold, at least about 90-fold, or at least about 100-fold, or more as compared to the corresponding control. In some embodiments of any one of the systems disclosed herein, the combination of the first therapeutic agent and the second therapeutic agent can effect enhanced reduction in the viral infection in the cell by at most about 100-fold, at most about 90-fold, at most about 80-fold, at most about 70-fold, at most about 60-fold, at most about 50-fold, at most about 40-fold, at most about 30-fold, at most about 20-fold, at most about 15-fold, at most about 10-fold, at most about 9-fold, at most about 8-fold, at most about 7-fold, at most about 6-fold, at most about 5-fold, at most about 4.5-fold, at most about 4-fold, at most about 3.5-fold, at most about 3-fold, at most about 2.5-fold, at most about 2-fold, at most about 1.9-fold, at most about 1.8-fold, at most about 1.7-fold, at most about 1.6-fold, at most about 1.5-fold, at most about 1.4-fold, at most about 1.3-fold, at most about 1.2-fold, at most about 1.1-fold, at most about 1-fold, at most about 0.9-fold, at most about 0.8-fold, at most about 0.7-fold, at most about 0.6-fold, at most about 0.5-fold, at most about 0.4-fold, at most about 0.3-fold, at most about 0.2-fold, at most about 0.1-fold, or less as compared to the corresponding control.

In some embodiments of any one of the systems disclosed herein, the first therapeutic agent (or a gene encoding thereof) and the second therapeutic agent (or a gene encoding thereof) can be parts of a same composition (e.g., the same therapeutic composition). The same composition can be used (e.g., administered to a cell or a subject in need thereof) for reducing viral injection. In some embodiments of any one of the systems disclosed herein, the first therapeutic agent (or a gene encoding thereof) and the second therapeutic agent can be in different compositions (e.g., different therapeutic compositions). The different compositions can be used (e.g., administered to a subject in need thereof at two different time points and/or two different administration modes/sites) for reducing viral injection. In some examples, a first composition comprising the first therapeutic agent can be used (e.g., administered to a cell or a subject in need thereof) prior to use of a second composition comprising the second therapeutic agent. In some examples, the second composition comprising the second therapeutic agent can be used (e.g., administered to a cell or a subject in need thereof) prior to use of the first composition comprising the first therapeutic agent. In some examples, the first composition comprising the first therapeutic agent and the second composition comprising the second therapeutic agent can be used (e.g., administered to a cell or a subject in need thereof) substantially simultaneously to each other.

In some embodiments of any one of the systems disclosed herein, the combination effect of the first therapeutic agent and the second therapeutic agent in reducing viral infection can be a synergistic effect or an additive effect. The additive effect can be a sum of a plurality of therapeutic effects of a plurality of therapeutic agents, when the therapeutic effect of each therapeutic agent is measured or tested individually. In contrast, the synergistic effect can be a therapeutic effect of the sum of the plurality of therapeutic agents, which therapeutic effect is higher in terms of degree or longer in terms of duration than the additive effect. For example, when administering the first therapeutic agent and the second therapeutic agent to subjects in need thereof, the synergistic effect of administering both administering the first therapeutic agent and the second therapeutic agent to a subject can be higher in terms of degree or longer in terms of duration than the sum of the therapeutic effects of the first therapeutic agent and the second therapeutic agent when they are administered and tested individually (i.e., additive effect).

In some embodiments of any one of the systems disclosed herein, a fold decrease in viral infection (e.g., viral replication) from the synergistic effect of the first therapeutic agent and the second therapeutic agent can be greater than a fold decrease in viral infection from the additive effect of the first therapeutic agent and the second therapeutic agent by at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold, at least about 1 fold, at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 30-fold, at least about 40-fold, at least about 50-fold, at least about 60-fold, at least about 70-fold, at least about 80-fold, at least about 90-fold, or at least about 100-fold, or more. In some embodiments of any one of the systems disclosed herein, the fold decrease in viral infection from the synergistic effect of the first therapeutic agent and the second therapeutic agent can be greater than the fold decrease in viral infection from the additive effect of the first therapeutic agent and the second therapeutic agent by at most about 100-fold, at most about 90-fold, at most about 80-fold, at most about 70-fold, at most about 60-fold, at most about 50-fold, at most about 40-fold, at most about 30-fold, at most about 20-fold, at most about 15-fold, at most about 10-fold, at most about 9-fold, at most about 8-fold, at most about 7-fold, at most about 6-fold, at most about 5-fold, at most about 4.5-fold, at most about 4-fold, at most about 3.5-fold, at most about 3-fold, at most about 2.5-fold, at most about 2-fold, at most about 1.9-fold, at most about 1.8-fold, at most about 1.7-fold, at most about 1.6-fold, at most about 1.5-fold, at most about 1.4-fold, at most about 1.3-fold, at most about 1.2-fold, at most about 1.1-fold, at most about 1-fold, at most about 0.9-fold, at most about 0.8-fold, at most about 0.7-fold, at most about 0.6-fold, at most about 0.5-fold, at most about 0.4-fold, at most about 0.3-fold, at most about 0.2-fold, at most about 0.1-fold, or less.

In some embodiments of any one of the systems disclosed herein, a duration of reduced viral infection (e.g., viral replication) from the synergistic effect of the first therapeutic agent and the second therapeutic agent can be greater than a duration of reduced viral infection from the additive effect of the first therapeutic agent and the second therapeutic agent by at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold, at least about 1 fold, at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 30-fold, at least about 40-fold, at least about 50-fold, at least about 60-fold, at least about 70-fold, at least about 80-fold, at least about 90-fold, or at least about 100-fold, or more. In some embodiments of any one of the systems disclosed herein, the duration of reduced viral infection from the synergistic effect of the first therapeutic agent and the second therapeutic agent can be greater than the duration of reduced viral infection from the additive effect of the first therapeutic agent and the second therapeutic agent by at most about 100-fold, at most about 90-fold, at most about 80-fold, at most about 70-fold, at most about 60-fold, at most about 50-fold, at most about 40-fold, at most about 30-fold, at most about 20-fold, at most about 15-fold, at most about 10-fold, at most about 9-fold, at most about 8-fold, at most about 7-fold, at most about 6-fold, at most about 5-fold, at most about 4.5-fold, at most about 4-fold, at most about 3.5-fold, at most about 3-fold, at most about 2.5-fold, at most about 2-fold, at most about 1.9-fold, at most about 1.8-fold, at most about 1.7-fold, at most about 1.6-fold, at most about 1.5-fold, at most about 1.4-fold, at most about 1.3-fold, at most about 1.2-fold, at most about 1.1-fold, at most about 1-fold, at most about 0.9-fold, at most about 0.8-fold, at most about 0.7-fold, at most about 0.6-fold, at most about 0.5-fold, at most about 0.4-fold, at most about 0.3-fold, at most about 0.2-fold, at most about 0.1-fold, or less.

In some embodiments of any one of the systems disclosed herein, the second therapeutic agent can be an antiviral agent. The antiviral agent may not be capable targeting (e.g., binding) a viral gene or a derivative thereof. The antiviral agent may induce antiviral effect (e.g., reducing viral infection) by one or more mechanisms other than directly binding to a target viral gene. In some embodiments, the antiviral agent may not comprise a guide nucleic acid (e.g., sgRNA) designed to exhibit complementarity to a target viral gene. Non-limiting examples of the antiviral agent can include a cytokine, a small molecule, a silencing nucleic acid (e.g., siRNA, microRNA, etc.), and an antibody or a derivative thereof.

In some embodiments of any one of the systems disclosed herein, the antiviral agent can be an immune regulator. In some cases, the immune regulator can induce immunostimulatory or immunomodulatory effect in a host subject. In some cases, the immune regulator can activate (e.g., directly activate or indirectly activate) hematopoietic cells, such as, e.g., hematopoietic stem cells or immune cells (e.g., T cells, natural killer (NK) cells, NK T cells, macrophages, B cells, etc.). In some cases, the immune regulator can activate an activity (e.g., anti-viral activity or response) of the hematopoietic cells or prolong the activity/response.

In some embodiments of any one of the systems disclosed herein, the immune regulator can be a polypeptide (e.g., a cytokine) or a gene encoding thereof (e.g., a DNA plasmid, messenger RNA (mRNA), etc.). In some cases, the immune regulator cytokine can be an interferon (IFN), a derivative thereof, or a functional variant thereof. Non-limiting examples of the interferon can include type I IFN (e.g., IFN-α, IFN-β), type II IFN (e.g., IFN-γ), and type III IFN (e.g., IFN-λ). IFNs can be important for inducing, activating, or prolonging immune response (e.g., innate immune response) to viral infection. In some cases, the immune regulator cytokine can be an interleukin (IL), a derivative thereof, or a functional variant thereof. One or more IL cytokines can induce production (e.g., expression) of one or more IFN cytokines, thereby to induce immunostimulatory or immunomodulatory effect in a host subject. Non-limiting examples of the IL can include L-1, IL-2, IL-3, IL-4, IL-5, IL-6, IL-7, IL-8, IL-10, IL-11, IL-12, IL-13, IL-14, IL-16, IL-17, IL-18, IL-19, IL-20, IL-21, IL-22, IL-23, IL-24, IL, 25, IL-26, IL-27, IL-28, IL-29, IL-30, IL-31, IL-32, IL-33, IL-34, IL-35, IL-36, IL-37, and IL-38.

In some embodiments of any one of the systems disclosed herein, the antiviral agent can be an inhibitor moiety (e.g., a small molecule) that reduces activity of (i) a protein of a host cell (e.g., a protease) or (ii) a viral protein (e.g., RdRP, non-structural proteins, etc.). In some cases, the inhibitor moiety can be an enzyme inhibitor, such as, for example, a serine protease (e.g., Transmembrane protease, serine 2 (TMPRSS2)) inhibitor (e.g., camostat salt (e.g., camostat mesylate), a metabolite of camostat, nafamostat, sepimostat, gabexate, etc.); a cysteine protease (e.g., cathepsin B, calpains 1 and 2) inhibitor (e.g., Balicatib, CA074, Calpeptin, E-64, E-64d, L006235, SID26681509, etc.); a dehydrogenase (e.g., inosine-5′-monophosphate dehydrogenase (IMPDH)) inhibitor (e.g., ribavirine); or a kinease (e.g., a tyrosine kinase, such as JAK) inhibitor (e.g., Baricitinib). In some cases, the inhibitor moiety can be an inhibitor of a viral protein. In some examples, the viral protein RdRP can be targeted and its activity reduced by the inhibitor moiety, such as, e.g., EIDD-1931, Remdesivir, favipiravir, etc. In some examples, a nonstructural viral protein (e.g., nonstructural protein 5A (NS5A)) can be targeted and its activity reduced by the inhibitor moiety, such as, e.g., Elbasvir, Velpatasvir, etc.

Additional non-limiting examples of the antiviral agent can include, but are not limited to, 5-substituted 2′-deoxyuridine analogues, nucleoside analogues, pyrophosphate analogues, nucleoside reverse transcriptase inhibitors, non-nucleoside reverse transcriptase inhibitors, integrase inhibitors, entry inhibitors, acyclic guanosine analogues, acyclic nucleoside phosphonate analogues, oligonucleotides, and antimitotic inhibitors.

In some embodiments of any one of the systems disclosed herein, the second therapeutic agent (e.g., the antiviral agent) can be capable targeting (e.g., binding) an endogenous gene of a host cell, to thereby reduce viral infection in the host cell. The endogenous gene can encode an endogenous protein involved in viral infection in the host cell. For example, the endogenous protein may be a protein involved in (e.g., required for) attachment of a virus to the cell, penetration of the virus into the cell, uncoating of capsid of the virus, replication of the virus in the cell, or lysis of the cell to release replicated virus particles. In some cases, the endogenous gene can encode a transmembrane protein, such as a transmembrane protease. Non-limiting examples of the transmembrane protein can include TMPRSS2, angiotensin-converting enzyme 2 (ACE2) receptor, and Angiotensin 11 receptor type 1 (AT1R). In some cases, the endogenous gene can encode a peptidase, e.g., an aminopeptidase. Non-limiting examples of the peptidase can include alanyl aminopeptidase (ANPEP) and carboxypeptidase. In some cases, the endogenous gene can encode a protein exhibiting a plurality of distinct enzymatic activities (e.g., 2 or 3 different enzymatic activities). Non-limiting examples of the distinct enzymatic activities can include dehydrogenase activity (e.g., methylenetetrahydrofolate dehydrogenase activity), cyclohydrolase activity (e.g., methenyltetrahydrofolate cyclohydrolase activity), and ligase activity (e.g., formate-tetrahydrofolate ligase). For example, the endogenous gene can be methylenetetrahydrofolate dehydrogenase 1 (MTHFD1). In some cases, the endogenous gene can encode an intracellular protein of the cell, such as MTHFD1.

In some embodiments of any one of the systems disclosed herein, the second therapeutic agent (e.g., the antiviral agent) can comprise (i) an additional heterologous polypeptide comprising an additional gene regulating moiety or (ii) an additional heterologous nucleic acid molecule, wherein the additional gene regulating moiety or the additional heterologous nucleic acid molecule can be configured to bind the endogenous gene (e.g., promoter sequence, exon, intron, etc.). In some cases, the second therapeutic agent can only comprise the additional heterologous polypeptide comprising the additional gene regulating moiety (e.g., TALEN, ZFN, etc.). In some cases, the second therapeutic agent can only comprise the additional heterologous nucleic acid molecule (e.g., siRNA, miRNA, etc.). In some cases, the second therapeutic agent can comprise both the additional heterologous polypeptide comprising the additional gene regulating moiety and the additional heterologous nucleic acid molecule, to form a complex (e.g., a CRISPR/Cas-sgRNA complex) that is designed to specifically bind the endogenous gene of the host cell, as disclosed herein.

In some embodiments of any one of the systems disclosed herein, the system can be for regulating (e.g., reducing, maintaining, or enhancing) expression of one or more target viral genes in a cell. In some embodiments, the system can be for reducing the expression of the one or more target viral genes in the cell. The target viral gene can encode a viral polypeptide or a fragment thereof. Examples of the viral polypeptide can include, but are not limited to, RNA-dependent RNA polymerase (RdRP), hemagglutinin (HA), nucleoprotein (N-protein), neuraminidase (NA), matrix protein (e.g., M1 or M2 proteins), NS1 protein, and nuclear export proteins, PB2, PB2, PB1-F2, PA, NP, M1, M2, NS1, and NEP/NS2. In some cases, the viral polypeptide can comprise RdRP or a fragment thereof or N-protein or a fragment thereof. In some cases, the viral polypeptide comprise hemagglutinin (HA), neuraminidase (NA), a fragment thereof, or a combination thereof.

In some embodiments, the system as disclosed herein can comprise a heterologous polypeptide comprising a gene regulating moiety. The gene regulating moiety can be configured to regulate expression of the one and/or more target viral genes in the cell.

In some embodiments, the system as disclosed herein can comprise at least one heterologous RNA polynucleotide configured to bind at least a portion of the one or more target viral genes in the cell. In some cases, the at least one heterologous RNA polynucleotide can be capable of (or configured to) forming a complex with a heterologous polypeptide comprising a gene regulating moiety to regulate expression of the one or more target viral genes in the cell.

In some embodiments, the gene regulating moiety can be an RNA-guided gene regulating moiety that binds at least a portion of the one or more target viral genes. In some cases, the system further comprises at least one heterologous RNA polynucleotide configured to bind to the at least a portion or a fragment of the target one or more viral genes. In some instances, the system comprises a plurality of heterologous RNA polynucleotides configured to bind to the at least a portion or a fragment of the target one or more viral genes. The plurality of heterologous RNA polynucleotides can comprise at least 1, 2, 3, 4, 5, 6, 7, 8, 9, 10, 11, 12, 13, 14, 15, 20, 21, 22, 23, 24, 25, 30, 40, 50, or more heterologous RNA polynucleotides. The plurality of heterologous RNA polynucleotides can comprise at most 50, 40, 30, 25, 24, 23, 22, 21, 20, 19, 18, 17, 16, 15, 14, 13, 12, 11, 10, 9, 8, 7, 6, 5, 4, 3, or 2 heterologous RNA polynucleotides.

In some embodiments, the systems described herein can target a genome of a virus. The virus can be a DNA virus or a RNA virus. The virus may be, for example, a double stranded DNA virus, a single stranded DNA virus, a double stranded RNA virus, a positive sense single stranded RNA virus, a negative sense single stranded RNA virus, a single stranded RNA-reverse transcribing virus (retrovirus) or a double stranded DNA reverse transcribing virus. Examples of DNA viruses cam include, but are not limited to, cytomegalo virus, Herpex Simplex, Epstein-Barr virus, Simian virus 40, Bovine papillomavirus, Adeno-associated virus, Adenovirus, Vaccinia virus, and Baculo virus. Examples of RNA viruses can include, but are not limited to. Coronavirus. Semliki Forest virus, Sindbis virus, Poko virus, Rabies virus, Influenza virus, SV5, Respiratory Syncytial virus. Venezuela equine encephalitis virus, Kunjin virus, Sendai virus, Vesicular stomatitisvirus, and Retroviruses.

Various specific viruses include, but are not limited to, Papovaviridae, Adenoviridae, Herpesviridae, Herpesvirales, Ascoviridae, Ampullaviridae, Asfarviridae, Baculoviridae, Fuselloviridae, Globuloviridae, Guttaviridae, Hytrosaviridae, Iridoviridae, Lipothrixviridae, Nimaviridae, Poxviridae, Tectiviridae, Corticoviridae, Sulfolobus, Caudovirales, Corticoviridae, Tectiviridaea, Ligamenvirales, Ampullaviridae, Bicaudaviridae, Clavaviridae, Fuselloviridae, Globuloviridae, Guttaviridae, Turriviridae, Ascovirus, Baculovirus, Hytrosaviridae, Iridoviridae, Polydnaviruses, Mimiviridae, Marseillevirus, Megavirus, Mavirus virophage, Sputnik virophage, Nimaviridae, Phycodnaviridae, pleolipoviruses, Plasmaviridae, Pandoraviridae, Dinodnavirus, Rhizidiovirus, Salterprovirus, Sphaerolipoviridae, Anelloviridae, Bidnaviridae, Circoviridae, Geminiviridae, Genomoviridae, Inoviridae, Microviridae, Nanoviridae, Parvoviridae, Spiraviridae, Amalgaviridae, Bimaviridae, Chrysoviridae, Cystoviridae, Endornaviridae, Hypoviridae, Megabimaviridae, Partitiviridae, Picobimaviridae, Quadriviridae, Reoviridae, Totiviridae, Nidovirales, Picomavirales, Tymovirales, Mononegavirales, Bomaviridae, Filoviridae, Mymonaviridae, Nyamiviridae, Paramyxoviridae, Pneumoviridae, Rhabdoviridae, Sunviridae, Anphevirus, Arlivirus, Chengtivirus, Crustavirus, Wastrivirus, Bunyavirales, Feraviridae, Fimoviridae, Hantaviridae, Jonviridae, Nairoviridae, Peribunyaviridae, Phasmaviridae, Phenuiviridae, Tospoviridae, Arenaviridae, Ophioviridae, Orthomyxoviridae, Deltavirus, Taastrup virus, Alpharetrovirus, Avian leukosis virus. Rous sarcoma virus, Betaretrovirus, Mouse mammary tumor virus, Gammaretrovirus, Murine leukemia virus, Feline leukemia virus, Bovine leukemia virus, Human T-lymphotropic virus, Epsilonretrovirus, Walleye dermal sarcoma virus, Lentivirus, Human immunodeficiency virus 1, Simian and Feline immunodeficiency viruses, Spumavirus. Simian foamy virus, Orthoretrovirinae, Spumaretrovirinae, Metaviridae, Pseudoviridae, Retroviridae, Hepadnaviridae, or Caulimoviridae.

In some embodiments, the systems described herein can target a viral genome comprising a coronavirus genome. In some embodiments, the at least one heterologous RNA polynucleotide can comprise one or more mismatches when optimally aligned with a selected viral genome (e.g., MERS or SARS viral genome). The at least one heterologous RNA polynucleotide can comprise at least 1, 2, 3, 4, 5, 6, 7, 8, 9, 10, or more mismatches when optimally aligned with the selected viral genome. The at least one heterologous RNA polynucleotide can comprise at most 10, 9, 8, 7, 6, 5, 4, 3, 2, or 1 mismatch when optimally aligned with the selected viral genome. In some cases, the at least one heterologous RNA polynucleotide comprises at most 2 mismatches when optimally aligned with MERS and/or SARS viral genome. In alternative embodiments, the at least one heterologous RNA polynucleotide may not or need not comprise any mismatch when optimally aligned with the selected viral genome. In some cases, a single mismatch of the at least one heterologous RNA polynucleotide with respective to a portion of the MERS and/or SARS viral genome can be a nucleotide (e.g., a single nucleotide or an oligonucleotide) insertion or deletion.

In some embodiments, the gene regulating moiety modulate the expression of the viral gene via the binding of the at least one heterologous RNA polynucleotide to the one or more target viral genes. In some instances, the gene regulating moiety can reduce the expression level of the one or more target viral genes when the one or more viral genes is targeted and bound by the at least one heterologous RNA polynucleotide. In some embodiments, the RNA polynucleotide-guided gene regulating moiety can be a RNA-guided nuclease. In some embodiments, the RNA polynucleotide-guided gene regulating moiety can be a RNA-guided nuclease exhibiting RNA cleaving activity. In some embodiments, the RNA polynucleotide-guided gene regulating moiety can be a CRISPR/Cas protein. In some embodiments, the system, gene regulating moiety, and/or heterologous RNA polynucleotide can be collectively or separately packaged and delivered into a cell.

In some embodiments, the at least one heterologous RNA polynucleotide as described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 9%, 97%, 98%, 99%, or more sequence identity to a sequence selected from SEQ ID NOs: 1-3203, 8001-11203, 12001-12040, 12201-12240, 13001-13022, and 13201-13222.

The at least one heterologous RNA polynucleotide as described in the present disclosure can comprise at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 1-3203, 8001-11203, 12001-12040, 12201-12240, 13001-13022, and 13201-13222.

In some embodiments, the at least one heterologous RNA polynucleotide as described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence selected from SEQ ID NOs: 4001-7203, 12101-12140, and 13101-13122.

The at least one heterologous RNA polynucleotide as described in the present disclosure can comprise at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 4001-7203, 12101-12140, and 13101-13122.

In some preferred embodiments, the at least one heterologous RNA polynucleotide described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence selected from SEQ ID NOs: 13101, 13102, 13103, 13104, 13105, and 13106.

In some preferred embodiments, the at least one heterologous RNA polynucleotide as described in the present disclosure can comprise at most about 100%, 99%, 98%, 97%, 96%, 95%, 94%, 93%, 92%, 91% 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 13101, 13102, 13103, 13104, 13105, and 13106.

In some cases, the at least one heterologous RNA polynucleotide can be configured to effect at least about 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more reduction in expression of one or more target viral genes encoding one or more viral proteins (e.g., RdRP and/or N-protein) or a fragment thereof. In some cases, the least one heterologous RNA polynucleotide can be configured to effect at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, or less in expression of one or more target viral genes encoding one or more viral proteins (e.g., RdRP and/or N-protein) or a fragment thereof.

In some embodiments, the systems described herein can target a genome of an influenza virus.

In some embodiments, the systems described herein can target a viral genome comprising an influenza viral genome. In some embodiments, the at least one heterologous RNA polynucleotide can comprise one or more mismatches when optimally aligned with a selected viral genome (e.g., influenza genome). The at least one heterologous RNA polynucleotide can comprise at least 1, 2, 3, 4, 5, 6, 7, 8, 9, 10, or more mismatches when optimally aligned with the selected viral genome. The at least one heterologous RNA polynucleotide can comprise at most 10, 9, 8, 7, 6, 5, 4, 3, 2, or 1 mismatch when optimally aligned with the selected viral genome. In some cases, the at least one heterologous RNA polynucleotide comprises at most 2 mismatches when optimally aligned with influenza viral genome. In alternative embodiments, the at least one heterologous RNA polynucleotide may not or need not comprise any mismatch when optimally aligned with the selected viral genome. In some cases, a single mismatch of the at least one heterologous RNA polynucleotide with respective to a portion of the influenza viral genome can be a nucleotide (e.g., a single nucleotide or an oligonucleotide) insertion or deletion.

In some embodiments, the gene regulating moiety modulate the expression of the viral gene via the binding of the at least one heterologous RNA polynucleotide to the one or more target viral genes. In some instances, the gene regulating moiety can reduce the expression level of the one or more target viral genes when the one or more viral genes is targeted and bound by the at least one heterologous RNA polynucleotide. In some embodiments, the RNA polynucleotide-guided gene regulating moiety can be a RNA-guided nuclease. In some embodiments, the RNA polynucleotide-guided gene regulating moiety can be a RNA-guided nuclease exhibiting RNA cleaving activity. In some embodiments, the RNA polynucleotide-guided gene regulating moiety can be a CRISPR/Cas protein. In some embodiments, the system, gene regulating moiety, and/or heterologous RNA polynucleotide can be collectively or separately packaged and delivered into a cell.

In some embodiments, the at least one heterologous RNA polynucleotides as described in the present disclosure can comprise any sequence in SEQ ID NOs: 1-3203, 4001-7203, and 8001-11203.

In some embodiments, the at least one heterologous RNA polynucleotide as described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence selected from SEQ ID NOs: 13301-13348, 13401-13448, and 13501-13548.

The at least one heterologous RNA polynucleotide as described in the present disclosure can comprise at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 13301-13348, 13401-13448, and 13501-13548.

In some cases, the at least one heterologous RNA polynucleotide can be configured to effect at least about 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more reduction in expression of one or more target viral genes encoding one or more viral proteins (e.g., PB2, PB2, PB1-F2, PA, HA, NP, NA, M1, M2, NS1, and/or NEP/NS2) or a fragment thereof. In some cases, the least one heterologous RNA polynucleotide can be configured to effect at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, or less in expression of one or more target viral genes encoding one or more viral proteins (e.g., PB2, PB2, PB1-F2, PA, HA, NP, NA, M1, M2, NS1, and/or NEP/NS2) or a fragment thereof.

In some embodiments, the at least one heterologous RNA polynucleotide as described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence selected from SEQ ID NOs: 13601-13606, 13607-13649, 13651-13664, 13671-13684, and 13701-13729.

The at least one heterologous RNA polynucleotide as described in the present disclosure can comprise at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 13601-13606, 13607-13649, 13651-13664, 13671-13684, and 13701-13729.

In some embodiments, the at least one heterologous RNA polynucleotide comprising a nucleic acid sequence of any one SEQ ID NOs: 13601-13606 targets an endogenous gene of the cell being infected by any one of strain of the virus described herein. In some embodiments, the endogenous gene is a member of the peptidase family M1 such as aminopeptidase N (ANPEP), aminopeptidase A, leukotriene A4 hydrolase, Ape2 aminopeptidase, Aap1′ aminopeptidase, pyroglutamyl-peptidase II, cytosol alanyl aminopeptidase, cystinyl aminopeptidase, aminopeptidase G, aminopeptidase B, aminopeptidase Ey, endoplasmic reticulum aminopeptidase 1, tricom interacting factor F2, tricorn interacting factor F3, arginyl aminopeptidase-like 1, ERAP2 aninopeptidase, aminopeptidase, aminopeptidase O, or Tata binding protein associated factor. In some embodiments, the endogenous gene targeted by the heterologous RNA polynucleotide is ANPEP. In some embodiments, the endogenous gene is a member of the minor histocompatibility antigen family such as AKAP13, APOBEC3B, ARHGDIB, BCAT2, BCL2A1, CD19, CENPM, CTSH, C19orf48, DDX3Y, DPH1, EB13, ERAP1, ERBB2, GEMIN4, HEATR1, ARHGAP45, HMHB1, HMSD, KDM5D, PUM3, RESF1, LY75, MR1, MTHFD1, MYO1G, NUP133, PDCD11, P14K2B, PKN3, PRCP, PTK2B, P2RX5, RPS4Y1, SLC1A5, SLC19A1, SON, SP110, SSR1, SWAP70, TMSB4Y, TOR3A, TRIM22, TRIM42, TRIP10, TYMP, UGT2B17, USP9Y, UTY, WNK1, or ZNF419. In some embodiments, the endogenous gene targeted by the heterologous RNA polynucleotide is MTHFD1. In some embodiments, the endogenous gene is a member of the type II transmembrane serine protease family such as HAT/DESC subfamily: HAT, DESC1, TMPRSS11A, HAT-like 2, HAT-like 3, HAT-like 4, HAT-like 5; Hepsin/TMPRSS subfamily: Hepsin, TMPRSS2, TMPRSS3, TMPRSS4, MSPL, Spinesin, Enteropeptidase; Matriptase subfamily: Matriptase, Matriptase-2, Matripase-3, Polyserease-1; and Corin subfamily Corin. In some embodiments, the endogenous gene targeted by the heterologous RNA polynucleotide is TMPRSS2.

In some embodiments, the at least one heterologous RNA polynucleotide comprising a nucleic acid sequence of any one of SEQ ID NOs: 13607-13649 or 13701-13729 can target the RdRP, N-protein, or the viral genome of any one strain of the coronavirus described herein. In some embodiments, a combination of the at least one heterologous RNA polynucleotide comprising a nucleic acid sequence of any one of SEQ ID NOs: 13651-13664 or 13671-13684 can target any one strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the RNA virus described herein. Non-limiting examples of the RNA virus include RNA virus families of Coronaviridae, Pneumoviridae, Paramyxoviridae, Caliciviridae, Hepeviridae, Picomaviridae, Filoviridae, Flaviviridae, Rhabdoviridae, or Togaviridae.

In some embodiments, the cell can be contacted with the system in vivo, ex vivo, or in vitro. In some embodiments, the cell can be contacted with the system in vivo. In some embodiments, the cell can be contacted with the system ex vivo. In some embodiments, the cell can be contacted with the system in vitro. In some cases, the cell can be contacted by the system in vivo via administration of the system to a subject comprising the cell. Alternatively, both the cell and the system can be administered to the subject (e.g., via the same route of administration or different routes of administration), and the contacting between the cell and the system can occur in vivo, e.g., in the blood or a different tissue. In other cases, the cell can be contacted by the system ex vivo or in vitro. For example, the cell can be contacted by the system in a reaction mixture. Upon such contacting, the cell in the reaction mixture can be analyzed to measure the extent of viral transfection in the cell. Alternatively, upon the contacting, the cell (e.g., with or without the system) can be administered to a subject for a cell therapy, thereby reducing a chance of viral transfection in the cell and the subject prior to the cell therapy. Examples of the cell therapy can include stem cell therapies (e.g., for tissue repair or regeneration, neurodegenerative diseases, etc.) and immune cell therapies (e.g., chimeric antigen receptor (CAR) T cell therapies), as disclosed herein.

In some embodiments, the method can comprise contacting the cell with a polynucleotide encoding the systems (e.g., the heterologous polypeptide and/or the at least one heterologous polynucleotide) described herein, where the contacted cell can express the systems encoded by the polynucleotide. In some embodiments, the method can comprise directly delivering the systems described herein to the contacted cell. In some embodiments, a polynucleotide encoding the gene regulating moiety can be introduced into the cell, where the cell subsequently expresses the gene regulating moiety. In some embodiments, the systems can reduce expression of the one or more target viral genes. In some cases, the method comprises administering the systems described herein to a subject in need thereof.

Another aspect of the present disclosure provides a composition comprising any one of the systems described herein. Another aspect of the present disclosure provides a kit comprising such composition. Another aspect of the present disclosure provides a reaction mixture comprising (i) any one of the systems described herein and (ii) a biological sample (e.g., urine, blood, cell, tissue, organ, organism, etc.).

Another aspect of the present disclosure provides a method for reducing expression of one or more target viral genes in a cell by contacting the cell with any of the systems (e.g., systems comprising the heterologous polypeptide and/or the at least one heterologous polynucleotide) described herein.

### III. Additional Aspects

A. Virus

Described herein, in some embodiments, are systems and methods for targeting viral genomes or viral genes. In some embodiments, the systems and methods described herein target the transcripts of the viral genomes or viral genes. In some cases, the systems and methods described herein target the viral genomes or viral genes for cleavage or degradation. In some cases, the systems and methods described herein target the transcripts of the viral genomes or viral genes for cleavage or degradation. In some embodiments, the systems and methods described herein cleave or degrade the target viral genomes viral genes. In some embodiments, the systems and methods described herein cleave or degrade the target viral genomes, viral genes, or viral gene segments, or fragments thereof. In some embodiments, the systems and methods decried herein cleave or degrade the transcripts of the target viral genomes, viral genes, or viral gene segments, or fragments thereof.

In some embodiments, the viral genomes or viral genes being targeted, cleaved, degraded, and/or edited (e.g., mutated) by the systems and methods described herein can be viral genomes or viral genes of coronavirus. In some cases, such targeting, cleaving, degrading, and/or editing can effect reduction of viral infection in the cell or a host comprising the cell. In some instances, the coronavirus can be selected from the group consisting of: alphacoronavirus, betacoronavirus, deltacoronavirus, and gammacoronavirus. Examples of alphacoronavirus can include, but are not limited to, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhea virus, Rhinolophus bat coronavirus HKU2, and Scotophilus bat coronavirus 512. Examples of betacoronavirus can include, but are not limited to, Betacoronavirus 1, Hedgehog coronavirus 1. Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus, Tylonycteris bat coronavirus HKU4. Examples of deltacoronavirus can include, but are not limited to, Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20. Examples of gammacoronavirus can include, but are not limited to, Avian coronavirus, Beluga whale coronavirus SW1. Additional examples of coronavirus can include MERS-CoV, SARS-CoV, and SARS-Cov-2 (i.e., SARS-COV-2).

In some embodiments, the viral genomes or viral genes being targeted by the systems and the methods described herein can be selected from any one of the reference viral genome described herein. In some embodiments, the crRNA complements and binds to a reference viral genome, e.g., a viral genome or a fragment thereof from the GenBank. Non-limiting examples of viral genomes from the GenBank can include GenBank Access ID: LC522972, LC522973, LC522974, LC522975, LR757995, LR7579%, LR757997, LR757998, MN908947, MN938384, MN975262, MN985325, MN988668, MN988669, MN988713, MN994467, MN994468, MN996527, MN996528, MN996529, MN996530, MN996531, MN997409, MT007544, MT019529, MT019530, MT019531, MT019532, MT019533, MT020781, MT020880, MT020881, MT027062, MT027063, MT027064, MT039873, MT039887, MT039888, MT039890, MT044257, MT044258, MT049951, MT066175, MT066176, MT072688, MT093571, MT093631, AB257344, AB354579, AB551247, AC_000192, AF029248, AF201929, AF207902, AF208066, AF208067, AF220295, AF304460, AF353511, AF391541, AF391542, AJ271965, AJ311317, AP006557, AP006558, AP006559, AP006560, AP006561, AX154950, AY274119, AY278487, AY278488, AY278489, AY278490, AY278491, AY278554, AY278741, AY279354, AY282752, AY283794, AY283795, AY283796, AY283797, AY283798, AY291315, AY291451, AY297028, AY304486, AY304488, AY304495, AY310120, AY313906, AY319651, AY321118, AY323977, AY338174, AY338175, AY338732, AY345986, AY345987, AY345988, AY348314, AY350750, AY351680, AY357075, AY357076, AY362698, AY362699, AY390556, AY391777, AY394850, AY394978, AY394979, AY394983, AY394985, AY394986, AY394987, AY394989, AY394990, AY394991, AY394992, AY394993, AY394994, AY394995, AY3949%, AY394997, AY394998, AY394999, AY395000, AY395001, AY395002, AY395003, AY427439, AY461660, AY463059, AY463060, AY485277, AY485278, AY502923, AY502924, AY502925, AY502926, AY502927, AY502928, AY502929, AY502930, AY502931, AY502932, AY508724, AY514485, AY515512, AY518894, AY545914, AY545915, AY545916, AY545917, AY545918, AY545919, AY559081, AY559082, AY559083, AY559084, AY559085, AY559086, AY559087, AY559088, AY559089, AY559090, AY559091, AY559092, AY559093, AY559094, AY559095, AY559096, AY559097, AY567487, AY568539, AY572034, AY572035, AY572038, AY585228, AY585229, AY595412, AY597011, AY613947, AY613948, AY613949, AY613950, AY641576, AY646283, AY654624, AY686863, AY686864, AY692454, AY700211, AY714217, AY772062, AY851295, AY864805, AY864806, AY884001, AY903459, AY903460, AY994055, CQ870486, CQ903009, CQ918584, CQ918585, CQ918598, CS037919, CS078029, CS079026, CS079027, CS079028, CS079029, CS116934, CS117114, CS124012, CS254197, CS460762, CS480537, CS569493, DI195813, DJ009246, DJ045279, DJ059765, DJ066921, DL008527, DL476508, DQ001338, DQ001339, DQ010921, DQ011855, DQ022305, DQ071615, DQ084199, DQ084200, DQ182595, DQ201447, DQ286389, DQ288927, DQ339101, DQ412042, DQ412043, DQ415896, DQ415897, DQ415898, DQ415899, DQ415900, DQ415901, DQ415902, DQ415903, DQ415904, DQ415905, DQ415906, DQ415907, DQ415908, DQ415909, DQ415910, DQ415911, DQ415912, DQ415913, DQ415914, DQ443743, DQ445911, DQ445912, DQ497008, DQ640652, DQ646405, DQ648794, DQ648856, DQ648857, DQ648858, DQ811784, DQ811785, DQ811786, DQ811787, DQ811788, DQ811789, DQ834384, DQ848678, DQ898174, DQ915164, EF065505, EF065506, EF065507, EF065508, EF065509, EF065510, EF065511, EF065512, EF065513, EF065514, EF065515, EF065516, EF185992, EF203064, EF203065, EF203066, EF203067, EF424615, EF424616, EF424617, EF424618, EF424619, EF424620, EF424621, EF424622, EF424623, EF424624, EF446615, EU022525, EU022526, EU074218, EU095850, EU111742, EU186072, EU371559, EU371560, EU371561, EU371562, EU371563, EU371564, EU418975, EU418976, EU420137, EU420138, EU420139, EU526388, EU637854, EU714028, EU714029, EU817497, FJ376619, FJ376620, FJ376621, FJ376622, FJ415324, FJ425184, FJ425185, FJ425186, FJ425187, FJ425188, FJ425189, FJ425190, FJ588686, FJ647218, FJ647219, FJ647220, FJ647221, FJ647222, FJ647223, FJ647224, FJ647225, FJ647226, FJ647227, FJ755618, F3807652, FJ882926, FJ882927, FJ882928, FJ882929, FJ882930, FJ882931, FJ882932, F3882933, FJ882934, FJ882935, FJ882936, FJ882937, FJ882938, FJ882939, FJ882940, FJ882941, FJ882942, FJ882943, FJ882944, FJ882945, FJ882946, FJ882947, FJ882948, FJ882949, FJ882950, FJ882951, FJ882952, FJ882953, FJ882954, FJ882955, FJ882956, FJ882957, FJ882958, FJ882959, FJ882960, FJ882961, FJ882962, FJ882963, FJ884686, FJ884687, FJ888351, FJ904713, FJ904714, FJ904715, FJ904716, FJ904717, FJ904718, FJ904719, FJ904720, FJ904721, FJ904722, FJ904723, FJ938051, FJ938052, FJ938053, FJ938054, FJ938055, FJ938056, FJ938057, FJ938058, FJ938059, FJ938060, FJ938061, FJ938062, FJ938063, FJ938064, FJ938065, FJ938066, FJ938067, FJ938068, FJ959407, FN430414, FN430415, FV537210, FV537211, FV537212, FV537213, FW503166, GQ152141, GQ153539, GQ153540, GQ153541, GQ153542, GQ153543, GQ153544, GQ153545, GQ153546, GQ153547, GQ153548, GQ427173, GQ427174, GQ427175, GQ427176, GQ477367, GQ504720, GQ504721, GQ504722, GQ504723, GQ504724, GQ504725, GU190215, GU393331, GU393332, GU393333, GU393334, GU393335, GU393336, GU393337, GU393338, GU553361, GU553362, GU553363, GU553364, GU553365, GU593319, GU937797, H1553383, HM034837, HM211098, HM211099, HM211100, HM211101, HM245923, HM245924, HM245925, HM245926, HM776941, HQ012367, HQ012368, HQ012369, HQ012370, HQ012371, HQ012372, HQ392469, HQ392470, HQ392471, HQ392472, HQ462571, HQ848267, HQ850618, HQ890526, HQ890527, HQ890528, HQ890529, HQ890530, HQ890531, HQ890532, HQ890533, HQ890534, HQ890535, HQ890536, HQ890537, HQ890538, HQ890539, HQ890540, HQ890541, HQ890542, HQ890543, HQ890544, HQ890545, HQ890546, HW269828, HW364297, HW375992, HZ038027, JF274479, JF292902, JF292903, JF292904, JF292905, JF292906, JF292907, JF292908, JF292909, JF292910, JF292911, JF292912, JF292913, JF292914, JF292915, JF292916, JF292917, JF292918, JF292919, JF292920, JF292921, JF292922, JF330898, JF330899, JF705860, JF732903, JF792616, JF792617, JF828980, JF828981, JF893452, JN129834, JN129835, JN183882, JN183883, JN547228, JN634064, JN825712, JN854286, JN856008, JN874559, JN874560, JN874561, JN874562, JQ023161, JQ023162, JQ065042, JQ065043, JQ065044, JQ065045, JQ065046, JQ065047, JQ065048, JQ065049, JQ088078, JQ173883, JQ282909, JQ316196, JQ404409, JQ404410, JQ408980, JQ408981, JQ410000, JQ765563, JQ765564, JQ765565, JQ765566, JQ765567, JQ765568, JQ765569, JQ765570, JQ765571, JQ765572, JQ765573, JQ765574, JQ765575, JQ900255, JQ900256, JQ900257, JQ900258, JQ900259, JQ900260, JQ977697, JQ977698, JQ989266, JQ989267, JQ989268, JQ989269, JQ989270, JQ989271, JQ989272, JQ989273, JX088695, JX104161, JX112709, JX162087, JX163923, JX163924, JX163925, JX163926, JX163927, JX163928, JX169866, JX169867, JX188454, JX195175, JX195176, JX195177, JX195178, JX261936, JX489155, JX503060, JX503061, JX504050, JX524137, JX524171, JX560761, JX647847, JX840411, JX860640, JX869059, JX897900, JX993987, JX993988, KC008600, KC013541, KC109141, KC119407, KC136209, KC140102, KC164505, KC175339, KC175340, KC175341, KC189944, KC196276, KC210145, KC210146, KC210147, KC461235, KC461236, KC461237, KC506155, KC545383, KC545386, KC667074, KC776174, KC869678, KC881005, KC881006, KC962433, KF186564, KF186565, KF186566, KF186567, KF192507, KF267450, KF268336, KF268337, KF268338, KF268339, KF272920, KF294380, KF294457, KF367457, KF377577, KF384500, KF411040, KF411041, KF4301%, KF430199, KF430200, KF430201, KF430202, KF430219, KF452322, KF452323, KF460437, KF468752, KF468753, KF468754, KF514388, KF514389, KF514390, KF514391, KF514392, KF514393, KF514394, KF514395, KF514396, KF514397, KF514398, KF514399, KF514400, KF514401, KF514402, KF514403, KF514404, KF514405, KF514406, KF514407, KF514408, KF514409, KF514410, KF514411, KF514412, KF514413, KF514414, KF514415, KF514416, KF514417, KF514418, KF514419, KF514420, KF514421, KF514422, KF514423, KF514430, KF514432, KF514433, KF530060, KF530061, KF530063, KF530064, KF530065, KF530066, KF530067, KF530068, KF530069, KF530070, KF530071, KF530072, KF530073, KF530074, KF530075, KF530076, KF530077, KF530078, KF530079, KF530080, KF530081, KF530082, KF530083, KF530084, KF530085, KF530086, KF530087, KF530088, KF530089, KF530090, KF530091, KF530092, KF530094, KF530095, KF530096, KF530097, KF530098, KF530099, KF530104, KF530105, KF530106, KF530107, KF530108, KF530109, KF530110, KF530111, KF530112, KF530113, KF530114, KF530115, KF530116, KF530117, KF530118, KF530119, KF530120, KF530121, KF530122, KF530123, KF530124, KF530125, KF530126, KF530127, KF530129, KF530130, KF530131, KF530132, KF530133, KF530134, KF530135, KF530136, KF530270, KF530271, KF569996, KF574761, KF600612, KF600613, KF600620, KF600627, KF600628, KF600630, KF600632, KF600634, KF600644, KF600645, KF600647, KF600651, KF600652, KF636752, KF650370, KF650371, KF650372, KF650373, KF650374, KF650375, KF663559, KF663560, KF663561, KF668605, KF686338, KF686339, KF686340, KF686341, KF686342, KF686343, KF686344, KF686345, KF686346, KF696629, KF745068, KF760557, KF761675, KF793824, KF793825, KF793826, KF804028, KF840537, KF850450, KF853202, KF906249, KF906250, KF906251, KF917527, KF923886, KF923887, KF923888, KF923889, KF923890, KF923891, KF923892, KF923893, KF923894, KF923895, KF923896, KF923897, KF923898, KF923899, KF923900, KF923901, KF923902, KF923903, KF923904, KF923905, KF923906, KF923907, KF923908, KF923909, KF923910, KF923911, KF923912, KF923913, KF923914, KF923915, KF923916, KF923917, KF923918, KF923919, KF923920, KF923921, KF923922, KF923923, KF923924, KF923925, KF931628, KF958702, KF961221, KF961222, KJ020932, KJ128295, KJ1135013, KJ156866, KJ156869, KJ156874, KJ156881, KJ156910, KJ156934, KJ156944, KJ156949, KJ156952, KJ158152, KJ184549, KJ196348, KJ361500, KJ361501, KJ361502, KJ361503, KJ399978, KJ408801, KJ425485, KJ425486, KJ425487, KJ425488, KJ425489, KJ425490, KJ425491, KJ425492, KJ425493, KJ425494, KJ425495, KJ425496, KJ425497, KJ425498, KJ425499, KJ425500, KJ425501, KJ425502, KJ425503, KJ425504, KJ425505, KJ425506, KJ425507, KJ425508, KJ425509, KJ425510, KJ425511, KJ425512, KJ435283, KJ435284, KJ435285, KJ435286, KJ462462, KJ473795, KJ473796, KJ473797, KJ473798, KJ473799, KJ473800, KJ473806, KJ473807, KJ473808, KJ473809, KJ473810, KJ473811, KJ473812, KJ473813, KJ473814, KJ473815, KJ473816, KJ473820, KJ473821, KJ473822, KJ477102, KJ477103, KJ481931, KJ526096, KJ556336, KJ567050, KJ569769, KJ584355, KJ584356, KJ584357, KJ584358, KJ584359, KJ584361, KJ588062, KJ588063, KJ588064, KJ601777, KJ601778, KJ601779, KJ601780, KJ614529, KJ620016, KJ623926, KJ645635, KJ645636, KJ645637, KJ645638, KJ645639, KJ645640, KJ645641, KJ645642, KJ645643, KJ645644, KJ645645, KJ645646, KJ645647, KJ645648, KJ645649, KJ645650, KJ645651, KJ645652, KJ645653, KJ645654, KJ645655, KJ645656, KJ645657, KJ645658, KJ645659, KJ645660, KJ645661, KJ645662, KJ645663, KJ645664, K1645665, KJ645666, KJ645667, KJ645668, KJ645669, KJ645670, KJ645671, KJ645672, KJ645673, KJ645674, KJ645675, KJ645676, KJ645677, KJ645678, KJ645679, KJ645680, KJ645681, KJ645682, K645683, K1645684, KM645685, KJ645686, KJ645687, KJ645688, KJ645689, KJ645690, KJ645691, KJ645692, KJ645693, KJ645694, KJ645695, KJ645696, KJ645697, KJ645698, KJ645699, KJ645700, KJ645701, KJ645702, KJ645703, KJ645704, KJ645705, KJ645706, KJ645707, KJ645708, KJ650098, KJ650295, KJ6502%, KJ650297, KJ662670, KJ713295, KJ713296, KJ713297, KJ713298, KJ713299, KJ769231, KJ777677, KJ777678, KJ778615, KJ778616, KJ813439, KJ829365, KJ958218, KJ958219, KJ960178, KJ960179, KJ960180, KM012168, KM015348, KM027255, KM027256, KM027257, KM027258, KM027259, KM027260, KM027261, KM027262, KM052365, KM077139, KM089829, KM189367, KM210277, KM210278, KM213963, KM242131, KM347965, KM349742, KM349743, KM349744, KM392224, KM392225, KM392226, KM392227, KM392228, KM392229, KM392230, KM392231, KM392232, KM403155, KM454473, KM586818, KM609203, KM609204, KM609205, KM609206, KM609207, KM609208, KM609209, KM609210, KM609211, KM609212, KM609213, KM820765, KM887144, KM975735, KM975736, KM975737, KM975738, KM975739, KM975740, KM975741, KP036502, KP036503, KP036504, KP036505, KP118880, KP118881, KP118882, KP118883, KP118884, KP118885, KP118886, KP118887, KP118888, KP118889, KP118890, KP118891, KP118892, KP118893, KP118894, KP143507, KP143508, KP143509, KP143510, KP143511, KP143512, KP162057, KP198610, KP198611, KP202848, KP209306, KP209307, KP209308, KP209309, KP209310, KP209311, KP209312, KP209313, KP223131, KP343691, KP403802, KP403954, KP641661, KP641662, KP641663, KP662631, KP688354, KP719927, KP719928, KP719929, KP719930, KP719931, KP719932, KP719933, KP728470, KP757890, KP757891, KP757892, KP765609, KP780179, KP790143, KP790144, KP790145, KP790146, KP868572, KP868573, KP886808, KP886809, KP887098, KP890336, KP981395, KP981644, KR003452, KR011263, KR011264, KR011265, KR011266, KR011756, KR061458, KR061459, KR078299, KR078300, KR095279, KR131621, KR150443, KR153325, KR153326, KR231009, KR265759, KR265760, KR265761, KR265762, KR265763, KR265764, KR265765, KR265766, KR265767, KR265768, KR265769, KR265770, KR265771, KR265772, KR265773, KR265774, KR265775, KR265776, KR265777, KR265778, KR265779, KR265780, KR265781, KR265782, KR265783, KR265784, KR265785, KR265786, KR265787, KR265788, KR265789, KR265790, KR265791, KR265792, KR265793, KR265794, KR265795, KR265796, KR265797, KR265798, KR265799, KR265800, KR265801, KR265802, KR265803, KR265804, KR265805, KR265806, KR265807, KR265808, KR265809, KR265810, KR265811, KR265812, KR265813, KR265814, KR265815, KR265816, KR265817, KR265818, KR265819, KR265820, KR265821, KR265822, KR265823, KR265824, KR265825, KR265826, KR265827, KR265828, KR265829, KR265830, KR265831, KR265832, KR265833, KR265834, KR265840, KR265841, KR265842, KR265843, KR265844, KR265845, KR265846, KR265847, KR265848, KR265849, KR265850, KR265851, KR265852, KR265853, KR265854, KR265855, KR265856, KR265857, KR265858, KR265859, KR265860, KR265861, KR265862, KR265863, KR265864, KR265865, KR270796, KR608272, KR610991, KR610992, KR610993, KR610994, KR809885, KR818832, KR818833, KR822424, KR873431, KR873434, KR873435, KR902510, KT006149, KT021227, KT021228, KT021229, KT021230, KT021231, KT021232, KT021233, KT021234, KT026453, KT026454, KT026455, KT026456, KT029139, KT121572, KT121573, KT121574, KT121575, KT121576, KT121577, KT121578, KT121579, KT121580, KT121581, KT156560, KT156561, KT199103, KT203557, KT225476, KT253269, KT253270, KT253271, KT253272, KT253324, KT253325, KT253326, KT253327, KT253328, KT266822, KT266906, KT323979, KT323980, KT326819, KT336560, KT368824, KT368825, KT368826, KT368827, KT368828, KT368829, KT368830, KT368831, KT368832, KT368833, KT368834, KT368835, KT368836, KT368837, KT368838, KT368839, KT368840, KT368841, KT368842, KT368843, KT368844, KT368845, KT368846, KT368847, KT368848, KT368849, KT368850, KT368851, KT368852, KT368853, KT368854, KT368855, KT368856, KT368857, KT368858, KT368859, KT368860, KT368861, KT368862, KT368863, KT368864, KT368865, KT368866, KT368867, KT368868, KT368869, KT368870, KT368871, KT368872, KT368873, KT368874, KT368875, KT368876, KT368877, KT368878, KT368879, KT368880, KT368881, KT368882, KT368883, KT368884, KT368885, KT368886, KT368887, KT368888, KT368889, KT368890, KT368891, KT368892, KT368893, KT368894, KT368895, KT368896, KT368897, KT368898, KT368899, KT368900, KT368901, KT368902, KT368903, KT368904, KT368905, KT368906, KT368907, KT368908, KT368909, KT368910, KT368911, KT368912, KT368913, KT368914, KT368915, KT368916, KT374050, KT374051, KT374052, KT374053, KT374054, KT374055, KT374056, KT374057, KT381613, KT381875, KT444582, KT591944, KT696544, KT736031, KT736032, KT751244, KT779555, KT779556, KT806044, KT806045, KT806046, KT806047, KT806048, KT806049, KT806051, KT806052, KT806053, KT806054, KT806055, KT852992, KT860508, KT861627, KT861628, KT877350, KT877351, KT886454, KT941120, KT946798, KU051641, KU051649, KU131570, KU182964, KU182965, KU215419, KU215420, KU215421, KU215422, KU215423, KU215424, KU215425, KU215426, KU215427, KU215428, KU242423, KU242424, KU252649, KU291448, KU291449, KU297956, KU308549, KU317090, KU356856, KU361187, KU361188, KU380331, KU521535, KU558701, KU558702, KU569509, KU646831, KU664503, KU665558, KU710264, KU710265, KU729220, KU762338, KU847996, KU851859, KU851860, KU851861, KU851862, KU851863, KU851864, KU886219, KU893861, KU893869, KU893870, KU893871, KU893872, KU893873, KU900738, KU900739, KU900740, KU900741, KU900742, KU900743, KU900744, KU973692, KU975389, KU981059, KU981060, KU981061, KU981062, KU982966, KU982967, KU982968, KU982969, KU982970, KU982971, KU982972, KU982973, KU982974, KU982975, KU982976, KU982977, KU982978, KU982979, KU982980, KU982981, KU984334, KX016034, KX022602, KX022603, KX022604, KX022605, KX034094, KX034095, KX034096, KX034097, KX034098, KX034099, KX034100, KX058031, KX058032, KX058033, KX058075, KX064280, KX066126, KX077987, KX083668, KX108937, KX108938, KX108939, KX108940, KX108941, KX108942, KX108943, KX108944, KX108945, KX108946, KX118627, KX154684, KX154685, KX154686, KX154687, KX154688, KX154689, KX154690, KX154691, KX154692, KX154693, KX154694, KX179500, KX185056, KX185057, KX185058, KX185059, KX219791, KX219792, KX219793, KX219794, KX219795, KX219796, KX219797, KX219798, KX219799, KX219800, KX219801, KX236000, KX236001, KX236002, KX236003, KX236004, KX236005, KX236006, KX236007, KX236008, KX236009, KX236010, KX236011, KX236012, KX236013, KX236014, KX236015, KX236016, KX247127, KX247128, KX247129, KX247130, KX252772, KX252773, KX252774, KX252775, KX252776, KX252777, KX252778, KX252779, KX252780, KX252781, KX252782, KX252783, KX252784, KX252785, KX252786, KX252787, KX252788, KX252789, KX252790, KX252791, KX258195, KX259248, KX259249, KX259250, KX259251, KX259252, KX259253, KX259254, KX259255, KX259256, KX259257, KX266757, KX272465, KX275390, KX275391, KX275392, KX275393, KX275394, KX289955, KX302860, KX302861, KX302862, KX302863, KX302864, KX302865, KX302866, KX302867, KX302868, KX302869, KX302870, KX302871, KX302872, KX302873, KX302874, KX302875, KX344031, KX348114, KX348115, KX348116, KX348117, KX364290, KX364291, KX364292, KX364293, KX364294, KX364295, KX364296, KX364297, KX364298, KX364299, KX364300, KX372249, KX372250, KX375805, KX375806, KX375807, KX375808, KX389094, KX400753, KX425847, KX432213, KX434788, KX434789, KX434790, KX442564, KX442565, KX443143, KX499468, KX512809, KX512810, KX534205, KX534206, KX538964, KX538965, KX538966, KX538967, KX538968, KX538969, KX538970, KX538971, KX538972, KX538973, KX538974, KX538975, KX538976, KX538977, KX538978, KX538979, KX550281, KX574227, KX580953, KX580958, KX640829, KX683006, KX722529, KX722530, KX791060, KX812523, KX812524, KX834351, KX834352, KX839246, KX839247, KX839248, KX839249, KX839250, KX839251, KX883635, KX900393, KX900394, KX900395, KX90039, KX900397, KX900398, KX900399, KX900400, KX900401, KX900402, KX900403, KX900404, KX900405, KX900406, KX900407, KX900408, KX900409, KX900410, KX900411, KX964649, KX981440, KX982264, KX998969, KY007139, KY007140, KY014281, KY014282, KY019623, KY019624, KY047602, KY063616, KY063617, KY063618, KY065120, KY070587, KY073744, KY073745, KY073746, KY073747, KY073748, KY1278, KY292377, KY293677, KY293678, KY352407, KY354363, KY354364, KY363867, KY363868, KY364365, KY369905, KY369906, KY369907, KY369908, KY369909, KY369910, KY369911, KY369912, KY369913, KY369914, KY370043, KY370044, KY370045, KY370046, KY370047, KY370048, KY370049, KY370050, KY370051, KY370052, KY370053, KY406735, KY407556, KY407557, KY407558, KY417142, KY417143, KY417144, KY417145, KY417146, KY417147, KY417148, KY417149, KY417150, KY417151, KY417152, KY419103, KY419104, KY419105, KY419106, KY419107, KY419109, KY419110, KY419111, KY419112, KY419113, KY420075, KY421672, KY421673, KY486713, KY486714, KY499261, KY499262, KY513724, KY513725, KY554967, KY554968, KY554969, KY554970, KY554971, KY554972, KY554973, KY554974, KY554975, KY566209, KY566210, KY566211, KY581684, KY581685, KY581686, KY581687, KY581688, KY581689, KY581690, KY581691, KY581692, KY581693, KY581694, KY581695, KY581696, KY581697, KY581698, KY581699, KY581700, KY620116, KY621348, KY626044, KY626045, KY649107, KY673148, KY673149, KY674914, KY674915, KY674916, KY674917, KY674918, KY674919, KY674920, KY674921, KY674941, KY674942, KY674943, KY684759, KY684760, KY688118, KY688119, KY688120, KY688121, KY688122, KY688123, KY688124, KY689142, KY770850, KY770851, KY770858, KY770859, KY770860, KY776700, KY776701, KY793536, KY799179, KY799582, KY805845, KY805846, KY825240, KY825241, KY825242, KY825243, KY829118, KY926512, KY928065, KY929405, KY929406, KY933089, KY933090, KY938558, KY963963, KY967356, KY967357, KY9967358, KY967359, KY967360, KY967361, KY983583, KY983585, KY983586, KY983587, KY983588, KY994645, KY996417, LC022792, LC053455, LC061272, LC061273, LC061274, LC063810, LC063811, LC063812, LC063813, LC063814, LC063815, LC063816, LC063817, LC063818, LC063819, LC063820, LC063821, LC063822, LC063823, LC063824, LC063825, LC063826, LC063827, LC063828, LC063829, LC063830, LC063831, LC063832, LC063833, LC063834, LC063835, LC063836, LC063837, LC063838, LC063839, LC063840, LC063841, LC063842, LC063843, LC063844, LC063845, LC063846, LC063847, LC119077, LC215871, LC216914, LC216915, LC260038, LC260039, LC260040, LC260041, LC260042, LC260043, LC260044, LC260045, LC364342, LC364343, LC364344, LC364345, LC364346, LC469308, LC494126, LC494127, LC494128, LC494129, LC494130, LC494131, LC494132, LC494133, LC494134, LC494135, LC494136, LC494137, LC494138, LC494139, LC494140, LC494141, LC494142, LC494143, LC494144, LC494145, LC494146, LC494147, LC494148, LC494149, LC494150, LC494151, LC494152, LC494153, LC494154, LC494155, LC494156, LC494157, LC494158, LC494159, LC494160, LC494161, LC494162, LC494163, LC494164, LC494165, LC494166, LC494167, LC494168, LC494169, LC494170, LC494171, LC494172, LC494173, LC494174, LC494175, LC494176, LC494177, LC494178, LC494179, LC494180, LC494181, LC494182, LC494183, LC494184, LC494185, LC494186, LC494187, LC494188, LC494189, LC494190, LC494191, LC494192, LG076103, LG076104, LG076105, LM645057, LM645058, LN610099, LP731408, LP731409, LP731410, LP731411, LP731412, LP731413, LP731414, LP731415, LP731416, LP731435, LP731436, LP731437, LP731438, LP731439, LP731459, LP731460, LP731461, LP731462, LP731463, LP731464, LP731465, LP731466, LP731467, LP731468, LP731469, LP731470, LP731471, LP731472, LP731473, LP731474, LP731475, LP731476, LP731477, LP731478, LP963762, LP994923, LQ068527, LQ068536, LQ068537, LQ110721, LQ110722, LQ289146, LQ302036, LQ410511, LQ410512, LQ410513, LQ410514, LQ410515, LQ410516, LQ410517, LQ410518, LQ410519, LQ905109, LT545990, LT897799, LT898408, LT898409, LT898410, LT898411, LT898412, LT898413, LT898414, LT898415, LT898416, LT898417, LT898418, LT898420, LT898421, LT898423, LT898425, LT898426, LT898427, LT898430, LT898431, LT898432, LT898433, LT898435, LT898436, LT898438, LT898439, LT898440, LT898441, LT898443, LT898444, LT898445, LT898446, LT898447, LT900498, LT900499, LT900500, LT900501, LT900502, LT905450, LT905451, LT906581, LT906582, LT906620, LY411674, LY411680, LY411681, LY505956, LY611929, LY646142, LY646143, M95169, MA347646, MA347647, MA347648, MA347649, MA347650, MA347651, MA347652, MA347653, MA347654, MA347655, MA347656, MA347657, MA347658, MA347659, MA347660, MA347661, MA347662, MA347663, MA347664, MA347665, MA347666, MA347667, MA347668, MA347669, MA347670, MA347671, MA347672, MA347673, MA347674, MA347675, MA347676, MA347677, MA347678, MA347679, MA741906, MA741912, MA741913, MA765825, MF000457, MF000458, MF000459, MF000460, MF041982, MF083115, MF094681, MF094682, MF094683, MF094684, MF094685, MF094686, MF094687, MF094688, MF095123, MF113046, MF167434, MF280390, MF281416, MF314143, MF346935, MF370205, MF373643, MF374983, MF374984, MF374985, MF375374, MF421319, MF421320, MF431742, MF431743, MF462814, MF508703, MF542265, MF577027, MF593268, MF593473, MF593476, MF598594, MF598595, MF598596, MF598597, MF598598, MF598599, MF598600, MF598601, MF598602, MF598603, MF598604, MF598605, MF598606, MF598607, MF598608, MF598609, MF598610, MF598611, MF598612, MF598613, MF598614, MF598615, MF598616, MF598617, MF598618, MF598619, MF598620, MF598621, MF598622, MF598623, MF598624, MF598625, MF598626, MF598627, MF598629, MF598630, MF598631, MF598632, MF598633, MF598634, MF598635, MF598636, MF598637, MF598638, MF598639, MF598640, MF598641, MF598643, MF598644, MF598645, MF598646, MF598647, MF598648, MF598649, MF598650, MF598651, MF598652, MF598653, MF598654, MF598655, MF598656, MF598657, MF598658, MF598659, MF598660, MF598661, MF598662, MF598663, MF598664, MF598665, MF598666, MF598667, MF598668, MF598669, MF598670, MF598671, MF598672, MF598673, MF598674, MF598675, MF598676, MF598677, MF598678, MF598679, MF598680, MF598681, MF598682, MF598683, MF598684, MF598685, MF598686, MF598687, MF598688, MF598689, MF598690, MF598691, MF598692, MF598693, MF598694, MF598695, MF598696, MF598697, MF598698, MF598699, MF598700, MF598701, MF598702, MF598703, MF598704, MF598705, MF598706, MF598707, MF598708, MF598709, MF598710, MF598711, MF598712, MF598713, MF598714, MF598715, MF598716, MF598717, MF598719, MF598720, MF598721, MF598722, MF618252, MF618253, MF642322, MF642323, MF642324, MF642325, MF737355, MF769416, MF769417, MF769418, MF769419, MF769420, MF769421, MF769422, MF769423, MF769424, MF769425, MF769426, MF769427, MF769428, MF769429, MF769430, MF769431, MF769432, MF769433, MF769434, MF769435, MF769436, MF769437, MF769438, MF769439, MF769440, MF769441, MF769442, MF769443, MF769444, MF782686, MF782687, MF807951, MF807952, MF882923, MF924724, MF924725, MG011340, MG011341, MG011342, MG011343, MG011344, MG011345, MG011346, MG011347, MG011348, MG011349, MG011350, MG011351, MG011352, MG011353, MG011354, MG011355, MG011356, MG011357, MG011358, MG011359, MG011360, MG011361, MG011362, MG021194, MG021451, MG021452, MG197709, MG197710, MG197711, MG197712, MG197713, MG197714, MG197715, MG197716, MG197717, MG197718, MG197719, MG197720, MG197721, MG197722, MG197723, MG197727, MG233398, MG242062, MG334554, MG334555, MG366483, MG366880, MG366881, MG366882, MG366883, MG428699, MG428701, MG428702, MG428703, MG428704, MG428705, MG428706, MG448607, MG470650, MG517474, MG518518, MG520075, MG520076, MG546330, MG546331, MG546687, MG546688, MG546689, MG546690, MG557844, MG596802, MG596803, MG605090, MG605091, MG693168, MG693169, MG693170, MG693172, MG738154, MG738155, MG742313, MG757138, MG757139, MG757140, MG757141, MG757142, MG757593, MG757594, MG757595, MG757596, MG757597, MG757598, MG757599, MG757600, MG757601, MG757602, MG757603, MG757604, MG757605, MG762674, MG763935, MG772808, MG772933, MG772934, MG781192, MG812375, MG812376, MG812377, MG812378, MG832584, MG837011, MG837012, MG837058, MG837130, MG837131, MG837132, MG837133, MG893511, MG912595, MG912596, MG912597, MG912598, MG912599, MG912600, MG912601, MG912602, MG912603, MG912604, MG912605, MG912606, MG912607, MG912608, MG913342, MG913343, MG916901, MG916902, MG916903, MG916904, MG923466, MG923467, MG923468, MG923469, MG923470, MG923471, MG923472, MG923473, MG923474, MG923475, MG923476, MG923477, MG923478, MG923479, MG923480, MG923481, MG923574, MG977444, MG977445, MG977447, MG977449, MG977451, MG977452, MG983755, MH004412, MH004413, MH004414, MH004415, MH004416, MH004417, MH004418, MH004419, MH004420, MH004421, MH006957, MH006958, MH006959, MH006960, MH006961, MH006962, MH006963, MH006964, MH006965, MH013216, MH013462, MH013463, MH013464, MH013465, MH013466, MH020185, MH021175, MH029552, MH043952, MH043953, MH043954, MH043955, MH052681, MH052682, MH052683, MH052684, MH052685, MH052687, MH052688, MH052689, MH056657, MH056658, MH061336, MH061337, MH061338, MH061339, MH061340, MH061341, MH061342, MH061343, MH107321, MH107322, MH1117940, MH121121, MH181793, MH243316, MH243318, MH243319, MH259485, MH259486, MH306207, MH310909, MH310910, MH310911, MH310912, MH371127, MH395139, MH432120, MH454272, MH532440, MH539766, MH539771, MH539772, MH581489, MH593900, MH615810, MH687934, MH687935, MH687936, MH687937, MH687938, MH687939, MH687940, MH687941, MH687942, MH687943, MH687944, MH687945, MH687946, MH687947, MH687948, MH687949, MH687950, MH687951, MH687952, MH687953, MH687954, MH687955, MH687956, MH687957, MH687958, MH687959, MH687960, MH687961, MH687962, MH687963, MH687964, MH687965, MH687966, MH687967, MH687968, MH687969, MH687970, MH687971, MH687972, MH687973, MH687974, MH687976, MH687977, MH687978, MH697599, MH708123, MH708124, MH708125, MH708243, MH708895, MH715491, MH726362, MH726363, MH726364, MH726365, MH726366, MH726367, MH726368, MH726369, MH726370, MH726371, MH726372, MH726373, MH726374, MH726375, MH726376, MH726377, MH726378, MH726379, MH726380, MH726381, MH726382, MH726383, MH726384, MH726385, MH726386, MH726387, MH726388, MH726389, MH726390, MH726391, MH726392, MH726393, MH726394, MH726395, MH726396, MH726397, MH726398, MH726399, MH726400, MH726401, MH726402, MH726403, MH726404, MH726405, MH726406, MH726407, MH726408, MH734114, MH734115, MH748550, MH779856, MH779857, MH779858, MH779859, MH779860, MH810163, MH817484, MH822886, MH878976, MH891584, MH891585, MH891586, MH891587, MH891588, MH891589, MH891590, MH910099, MH924835, MH938448, MH938449, MH938450, MH940245, MK005882, MK032177, MK032178, MK032179, MK032180, MK032181, MK032689, MK032690, MK032691, MK032692, MK039552, MK039553, MK052676, MK062179, MK062180, MK062181, MK062182, MK062183, MK062184, MK071267, MK071619, MK071620, MK071621, MK071622, MK071623, MK071624, MK071625, MK071626, MK071627, MK071628, MK071629, MK071630, MK071631, MK071632, MK071633, MK071634, MK071635, MK071636, MK071637, MK071638, MK071639, MK129253, MK138353, MK138516, MK140811, MK140812, MK140813, MK140814, MK142676, MK163627, MK167038, MK204393, MK204411, MK211169, MK211369, MK211370, MK211371, MK211372, MK211373, MK211374, MK211375, MK211376, MK211377, MK211378, MK211379, MK217372, MK217373, MK217374, MK217375, MK250953, MK280984, MK288006, MK303619, MK303620, MK303621, MK303622, MK303623, MK303624, MK303625, MK309398, MK329221, MK330604, MK330605, MK334043, MK334044, MK334045, MK334046, MK334047, MK355396, MK357908, MK357909, MK359255, MK392335, MK409657, MK409658, MK409659, MK423876, MK423877, MK462243, MK462244, MK462245, MK462246, MK462247, MK462248, MK462249, MK462250, MK462251, MK462252, MK462253, MK462254, MK462255, MK462256, MK472067, MK472068, MK472069, MK472070, MK472071, MK482396, MK482397, MK483839, MK492263, MK558089, MK559454, MK559455, MK559456, MK564474, MK564475, MK572803, MK574042, MK574043, MK581200, MK581201, MK581202, MK581203, MK581204, MK581205, MK581206, MK581207, MK581208, MK584552, MK606368, MK606369, MK644086, MK644601, MK644602, MK644603, MK644604, MK644605, MK651076, MK673545, MK679660, MK690502, MK702008, MK720944, MK720945, MK720946, MK728875, MK796238, MK796425, MK841494, MK841495, MK862249, MK878536, MK907286, MK907287, MK937828, MK937829, MK937830, MK937831, MK937832, MK937833, MK967708, MK977618, MK993519, MN025260, MN026164, MN037494, MN056942, MN065811, MN096598, MN120513, MN120514, MN165107, MN197549, MN249445, MN306018, MN306036, MN306040, MN306041, MN306042, MN306043, MN306046, MN306053, MN310476, MN310478, MN315264, MN365232, MN365233, MN369046, MN486588, MN507638, MN512434, MN512435, MN512436, MN512437, MN512438, MN514962, MN514963, MN514964, MN514965, MN514966, MN514967, MN599049, MN611517, MN611518, MN611519, MN611520, MN611521, MN611522, MN611523, MN611524, MN611525, MN723542, MN723543, MN723544, MN996532, NC_001451, NC_001846, NC_002306, NC_002645. NC_003045, NC_003436, NC_004718, NC_005831, NC_006213, NC_006577, NC_009019, NC_009020, NC_009021, NC_009657, NC_009988, NC_010437, NC_010438, NC_010646, NC_010800, NC_011547, NC_011549, NC_011550, NC_012936, NC_014470, NC_016990, NC_016991, NC_016992, NC_016993, NC_016994, NC_016995, NC_016996, NC_017083, NC_018871, NC_019843, NC_022103, NC_022643, NC_023760. NC_025217, NC_026011, NC_028752, NC_028806, NC_028811, NC_028814, NC_028824, NC_028833, NC_030292, NC_030886, NC_032107, NC_032730, NC_034440, NC_034972, NC_038294, NC_038861. NC_039207, NC_039208, or U00735. In some embodiments, the reference viral genome of the GenBank Access IDs described herein is associated with any one of the alphacoronavirus, betacoronavirus, deltacoronavirus, or gammacoronavirus described herein. herein In some embodiments, the crRNA complements and binds to the reference viral genome of the GenBank Access IDs described herein. Non-limiting examples of the reference viral genome or a fragment thereof can include SEQ ID Nos listed in Table 4 and SEQ ID Nos: 13101-13122.

In some embodiments, the viral genomes being targeted, cleaved, or degraded by the systems and methods described herein can be either coding regions (i.e. viral genes), non-coding regions, or open reading frames. In some embodiments, the viral genomes being targeted, cleaved, or degraded by the systems and methods described can span across coding regions, non-coding regions, and opening reading frames. Exemplary viral coding regions, viral genes, or viral open reading frames that can be targeted and bound by the systems and methods described herein include orf1a, orf1ab, S (spike), 3a, 3b, E (envelope protein), M (matrix protein), p6, 7a, 7b, 8b, 9b, N (nucleocapsid), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRP), nsp13 (RNA 5′-triphosphatase), nsp14 (3′-to-5′ exonuclease), nsp15 (endoRNAse), and nsp16 (2′-O-ribose methyltransferase).

In some embodiments, the viral genomes, viral genes, or viral gene segments being targeted, cleaved, or degraded by the systems and methods described herein can be viral genomes, viral genes, or viral gene segments of influenza viruses, any type thereof, any subtype thereof, any clade thereof, or any sub-clade thereof. In some embodiments, the influenza virus is selected from the genera consisting of Influenza virus A, Influenza virus B, Influenza virus C and Influenza virus D. In further embodiments, the influenza A virus is of the subtype H1N1, H1N2, H2N2 or H3N2. In further embodiments, the influenza B virus of the B/Yamagata/16/88-like lineage or the B/Victoria/2/87-like lineage.

In some embodiments, the viral genomes, viral genes, or viral gene segments being targeted by the systems and the methods described herein can be selected from any genome registered on GenBank (as of Mar. 12, 2020). In some embodiments, the viral genomes, viral genes, or viral gene segments being targeted by the systems and the methods described herein can be selected from any genome (i.e. thousands of influenza A viral (IAV) genome sequences from epidemic and pandemic strains publicly available) registered on a public database (as of Mar. 12, 2020), such as influenza research database (IRD). In some embodiments, the viral genomes, viral genes, or viral gene segments being targeted by the systems and the methods described herein can be an influenza virus, such as influenza A, influenza B, influenza C, and influenza D. In some embodiments, the viral genomes, viral genes, or viral gene segments being targeted by the systems and the methods described herein can be an influenza A. Examples of an influenza can include, but are not limited to, influenza A types H1, H3, H5, H7, and H9 viruses. In some embodiments, the viral genomes, viral genes, or viral gene segments being targeted by the systems and the methods described herein can be selected from any strain of Column 1 of Table 6.

TABLE 6
Exemplary Influenza Strain Counts of Segment 1 Sequences

| Flu Strain Analyzed | Segment 1 Sequences Analyzed |
|---|---|
| Unknown | 2 |
| N5 | 1 |
| H10N8 | 111 |
| H10N9 | 43 |
| H10N1 | 63 |
| H10N2 | 46 |
| H10N3 | 110 |
| H10N4 | 63 |
| H10N5 | 132 |
| H10N6 | 63 |
| H10N7 | 631 |
| H4Nx | 1 |
| H18N11 | 4 |
| H6N3 | 14 |
| H2N4 | 2 |
| H2N5 | 22 |
| H2N6 | 5 |
| H2N7 | 32 |
| H2N1 | 46 |
| H2N2 | 166 |
| H2N3 | 242 |
| H2N8 | 15 |
| H2N9 | 77 |
| H8N8 | 2 |
| H6N4 | 13 |
| H8N6 | 1 |
| H8N4 | 160 |
| HxN1 | 1 |
| H8N2 | 3 |
| H8N3 | 1 |
| H8N1 | 1 |
| H14N7 | 2 |
| H7 | 3 |
| H3N2v | 1 |
| H17N10 | 3 |
| H9N6 | 5 |
| H11 | 7 |
| H12 | 1 |
| H91 | 1 |
| Mixed | 34 |
| H9N8 | 6 |
| H14N2 | 2 |
| H3 | 59 |
| H1 | 22 |
| H6 | 2 |
| H5N6 | 349 |
| H4 | 2 |
| H5 | 4 |
| H5N7 | 19 |
| H4N3 | 20 |
| mixed | 1221 |
| H4N1 | 14 |
| H4N6 | 1410 |
| H11N7 | 10 |
| H5N2 | 1082 |
| H3N9 | 14 |
| H3N8 | 1538 |
| H12N9 | 5 |
| H4N4 | 21 |
| H3N1 | 97 |
| H3N3 | 31 |
| H3N2 | 18955 |
| H3N5 | 21 |
| H3N4 | 5 |
| H3N7 | 26 |
| H3N6 | 239 |
| H4N8 | 254 |
| H4N9 | 26 |
| H5N9 | 37 |
| H5N8 | 238 |
| H4N2 | 169 |
| unknown | 4 |
| H5N5 | 43 |
| H5N4 | 5 |
| H5N3 | 118 |
| H4N7 | 7 |
| H5N1 | 2233 |
| H4N5 | 14 |
| H6N8 | 228 |
| H6N9 | 10 |
| H7N9 | 870 |
| H7N8 | 30 |
| H7N5 | 5 |
| H6N1 | 416 |
| H7N7 | 287 |
| H7N6 | 60 |
| H7N1 | 157 |
| H6N5 | 80 |
| H6N6 | 232 |
| H6N7 | 5 |
| H?N4 | 2 |
| H12N6 | 6 |
| H11N8 | 28 |
| N8 | 4 |
| H3Nx | 2 |
| H15N6 | 1 |
| H7N4 | 32 |
| N1 | 13 |
| N2 | 10 |
| N3 | 2 |
| N4 | 3 |
| H6N2 | 566 |
| N6 | 12 |
| N7 | 2 |
| H1N9 | 61 |
| H1N8 | 36 |
| H1N3 | 74 |
| H1N2 | 1781 |
| H1N1 | 15575 |
| H1N7 | 10 |
| H1N6 | 41 |
| H1N5 | 13 |
| H1N4 | 9 |
| H9N3 | 4 |
| H9N2 | 1579 |
| H9N1 | 27 |
| H9N7 | 9 |
| H7N3 | 678 |
| H9N5 | 21 |
| H9N4 | 2 |
| H9N9 | 15 |
| H7N2 | 269 |
| H11N5 | 9 |
| H12N8 | 13 |
| H11N6 | 15 |
| H11N1 | 38 |
| H11N3 | 44 |
| H11N2 | 131 |
| H12N2 | 5 |
| H12N3 | 20 |
| H12N1 | 16 |
| H11N9 | 411 |
| H12N7 | 9 |
| H12N4 | 44 |
| H12N5 | 215 |
| H1N2v | 6 |
| H16N8 | 1 |
| H16N3 | 256 |
| H15N9 | 8 |
| H15N8 | 1 |
| H14N8 | 2 |
| H14N4 | 2 |
| H14N5 | 4 |
| H14N6 | 5 |
| H15N2 | 1 |
| H15N5 | 6 |
| H15N4 | 1 |
| H15N7 | 1 |
| H14N3 | 19 |
| H13N6 | 238 |
| H13N3 | 3 |
| H13N2 | 91 |
| H13N9 | 67 |
| H13N8 | 119 |

In some embodiments, the target viral genome segment comprises a sequence having at least 90% sequence identity to a sequence selected from SEQ ID NOs:13501-13548. In some embodiments, the target viral genome segment comprises a sequence having at least 95% sequence identity to a sequence selected from SEQ ID NOs:13501-13548. In some embodiments, the target viral genome segment comprises a sequence having 100% sequence identity to a sequence selected from SEQ ID NOs:13501-13548.

In some embodiments, the target viral genome segment comprises at least one non-coding region or a fragment thereof. The at least one non-coding region may be a 5′ end or a 3′ end of a segment (such as Segment I, Segment II, Segment 111, Segment IV, Segment V, Segment VI, Segment VII, or Segment VIII) that comprise the influenza A viral (IAV) segmented RNA genome. The at least one non-coding region may have a length of, or of about, 10, 11, 12, 13, 14, 15, 16, 17, 18, 19, 20, 21, 22, 23, 24, or 25 base pairs (bp), or any range between any two of the foregoing values. The at least one non-coding region may have a length of from 10 to 25, from 10 to 22, from 10 to 20, or from 12 to 18, or from 14 to 17 bp. The at least one non-coding region may be associated with packaging an influenza A viral RNA into a budding virion. One of skill in the art will understand that a coding region adjacent to the at least one non-coding region (NCR) may serve not only to encode one or more viral proteins, but also as a packing signal for an influenza viral genome segment. One of skill in the art will also understand that the at least one non-coding region (NCR) may by itself be shown to be sufficient for packaging an influenza A viral RNA into a budding virion, or that the efficiency of packaging increases when at least a portion of a coding region adjacent to the at least one NCR is included. In some embodiments, the target viral genome segment further comprises a fragment of a coding region adjacent to the at least one non-coding region.

In some embodiments, the viral genomes being targeted, cleaved, or degraded by the systems and methods described herein can be either coding regions (i.e. viral genes, or viral gene segments), non-coding regions, or open reading frames. In some embodiments, the viral genomes being targeted, cleaved, or degraded by the systems and methods described can span across coding regions, non-coding regions, and opening reading frames.

In some embodiments, the at least one heterologous RNA polynucleotide comprising a nucleic acid sequence of any one SEQ ID NOs: 13601-13606 targets an endogenous gene of the cell being infected by any one of strain of the virus described herein. In some embodiments, the endogenous gene is a member of the peptidase family M1 such as aminopeptidase N (ANPEP), aminopeptidase A, leukotriene A4 hydrolase, Ape2 aminopeptidase, Aap1′ aminopeptidase, pyroglutamyl-peptidase II, cytosol alanyl aminopeptidase, cystinyl aminopeptidase, aminopeptidase G, aminopeptidase B, aminopeptidase Ey, endoplasmic reticulum aminopeptidase 1, tricom interacting factor F2, tricorn interacting factor F3, arginyl aminopeptidase-like 1, ERAP2 aminopeptidase, aminopeptidase, aminopeptidase O, or Tata binding protein associated factor. In some embodiments, the endogenous gene targeted by the heterologous RNA polynucleotide is ANPEP. In some embodiments, the endogenous gene is a member of the minor histocompatibility antigen family such as AKAP13, APOBEC3B, ARHGDIB, BCAT2, BCL2A1, CD19, CENPM, CTSH, C19orf48, DDX3Y, DPH1, EB13, ERAP1, ERBB2, GEMIN4, HEATR1, ARHGAP45, HMHB1, HMSD, KDM5D, PUM3, RESF1, LY75, MR1, MTHFD1, MYO1G, NUP133, PDCD11, PI4K2B, PKN3, PRCP, PTK2B, P2RX5, RPS4Y1, SLC1A5, SLC19A1, SON, SP110, SSR1, SWAP70, TMSB4Y, TOR3A, TRIM22, TRIM42, TRIP10, TYMP, UGT2B17, USP9Y, UTY, WNK1, or ZNF419. In some embodiments, the endogenous gene targeted by the heterologous RNA polynucleotide is MTHFD1. In some embodiments, the endogenous gene is a member of the type II transmembrane serine protease family such as HAT/DESC subfamily: HAT. DESC1, TMPRSS11A, HAT-like 2, HAT-like 3, HAT-like 4, HAT-like 5; Hepsin/TMPRSS subfamily: Hepsin, TMPRSS2, TMPRSS3, TMPRSS4, MSPL, Spinesin, Enteropeptidase; Matriptase subfamily: Matriptase, Matriptase-2, Matripase-3, Polyserease-1; and Corin subfamily Corin. In some embodiments, the endogenous gene targeted by the heterologous RNA polynucleotide is TMPRSS2.

In some embodiments, the at least one heterologous RNA polynucleotide comprising a nucleic acid sequence of any one of SEQ ID NOs: 13607-13649 or 13701-13729 can target the RdRP, N-protein, or the viral genome of any one strain of the coronavirus described herein. In some embodiments, a combination of the at least one heterologous RNA polynucleotide comprising a nucleic acid sequence of any one of SEQ ID NOs: 13651-13664 or 13671-13684 can target any one strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the RNA virus described herein. Non-limiting examples of the RNA virus include RNA virus families of Coronaviridae, Pneumoviridae, Paramyxoviridae, Caliciviridae, Hepeviridae, Picomaviridae, Filoviridae, Flaviviridae, Rhabdoviridae, or Togaviridae.

In some embodiments, the viral genomes, viral genes, or the transcripts of the viral genomes and viral genes targeted, cleaved, or degraded can be deoxyribonucleic acid (DNA). In some cases, the DNA can be single-stranded or doubled-stranded. In some embodiments, the viral genomes, viral genes, or the transcripts of the viral genomes and viral genes targeted, cleaved, or degraded can be ribonucleic acid (RNA). In some instances, the RNA can be mRNA, rRNA, SRP RNA, tRNA, tmRNA, snRNA, snoRNA, gRNA, aRNA, crRNA, lncRNA, miRNA, ncRNA, piRNA, siRNA, or shRNA. In some embodiments, the target RNA is an mRNA.

In some embodiments, the systems and methods described herein target and bind to at least a portion of the target viral genomes or viral genes. In some embodiments, the systems and methods described herein target and bind to at least a portion of the target viral genomes or viral genes for cleavage or degradation. In some embodiments, the portion of the viral genomes or viral genes being targeted and bound by the systems and methods described herein is between 5 nt to 100 nt. In some embodiments, the portion of the viral genomes or viral genes being targeted and bound by the systems and methods described herein is between 5 nucleotides (nt) to 10 nt, 5 nt to 15 nt, 5 nt to 20 nt, 5 nt to 25 nt, 5 nt to 30 nt, 5 nt to 40 nt, 5 nt to 50 nt, 5 nt to 60 nt, 5 nt to 70 nt, 5 nt to 80 nt, 5 nt to 100 nt, 10 nt to 15 nt, 10 nt to 20 nt, 10 nt to 25 nt, 10 nt to 30 nt, 10 nt to 40 nt, 10 nt to 50 nt, 10 nt to 60 nt, 1.0 nt to 70 nt, 10 nt to 80 nt, 10 nt to 100 nt, 15 nt to 20 nt, 15 nt to 25 nt, 15 nt to 30 nt, 15 nt to 40 nt, 15 nt to 50 nt, 15 nt to 60 nt, 15 nt to 70 nt, 15 nt to 80 nt, 15 nt to 100 nt, 20 nt to 25 nt, 20 nt to 30 nt, 20 nt to 40 nt, 20 nt to 50 nt, 20 nt to 60 nt, 20 nt to 70 nt, 20 nt to 80 nt, 20 nt to 100 nt, 25 nt to 30 nt, 25 nt to 40 nt, 25 nt to 50 nt, 25 nt to 60 nt, 25 nt to 70 nt, 25 nt to 80 nt, 25 nt to 100 nt, 30 nt to 40 nt, 30 nt to 50 nt, 30 nt to 60 nt, 30 nt to 70 nt, 30 nt to 80 nt, 30 nt to 100 nt, 40 nt to 50 nt, 40 nt to 60 nt, 40 nt to 70 nt, 40 nt to 80 nt, 40 nt to 100 nt, 50 nt to 60 nt, 50 nt to 70 nt, 50 nt to 80 nt, 50 nt to 100 nt, 60 nt to 70 nt, 60 nt to 80 nt, 60 nt to 100 nt, 70 nt to 80 nt, 70 nt to 100 nt, or 80 nt to 100 nt. In some embodiments, the portion of the viral genomes or viral genes being targeted and bound by the systems and methods described herein is comprises at least about 5 nt, 10 nt, 15 nt, 20 nt, 25 nt, 30 nt, 40 nt, 50 nt, 60 nt, 70 nt, 80 nt, 100 nt, or more. In some embodiments, the portion of the viral genomes or viral genes being targeted and bound by the systems and methods described herein comprises about 5 nt, 10 nt, 15 nt, 20 nt, 25 nt, 30 nt, 40 nt, 50 nt, 60 nt, 70 nt, or 80 nt. In some embodiments, the portion of the viral genomes or viral genes being targeted and bound by the systems and methods described herein comprises at most about 100 nt, 90 nt, 80 nt, 70 nt, 60 nt, 50 nt, 40 nt, 30 nt, 25 nt, 20 nt, 15 nt, 10 nt, 5 nt, or less.

In some embodiments, the portion of the viral genomes or viral genes being targeted and bound by the systems and methods described herein is at least 5 nt to 100 nt. In some embodiments, the portion of the viral genomes or viral genes being targeted and bound by the systems and methods described herein is at least 5 nt to 10 nt, 5 nt to 15 nt, 5 nt to 20 nt, 5 nt to 25 nt, 5 nt to 30 nt, 5 nt to 40 nt, 5 nt to 50 nt, 5 nt to 60 nt, 5 nt to 70 nt, 5 nt to 80 nt, 5 nt to 100 nt, 10 nt to 15 nt, 10 nt to 20 nt, 10 nt to 25 nt, 10 nt to 30 nt, 10 nt to 40 nt, 10 nt to 50 nt, 10 nt to 60 nt, 10 nt to 70 nt, 10 nt to 80 nt, 10 nt to 100 nt, 15 nt to 20 nt, 15 nt to 2.5 nt, 15 nt to 30 nt, 15 nt to 40 nt, 15 nt to 50 nt, 15 nt to 60 nt, 15 nt to 70 nt, 15 nt to 80 nt, 15 nt to 100 nt, 20 nt to 25 nt, 20 nt to 30 nt, 20 nt to 40 nt, 20 nt to 50 nt, 20 nt to 60 nt, 20 nt to 70 nt, 20 nt to 80 nt, 20 nt to 100 nt, 25 nt to 30 nt, 25 nt to 40 nt, 25 nt to 50 nt, 25 nt to 60 nt, 25 nt to 70 nt, 25 nt to 80 nt, 25 nt to 100 nt, 30 nt to 40 nt, 30 nt to 50 nt, 30 nt to 60 nt, 30 nt to 70 nt, 30 nt to 80 nt, 30 nt to 100 nt, 40 nt to 50 nt, 40 nt to 60 nt, 40 nt to 70 nt, 40 nt to 80 nt, 40 nt to 100 nt, 50 nt to 60 nt, 50 nt to 70 nt, 50 nt to 80 nt, 50 nt to 100 nt, 60 nt to 70 nt, 60 nt to 80 nt, 60 nt to 100 nt, 70 nt to 80 nt, 70 nt to 100 nt, or 80 nt to 100 nt. In some embodiments, the portion of the viral genomes or viral genes being targeted and bound by the systems and methods described herein is at most 5 nt to 100 nt. In some embodiments, the portion of the viral genomes or viral genes being targeted and bound by the systems and methods described herein is at most 5 nt to 10 nt, 5 nt to 15 nt, 5 nt to 20 nt, 5 nt to 25 nt, 5 nt to 30 nt, 5 nt to 40 nt, 5 nt to 50 nt, 5 nt to 60 nt, 5 nt to 70 nt, 5 nt to 80 nt, 5 nt to 100 nt, 10 nt to 15 nt, 10 nt to 20 nt, 10 nt to 25 nt, 10 nt to 30 nt, 10 nt to 40 nt, 10 nt to 50 nt, 10 nt to 60 nt, 10 nt to 70 nt, 10 nt to 80 nt, 10 nt to 100 nt, 15 nt to 20 nt, 15 nt to 25 nt, 15 nt to 30 nt, 15 nt to 40 nt, 15 nt to 50 nt, 15 nt to 60 nt, 15 nt to 70 nt, 15 nt to 80 nt, 15 nt to 100 nt, 20 nt to 25 nt, 20 nt to 30 nt, 20 nt to 40 nt, 20 nt to 50 nt, 20 nt to 60 nt, 20 nt to 70 nt, 20 nt to 80 nt, 20 nt to 100 nt, 25 nt to 30 nt, 25 nt to 40 nt, 25 nt to 50 nt, 25 nt to 60 nt, 25 nt to 70 nt, 25 nt to 80 nt, 25 nt to 100 nt, 30 nt to 40 nt, 30 nt to 50 nt, 30 nt to 60 nt, 30 nt to 70 nt, 30 nt to 80 nt, 30 nt to 100 nt, 40 nt to 50 nt, 40 nt to 60 nt, 40 nt to 70 nt, 40 nt to 80 nt, 40 nt to 100 m, 50 nt to 60 nt, 50 nt to 70 nt, 50 nt to 80 nt, 50 nt to 100 nt, 60 nt to 70 nt, 60 nt to 80 nt, 60 nt to 100 nt, 70 nt to 80 nt, 70 nt to 100 nt, or 80 nt to 100 nt.

B. Gene Regulating Moiety

In some instances, the systems and methods described herein comprise a gene regulating moiety to regulate the expressions of viral genomes or viral genes. In some embodiments, the gene regulating moiety comprises a CRISPR-Cas polypeptide. In some embodiments, the gene regulating moiety can be, for example, Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or a functional fragment thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Casl, CaslB, Cas2, Cas3, Cas4, Cas5, Cas5e (CasD), Cas6, Cas6e, Cas6f, Cas7, Cas8a, Cas8a1, Cas8a2, Cas8b, Cas8c, Csnl, Csxl2, Cas10, Cas10d, CaslO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csxl7, Csxl4, CsxlO, Csxl6, CsaX, Csx3, Csxl, Csxl5, Csfl, Csf2, Csf3, Csf4, or Cul966; any derivative thereof; any variant thereof; or any fragment thereof. In some embodiments, Cas13 can include, but are not limited to, Cas13a, Cas13b, Cas13c, and Cas 13d (e.g., CasRx). CRISPR/Cas can be DNA and/or RNA cleaving, or can exhibit reduced cleavage activity. Gene regulating moiety can be configured to complex with at least one heterologous RNA polynucleotide. In some cases, the gene regulating moiety can be fused with a transcription activator or transcription repressor.

Any suitable nuclease (e.g., endonuclease) can be used in as the gene regulating moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases: RNA-binding proteins (RBP); CRISPR-associated RNA binding proteins; recombinases; flippases; transposases; Argonaute (Ago) proteins (e.g., prokarvotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

A gene regulating moiety as disclosed herein can be coupled (e.g., linked or fused) to additional peptide sequences which are not involved in regulating gene expression, for example linker sequences, targeting sequences, etc. The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

A gene regulating moiety as disclosed herein can be a part of a fusion construct (e.g., a fusion protein). As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity (e.g., a reverse transcriptase activity), ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. A fusion protein can be a non-native sequence in a Cas protein.

In some embodiments, the gene regulating moiety can be fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the protein. In some cases, the gene regulating moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-κB p65 subunit. Epstein-Barr virus R transactivator (Rta) and are described in Chavez et al., Nat Methods, 2015, 12(4):326-328 and U.S. Patent App. Publ. No. 20140068797. Non-limiting exemplary repression domains include the KRAB (Kruppel-associated box) domain of Kox1, the Mad mSIN3 interaction domain (SID), ERF repressor domain (ERD), and are described in Chavez et al., Nat Methods, 2015, 12(4):326-328 and U.S. Patent App. Publ. No. 20140068797. In some embodiments, the gene regulating moiety includes one or more tandem repressor domains located at the amino terminus of the protein.

In some embodiments, the nuclease disclosed herein can be a protein that lacks nucleic acid cleavage activity. In some cases, a Cas protein is a dead Cas protein. A dead Cas protein can be a protein that lacks nucleic acid cleavage activity. A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

In some embodiments, a dCas (e.g., dCas9) polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell.

In some embodiments, the gene regulating moiety can comprise a catalytically inactive Cas polypeptide, where the nuclease activity of the Cas polypeptide is eliminated or substantially eliminated.

In some instances, the gene regulating moiety can comprise a catalytically inactivated Cas9 (dCas9), any derivative thereof; any variant thereof: or any fragment thereof.

In some instances, the gene regulating moiety can comprise a catalytically inactivated Cas12 (dCas12), any derivative thereof; any variant thereof: or any fragment thereof.

In some instances, the gene regulating moiety can comprise a catalytically inactivated Cas13 (dCas13); any derivative thereof; any variant thereof; or any fragment thereof.

In some embodiments, the gene regulating moiety can be complexed with the at least one heterologous nucleic acid polynucleotide as described herein. In some embodiments, the at least one heterologous nucleic acid polynucleotide can be either heterologous DNA polynucleotide or heterologous RNA polynucleotide. In some embodiments, the gene regulating moiety can be complexed with at least one heterologous RNA polynucleotide. In some cases, the complexing with the at least one heterologous RNA polynucleotide direct and target the gene regulating moiety to the portion of the viral genomes, viral genes, or transcripts of the viral genomes or viral genes targeted. In some cases, the complexing with the at least one heterologous RNA polynucleotide direct and target the gene regulating moiety to the portion of the viral genomes, viral genes, or transcripts of the viral genomes or viral genes targeted for cleavage or degradation. In some cases, the complexing with the at least one heterologous RNA polynucleotide direct and target the gene regulating moiety to the portion of the viral genomes, viral genes, or transcripts of the viral genomes or viral genes, where the gene regulating moiety reduces the expression of the viral genomes or viral genes in a cell.

In some embodiments, the gene regulating moiety can reduce the expression of the viral genomes or viral genes between about 0.1 fold to about 10,000 fold. In some embodiments, the gene regulating moiety can reduce the expression of the viral genomes or viral genes between about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of the viral genomes or viral genes between about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the viral genomes or viral genes between at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the viral genomes or viral genes between at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of the viral genomes or viral genes by at least about 0.1 fold to about 10,000 fold. In some embodiments, the gene regulating moiety can reduce the expression of the viral genomes or viral genes by at least about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of the viral genomes or viral genes by at least about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the viral genomes or viral genes by at least at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the viral genomes or viral genes by at least at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of the viral genomes or viral genes by at most about 0.1 fold to about 10,000 fold. In some embodiments, the gene regulating moiety can reduce the expression of the viral genomes or viral genes by at most about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of the viral genomes or viral genes by at most about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the viral genomes or viral genes by at most at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the viral genomes or viral genes by at most at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of viral RdRP between about 0.1 fold to about 10,000 fold. In some embodiments, the gene regulating moiety can reduce the expression of viral RdRP between about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of viral RdRP between about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of viral RdRP between at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of viral RdRP between at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of viral RdRP by at least about 0.1 fold to about 10,000 fold. In some embodiments, the gene regulating moiety can reduce the expression of viral RdRP by at least about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of viral RdRP by at least about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of viral RdRP by at least at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of viral RdRP by at least at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of viral RdRP by at most about 0.1 fold to about 10,000 fold. In some embodiments, the gene regulating moiety can reduce the expression of viral RdRP by at most about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of viral RdRP by at most about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of viral RdRP by at most at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of viral RdRP by at most at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of viral N protein between about 0.1 fold to about 10,000 fold. In some embodiments, the gene regulating moiety can reduce the expression of viral N protein between about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of viral N protein between about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of viral N protein between at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of viral N protein between at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of viral N protein by at least about 0.1 fold to about 10,000 fold. In some embodiments, the gene regulating moiety can reduce the expression of viral N protein by at least about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of viral N protein by at least about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of viral N protein by at least at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of viral N protein by at least at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the expression of viral N protein by at most about 0.1 fold to about 10,000 fold. In some embodiments, the gene regulating moiety can reduce the expression of viral N protein by at most about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of viral N protein by at most about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of viral N protein by at most at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of viral N protein by at most at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the reduction of expression can be detected by measuring the amount of RNA encoding the targeted viral genome or viral gene in the cell and/or measuring the amount of polypeptide (e.g., the RdRP, N-protein, or a fragment thereof) in the cell that is encoded by the targeted viral gene.

In some embodiments, the gene regulating moiety can reduce the transfection of influenza virus between about 0.1 fold to about 10,000 fold. In some embodiments, the gene regulating moiety can reduce the transfection of influenza virus between about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the transfection of influenza virus between about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the transfection of influenza virus between at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the transfection of influenza virus between at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the transfection of influenza virus by at least about 0.1 fold to about 10,000 fold. In some embodiments, the gene regulating moiety can reduce the transfection of influenza virus by at least about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the transfection of influenza virus by at least about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the transfection of influenza virus by at least at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the transfection of influenza virus by at least at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the gene regulating moiety can reduce the transfection of influenza virus by at most about 0.1 fold to about 10,000 fold. In some embodiments, the gene regulating moiety can reduce the transfection of influenza virus by at most about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the transfection of influenza virus by at most about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the transfection of influenza virus by at most at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the transfection of influenza virus by at most at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some embodiments, the reduction of expression can be detected by measuring the amount of RNA encoding the targeted viral genome or viral gene or viral gene segments in the cell and/or measuring the amount of polypeptide (e.g. PB2, PB2, PB1-F2, PA, HA, NP, NA, M1, M2, NS1, and/or NEP/NS2, or a fragment thereof) in the cell that is encoded by the targeted viral gene, or targeted viral gene segments.

In some embodiments, the at least one heterologous RNA polynucleotide comprising a nucleic acid sequence of any one SEQ ID NOs: 13601-13606 targets an endogenous gene of the cell being infected by any one of strain of the virus described herein. In some embodiments, the endogenous gene is a member of the peptidase family M1 such as aminopeptidase N (ANPEP), aminopeptidase A, leukotriene A4 hydrolase, Ape2 aminopeptidase, Aap1′ aminopeptidase, pyroglutamyl-peptidase II, cytosol alanyl aminopeptidase, cystinyl aminopeptidase, aminopeptidase G, aminopeptidase B, aminopeptidase Ey, endoplasmic reticulum aminopeptidase 1, tricom interacting factor F2, tricom interacting factor F3, arginyl aminopeptidase-like 1, ERAP2 aminopeptidase, aminopeptidase, aminopeptidase O, or Tata binding protein associated factor. In some embodiments, the endogenous gene targeted by the heterologous RNA polynucleotide is ANPEP. In some embodiments, the endogenous gene is a member of the minor histocompatibility antigen family such as AKAP13, APOBEC3B, ARHGDIB, BCAT2, BCL2A1, CD19, CENPM, CTSH, C19orf48, DDX3Y, DPH1, EBI3, ERAP1, ERBB2, GEMIN4, HEATR1, ARHGAP45, HMHB1, HMSD, KDM5D, PUM3, RESF1, LY75, MR1, MTHFD1, MYO1G, NUP133, PDCD11, PI4K2B, PKN3, PRCP, PTK2B, P2RX5, RPS4Y1, SLC1A5, SLC19A1, SON, SP110, SSR1, SWAP70, TMSB4Y, TOR3A, TRIM22, TRIM42, TRIP10, TYMP, UGT2B17, USP9Y, UTY, WNK1, or ZNF419. In some embodiments, the endogenous gene targeted by the heterologous RNA polynucleotide is MTHFD1. In some embodiments, the endogenous gene is a member of the type II transmembrane serine protease family such as HAT/DESC subfamily: HAT, DESC1, TMPRSS11A, HAT-like 2, HAT-like 3, HAT-like 4, HAT-like 5; Hepsin/TMPRSS subfamily: Hepsin, TMPRSS2, TMPRSS3, TMPRSS4, MSPL, Spinesin, Enteropeptidase; Matriptase subfamily: Matriptase, Matriptase-2, Matripase-3, Polyserease-1; and Corin subfamily Corin. In some embodiments, the endogenous gene targeted by the heterologous RNA polynucleotide is TMPRSS2.

In some embodiments, the at least one heterologous RNA polynucleotide comprising a nucleic acid sequence of any one of SEQ ID NOs: 13607-13649 or 13701-13729 can target the RdRP. N-protein, or the viral genome of any one strain of the coronavirus described herein. In some embodiments, a combination of the at least one heterologous RNA polynucleotide comprising a nucleic acid sequence of any one of SEQ ID NOs: 13651-13664 or 13671-13684 can target any one strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the RNA virus described herein. Non-limiting examples of the RNA virus include RNA virus families of Coronaviridae, Pneumoviridae, Paramyxoviridae, Caliciviridae, Hepeviridae, Picomaviridae, Filoviridae, Flaviviridae, Rhabdoviridae, or Togaviridae.

C. Heterologous Nucleic Acid Polynucleotide

In some cases, the systems and methods described herein comprise at least one heterologous nucleic acid polynucleotide. In some cases, the systems and methods described herein comprise a plurality of heterologous nucleic acids. In some embodiments, the polynucleotide can be deoxyribonucleic acid (DNA). In some cases, the DNA sequence can be single-stranded or doubled-stranded. In some embodiments, the at least one heterologous nucleic acid polynucleotide can be ribonucleic acid (RNA).

In some embodiments, the gene regulating moiety can be complexed with the at least one heterologous RNA polynucleotide. The at least one heterologous RNA polynucleotide can comprise a nucleic-acid targeting region that comprises a complementary sequence to a nucleic acid sequence on the targeted polynucleotide such as the targeted viral genomes, viral genes, or transcripts of the viral genomes or viral genes to confer the sequence specificity of gene regulating moiety-dependent targeting. In some embodiments, the at least one heterologous RNA polynucleotide can be guide nucleic acid (or guide RNA) comprising two separate nucleic acid molecules, which can be referred to as a double guide nucleic acid or a single nucleic acid molecule, which can be referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence of a targeted polynucleotide can be identified by identifying a PAM within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility. % GC, frequency of genomic occurrence, methylation status, presence of SNPs, and the like.

The percent complementarity between the nucleic acid-targeting sequence (e.g., a spacer sequence of the at least one heterologous polypeptide as disclosed herein) and the target nucleic acid (e.g., a protospacer sequence of the one or more target viral genes as disclosed herein) can be at least 50%, at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp.

In some embodiments, the dsRNA duplex of the Cas protein-binding segment can have a length of 36 base pairs. The percent complementarity between the nucleotide sequences that hybridize to form the dsRNA duplex of the protein-binding segment can be at least about 60%. For example, the percent complementarity between the nucleotide sequences that hybridize to form the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The linker (e.g., that links a crRNA and a tracrRNA in a single guide nucleic acid) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a DNA-targeting RNA is 4 nt.

Guide nucleic acids of the disclosure can include modifications or sequences that provide for additional desirable features (e.g., modified or regulated stability, subcellular targeting; tracking with a fluorescent label; a binding site for a protein or protein complex; and the like). Examples of such modifications include, for example, a 5′ cap (a 7-methylguanylate cap (m7G)); a 3′ polyadenylated tail (a 3′ poly(A) tail); a riboswitch sequence (e.g., to allow for regulated stability and/or regulated accessibility by proteins and/or protein complexes); a stability control sequence: a sequence that forms a dsRNA duplex (a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof.

A guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base portion of the nucleotide can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds can be suitable. In addition, linear compounds can have internal nucleotide base complementarity and can therefore fold in a manner as to produce a fully or partially double-stranded compound. Further, within guide nucleic acids, the phosphate groups can commonly be referred to as forming the internucleoside backbone of the guide nucleic acid. The linkage or backbone of the guide nucleic acid can be a 3′ to 5′ phosphodiester linkage.

A guide nucleic acid can comprise a modified backbone and/or modified internucleoside linkages. Modified backbones can include those that retain a phosphorus atom in the backbone and those that do not have a phosphorus atom in the backbone.

Suitable modified guide nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3 to 3′, a 5′ to 5′ or a 2′ to 2′ linkage. Suitable guide nucleic acids having inverted polarity can comprise a single 3′ to 3′ linkage at the 3′-most internucleotide linkage (such as a single inverted nucleoside residue in which the nucleobase is missing or has a hydroxyl group in place thereof). Various salts (e.g., potassium chloride or sodium chloride), mixed salts, and free acid forms can also be included.

A guide nucleic acid can comprise one or more phosphorothioate and/or heteroatom internucleoside linkages, in particular —CH2-NH—O—CH2-, —CH2-N(CH3)-O—CH2- (a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-).

A guide nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage replaces a phosphodiester linkage.

A guide nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A guide nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A guide nucleic acid can comprise linked morpholino units (morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be non-ionic mimics of guide nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2 oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A guide nucleic acid can comprise one or more substituted sugar moieties. Suitable polynucleotides can comprise a sugar substituent group selected from: OH; F: O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl. Particularly suitable are O((CH2)nO) mCH3, O(CH2)nOCH3, O(CH2)nNH2, O(CH2)nCH3, O(CH2)nONH2, and O(CH2)nON((CH2)nCH3)2, where n and m are from 1 to about 10. A sugar substituent group can be selected from: C1 to C10 lower alkyl, substituted lower alkyl, alkenyl, alkynyl, alkaryl, aralkyl, O-alkaryl or O-aralkyl. SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O—CH2 CH2OCH3, also known as 2′-O-(2-methoxyethyl) or 2′-MOE, an alkoxyalkoxy group). A further suitable modification can include 2′-dimethylaminooxyethoxy, (a O(CH2)2ON(CH3)2 group, also known as 2′-DMAOE), and 2′-dimethylaminoethoxyethoxy (also known as 2′-O-dimethyl-amino-ethoxy-ethyl or 2′-DMAEOE), 2′-O—CH2-O—CH2-N(CH3)2.

Other suitable sugar substituent groups can include methoxy (—O—CH3), aminopropoxy (—O CH2 CH2NH2), allyl (—CH2-CH═CH2), —O-allyl (—O—CH2-CH═CH2) and fluoro (F). 2′-sugar substituent groups can be in the arabino (up) position or ribo (down) position. A suitable 2′-arabino modification is 2′-F. Similar modifications can also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds can also have sugar mimetics such as cyclobutyl moieties in place of the pentofuranosyl sugar.

A guide nucleic acid can also include nucleobase (or “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties can include those in which the purine or pyrimidine base is replaced with other heterocycles, for example: 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and O-6 substituted purines, including 2-aminopropyladenine, 5-propynyluracil and 5-propynylcytosine, 5-methylcytosine substitutions can increase nucleic acid duplex stability by 0.6-1.2° C. and can be suitable base substitutions (e.g., when combined with 2′-O-methoxyethyl sugar modifications).

A modification of a guide nucleic acid can comprise chemically linking to the guide nucleic acid one or more moieties or conjugates that can enhance the activity, cellular distribution or cellular uptake of the guide nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

In some embodiments, the at least one heterologous RNA polynucleotide can bind to at least a portion of the viral genomes, viral genes, or transcripts of the viral genomes and viral genes. In some cases, the at least one heterologous RNA polynucleotide is capable forming a complex with the gene regulating moiety to direct the gene regulating moiety to target the portion of the viral genomes, viral genes, or transcripts of the viral genomes and viral genes. In some embodiments, the at least one heterologous RNA polynucleotide is capable forming a complex with the gene regulating moiety to direct the gene regulating moiety to target the portion of the viral genomes, viral genes, or transcripts of the viral genomes and viral genes for cleavage or degradation. In some instances, the at least one heterologous RNA polynucleotide is capable forming a complex with the gene regulating moiety to direct the gene regulating moiety to reduce expression of the targeted portion of the viral genomes or viral genes.

In some embodiments, the at least one heterologous RNA polynucleotide can be complementary and bind to the viral genomes, viral genes, or transcripts of the viral genomes and viral genes as described herein. In some embodiments, the at least one heterologous RNA polynucleotide can be complementary and bind to viral genomes, viral genes, or transcripts of the viral genomes and viral genes of coronavirus. In some embodiments, the at least one heterologous RNA polynucleotide can be complementary and bind to viral genomes, viral genes, or transcripts of the viral genomes and viral genes of alphacoronavirus, betacoronavirus, deltacoronavirus, or gammacoronavirus. Examples of alphacoronavirus can include, but are not limited to, Bat coronavirus CDPHE15, Bat coronavirus HKU10, Human coronavirus 229E, Human coronavirus NL63, Miniopterus bat coronavirus 1. Miniopterus bat coronavirus HKU8, Mink coronavirus 1, Porcine epidemic diarrhoea virus, Rhinolophus bat coronavirus HKU2, and Scotophilus bat coronavirus 512. Examples of betacoronavirus can include, but are not limited to, Betacoronavirus 1, Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus. Tylonycteris bat coronavirus HKU4. Examples of deltacoronavirus can include, but are not limited to, Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20. Examples of gammacoronavirus can include, but are not limited to, Avian coronavirus, Beluga whale coronavirus SW1. Additional examples of coronavirus can include MERS-CoV, SARS-CoV, and SARS-Cov-2 (i.e., SARS-COV-2). In some embodiments, the at least one heterologous RNA polynucleotide can be complementary and bind to the viral genomes, viral genes, or transcripts of the viral genomes and viral genes of SARS-COV-2. Exemplary SARS-COV-2 viral genes that can be targeted and bound by the at least one heterologous RNA polynucleotide can include orf1a, orf1ab, S (spike), 3a, 3b, E (envelope protein), M (matrix protein), p6, 7a, 7b, 8b, 9b, N (nucleocapsid), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRP), nsp13 (RNA 5′-triphosphatase), nsp14 (3′-to-5′ exonuclease), nsp15 (endoRNAse), and nsp16 (2′-O-ribose methyltransferase).

In some embodiments, the at least one heterologous RNA polynucleotide complementary to the viral genomes, viral genes, or transcripts of the viral genomes and viral genes of SARS-COV-2 comprises at most 5 mismatches when optimally aligned with MERS and/or SARS viral genomes. In some embodiments, the at least one heterologous RNA polynucleotide complementary to the viral genomes, viral genes, or transcripts of the viral genomes and viral genes of SARS-COV-2 comprises at most 4 mismatches when optimally aligned with MERS and/or SARS viral genomes. In some embodiments, the at least one heterologous RNA polynucleotide complementary to the viral genomes, viral genes, or transcripts of the viral genomes and viral genes of SARS-COV-2 comprises at most 3 mismatches when optimally aligned with MERS and/or SARS viral genomes. In some embodiments, the at least one heterologous RNA polynucleotide complementary to the viral genomes, viral genes, or transcripts of the viral genomes and viral genes of SARS-COV-2 comprises at most 2 mismatches when optimally aligned with MERS and/or SARS viral genomes. In some embodiments, the at least one heterologous RNA polynucleotide complementary to the viral genomes, viral genes, or transcripts of the viral genomes and viral genes of SARS-COV-2 comprises at most 1 mismatches when optimally aligned with MERS and/or SARS viral genomes. In some embodiments, the at least one heterologous RNA polynucleotide complementary to the viral genomes, viral genes, or transcripts of the viral genomes and viral genes of SARS-COV-2 comprises no mismatch when optimally aligned with MERS and/or SARS viral genomes. In some embodiments, the mismatches can be insertions, deletions, synonymous mutations, non-synonymous mutations, or combinations thereof.

In some embodiments, the at least one heterologous RNA polynucleotide can be complementary and bind to the viral genomes, viral genes, or viral gene segments, or transcripts of the viral genomes and viral genes and viral gene segments as described herein. In some embodiments, the at least one heterologous RNA polynucleotide can be complementary and bind to viral genomes, viral genes, or transcripts of the viral genomes and viral genes of influenza virus. In some embodiments, the at least one heterologous RNA polynucleotide can be complementary and bind to viral genomes, viral genes, or transcripts of the viral genomes and viral genes of an influenza virus, such as influenza A, influenza B, influenza C, and influenza D. In some embodiments, the at least one heterologous RNA polynucleotide can be complementary and bind to viral genomes, viral genes, or transcripts of the viral genomes and viral genes of influenza A. Examples of an influenza can include, but are not limited to, influenza A types H1, H3, H5, H7, and H9 viruses. In some embodiments, the at least one heterologous RNA polynucleotide can be complementary and bind to viral genomes, viral genes, or transcripts of the viral genomes and viral genes can be selected from any strain of Column 1 of Table 6.

In some embodiments, the at least one heterologous RNA polynucleotide comprising a nucleic acid sequence of any one SEQ ID NOs: 13601-13606 targets an endogenous gene of the cell being infected by any one of strain of the virus described herein. In some embodiments, the endogenous gene is a member of the peptidase family M1 such as aminopeptidase N (ANPEP), aminopeptidase A, leukotriene A4 hydrolase, Ape2 aminopeptidase, Aap1′ aminopeptidase, pyroglutamyl-peptidase II, cytosol alanyl aminopeptidase, cystinyl aminopeptidase, aminopeptidase G, aminopeptidase B, aminopeptidase Ey, endoplasmic reticulum aminopeptidase 1, tricom interacting factor F2, tricorn interacting factor F3, arginyl aminopeptidase-like 1, ERAP2 aminopeptidase, aminopeptidase, aminopeptidase O, or Tata binding protein associated factor. In some embodiments, the endogenous gene targeted by the heterologous RNA polynucleotide is ANPEP. In some embodiments, the endogenous gene is a member of the minor histocompatibility antigen family such as AKAP13, APOBEC3B, ARHGDIB, BCAT2, BCL2A1, CD19, CENPM, CTSH, C19orf48, DDX3Y, DPH1, EB13, ERAP1, ERBB2, GEMIN4, HEATR1, ARHGAP45, HMHB1, HMSD, KDM5D, PUM3, RESF1, LY75, MR1, MTHFD1, MYO1G, NUP133, PDCD11, PI4K2B. PKN3, PRCP, PTK2B, P2RX5, RPS4Y1, SLC1A5, SLC19A1, SON, SP110, SSR1, SWAP70, TMSB4Y, TOR3A, TRIM22, TRIM42, TRIP10, TYMP, UGT2B17, USP9Y, UTY, WNK1, or ZNF419. In some embodiments, the endogenous gene targeted by the heterologous RNA polynucleotide is MTHFD1. In some embodiments, the endogenous gene is a member of the type II transmembrane serine protease family such as HAT/DESC subfamily: HAT, DESC1, TMPRSS11A, HAT-like 2, HAT-like 3, HAT-like 4, HAT-like 5: Hepsin/TMPRSS subfamily: Hepsin, TMPRSS2, TMPRSS3, TMPRSS4, MSPL, Spinesin, Enteropeptidase; Matriptase subfamily: Matriptase, Matriptase-2, Matripase-3, Polyserease-1; and Corin subfamily Corin. In some embodiments, the endogenous gene targeted by the heterologous RNA polynucleotide is TMPRSS2.

In some embodiments, the at least one heterologous RNA polynucleotide comprising a nucleic acid sequence of any one of SEQ ID NOs: 13607-13649 or 13701-13729 can target the RdRP, N-protein, or the viral genome of any one strain of the coronavirus described herein. In some embodiments, a combination of the at least one heterologous RNA polynucleotide comprising a nucleic acid sequence of any one of SEQ ID NOs: 13651-13664 or 13671-13684 can target any one strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the virus described herein. In some embodiments, a combination of the at least one heterologous RNA polynucleotide comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the RNA virus described herein. Non-limiting examples of the RNA virus include RNA virus families of Coronaviridae, Pneumoviridae, Paramyxoviridae, Caliciviridae, Hepeviridae, Picomaviridae, Filoviridae, Flaviviridae, Rhabdoviridae, or Togaviridae.

In some embodiments, the systems and methods described herein comprise complexing the at least one heterologous RNA polynucleotide with the gene regulating moiety. In some embodiments, the systems and methods described herein comprise complexing at least two different heterologous RNA polynucleotides with the gene regulating moiety. In some embodiments, the systems and methods described herein comprise complexing at least three different heterologous RNA polynucleotides with the gene regulating moiety. In some embodiments, the systems and methods described herein comprise complexing at least four different heterologous RNA polynucleotides with the gene regulating moiety. In some embodiments, the systems and methods described herein comprise complexing at least five different heterologous RNA polynucleotides with the gene regulating moiety. In some embodiments, the systems and methods described herein comprise complexing at least six different heterologous RNA polynucleotides with the gene regulating moiety.

In some embodiments, the systems and methods described herein comprise complexing at most one heterologous RNA polynucleotide with the gene regulating moiety. In some embodiments, the systems and methods described herein comprise complexing at most two different heterologous RNA polynucleotides with the gene regulating moiety. In some embodiments, the systems and methods described herein comprise complexing at most three different heterologous RNA polynucleotides with the gene regulating moiety. In some embodiments, the systems and methods described herein comprise complexing at most four different heterologous RNA polynucleotides with the gene regulating moiety. In some embodiments, the systems and methods described herein comprise complexing at most five different heterologous RNA polynucleotides with the gene regulating moiety. In some embodiments, the systems and methods described herein comprise complexing at most six different heterologous RNA polynucleotides with the gene regulating moiety.

In some embodiments, the systems and methods described herein comprise complexing the one heterologous RNA polynucleotide with the gene regulating moiety. In some embodiments, the systems and methods described herein comprise complexing two different heterologous RNA polynucleotides with the gene regulating moiety. In some embodiments, the systems and methods described herein comprise complexing three different heterologous RNA polynucleotides with the gene regulating moiety. In some embodiments, the systems and methods described herein comprise complexing four different heterologous RNA polynucleotides with the gene regulating moiety. In some embodiments, the systems and methods described herein comprise complexing five different heterologous RNA polynucleotides with the gene regulating moiety. In some embodiments, the systems and methods described herein comprise complexing six different heterologous RNA polynucleotides with the gene regulating moiety.

In some embodiments, the systems and methods described herein comprise complexing the at least one heterologous RNA polynucleotide with the gene regulating moiety. In some embodiments, the systems and methods described herein comprise complexing a plurality of the heterologous RNA polynucleotides with the gene regulating moiety. In some cases, the plurality of the heterologous RNA polynucleotides comprises one, two, three, four, five, six, seven, eight, nine, ten, 20, 30, 50, 100, or more different heterologous RNA polynucleotides. In some embodiments, the plurality of the heterologous RNA polynucleotides collectively bind to at least 50%, 60%, 70%, 80%, 90%, 95%, 99%, or more of different types of coronavirus selected from the group consisting of alphacoronavirus, betacoronavirus, deltacoronavirus, and gammacoronavirus.

The term “collectively bind” as used herein generally refers to binding of a plurality of polynucleotides (e.g., crRNAs as disclosed herein) to a pool (or a set) of targets as a group. The pool of targets can be a pool of different viral genomes, different viral genes, or transcripts of the different viral genomes or viral genes. For example, the pool of targets can be a set of different strains of the coronavirus. A number or proportion of the pool of targets that is collectively bound (or collectively targeted) by the plurality of polynucleotides can be approximately equal to the number of proportion of individual targets of the pool of targets that is bound (or targeted) by at least one of the plurality of polynucleotides. For example, a plurality of polynucleotides can collectively bind 50% of a pool of targets when 50% of the pool of targets are bound (or targeted) by one or more polynucleotide of the plurality of polynucleotides. In some cases, an individual polynucleotide of the plurality of polynucleotides can be configured to bind only one of the pool of targets. Alternatively, an individual polynucleotide of the plurality of polynucleotides can be configured to bind to a plurality of targets of the pool of targets. A non-limiting example of the pool of targets can be a pool of genomes (or fragments thereof) of different viruses, e.g., different coronavirus types and/or strains. The terms “binds” and “targets” can be used interchangeably herein.

D. crRNA

Described herein, in some embodiments, are heterologous RNA polynucleotides comprising crRNAs. In some embodiments, the crRNA comprises targeting sequence that is complementary to the target viral genomes, viral genes, or transcripts of the viral genomes or viral genes as described herein.

In some embodiments, the crRNA comprises 5 nt to 100 nt. In some embodiments, the crRNA comprises 5 nt to 6 nt, 5 nt to 7 nt, 5 nt to 8 nt, 5 nt to 9 nt, 5 nt to 10 nt, 5 nt to 15 nt, 5 nt to 20 nt, 5 nt to 25 nt, 5 nt to 50 nt, 5 nt to 100 nt, 6 nt to 7 nt, 6 nt to 8 nt, 6 nt to 9 nt, 6 nt to 10 nt, 6 nt to 15 nt, 6 nt to 20 nt, 6 nt to 25 nt, 6 nt to 50 nt, 6 nt to 100 nt, 7 nt to 8 nt, 7 nt to 9 nt, 7 nt to 10 nt, 7 nt to 15 nt, 7 nt to 20 nt, 7 nt to 25 nt, 7 nt to 50 nt, 7 nt to 100 nt, 8 nt to 9 nt, 8 nt to 10 nt, 8 nt to 15 nt, 8 nt to 20 nt, 8 nt to 25 nt, 8 nt to 50 nt, 8 nt to 100 nt, 9 nt to 10 nt, 9 nt to 15 nt, 9 nt to 20 nt, 9 nt to 25 nt, 9 nt to 50 nt, 9 nt to 100 nt, 10 nt to 15 nt, 10 nt to 20 nt, 10 nt to 25 nt, 10 nt to 50 nt, 0 nt to 100 nt, 15 nt to 20 nt, 5 nt to 25 nt, 15 nt to 50 nt, 15 nt to 100 nt, 20 nt to 25 nt, 20 nt to 50 nt, 20 nt to 100 nt, 25 nt to 50 nt, 25 nt to 100 nt, or 50 nt to 100 nt. In some embodiments, the crRNA comprises 5 nt, 6 nt, 7 nt, 8 nt, 9 nt, 10 nt, 15 nt, 20 nt, 25 nt, 50 nt, or 100 nt. In some embodiments, the crRNA comprises at least 5 nt, 6 nt, 7 nt, 8 nt, 9 nt, 10 nt, 15 nt, 20 nt, 25 nt, or 50 nt. In some embodiments, the crRNA comprises at most 6 nt, 7 nt, 8 nt, 9 nt, 10 nt, 15 nt, 20 nt, 25 nt, 50 nt, or 100 nt. In some embodiments, the crRNA comprises at least 5 nt to 100 nt. In some embodiments, the crRNA comprises at least 5 nt to 6 nt, 5 nt to 7 nt, 5 nt to 8 nt, 5 nt to 9 nt, 5 nt to 10 nt, 5 nt to 15 nt, 5 nt to 20 nt, 5 nt to 25 nt, 5 nt to 50 nt, 5 nt to 100 nt, 6 nt to 7 nt, 6 nt to 8 nt, 6 nt to 9 nt, 6 nt to 10 nt, 6 nt to 15 nt, 6 nt to 20 nt, 6 nt to 25 nt, 6 nt to 50 nt, 6 nt to 100 nt, 7 nt to 8 nt, 7 nt to 9 nt, 7 nt to 10 nt, 7 nt to 15 nt, 7 nt to 20 nt, 7 nt to 25 nt, 7 nt to 50 nt, 7 nt to 100 nt, 8 nt to 9 nt, 8 nt to 10 nt, 8 nt to 15 nt, 8 nt to 20 nt, 8 nt to 25 nt, 8 nt to 50 nt, 8 nt to 100 nt, 9 nt to 10 nt, 9 nt to 15 nt, 9 nt to 20 nt, 9 nt to 25 nt, 9 nt to 50 nt, 9 nt to 100 nt, 10 nt to 15 nt, 10 nt to 20 nt, 10 nt to 25 nt, 10 nt to 50 nt, 10 nt to 100 nt, 15 nt to 20 nt, 15 nt to 25 nt, 15 nt to 50 nt, 15 nt to 100 nt, 20 nt to 25 nt, 20 nt to 50 nt, 20 nt to 100 nt, 25 nt to 50 nt, 25 nt to 100 nt, or 50 nt to 100 nt. In some embodiments, the crRNA comprises at least 5 nt, 6 nt, 7 nt, 8 nt, 9 nt, 10 nt, 15 nt, 20 nt, 25 nt, 50 nt, or 100 nt. In some embodiments, the crRNA comprises at least at least 5 nt, 6 nt, 7 nt, 8 nt, 9 nt, 10 nt, 15 nt, 20 nt, 25 nt, or 50 nt. In some embodiments, the crRNA comprises at least at most 6 nt, 7 nt, 8 nt, 9 nt, 10 nt, 15 nt, 20 nt, 25 nt, 50 nt, or 100 nt. In some embodiments, the crRNA comprises at most 5 nt to 100 nt. In some embodiments, the crRNA comprises at most 5 nt to 6 nt, 5 nt to 7 nt, 5 nt to 8 nt, 5 nt to 9 nt, 5 nt to 10 nt, 5 nt to 15 nt, 5 nt to 20 nt, 5 nt to 25 nt, 5 nt to 50 nt, 5 nt to 100 nt, 6 nt to 7 nt, 6 nt to 8 nt, 6 nt to 9 nt, 6 nt to 10 nt, 6 nt to 15 nt, 6 nt to 20 nt, 6 nt to 25 nt, 6 nt to 50 nt, 6 nt to 100 nt, 7 nt to 8 nt, 7 nt to 9 nt, 7 nt to 10 nt, 7 nt to 15 nt, 7 nt to 20 nt, 7 nt to 25 nt, 7 nt to 50 nt, 7 nt to 100 nt, 8 nt to 9 nt, 8 nt to 10 nt, 8 nt to 15 nt, 8 nt to 20 nt, 8 nt to 25 nt, 8 nt to 50 nt, 8 nt to 100 nt, 9 nt to 10 nt, 9 nt to 15 nt, 9 nt to 20 nt, 9 nt to 25 nt, 9 nt to 50 nt, 9 nt to 100 nt, 10 nt to 15 nt, 10 nt to 20 nt, 10 nt to 25 nt, 10 nt to 50 nt, 10 nt to 100 nt, 15 nt to 20 nt, 15 nt to 25 nt t nt to 50 nt, 15 nt to 100 nt, 20 nt to 25 nt, 20 nt to 50 nt, 20 nt to 100 nt, 25 nt to 50 nt, 25 nt to 100 nt, or 50 nt to 100 nt. In some embodiments, the crRNA comprises at most 5 nt, 6 nt, 7 nt, 8 nt, 9 nt, 10 nt, 15 nt, 20 nt, 25 nt, 50 nt, or 100 nt. In some embodiments, the crRNA comprises at most at least 5 nt, 6 nt, 7 nt, 8 nt, 9 nt, 10 nt, 15 nt, 20 nt, 25 nt, or 50 nt. In some embodiments, the crRNA comprises at most at most 6 nt, 7 nt, 8 nt, 9 nt, 10 nt, 15 nt, 20 nt, 25 nt, 50 nt, or 100 nt.

In some embodiments, the crRNA complements and binds to any one of the reference viral genome as indicated by the GenBank Access ID numbers. In some embodiments, the crRNA shares at least 50%, 60%, 70%, 80%, 90%, 95%, or 99% of a portion of any one of the reference viral genome as indicated by the GenBank Access ID numbers.

In some embodiments, the crRNA complements and binds to the viral coding regions, viral genes, or viral open reading frames of SARS-COV-2. In some embodiments, the crRNA shares at least 50%, 60%, 70%, 80%, 90%, 95%, or 99% of a portion of the viral coding regions, viral genes, or viral open reading frames of SARS-COV-2. Exemplary COVI-19 genes that can be targeted and bound by the crRNA include orf1a, orf1ab, S (spike), 3a, 3b, E (envelope protein), M (matrix protein), p6, 7a, 7b, 8b, 9b, N (nucleocapsid), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRP), nsp13 (RNA 5′-triphosphatase), nsp14 (3′-to-5′ exonuclease), nsp15 (endoRNAse), and nsp16 (2′-O-ribose methyltransferase). In some cases, the crRNA described herein can target and bind to a portion of the RdRP or a portion of the N protein.

As disclosed herein, a plurality of types of coronavirus can be selected from the group consisting of alphacoronavirus, betacoronavirus, deltacoronavirus, and gammacoronavirus. The plurality of types of coronavirus can be a collection of at least 100, 200, 300, 400, 500, 1000, 1500, 2000, 2500, 3000, or more different strains of coronavirus.

In some embodiments, a single crRNA can bind to (or bind to genomes or gene fragments thereof from) at least 20%, 25%, 30%, 35%, 40%, 45%, 50%, 60%, 70%, 80%, 90%, 95%, 99%, or more of the different types of coronavirus selected from the group consisting of alphacoronavirus, betacoronavirus, deltacoronavirus, and gammacoronavirus. In preferred embodiments, the single cRNA can collectively bind to at least 30%, 31%, 32%, 33%, 34%, 35%, 36%, 37%, 38%, 39%, 40%, 45%, 50%, or more of a collection of at least 1000, 2000, 3000, or more genomes from the different types of coronavirus.

In some embodiments, at least 2, 3, 4, 5, 6, 7, 8, 9, 10, 20, 25, 30, 40, 50, or more crRNA can collectively bind to (or collectively bind to genomes or gene fragments thereof from) at least 40%, 45%, 50%, 55%, 60%, 65%, 70%, 75%, 80%, 85%, 90%, 95%, 99%, or more of different types of coronavirus selected from the group consisting of alphacoronavirus, betacoronavirus, deltacoronavirus, and gammacoronavirus. In some embodiments, at most 50, 40, 30, 25, 20, 10, 9, 8, 7, 6, 5, 4, 3, or 2 crRNA can collectively bind to (or collectively bind to genomes or gene fragments thereof from) at least 40%, 45%, 50%, 55%, 60%, 65%, 70%, 75%, 80%, 85%, 90%, 95%, 99%, or more of different types of coronavirus selected from the group consisting of alphacoronavirus, betacoronavirus, deltacoronavirus, and gammacoronavirus. In some embodiments, 2, 3, 4, 5, 6, 7, 8, 9, 10, 20, 25, 30, 40, 50, or more crRNA can collectively bind to (or collectively bind to genomes or gene fragments thereof from) at least 40%, 45%, 50%, 55%, 60%, 65%, 70%, 75%, 80%, 85%, 90%, 95%, 99%, or more of different types of coronavirus selected from the group consisting of alphacoronavirus, betacoronavirus, deltacoronavirus, and gammacoronavirus.

In some embodiments, at most two crRNAs (e.g., 2 crRNAs) can collectively bind to at least 40%, 45%, 50%, 60%, 70%, 80%, 90%, 95%, 99%, or more of different types of coronavirus selected from the group consisting of alphacoronavirus, betacoronavirus, deltacoronavirus, and gammacoronavirus. In preferred embodiments, at most two crRNAs can collectively bind to at least 45% or more of a collection of at least 1000, 2000, 3000, or more genomes from the different types of coronavirus.

In some embodiments, at most three crRNAs (e.g., 3 crRNAs) can collectively bind to at least 50%, 55%, 60%, 70%, 80%, 90%, 95%, 99%, or more of different types of coronavirus selected from the group consisting of alphacoronavirus, betacoronavirus, deltacoronavirus, and gammacoronavirus. In preferred embodiments, at most three crRNAs can collectively bind to at least 60% or more of a collection of at least 1000, 2000, 3000, or more genomes from the different types of coronavirus.

In some embodiments, at most four crRNAs (e.g., 4 crRNAs) can collectively bind to at least 70%, 75%, 80%, 85%, 90%, 95%, 99%, or more of different types of coronavirus selected from the group consisting of alphacoronavirus, betacoronavirus, deltacoronavirus, and gammacoronavirus. In preferred embodiments, at most four crRNAs can collectively bind to at least 80% or more of a collection of at least 1000, 2000, 3000, or more genomes from the different types of coronavirus.

In some embodiments, at most five crRNAs (e.g., 5 crRNAs) can collectively bind to at least 80%, 81%, 82%, 83%, 84%, 85%, 86%, 87%, 88%, 89%, 90%, 95%, 99%, or more of different types of coronavirus selected from the group consisting of alphacoronavirus, betacoronavirus, deltacoronavirus, and gammacoronavirus. In preferred embodiments, at most five crRNAs can collectively bind to at least 85% or more of a collection of at least 1000, 2000, 3000, or more genomes from the different types of coronavirus.

In some embodiments, at most six crRNAs (e.g., 6 crRNAs) can collectively bind to at least 90%, 91%, 92%, 93%, 94%, 95%, 96%, 97%, 98%, 99%, or more of different types of coronavirus selected from the group consisting of alphacoronavirus, betacoronavirus, deltacoronavirus, and gammacoronavirus. In preferred embodiments, at most six crRNAs can collectively bind to at least 90% or more of a collection of at least 1000, 2000, 3000, or more genomes from the different types of coronavirus.

In some embodiments, at most ten crRNAs (e.g., 10 crRNAs) can collectively bind to at least 92%, 93%, 94%, 95%, 96%, 97%, 98%, 99%, or more of different types of coronavirus selected from the group consisting of alphacoronavirus, betacoronavirus, deltacoronavirus, and gammacoronavirus. In preferred embodiments, at most ten crRNAs can collectively bind to at least 95% or more of a collection of at least 1000, 2000, 3000, or more genomes from the different types of coronavirus.

In some embodiments, at most fourteen crRNAs (e.g., 14 crRNAs SEQ ID NOs: 13651-13664) can collectively bind to at least 95%, 96%, 97%, 98%, 99%, 99.5%, or more of different types of coronavirus selected from the group consisting of alphacoronavirus, betacoronavirus, deltacoronavirus, and gammacoronavirus. In preferred embodiments, at most fourteen crRNAs can collectively bind to at least 99% or more of a collection of at least 1000, 2000, 3000, or more genomes from the different types of coronavirus.

In some embodiments, at most twenty-two (e.g., 22 crRNAs) can collectively bind to at least 99.9% (e.g., 100%) of different types of coronavirus selected from the group consisting of alphacoronavirus, betacoronavirus, deltacoronavirus, and gammacoronavirus. In preferred embodiments, at most twenty-two crRNAs can collectively bind to at least 99.9% or more of a collection of at least 1000, 2000, 3000, or more genomes from the different types of coronavirus.

Table 1 and Table 5 illustrate exemplary crRNAs, where any combinations of 2, 4, 6, 8, 10, 15, or 20 of the cRNAs can target at least 90% of different types of coronavirus selected from the group consisting of alphacoronavirus, betacoronavirus, deltacoronavirus, and gammacoronavirus.

In some embodiments, the crRNA as described in the present disclosure can comprise any sequence in SEQ ID NOs: 1-3203, 4001-7203, and 8001-11203.

In some embodiments, the crRNA as described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence selected from SEQ ID NOs: 13301-13348, 13401-13448, and 13501-13548.

The at least one crRNA as described in the present disclosure can comprise at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 13301-13348, 13401-13448, and 13501-13548.

In some cases, the at least one crRNA can be configured to affect at least about 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more reduction in expression of one or more target viral genes encoding one or more viral proteins (e.g., PB2, PB2, PB1-F2, PA, HA, NP, NA, M1, M2, NS1, and/or NEP/NS2) or a fragment thereof. In some cases, the least one heterologous RNA polynucleotide can be configured to effect at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, or less in expression of one or more target viral genes encoding one or more viral proteins (e.g., PB2, PB2, PB1-F2, PA, HA, NP, NA, M1, M2, NS1, and/or NEP/NS2) or a fragment thereof.

In some embodiments, the at least one crRNA as described in the present disclosure can comprise at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a sequence selected from SEQ ID NOs: 13601-13606, 13607-13649, 13651-13664, 13671-13684, and 13701-13729.

The at least one crRNA as described in the present disclosure can comprise at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, or less sequence identity to a sequence selected from SEQ ID NOs: 13601-13606, 13607-13649, 13651-13664, 13671-13684, and 13701-13729.

In some embodiments, the at least one crRNA comprising a nucleic acid sequence of any one SEQ ID NOs: 13601-13606 targets an endogenous gene of the cell being infected by any one of strain of the virus described herein. In some embodiments, the endogenous gene is a member of the peptidase family M1 such as aminopeptidase N (ANPEP), aminopeptidase A, leukotriene A4 hydrolase, Ape2 aminopeptidase, Aap1′ aminopeptidase, pyroglutamyl-peptidase II, cytosol alanyl aminopeptidase, cystinyl aminopeptidase, aminopeptidase G, aminopeptidase B, aminopeptidase Ey, endoplasmic reticulum aminopeptidase 1, tricorn interacting factor F2, tricom interacting factor F3, arginyl aminopeptidase-like 1, ERAP2 aminopeptidase, aminopeptidase, aminopeptidase O, or Tata binding protein associated factor. In some embodiments, the endogenous gene targeted by the heterologous RNA polynucleotide is ANPEP. In some embodiments, the endogenous gene is a member of the minor histocompatibility antigen family such as AKAP13, APOBEC3B, ARHGDIB, BCAT2, BCL2A1, CD19, CENPM, CTSH, C19orf48, DDX3Y, DPH1, EBI3, ERAP1, ERBB2, GEMIN4, HEATR1, ARHGAP45, HMHB1, HMSD, KDM5D, PUM3, RESF1, LY75, MR1, MTHFD1, MYO1G, NUP133, PDCD11, PI4K2B, PKN3, PRCP, PTK2B, P2RX5, RPS4Y1, SLC1A5, SLC19A1, SON, SP110, SSR1, SWAP70, TMSB4Y, TOR3A, TRIM22, TRIM42, TRIP10, TYMP, UGT2B17, USP9Y, UTY, WNK1, or ZNF419. In some embodiments, the endogenous gene targeted by the heterologous RNA polynucleotide is MTHFD1. In some embodiments, the endogenous gene is a member of the type II transmembrane serine protease family such as HAT/DESC subfamily: HAT, DESC1, TMPRSS11A, HAT-like 2, HAT-like 3, HAT-like 4, HAT-like 5; Hepsin/TMPRSS subfamily: Hepsin, TMPRSS2, TMPRSS3, TMPRSS4, MSPL, Spinesin, Enteropeptidase; Matriptase subfamily: Matriptase, Matriptase-2, Matripase-3, Polyserease-1; and Corin subfamily Corin. In some embodiments, the endogenous gene targeted by the heterologous RNA polynucleotide is TMPRSS2.

In some embodiments, the at least one crRNA comprising a nucleic acid sequence of any one of SEQ ID NOs: 13607-13649 or 13701-13729 can target the RdRP, N-protein, or the viral genome of any one strain of the coronavirus described herein. In some embodiments, a combination of the at least one crRNA comprising a nucleic acid sequence of any one of SEQ ID NOs: 13651-13664 or 13671-13684 can target any one strain of the virus described herein. In some embodiments, a combination of the at least one crRNA comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the virus described herein. In some embodiments, a combination of the at least one crRNA comprising a nucleic acid sequence of any one of SEQ ID NOs: 13651-13664 or 13671-13684 can target viral genome of at least about 500, 1000, 5000, 10000, 15000, 20000, 21000, 22000, 23000, 24000, 25000, 30000, or more strains of virus. of any one of the strain of the RNA virus described herein. Non-limiting examples of the RNA virus include RNA virus families of Coronaviridae, Pneumoviridae. Paramyxoviridae, Caliciviridae, Hepeviridae, Picomaviridae, Filoviridae, Flaviviridae, Rhabdoviridae, or Togaviridae.

In some embodiments, one or more strains of a virus as disclosed herein can be one or more reference strains of a reference virus. In some cases, a plurality of strains of a plurality of virus families can be a plurality of references strains of a plurality of reference virus families.

E. Delivery

Described herein, in some embodiments, are methods of delivering the systems described herein into a cell, thus reducing viral expression by contacting the cell with the systems. In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other exemplary cells can include lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle: bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells. Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion). Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell. Oxyphil cell, Adrenal gland cells, chromaffin cells, Ley dig cell of testes. Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney. Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues). Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell. B cell, Natural killer cell, Reticulocyte. Stem cells and committed progenitors for the blood and immune system (various types). Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovanan follicle cell, Sertoli cell (in testis), Thymus epithelial cell, Interstitial cells, and Interstitial kidney cells.

In some embodiments, the gene regulating moiety and the heterologous RNA polynucleotide can be delivered into the cell. In some embodiments, polynucleotides encoding the gene regulating moiety and/or the heterologous RNA polynucleotide can be delivered into the cell via the use of expression vectors. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, gene gun, electroporation, and the like. Methods for producing cells comprising vectors and/or exogenous nucleic acids are suitable for methods herein. One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated systems are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some embodiments, dimyristyl phosphatidylcholine (“DMPC”) is obtained from Sigma, St. Louis, Mo.: in some embodiments, dicetyl phosphate (“DCP”) is obtained from K & K Laboratories (Plainview, N.Y.); cholesterol (“Choi”), in some embodiments, is obtained from Calbiochem-Behring: dimyristyl phosphatidylglycerol (“DMPG”) and other lipids are often obtained from Avanti Polar Lipids. Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, systems that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, the systems described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the systems described herein can be administered to the subject in need thereof via the use of genetically engineered cells generated by introduction of the systems first into allogeneic or autologous cells. The genetically engineered cells can the confer the treatment effects of the systems to the subject infected by the coronavirus.

In some embodiments, the systems or the polynucleotides encoding the systems can be delivered cells to reduce or eliminate expression of viral genes, where these cells can then be subsequently administered to a subject in need thereof for treatment purposes. In some cases, these cells can be autologous (i.e. transplantation using the subject's own cells) or allogenic (i.e. transplantation using cells from a donor where the donor's human leukocyte antigens or HLAs are compatible to the subject's HLA).

In an example, any cell of the present disclosure can be engineered to express a chimeric antigen receptor (CAR). The term “chimeric antigen receptor” or alternatively a “CAR” may be used herein to generally refer to a recombinant polypeptide construct comprising at least an extracellular antigen binding domain, a transmembrane domain, and a cytoplasmic signaling domain (also referred to herein as “an intracellular or intrinsic signaling domain”) comprising a functional signaling domain derived from a stimulatory molecule. In some cases, the stimulatory molecule may be the zeta chain associated with the T cell receptor complex. In some cases, the intracellular signaling domain further comprises one or more functional signaling domains derived from at least one costimulatory molecule. In some cases, the costimulatory molecule may comprise 4-1BB (i.e., CD137), CD27, and/or CD28. In one aspect, the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane.

The CAR, as used herein, may be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 ζ receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3ζ-CD28-OX40, or CD3ζ-CD28-41BB). Fourth-generation CARs modify second- or third-generation CARs by the addition of an activating cytokine (e.g., IL-12, IL-23, or IL-27) to the intracellular signaling portion of the CAR (e.g., between one or more of the costimulatory domains and the CD3(ITAM domain) or under the control of a CAR-induced promoter (e.g., the NFAT/IL-2 minimal promoter). Any of the cells disclosed herein (e.g., stem cells) can be engineered to express a heterologous polynucleotide comprising the CAR or a heterologous polynucleotide encoding such heterologous polynucleotide.

F. Therapeutic Applications

Disclosed herein, in some embodiments, are methods of treating a coronaviral disease or condition, or a symptom of the coronaviral disease or condition, in a subject, comprising administrating of therapeutic effective amount of the systems or pharmaceutical compositions described herein to the subject. In some embodiments, the method reduces expression of a target viral gene in a cell, comprising steps of: contacting the cell with the systems as described in the instant disclose; upon the contacting, the system reduces expression of the target viral gene in the cell. In some embodiments, the contacting occurs in vivo, ex vivo, or in vitro. In some embodiments, the contacting occurs in vivo. In some embodiments, the contacting occurs ex vivo. In some embodiments, the contacting occurs in vitro. In some embodiments, the system can be expressed in the cell vial delivery of polynucleotides encoding the system to the cell. In some embodiment, the system or can directly administered to the subject.

In some embodiments, the system can be administered to the subject alone (e.g., standalone treatment). In some embodiments, the system is administered in combination with an additional agent. In some embodiments, the system is a first-line treatment for the disease or condition. In some embodiments, the system is a second-line, third-line, or fourth-line treatment, for the coronaviral disease or condition. In some embodiments, the systems are useful for the treatment of the coronaviral disease or condition, or symptom of the coronaviral disease or condition, disclosed herein. In some embodiments, the system comprises a gene regulating moiety and at least one heterologous RNA polynucleotide.

In general, methods disclosed herein comprise administering a system by oral administration. However, in some instances, methods comprise administering a system by intraperitoneal injection. In some instances, methods comprise administering a system in the form of an anal suppository. In some instances, methods comprise administering a system by intravenous (“i.v.”) administration. It is conceivable that one can also administer systems disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

An effective dose and dosage of the systems to prevent or treat the coronaviral disease or condition disclosed herein is defined by an observed beneficial response related to the coronal viral disease or condition, or symptom of the disease or condition. In some cases, the beneficial response comprises reduction of expression of viral genomes or viral genes as determined by the methods describe herein. Additional beneficial response comprises preventing, alleviating, arresting, or curing the coronaviral disease or condition, or symptom of the coronaviral disease or condition. An “improvement,” as used herein refers to reduction in expression of viral genomes or viral genes in the cells or in a sample obtained from the subject. In instances where the system is not therapeutically effective or is not providing a sufficient alleviation of the disease or condition, or symptom of the disease or condition, then the dosage amount and/or route of administration can be changed, or an additional agent can be administered to the subject, along with the system. In some embodiments, as a patient is started on a regimen of a system, the patient is also weaned off (e.g., step-wise decrease in dose) a second treatment regimen.

Suitable dose and dosage administrated to a subject is determined by factors including, but no limited to, the particular system, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some embodiments, the administration of the system is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In certain embodiments, where the patient's condition does not improve, upon the doctor's discretion the administration of system is administered chronically, that is, for an extended period of time, including throughout the duration of the patient's life in order to ameliorate or otherwise control or limit the symptoms of the patient's disease or condition. In certain embodiments wherein a patient's status does improve, the dose of system being administered can be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered can be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the system described herein lies within a range of circulating concentrations that include the ED50 with minimal toxicity. In certain embodiments, the daily dosage range and/or the unit dosage amount varies within this range depending upon the dosage form employed and the route of administration utilized.

A system can be used alone or in combination with an additional agent. In some cases, an “additional agent” as used herein is administered alone. The system and the additional agent can be administered together or sequentially. The combination therapies can be administered within the same day, or can be administered one or more days, weeks, months, or years apart.

G. Pharmaceutical Composition

A pharmaceutical composition, as used herein, refers to a mixture of a system described herein, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the systems include two or more system (e.g., one or more systems and one or more additional agents) as discussed herein. In practicing the methods of treatment or use provided herein, therapeutically effective amounts of systems described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the system used and other factors. The systems can be used singly or in combination with one or more systems as components of mixtures.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations

Pharmaceutical compositions comprising a system described herein are manufactured in a conventional manner, such as, by way of example only, by means of conventional mixing, dissolving, granulating, dragee-making, levigating, emulsifying, encapsulating, entrapping or compression processes.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a system as discussed herein, e.g., system is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a system described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections can involve bolus injection or continuous infusion. Formulations for injection can be presented in unit dosage form, e.g., in ampoules or in multi-dose containers, with an added preservative. The pharmaceutical composition described herein can be in a form suitable for parenteral injection as a sterile suspensions, solutions or emulsions in oily or aqueous vehicles, and can contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a system is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, such as, by way of example only, gelatin for use in an inhaler or insufflator can be formulated containing a powder mix of the system described herein and a suitable powder base such as lactose or starch.

Representative intranasal formulations including a system described herein are prepared as solutions in saline, employing benzyl alcohol or other suitable preservatives, fluorocarbons, and/or other solubilizing or dispersing agents known in the art. Preferably these systems and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

H. Kit

Disclosed herein, in some embodiments, are kits comprising the systems described herein. In some embodiments, the kits disclosed herein can be used to diagnose and/or treat a disease or condition in a subject; or select a patient for treatment and/or monitor a treatment disclosed herein. In some embodiments, the kit comprises the systems described herein, which can be used to perform the methods described herein. Kits comprise an assemblage of materials or components, including at least one of the systems. Thus, in some embodiments the kit contains a system including of the pharmaceutical composition, for the treatment of coronaviral infection. In other embodiments, the kits contains all of the components necessary and/or sufficient to perform an assay for detecting and measuring coronaviral markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit comprises the systems (e.g., primers, probes, antibodies) described herein. The disclosure provides kits suitable for assays such as enzyme-linked immunosorbent assay (ELISA), single-molecular array (Simoa). PCR, and qPCR. The exact nature of the components configured in the kit depends on its intended purpose. For example, some kits can be configured for the purpose of treating a disease or condition disclosed herein in a subject. In some embodiments, the kit can be configured particularly for the purpose of treating mammalian subjects. In some embodiments, the kit can be configured particularly for the purpose of treating human subjects. In further embodiments, the kit can be configured for veterinary applications, treating subjects such as, but not limited to, farm animals, domestic animals, and laboratory animals. In some embodiments, the kit can be configured to select a subject for a therapeutic agent, such as those disclosed herein. In some embodiments, the kit is configured to select a subject for treatment for coronaviral infection.

In some cases, instructions for use can be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form: they can be provided at room, refrigerated or frozen temperatures. The components are typically contained in suitable packaging material(s). As employed herein, the phrase “packaging material” refers to one or more physical structures used to house the contents of the kit, such as systems and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### EMBODIMENTS

Embodiment 1. A composition for regulating expression of one or more target viral genes in a cell, comprising: a heterologous polypeptide comprising a gene regulating moiety, wherein said gene regulating moiety is configured to regulate expression of said one or more target viral genes in said cell, and wherein said one or more target viral genes encode (i) a viral RNA-dependent RNA polymerase (RdRP) or a fragment thereof or (ii) a viral nucleocapsid protein (N-protein) or a fragment thereof.

Embodiment 2. The composition of embodiment 1, wherein said gene regulating moiety is an RNA-guided gene regulating moiety that binds at least a portion of said one or more target viral genes.

Embodiment 3. The composition of embodiment 2, further comprising at least one heterologous RNA polynucleotide configured to bind said at least said portion of said one or more target viral genes.

Embodiment 4. The composition of embodiment 3, wherein said at least one heterologous RNA polynucleotide comprises a plurality of heterologous RNA polynucleotides configured to bind a plurality of different portions of said one or more target viral genes.

Embodiment 5. The composition of embodiment 4, wherein said plurality of heterologous RNA polynucleotides comprises at least three different RNA polynucleotides.

Embodiment 6. The composition of embodiment 5, wherein said plurality of heterologous RNA polynucleotides comprises at least five different RNA polynucleotides.

Embodiment 7. The composition of embodiment 3, wherein said one or more target viral genes are from one or more coronaviral genomes, and wherein a single heterologous RNA polynucleotide of said at least one heterologous RNA polynucleotide is configured to target at least 35% of different types of coronaviruses selected from the group consisting of: alphacoronavirus, betacoronavirus, deltacoronavirus, and gammacoronavirus.

Embodiment 8. The composition of embodiment 4, wherein a plurality of heterologous RNA polynucleotides of said at least one heterologous RNA polynucleotide is configured to collectively target at least 45% of said different types of coronaviruses.

Embodiment 9. The composition of embodiment 8, wherein said plurality of heterologous RNA polynucleotides is configured to collectively target at least 80% of said different types of coronaviruses.

Embodiment 10. The composition of embodiment 9, wherein at most six heterologous RNA polynucleotides of said plurality are configured to collectively target at least 80% of said different types of coronaviruses.

Embodiment 11. The composition of embodiment 9, wherein at most ten heterologous RNA polynucleotides of said plurality are configured to collectively target at least 90% of said different types of coronaviruses.

Embodiment 12. The composition of embodiment 3, wherein each of said at least one heterologous RNA polynucleotide has a length ranging from 10 to 30 nucleotides.

Embodiment 13. The composition of embodiment 12, wherein said at least one heterologous RNA polynucleotide comprises at most 2 mismatches when optimally aligned with MERS and/or SARS viral genome.

Embodiment 14. The composition of embodiment 3, wherein each of said at least one heterologous RNA polynucleotide has at least 90% sequence identity to a sequence selected from SEQ ID NOs: 4001-7203, 12101-12140, and 13101-13122.

Embodiment 15. The composition of embodiment 3, wherein said one or more target viral genes are from one or more coronaviral genomes, wherein the one or more target viral genes encodes RdRP or N-protein.

Embodiment 16. The composition of embodiment 15, wherein said at least one heterologous RNA polynucleotide is configured to affect about 80% reduction in said expression of said one or more target viral genes encoding the viral RdRP or a fragment thereof.

Embodiment 17. The composition of embodiment 15, wherein said at least one heterologous RNA polynucleotide is configured to affect about 70% reduction in said expression of said one or more target viral genes encoding said viral N-protein or a fragment thereof.

Embodiment 18. The composition of embodiment 2, wherein said RNA-guided gene regulating moiety is a CRISPR/Cas protein selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Casl Od, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3. Csf4, a modification thereof, and a combination thereof.

Embodiment 19. The composition of embodiment 2, wherein said RNA-guided gene regulating moiety exhibits RNA cleaving activity.

Embodiment 20. The composition of embodiment 1, wherein (i) said composition or (ii) one or more polynucleotides encoding said composition is packaged in a delivery vehicle selected from the group consisting of: a liposome, a nanoparticle, and a viral capsule.

Embodiment 21. A method of reducing expression of one or more target viral genes in a cell, comprising: contacting the cell with the composition of any one of embodiments 1-20, wherein upon said contacting, said composition effects reduced expression of said one or more target viral genes in said cell.

Embodiment 22. The method of embodiment 21, wherein said contacting occurs in vivo, ex vivo, or in vitro.

Embodiment 23. The method of embodiment 21, further comprising expressing said composition in said cell.

Embodiment 24. The method of embodiment 21, further comprising administering (i) said composition or (ii) one or more polynucleotides encoding said composition to a subject in need thereof, thereby reducing said expression of said one or more target viral genes in said subject.

Embodiment 25. The method of embodiment 21, further comprising, subsequent to said contacting, administering said cell to a subject in need thereof.

Embodiment 26. A kit comprising the composition of any one of embodiments 1-20.

Embodiment 27. A kit comprising one or more polynucleotides encoding the composition of any one of embodiments 1-19.

Embodiment 28. A reaction mixture comprising the composition of any one of embodiments 1-20 and a biological sample.

Embodiment 29. The reaction mixture of embodiment 28, wherein said biological sample is selected from the group consisting of: urine, blood, cell, tissue, organ, organism, and a combination thereof.

Embodiment 30. A composition for regulating expression of one or more target viral genes in a cell, comprising: at least one heterologous RNA polynucleotide configured to bind at least a portion of said one or more target viral genes in said cell, wherein said one or more target viral genes encodes (i) a viral RNA-dependent RNA polymerase (RdRP) or a fragment thereof or (ii) a viral nucleocapsid protein (N-protein) or a fragment thereof, and wherein said at least one heterologous RNA polynucleotide is capable of forming a complex with a heterologous polypeptide comprising a gene regulating moiety to regulate expression of said one or more target viral genes in said cell.

Embodiment 31. The composition of embodiment 30, wherein said at least one heterologous RNA polynucleotide comprises a plurality of heterologous RNA polynucleotides configured to bind a plurality of different portions of said one or more target viral genes.

Embodiment 32. The composition of embodiment 31, wherein said plurality of heterologous RNA polynucleotides comprises at least three different RNA polynucleotides.

Embodiment 33. The composition of embodiment 32, wherein said plurality of heterologous RNA polynucleotides comprises at least five different RNA polynucleotides.

Embodiment 34. The composition of embodiment 30, wherein said one or more target viral genes are from one or more coronaviral genomes, and wherein a single heterologous RNA polynucleotide of said at least one heterologous RNA polynucleotide is configured to target at least 35% of different types of coronaviruses selected from the group consisting of: alphacoronavirus, betacoronavirus, deltacoronavirus, and gammacoronavirus.

Embodiment 35. The composition of embodiment 34, wherein a plurality of heterologous RNA polynucleotides of said at least one heterologous RNA polynucleotide is configured to collectively target at least 45% of said different types of coronaviruses.

Embodiment 36. The composition of embodiment 35, wherein said plurality of heterologous RNA polynucleotides is configured to collectively target at least 80% of said different types of coronaviruses.

Embodiment 37. The composition of embodiment 36, wherein at most six heterologous RNA polynucleotides of said plurality are configured to collectively target at least 80% of said different types of coronaviruses.

Embodiment 38. The composition of embodiment 36, wherein at most ten heterologous RNA polynucleotides of said plurality are configured to collectively target at least 90% of said different types of coronaviruses.

Embodiment 39. The composition of embodiment 30, wherein each of said at least one heterologous RNA polynucleotide has a length ranging from 10 to 30 nucleotides.

Embodiment 40. The composition of embodiment 39, wherein said at least one heterologous RNA polynucleotide comprises at most 2 mismatches when optimally aligned with MERS and/or SARS viral genome.

Embodiment 41. The composition of embodiment 30, wherein each of said at least one heterologous RNA polynucleotide has at least 90% sequence identity to a sequence selected from SEQ ID NOs: 4001-7203, 12101-12140, and 13101-13122.

Embodiment 42. The composition of embodiment 30, wherein said at least one heterologous RNA polynucleotide is configured to affect about 70% reduction in said expression of said one or more target viral genes in said cell as compared to one or more non-targeting heterologous RNA polynucleotides.

Embodiment 43. The composition of embodiment 42, wherein said at least one heterologous RNA polynucleotide is configured to affect about 75% reduction in said expression of said one or more target viral genes encoding the viral RdRP or a fragment thereof.

Embodiment 44. The composition of embodiment 42, wherein said at least one heterologous RNA polynucleotide is configured to affect about 80% reduction in said expression of said one or more target viral genes encoding said viral N-protein or a fragment thereof.

Embodiment 45. The composition of embodiment 30, further comprising said heterologous polypeptide comprising said heterologous gene regulating moiety.

Embodiment 46. The composition of embodiment 30, wherein said gene regulating moiety is a CRISPR/Cas protein selected from the group consisting of C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, and a combination thereof.

Embodiment 47. The composition of embodiment 30, wherein said gene regulating moiety exhibits RNA cleaving activity.

Embodiment 48. The composition of embodiment 30, wherein (i) said composition or (ii) one or more polynucleotides encoding said composition is packaged in a delivery vehicle selected from the group consisting of a liposome, a nanoparticle, and a viral capsule.

Embodiment 49. A method of reducing expression of one or more target viral genes in a cell, comprising: contacting the cell with the composition of any one of embodiments 30-48, wherein upon said contacting, said composition effects reduced expression of said one or more target viral genes in said cell.

Embodiment 50. The method of embodiment 49, wherein said contacting occurs in vivo, ex vivo, or in vitro.

Embodiment 51. The method of embodiment 49, further comprising expressing said composition in said cell.

Embodiment 52. The method of embodiment 49, further comprising administering (i) said composition or (ii) one or more polynucleotides encoding said composition to a subject in need thereof, thereby reducing said expression of said one or more target viral genes in said subject.

Embodiment 53. The method of embodiment 49, further comprising, subsequent to said contacting, administering said cell to a subject in need thereof.

Embodiment 54. A kit comprising the composition of any one of embodiments 30-48.

Embodiment 55. A kit comprising one or more polynucleotides encoding the composition of any one of embodiments 3047.

Embodiment 56. A nucleic acid composition comprising a RNA-binding guide RNA, wherein said RNA-binding guide RNA comprises a targeting sequence that has a length ranging from 10 to 30 nucleotides and is characterized by one or more members selected from the group consisting of: (i) said targeting sequence exhibits at least 90% sequence identity to any one of SEQ ID NOs: 4001-7203, 12101-12140, and 13101-13122; (ii) said targeting sequence is configured to bind a polynucleotide fragment within a coronavirus genome, wherein said polynucleotide fragment has a length of about 10 to 30 nucleotides and exhibits at least 95% sequence identity to a conserved region of said coronavirus genome that encodes (1) a RNA-dependent RNA polymerase (RdRP) or a fragment thereof or (2) a nucleocapsid protein (N-protein) or a fragment thereof, and (iii) said targeting sequence comprises at most 2 mismatches when optimally aligned with MERS and/or SARS viral genome.

Embodiment 57. The nucleic acid composition of embodiment 56, wherein, in (i), said targeting sequence exhibits at least 95% sequence identity to any one of SEQ ID NOs: 4001-7203, 12101-12140, and 13101-13122.

Embodiment 58. The nucleic acid composition of embodiment 56, wherein, in (ii), said conserved region encodes (1) said RdRP or said fragment thereof and (2) said N-protein or said fragment thereof.

Embodiment 59. The nucleic acid composition of embodiment 56, wherein, in (ii), said polynucleotide fragment exhibits at least 90% sequence identity to any one of SEQ ID NOs: 4001-7203, 12101-12140, and 13101-13122.

Embodiment 60. The nucleic acid composition of embodiment 59, wherein said polynucleotide fragments directed to said RdRP or said fragment thereof and exhibits at least 90% sequence identity to any one of SEQ ID NOs: 12101-12120.

Embodiment 61. The nucleic acid composition of embodiment 59, wherein said polynucleotide fragments directed to said N-protein or said fragment thereof and exhibits at least 90% sequence identity to any one of SEQ ID NOS: 12121-12140.

Embodiment 62. The nucleic acid composition of embodiment 56, wherein, in (iii), said targeting sequence comprises at most 1 mismatch when optimally aligned with MERS and/or SARS viral genome.

Embodiment 63. The nucleic acid composition of embodiment 56, wherein, in (iii), a mismatch of said at most 2 mismatches is an insertion or a deletion.

Embodiment 64. The nucleic acid composition of embodiment 56, wherein said targeting sequence is characterized by two or more members selected from the group consisting of (i), (ii), and (iii).

Embodiment 65. The nucleic acid composition of embodiment 64, wherein said targeting sequence is characterized by all members of (i), (ii), and (iii).

Embodiment 66. The nucleic acid composition of any one of embodiments 56-65, further comprising a plurality of RNA-binding guide RNAs comprising said RNA-binding guide RNA, wherein each of said plurality of RNA-binding guide RNAs is characterized by one or more members selected from the group consisting of: (i), (ii), and (iii).

Embodiment 67. The nucleic acid composition of embodiment 66, wherein said plurality comprises at least two RNA-binding guide RNAs.

Embodiment 68. The nucleic acid composition of embodiment 67, wherein said plurality comprises at least six RNA-binding guide RNAs.

Embodiment 69. A nucleic acid composition comprising a RNA-binding guide RNA, wherein said RNA-binding guide RNA comprises a targeting sequence that has a length ranging from 10 to 30 nucleotides and is selected from the group consisting of:

(i) a RNA polynucleotide having at least 90% sequence identity to SEQ ID NO: 13101;

(ii) a RNA polynucleotide having at least 90% sequence identity to SEQ ID NO: 13102;

(iii) a RNA polynucleotide having at least 90% sequence identity to SEQ ID NO: 13103;

(iv) a RNA polynucleotide having at least 90% sequence identity to SEQ ID NO: 13104;

(v) a RNA polynucleotide having at least 90% sequence identity to SEQ ID NO: 13105; and

(vi) a RNA polynucleotide having at least 90% sequence identity to SEQ ID NO: 13106.

Embodiment 70. The nucleic acid composition of embodiment 69, wherein:

(i) said RNA polynucleotide has at least 95% sequence identity to SEQ ID NO: 13101;

(ii) said RNA polynucleotide has at least 95% sequence identity to SEQ ID NO: 13102;

(iii) said RNA polynucleotide has at least 95% sequence identity to SEQ ID NO: 13103;

(iv) said RNA polynucleotide has at least 95% sequence identity to SEQ ID NO: 13104;

(v) said RNA polynucleotide has at least 95% sequence identity to SEQ ID NO: 13105; or

(vi) said RNA polynucleotide has at least 95% sequence identity to SEQ ID NO: 13106.

Embodiment 71. The nucleic acid composition of embodiment 69 or 70, further comprising a plurality of different RNA-binding guide RNAs comprising said RNA-binding guide RNA, wherein each RNA-binding guide RNAs of said plurality has a targeting sequence selected from the group consisting of; (i), (ii), (iii), (iv), (v), and (vi).

Embodiment 72. The nucleic acid composition of embodiment 71, wherein said plurality comprises two different guide RNAs.

Embodiment 73. The nucleic acid composition of embodiment 72, wherein said plurality comprises three different guide RNAs.

Embodiment 74. The nucleic acid composition of embodiment 73, wherein said plurality comprises four different guide RNAs.

Embodiment 75. The nucleic acid composition of embodiment 74, wherein said plurality comprises five different guide RNAs.

Embodiment 76. The nucleic acid composition of embodiment 75, wherein said plurality comprises six different guide RNAs.

Embodiment 77. A nucleic acid composition comprising a plurality of RNA-binding guide RNAs, wherein no more than six members of said plurality collectively exhibits specific binding to at least 50% of different types of coronaviruses selected from the group consisting of; alphacoronavirus, betacoronavirus, deltacoronavirus, and gammacoronavirus.

Embodiment 78. A nucleic acid composition comprising a plurality of RNA-binding guide RNAs, wherein no more than six members of said plurality collectively exhibits specific binding to at least 50% of different types of coronaviruses selected from the group consisting of coronaviruses listed by GenBank Access ID.

Embodiment 79. The nucleic acid composition of embodiment 77 or 78, wherein said different types of coronaviruses are selected from the group consisting of coronaviruses listed by GenBank Access ID.

Embodiment 80. The nucleic acid composition of embodiment 77 or 78, wherein a single member of said plurality exhibits specific binding to at least 35% of said different types of coronaviruses.

Embodiment 81. The nucleic acid composition of embodiment 77 or 78, wherein no more than two members of said plurality collectively exhibits specific binding to at least 45% of said different types of coronaviruses.

Embodiment 82. The nucleic acid composition of embodiment 77 or 78, wherein no more than three members of said plurality collectively exhibits specific binding to at least 65% of said different types of coronaviruses.

Embodiment 83. The nucleic acid composition of embodiment 77 or 78, wherein no more than four members of said plurality collectively exhibits specific binding to at least 80% of said different types of coronaviruses.

Embodiment 84. The nucleic acid composition of embodiment 77 or 78, wherein no more than five members of said plurality collectively exhibits specific binding to at least 85% of said different types of coronaviruses.

Embodiment 85. The nucleic acid composition of embodiment 77 or 78, wherein no more than six members of said plurality collectively exhibits specific binding to at least 90% of said different types of coronaviruses.

Embodiment 86. The nucleic acid composition of embodiment 77 or 78, wherein no more than seven members of said plurality collectively exhibits specific binding to at least 93% of said different types of coronaviruses.

Embodiment 87. The nucleic acid composition of embodiment 77 or 78, wherein no more than eight members of said plurality collectively exhibits specific binding to at least 95% of said different types of coronaviruses.

Embodiment 88. The nucleic acid composition of embodiment 77 or 78, wherein no more than nine members of said plurality collectively exhibits specific binding to at least 96% of said different types of coronaviruses.

Embodiment 89. The nucleic acid composition of embodiment 77 or 78, wherein no more than ten members of said plurality collectively exhibits specific binding to at least 97% of said different types of coronaviruses.

Embodiment 90. The nucleic acid composition of embodiment 77 or 78, wherein no more than eleven members of said plurality collectively exhibits specific binding to at least 98% of said different types of coronaviruses.

Embodiment 91. The nucleic acid composition of embodiment 77 or 78, wherein no more than fourteen members of said plurality collectively exhibits specific binding to at least 99% of said different types of coronaviruses.

Embodiment 92. The nucleic acid composition of embodiment 77 or 78, wherein no more than sixteen members of said plurality collectively exhibits specific binding to at least 99.5% of said different types of coronaviruses.

Embodiment 93. The nucleic acid composition of embodiment 77, wherein said plurality of RNA-binding guide RNAs are selected from the group consisting of the sequences in Table 4.

Embodiment 94. The nucleic acid composition of embodiment 78, wherein said plurality of RNA-binding guide RNAs are selected from the group consisting of the sequences in Table 4.

Embodiment 95. A composition for reducing transfection of a virus in a population of cells, comprising: a heterologous polypeptide comprising a gene regulating moiety, wherein said gene regulating moiety is configured to regulate expression of a target viral genome segment of said virus in a cell of said population of cells, and wherein said target viral genome segment comprises at least one non-coding region or a fragment thereof.

Embodiment 96. The composition of embodiment 95, wherein said gene regulating moiety is an RNA-guided gene regulating moiety that binds at least a portion of said target viral genome segment.

Embodiment 97. The composition of embodiment 96, further comprising at least one heterologous RNA polynucleotide configured to bind said at least said portion of said target viral genome segment.

Embodiment 98. The composition of embodiment 97, wherein said at least one heterologous RNA polynucleotide comprises a plurality of heterologous RNA polynucleotides configured to bind a plurality of different portions of said target viral genome segment.

Embodiment 99. The composition of embodiment 98, wherein said plurality of heterologous RNA polynucleotides comprises at least three different RNA polynucleotides.

Embodiment 10. The composition of embodiment 99, wherein said plurality of heterologous RNA polynucleotides comprises at least six different RNA polynucleotides.

Embodiment 101. The composition of embodiment 95, wherein said target viral genome segment is from an influenza virus, and said heterologous polypeptide is configured to effect reduced transfection of said influenza virus in said population of cells.

Embodiment 102. The composition of embodiment 101, wherein said at least one non-coding region is conserved across a plurality of influenza virus types selected from the group consisting of: influenza A, influenza B, influenza C, and influenza D.

Embodiment 103. The composition of embodiment 101, wherein said at least one non-coding region is conserved across a plurality of influenza A subtypes.

Embodiment 104. The composition of embodiment 101, wherein said target viral genome segment comprises a sequence having at least 90% sequence identity to a sequence selected from SEQ ID NOs: 13501-13548.

Embodiment 105. The composition of embodiment 101, wherein said heterologous polypeptide is configured to effect at least a 50% reduction in said transfection of said influenza virus in said population of cells.

Embodiment 106. The composition of embodiment 105, wherein said heterologous polypeptide is configured to effect at least a 60% reduction in said transfection of said influenza virus in said population of cells.

Embodiment 107. The composition of embodiment 106, wherein said heterologous polypeptide is configured to effect at least a 70% reduction in said transfection of said influenza virus in said population of cells.

Embodiment 108. The composition of embodiment 95, wherein said target viral genome segment comprises a first non-coding region and a second non-coding region that is different from said first non-coding region, and wherein said first and second non-coding regions are operatively coupled to a same viral polypeptide.

Embodiment 109. The composition of embodiment 108, wherein a polynucleotide sequence encoding said same viral polypeptide or a fragment thereof is flanked by said first and second non-coding regions.

Embodiment 110. The composition of embodiment 109, wherein said same viral polypeptide is selected from the group consisting of: a neuraminidase and a hemagglutinin.

Embodiment 111. The composition of embodiment 109, wherein said same viral polypeptide is said neuraminidase.

Embodiment 112. The composition of embodiment 95, wherein said target viral genome segment comprises (i) a first non-coding region operatively coupled to a first viral polypeptide and (ii) a second non-coding region operatively coupled to a second viral polypeptide, wherein said first and second viral polypeptides are different.

Embodiment 113. The composition of embodiment 112, wherein said first viral polypeptide is a neuraminidase and said second viral polypeptide is selected from the group consisting of; a polymerase, a hemagglutinin, a nucleoprotein, a matrix protein, a non-structural protein, and a nuclear export protein.

Embodiment 114. The composition of embodiment 112, wherein said first viral polypeptide is a hemagglutinin and said second viral polypeptide is selected from the group consisting of: a polymerase, a nucleoprotein, a matrix protein, a non-structural protein, and a nuclear export protein.

Embodiment 115. The composition of embodiment 97, wherein each of said at least one heterologous RNA polynucleotide has a length ranging from 10 to 30 nucleotides.

Embodiment 116. The composition of embodiment 96, wherein said RNA-guided gene regulating moiety is a CRISPR/Cas protein selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas 11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, and a combination thereof.

Embodiment 117. The composition of embodiment 96, wherein said RNA-guided gene regulating moiety exhibits RNA cleaving activity.

Embodiment 118. The composition of embodiment 95, wherein (i) said composition or (ii) one or more polynucleotides encoding said composition is packaged in a delivery vehicle selected from the group consisting of: a liposome, a nanoparticle, and a viral capsule.

Embodiment 119. A method of reducing transfection of a virus in a population of cells, comprising: contacting the population of cells with the composition of any one of embodiments 1-24, wherein upon said contacting, said composition effects reduced transfection of said virus in said population of cells.

Embodiment 120. The method of embodiment 119, wherein said contacting occurs in vivo, ex vivo, or in vitro.

Embodiment 121. The method of embodiment 119, further comprising expressing said composition in said cell.

Embodiment 122. The method of embodiment 119, further comprising administering (i) said composition or (ii) one or more polynucleotides encoding said composition to a subject in need thereof, thereby reducing said expression of said target viral genome segment in said subject.

Embodiment 123. The method of embodiment 119, further comprising, subsequent to said contacting, administering said cell to a subject in need thereof.

Embodiment 124. A kit comprising the composition of any one of embodiments 95-118.

Embodiment 125. A kit comprising one or more polynucleotides encoding the composition of any one of embodiments 95-118.

Embodiment 126. A reaction mixture comprising the composition of any one of embodiments 95-118 and a biological sample.

Embodiment 127. The reaction mixture of embodiment 126, wherein said biological sample is selected from the group consisting of: urine, blood, cell, tissue, organ, organism, and a combination thereof.

Embodiment 128. A composition for reducing transfection of a virus in a population of cells, comprising: at least one heterologous RNA polynucleotide configured to bind a target viral genome segment of said virus in a cell of said population of cells, wherein said target viral genome segment comprises at least one non-coding region or a fragment thereof, wherein said at least one heterologous RNA polynucleotide is capable of forming a complex with a heterologous polypeptide comprising a gene regulating moiety to reduce transfection of said virus in said population of cells.

Embodiment 129. The composition of embodiment 128, wherein said at least one heterologous RNA polynucleotide comprises a plurality of heterologous RNA polynucleotides configured to bind a plurality of different portions of said target viral genome segment.

Embodiment 130. The composition of embodiment 129, wherein said plurality of heterologous RNA polynucleotides comprises at least three different RNA polynucleotides.

Embodiment 131. The composition of embodiment 130, wherein said plurality of heterologous RNA polynucleotides comprises at least six different RNA polynucleotides.

Embodiment 132. The composition of embodiment 129, wherein said target viral genome segment is from an influenza virus.

Embodiment 133. The composition of embodiment 132, wherein said at least one non-coding region is conserved across a plurality of influenza virus types selected from the group consisting of: influenza A, influenza B, influenza C, and influenza D.

Embodiment 134. The composition of embodiment 132, wherein said at least one non-coding region is conserved across a plurality of influenza A subtypes.

Embodiment 135. The composition of embodiment 130, wherein said heterologous polypeptide is configured to effect at least a 50% reduction in said transfection of said influenza virus in said population of cells.

Embodiment 136. The composition of embodiment 135, wherein said heterologous polypeptide is configured to effect at least a 60% reduction in said transfection of said influenza virus in said population of cells.

Embodiment 137. The composition of embodiment 136, wherein said heterologous polypeptide is configured to effect at least a 70% reduction in said transfection of said influenza virus in said population of cells.

Embodiment 138. The composition of embodiment 129, wherein said target viral genome segment comprises a first non-coding region and a second non-coding region that is different from said first non-coding region, and wherein said first and second non-coding regions are operatively coupled to a same viral polypeptide.

Embodiment 139. The composition of embodiment 138, wherein a polynucleotide sequence encoding said same viral polypeptide or a fragment thereof is flanked by said first and second non-coding regions.

Embodiment 140. The composition of embodiment 138, wherein said same viral polypeptide is selected from the group consisting of: a neuraminidase and a hemagglutinin.

Embodiment 141. The composition of embodiment 138, wherein said same viral polypeptide is said neuraminidase.

Embodiment 142. The composition of embodiment 128, wherein said target viral genome segment comprises (i) a first non-coding region operatively coupled to a first viral polypeptide and (ii) a second non-coding region operatively coupled to a second viral polypeptide, wherein said first and second viral polypeptides are different.

Embodiment 143. The composition of embodiment 142, wherein said first viral polypeptide is a neuraminidase and said second viral polypeptide is selected from the group consisting of: a polymerase, a hemagglutinin, a nucleoprotein, a matrix protein, a non-structural protein, and a nuclear export protein.

Embodiment 144. The composition of embodiment 143, wherein said first viral polypeptide is a hemagglutinin and said second viral polypeptide is selected from the group consisting of: a polymerase, a nucleoprotein, a matrix protein, a non-structural protein, and a nuclear export protein.

Embodiment 145. The composition of embodiment 129, wherein each of said at least one heterologous RNA polynucleotide has a length ranging from 10 to 30 nucleotides.

Embodiment 146. The composition of embodiment 128, further comprising said heterologous polypeptide comprising said gene regulating moiety.

Embodiment 147. The composition of embodiment 146, wherein said gene regulating moiety is an RNA-guided gene regulating moiety that binds at least a portion of said target viral genome segment.

Embodiment 148. The composition of embodiment 129, wherein said gene regulating moiety is a CRISPR/Cas protein selected from the group consisting of: C2C1, C2C2, C2C3, Cas1, Cas1B, Cas2, Cas3, Cas4, Cas5, Cas5e, Cas6, Cas6e, Cas6f, Cas7, Cas8a, Cas8a1, Cas8a2, Cas8b, Cas8c, Cas9, Cas10, Cas10d, Cas10, Cas10d, Cas 11, Cas12, Cas13, Cas14, CasF, CasG, CasH, CasX, CaxY, Cpf1, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, a modification thereof, and a combination thereof.

Embodiment 149. The composition of embodiment 129, wherein said gene regulating moiety exhibits RNA cleaving activity.

Embodiment 150. The composition of embodiment 129, wherein (i) said composition or (ii) one or more polynucleotides encoding said composition is packaged in a delivery vehicle selected from the group consisting of: a liposome, a nanoparticle, and a viral capsule.

Embodiment 151. A method of reducing transfection of a virus in a population of cells, comprising: contacting the population of cells with the composition of any one of embodiments 34-56, wherein upon said contacting, said composition effects reduced transfection of said virus in said population of cells.

Embodiment 152. The method of embodiment 151, wherein said contacting occurs in vivo, ex vivo, or in vitro.

Embodiment 153. The method of embodiment 151, further comprising expressing said composition in said cell.

Embodiment 154. The method of embodiment 151, further comprising administering (i) said composition or (ii) one or more polynucleotides encoding said composition to a subject in need thereof, thereby reducing said expression of said target viral genome segment in said subject.

Embodiment 155. The method of embodiment 151, further comprising, subsequent to said contacting, administering said cell to a subject in need thereof.

Embodiment 156. A kit comprising the composition of any one of embodiments 128-150.

Embodiment 157. A kit comprising one or more polynucleotides encoding the composition of any one of embodiments 128-150.

Embodiment 158. A nucleic acid composition comprising a RNA-binding guide RNA, wherein said RNA-binding guide RNA comprises a targeting sequence that has a length ranging from 10 to 30 nucleotides; and wherein said RNA-binding guide RNA has at least 90% sequence identity to a sequence selected from SEQ ID NOs:13301-13348.

Embodiment 159. The nucleic acid composition of embodiment 150, further comprising a plurality of RNA-binding guide RNAs comprising said RNA-binding guide RNA, wherein each of said plurality of RNA-binding guide RNAs has at least 90% sequence identity to a sequence selected from SEQ ID NOs:13301-13348.

Embodiment 160. The nucleic acid composition of embodiment 159, wherein said plurality of RNA-binding guide RNAs comprise at most six RNA-binding guide RNAs.

Embodiment 161. A nucleic acid composition comprising a RNA-binding guide RNA, wherein said RNA-binding guide RNA comprises a targeting sequence that has a length ranging from 10 to 30 nucleotides and is selected from the group consisting of:

(i) a RNA polynucleotide having at least 90% sequence identity to SEQ ID NO: 13319;
(ii) a RNA polynucleotide having at least 90% sequence identity to SEQ ID NO: 13320;
(iii) a RNA polynucleotide having at least 90% sequence identity to SEQ ID NO: 13321;
(iv) a RNA polynucleotide having at least 90% sequence identity to SEQ ID NO: 13322;
(v) a RNA polynucleotide having at least 90% sequence identity to SEQ ID NO: 13323;
(vi) a RNA polynucleotide having at least 90% sequence identity to SEQ ID NO: 13324;
(vii) a RNA polynucleotide having at least 90% sequence identity to SEQ ID NO: 13331;
(viii) a RNA polynucleotide having at least 90% sequence identity to SEQ ID NO: 13332;
(ix) a RNA polynucleotide having at least 90% sequence identity to SEQ ID NO: 13333;
(x) a RNA polynucleotide having at least 90% sequence identity to SEQ ID NO: 13334;
(xi) a RNA polynucleotide having at least 90% sequence identity to SEQ ID NO: 13335; and
(xii) a RNA polynucleotide having at least 90% sequence identity to SEQ ID NO: 13336.

Embodiment 162. The nucleic acid composition of embodiment 161, comprising a plurality of different RNA binding guide RNAs comprising said RNA-binding guide RNA, wherein each RNA-binding guide RNA of said plurality has a targeting sequence selected from the group consisting of: (i) through (xii).

Embodiment 163. The nucleic acid composition of embodiment 162, wherein said plurality comprises at least two different guide RNAs.

Embodiment 164. The nucleic acid composition of embodiment 163, wherein said plurality comprises at least three different guide RNAs.

Embodiment 165. The nucleic acid composition of embodiment 164, wherein said plurality comprises at least four different guide RNAs.

Embodiment 166. The nucleic acid composition of embodiment 165, wherein said plurality comprises at least five different guide RNAs.

Embodiment 167. The nucleic acid composition of embodiment 166, wherein said plurality comprises at least six different guide RNAs.

Embodiment 168. The nucleic acid composition of embodiment 163, wherein said at least two different guide RNAs are selected from the group consisting of (i)-(vi).

Embodiment 169. The nucleic acid composition of embodiment 163, wherein said at least two different guide RNAs are selected from the group consisting of (vii)-(xii).

Embodiment 170. The nucleic acid composition of embodiment 163, wherein said at least two different guide RNAs comprises (1) a first guide RNA selected from the group consisting of (i)-(vi) and (2) a second guide RNA selected from the group consisting of (vii)-(xii).

### EXAMPLES

The following examples are given for the purpose of illustrating various embodiments of the disclosure and are not meant to limit the present disclosure in any fashion. The present examples, along with the methods and systems described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Design Prophylactic Antiviral CRISPR in huMAN Cells (PAC-MAN) as a Form of Genetic Vaccine to Target Circulating SARS-COV-2 and Potentially all Sequenced Coronaviruses

With a rapidly growing number of cases and deaths around the world caused by the coronaviruses like SARS-COV-2 and the ongoing threats of Severe Acute Respiratory Syndrome (SARS), Middle East Respiratory Syndrome (MERS), and any other coronavirus strains evolving and becoming able to suddenly transfer to humans from diverse animal hosts that act as viral reservoirs, there remains a pressing need to effectively detect and target the viral genomes for degradation to prevent the expression of viral proteins. Recent reports further showed two strains (L and S) of SARS-COV-2 with different genome sequences are circulating and likely evolving, further highlighting the need for a pan-coronavirus vaccination or treatment strategy.

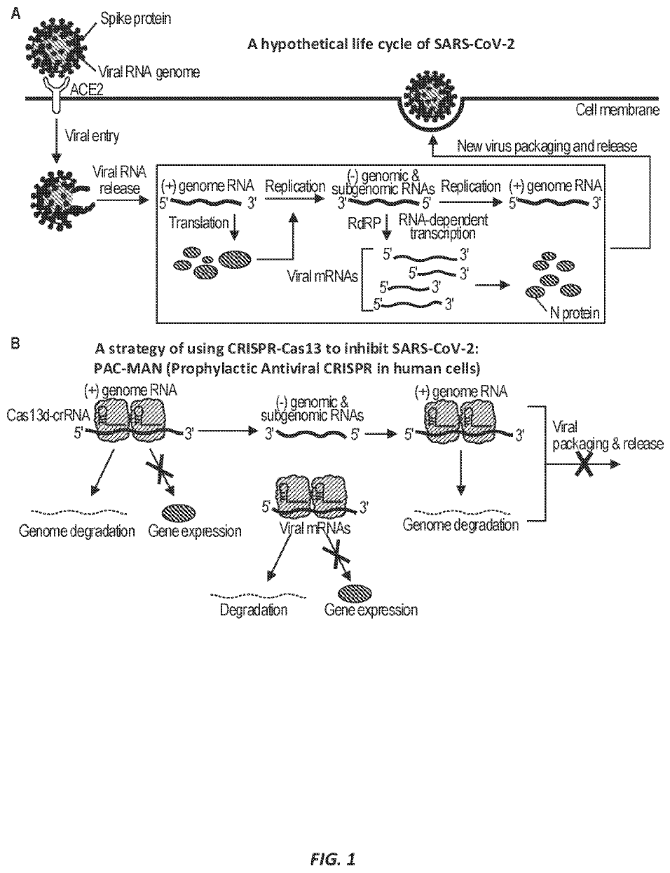

The SARS-COV-2 coronavirus belongs to a family of positive-sense RNA viruses, which typically infect lung epithelial cells and upper respiratory track cells and cause respiratory disease. The SARS-COV-2 life cycle can be similar to closely related coronaviruses such as SARS. As such, the SARS-COV-2 virus can enter the cell and release the viral genome into the cytoplasm (FIG. 1A), and creates a negative-sense template strand from which viral mRNAs and a new copy of the positive sense viral genome are created. Whereas traditional vaccines work by priming the human immune system to recognize viral proteins and decrease viral entry into cells, the alternative vaccine approach proposed herein relies on recognizing and degrading the intracellular viral genome and its resulting viral mRNAs (FIG. 1B). By targeting the positive genome and viral mRNAs, the simultaneous degradation of viral genome templates for replication and inhibition of viral gene expression can limit viral packaging and spreading.

To inhibit RNA viruses in human cells, the CRISPR-Cas13d system derived from Ruminococcus flavefaciens XPD3002, a recently discovered RNA-guided RNA endonuclease, can be used. Cas13d uses CRISPR-associated RNAs (crRNAs) that contain designable 22 nucleotide (nt) spacer regions that can direct the Cas13d protein to specific RNA molecules for targeted RNA degradation. This provides a potential mechanism for targeting SARS-COV-2 for specific viral RNA genome degradation and viral gene expression inhibition. Because of its small size (2.8 kb), high specificity, and strong catalytic activity, the Cas13d can target and destroy RNA viruses including SARS-COV-2.

Described herein is a CRISPR strategy named Prophylactic Antiviral CRISPR in huMAN cells (PAC-MAN) developed as a form of genetic vaccine to target circulating SARS-COV-2 and potentially all sequenced coronaviruses. A bioinformatic pipeline defined highly conserved regions across the coronavirus genomes and flagged these regions for genome degradation and viral gene inhibition by the use of CRISPR-dCas13d. A panel of designed crRNA pools was screened against SARS-COV-2 fragments for how well the crRNAs may target conserved viral regions in order to identify the most effective crRNAs. Such pools of crRNAs can then be part of a pan-coronavirus treatment regiments.

### Example 2. High-Through Guide Design

47 complete 2019-nCov assemblies were obtained from National Center for Biotechnology Information. All the other 3.108 Coronavirinae complete genome sequences were obtained from ViPR (Virus Pathogen Resource) database. After excluding very short genomes, a total 3150 Coronavirinae virus genomes were collected.

All the complete SARS-COV-2 genome sequences were aligned with the SARS and MERS NCBI Refseq sequences by MAFFT using the --auto flag. A consensus nucleotide (which was shared by >50% of non-gap characters at the given position) was assigned to each position in the alignment; not every position had a consensus nucleotide. The percent conservation at each position was calculated as the percentage of sequences matching the consensus at that position. To aid interpretation, the resulting plot of percentage conservation was smoothed using a moving average with a window size of 1,001 bp centered at each position. Positions corresponding to gap characters in the SARS-COV-2 reference sequence were omitted.

A comprehensive computational workflow was developed to design guide RNAs for Cas13d. In order to design all possible guides for the three pathogenic RNA viruses (2019-nCoV, MERS and SARS), first the reference genomes of SARS, MERS, SARS-COV-2, along with 47 SARS-COV-2 genomes were aligned by MAFFT using the --auto flag. Guide candidates were then identified by using a sliding window to extract all 22 bp sequences with perfect identity among the SARS-COV-2 genomes. Each guide candidate was annotated with the number of mismatches relative to the SARS and MERS genomes, as well as the GC content. 3802 guide candidates were selected with perfect match against the 47 SARS-COV-2 genomes and with <1 MERS mismatch or <1 SARS mismatch.

In addition, the vertical coverage of pooled guides was maximized so as to target a maximal set of the 3150 Coronavirinae viruses with a minimal set of guides. A sliding window was used to extract all unique 22 nt sequences in the Coronavirinae genomes, yielding 6,480,955 unique guide candidates. A hash table was used to map each guide candidate to the number of Coronavirinae genomes that it targets with a perfect match. A greedy approach was used to identify a minimal covering set of guides. In each iteration, the guides were sorted in descending order by the number of genomes targeted. The guide that targeted the most genomes was added to the minimal set of guides, and all of the genomes it targeted were removed from the hash table values. If multiple guides were tied for targeting the most genomes, the guide were ranked by the length and quantity of stretches of repeated bases, and the lowest ranking guide (i.e. the one with the fewest, shortest stretches of repeated bases) was added to the set of minimal guides. Using this approach, a set of 23 guides was identified that was able to target all 3150 Coronavirinae virus genomes (including 47 SARS-COV-2 genomes).

In order to characterize 22 nt crRNA specificity, both sets of guides were confirmed to not target any sequences in the human transcriptome. Bowtie was used to align guides to the human transcriptome (HG38; including non-coding RNA) and removed guides that mapped to the human transcriptome with fewer than 2 mismatches.

### Example 3. Computational Design and Analysis of Targetable Regions on SARS-COV-2 for High-Through Put crRNA Design

To create effective and specific crRNA sequences to target and cleave SARS-COV-2, bioinformatic analysis was performed by aligning 47 genomes of published SARS-COV-2 genomes and SARS and MERS genomes isolated from patients at the nucleotide resolution. SARS-COV-2 has a single-stranded RNA genome with ˜30,000 nucleotides which encodes 12 putative, functional open reading frames.

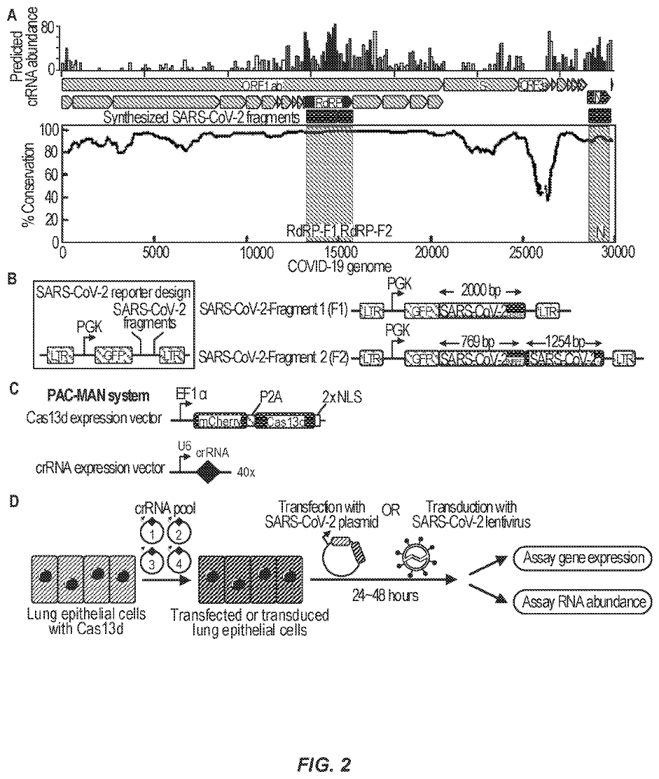

The bioinformatic analysis identified regions with a high conservation among 47 SARS-COV-2 genomes and genomes of SARS and MERS (FIG. 2A). The highly conserved regions contain RNA-dependent RNA polymerase (RdRP) in the polypeptide ORF1ab region, which encodes RNA-dependent RNA polNmerase that maintains the proliferation of all coronavinses, and Nucleocapsid (N) gene at the 3′ end of the genome, which encodes capsid proteins for viral packaging.

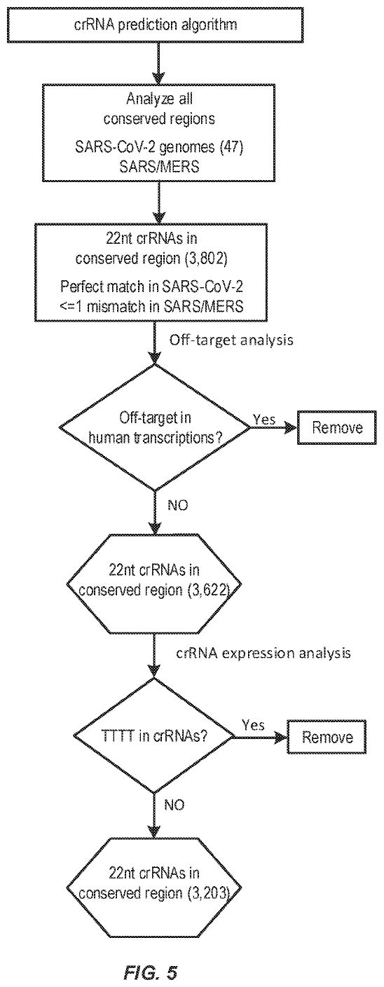

To target these two conserved regions, a collection of all 3,802 crRNAs was generated in silico based on the work-flow depicted in FIG. 5. After off-target analysis by aligning to the human transcriptome (<=1 mismatch) and ruling out poly-T sequences that may prevent crRNA expression, a collection of crRNAs was obtained (SEQ ID NO: 1-3203, 4001-7203, and 8001-11203).

40 crRNAs were synthesized, among which 20 crRNAs targeted the conserved sequences of RdRP, and 20 crRNAs targeted N (nucleocapsid) gene (Table 1; SEQ ID NO: 12001-12040, 12101-12140, and 12201-12240) for crRNA sequences and coordinates on SARS-COV-2). These regions were targeted to avoid mutational escape from viruses and to significantly reduce both virus genome copies and viral mRNA templates for expressing essential viral proteins.

TABLE 1
Sequences of 40 crRNA targeting SARS-CoV-2

| | | | | | Target viral | | | |
|---|---|---|---|---|---|---|---|---|
| | Spacer | | | | genome | | FIG. | FIG. |
| SEQ ID | DNA | SEQ ID | Spacer RNA | SEQ ID | RNA | Target | 3A-D | 3E |
| NO | sequence | NO | sequence | NO | sequence | gene | pool | pool |
| SEQ ID | tcctgagcaaa | SEQ ID | uccugagcaaa | SEQ ID | aacacuucuuc | RdRP | G1 | F1 |
| NO: | gaagaagtgtt | NO: | gaagaaguguu | NO: | uuugcucagga | | | |
| 12001 | | 12101 | | 12201 | | | | |
| SEQ ID | tgtctgatatca | SEQ ID | ugucugauauc | SEQ ID | caacaaugugu | RdRP | G1 | |
| NO: | cacattgttg | NO: | acacauuguug | NO: | gauaucagaca | | | |
| 12002 | | 12102 | | 12202 | | | | |
| SEQ ID | tctgatcccaat | SEQ ID | ucugaucccaa | SEQ ID | uauuuuaaaua | RdRP | G1 | |
| NO: | atttaaaata | NO: | uauuuaaaaua | NO: | uugggaucaga | | | |
| 12003 | | 12103 | | 12203 | | | | |
| SEQ ID | gtctcttaacta | SEQ ID | gucucuuaacu | SEQ ID | ucuuacuuugu | RdRP | G1 | |
| NO: | caaagtaaga | NO: | acaaaguaaga | NO: | aguuaagagac | | | |
| 12004 | | 12104 | | 12204 | | | | |
| SEQ ID | ggcgtacacgt | SEQ ID | ggcguacacgu | SEQ ID | aacuuagguga | RdRP | G2 | |
| NO: | tcacctaagtt | NO: | ucaccuaaguu | NO: | acguguacgcc | | | |
| 12005 | | 12105 | | 12205 | | | | |
| SEQ ID | gattcatttgag | SEQ ID | gauucauuuga | SEQ ID | ccuacuauaac | RdRP | G2 | |
| NO: | ttatagtagg | NO: | guuauaguagg | NO: | ucaaaugaauc | | | |
| 12006 | | 12106 | | 12206 | | | | |
| SEQ ID | tacggtgcga | SEQ ID | uacggugcgag | SEQ ID | aagaauagagc | RdRP | G2 | |
| NO: | gctctattctt | NO: | cucuauucuu | NO: | ucgcaccgua | | | |
| 12007 | | 12107 | | 12207 | | | | |
| SEQ ID | gataatcccaa | SEQ ID | gauaaucccaac | SEQ ID | caccuuauggg | RdRP | G2 | |
| NO: | cccataaggtg | NO: | ccauaaggug | NO: | uugggauuauc | | | |
| 12008 | | 12108 | | 12208 | | | | |
| SEQ ID | cttgagcacac | SEQ ID | cuugagcacac | SEQ ID | uuagcuaauga | RdRP | G3 | |
| NO: | tcattagctaa | NO: | ucauuagcuaa | NO: | gugugcucaag | | | |
| 12009 | | 12109 | | 12209 | | | | |
| SEQ ID | tagcagggtca | SEQ ID | uagcaggguca | SEQ ID | guguaugcugc | RdRP | G3 | |
| NO: | gcagcataca | NO: | gcagcauacac | NO: | ugacccugcua | | | |
| 12010 | c | 12110 | | 12210 | | | | |
| SEQ ID | aaacattaaag | SEQ ID | aaacauuaaag | SEQ ID | cauugugcaaa | RdRP | G3 | |
| NO: | tttgcacaatg | NO: | uuugcacaaug | NO: | cuuuaauguuu | | | |
| 12011 | | 12111 | | 12211 | | | | |
| SEQ ID | gacaacaatta | SEQ ID | gacaacaauua | SEQ ID | uuccuaaaaac | RdRP | G3 | F1 |
| NO: | gtttttaggaa | NO: | guuuuuaggaa | NO: | uaauuguuguc | | | |
| 12012 | | 12112 | | 12212 | | | | |
| SEQ ID | atatatgtggta | SEQ ID | auauauguggu | SEQ ID | ggugacauggu | RdRP | G4 | |
| NO: | ccatgtcacc | NO: | accaugucacc | NO: | accacauauau | | | |
| 12013 | | 12113 | | 12213 | | | | |
| SEQ ID | attaccttcatc | SEQ ID | auuaccuucau | SEQ ID | aaggcauuuug | RdRP | G4 | |
| NO: | aaaatgcctt | NO: | caaaaugccuu | NO: | augaagguaau | | | |
| 12014 | | 12114 | | 12214 | | | | |
| SEQ ID | cttgattatcta | SEQ ID | cuugauuaucu | SEQ ID | guacugacauu | RdRP | G4 | |
| NO: | atgtcagtac | NO: | aaugucaguac | NO: | agauaaucaag | | | |
| 12015 | | 12115 | | 12215 | | | | |
| SEQ ID | aagaatctaca | SEQ ID | aagaaucuacaa | SEQ ID | ggaguuccugu | RdRP | G4 | F1 |
| NO: | acaggaactc | NO: | caggaacucc | NO: | uguagauucuu | | | |
| 12016 | c | 12116 | | 12216 | | | | |
| SEQ ID | atcataagtgta | SEQ ID | aucauaagugu | SEQ ID | aaaacagaugg | RdRP | G5 | F1 |
| NO: | ccatctgtttt | NO: | accaucuguuu | NO: | uacacuuauga | | | |
| 12017 | | 12117 | u | 12217 | u | | | |
| SEQ ID | agcaaaattca | SEQ ID | agcaaaauuca | SEQ ID | aaaggaccuca | RdRP | G5 | |
| NO: | tgaggtccttt | NO: | ugagguccuuu | NO: | ugaauuuugcu | | | |
| 12018 | | 12118 | | 12218 | | | | |
| SEQ ID | aacattttgctt | SEQ ID | aacauuuugcu | SEQ ID | uuuaugucuga | RdRP | G5 | |
| NO: | cagacataaa | NO: | ucagacauaaa | NO: | agcaaaauguu | | | |
| 12019 | | 12119 | | 12219 | | | | |
| SEQ ID | cactattagcat | SEQ ID | cacuauuagca | SEQ ID | acaacugcuua | RdRP | G5 | F1 |
| NO: | aagcagttgt | NO: | uaagcaguugu | NO: | ugcuaauagug | | | |
| 12020 | | 12120 | | 12220 | | | | |
| SEQ ID | tcttctttttgtcc | SEQ ID | ucuucuuuuug | SEQ ID | ccuaaaaagga | RdRP | G6 | F2 |
| NO: | tttttagg | NO: | uccuuuuuagg | NO: | caaaaagaaga | | | |
| 12021 | | 12121 | | 12221 | | | | |
| SEQ ID | gaatgttttgtat | SEQ ID | gaauguuuugu | SEQ ID | uauugacgcau | RdRP | G6 | |
| NO: | gcgtcaata | NO: | augcgucaaua | NO: | acaaaacauuc | | | |
| 12022 | | 12122 | | 12222 | | | | |
| SEQ ID | tttggatctttgt | SEQ ID | uuuggaucuuu | SEQ ID | aauuggaugac | RdRP | G6 | |
| NO: | catccaatt | NO: | gucauccaauu | NO: | aaagauccaaa | | | |
| 12023 | | 12123 | | 12223 | | | | |
| SEQ ID | ccacgttcccg | SEQ ID | ccacguucccg | SEQ ID | agucacaccuu | RdRP | G6 | |
| NO: | aaggtgtgact | NO: | aaggugugacu | NO: | cgggaacgugg | | | |
| 12024 | | 12124 | | 12224 | | | | |
| SEQ ID | aaattgtgcaat | SEQ ID | aaauugugcaa | SEQ ID | uuggccgcaaa | N | G7 | F2 |
| NO: | ttgcggccaa | NO: | uuugcggccaa | NO: | uugcacaauuu | | | |
| 12025 | | 12125 | | 12225 | | | | |
| SEQ ID | aatcagttcctt | SEQ ID | aaucaguuccu | SEQ ID | cuaaucagaca | N | G7 | |
| NO: | gtctgattag | NO: | ugucugauuag | NO: | aggaacugauu | | | |
| 12026 | | 12126 | | 12226 | | | | |
| SEQ ID | cttgggtttgtt | SEQ ID | cuuggguuugu | SEQ ID | cgugguccaga | N | G7 | |
| NO: | ctggaccacg | NO: | ucuggaccacg | NO: | acaaacccaag | | | |
| 12027 | | 12127 | | 12227 | | | | |
| SEQ ID | gtggcagtac | SEQ ID | guggcaguacg | SEQ ID | cucggcaaaaa | N | G7 | |
| NO: | gtttttgccgag | NO: | uuuuugccgag | NO: | cguacugccac | | | |
| 12028 | | 12128 | | 12228 | | | | |
| SEQ ID | gcctcagcag | SEQ ID | gccucagcagc | SEQ ID | cuaagaaaucu | N | G8 | |
| NO: | cagatttcttag | NO: | agauuucuuag | NO: | gcugcugaggc | | | |
| 12029 | | 12129 | | 12229 | | | | |
| SEQ ID | gttgttgttggc | SEQ ID | guaguuguug | SEQ ID | ucagguaaagg | N | G8 | F2 |
| NO: | ctttaccaga | NO: | gccuuuaccag | NO: | ccaacaacaac | | | |
| 12030 | | 12130 | a | 12230 | | | | |
| SEQ ID | gcctggagttg | SEQ ID | gccuggaguug | SEQ ID | uucaagaaauu | N | G8 | |
| NO: | aatttcttgaa | NO: | aauuucuugaa | NO: | caacuccaggc | | | |
| 12031 | | 12131 | | 12231 | | | | |
| SEQ ID | gaagaggctt | SEQ ID | gaagaggcuug | SEQ ID | gaggcggcagu | N | G8 | |
| NO: | gactgccgcct | NO: | acugccgccuc | NO: | caagccucuuc | | | |
| 12032 | c | 12132 | | 12232 | | | | |
| SEQ ID | ttttggcaatgtt | SEQ ID | uuuuggcaaug | SEQ ID | ucaaggaacaa | N | G9 | |
| NO: | gttccttga | NO: | uuguuccuuga | NO: | cauugccaaaa | | | |
| 12033 | | 12133 | | 12233 | | | | |
| SEQ ID | aggattgcgg | SEQ ID | aggauugcggg | SEQ ID | ucacauuggca | N | G9 | |
| NO: | gtgccaatgtg | NO: | ugccaauguga | NO: | cccgcaauccu | | | |
| 12034 | a | 12134 | | 12234 | | | | |
| SEQ ID | aggctccctca | SEQ ID | aggcucccuca | SEQ ID | uggguugcaac | N | G9 | F2 |
| NO: | gttgcaaccca | NO: | guugcaaccca | NO: | ugagggagccu | | | |
| 12035 | | 12135 | | 12235 | | | | |
| SEQ ID | ggtagctcttc | SEQ ID | gguagcucuuc | SEQ ID | uggcuacuacc | N | G9 | |
| NO: | ggtagtagcca | NO: | gguaguagcca | NO: | gaagagcuacc | | | |
| 12036 | | 12136 | | 12236 | | | | |
| SEQ ID | aacgccttgtc | SEQ ID | aacgccuuguc | SEQ ID | anucccucgag | N | G10 | |
| NO: | ctcgagggaat | NO: | cucgagggaau | NO: | gacaaggcguu | | | |
| 12037 | | 12137 | | 12237 | | | | |
| SEQ ID | tgaaccaaga | SEQ ID | ugaaccaagac | SEQ ID | aauaauacagc | N | G10 | |
| NO: | cgcagtattatt | NO: | gcaguauuauu | NO: | gucuugguuca | | | |
| 12038 | | 12138 | | 12238 | | | | |
| SEQ ID | ccccactgcgt | SEQ ID | ccccacugcgu | SEQ ID | cagaauggaga | N | G10 | |
| NO: | tctccattctg | NO: | ucuccauucug | NO: | acgcagugggg | | | |
| 12039 | | 12139 | | 12239 | | | | |
| SEQ ID | ttgaatctgag | SEQ ID | uagaaucugag | SEQ ID | uuugguggacc | N | G10 | F2 |
| NO: | ggtccaccaa | NO: | gguccaccaaa | NO: | cucagauucaa | | | |
| 12040 | a | 12140 | | 12240 | | | | |

### Example 4. Cas13d PAC-MAN is Capable of Inhibiting Coronavirus Fragment Expression in Human Lung Epithelial Cells

The crRNA plasmids were cloned using standard restriction-ligation cloning. Forward and reverse oligos for each spacer were annealed and inserted into backbone using T4 DNA Ligase (NEB). Assembled oligos were either inserted into a pHR or pUC 19 backbone, obtained and modified from Addgene (#121514 and #109054, respectively). Spacer sequences for all crRNAs can be found in Table 1.

To clone SARS-COV-2 reporters, pHR-PGK-scFv GCN4-sfGFP (pSLQ1711) was digested with EcoR1 and SKf1 and gel purified. The PGK promoter and sfGFP were PCR amplified from pSLQ1711 and gel purified. The COV1 and COV2 fragments were synthesize from Integrated DNA Technologies. At last, the PGK, sfGFP, and COV1/COV2 fragment were ligated into the linearized pSLQ1711 vector.

At day 1, one confluent 100 mm dish of HEK293T cells were seeded into three 150 mm dish format. At day 2, cells were ˜50-70% confluent at the time of transfection. For each dish, 27.18 μg of pHR vector containing the construct of interest, 23.76 μg of dR8.91 and 2.97 μg of pMD2.G were mixed in 4.5 mL of Opti-MEM reduced serum media with 150 μL of Mirus TransIT-LT1 reagent and incubated at room temperature for 30 minutes. The transfection complex solution was distributed evenly to HEK293T cultures dropwise. At day 5, lentiviruses were harvested from the supernatant with a sterile syringe and filtered through a 0.22-μm polyvinylidene fluoride filter.

For A549 cells, lentivirus precipitation solution was added and precipitated as per the manufacturer's protocol. One well of a 6-well plate of A549 cells at ˜50% confluency were transduced with the precipitated lentivirus. After 2-3 days of growth, the cell supernatant containing virus was removed and the cells were expanded. Cells were then sorted for mCherry+ cells using a cell sorter.

To evaluate whether Cas13d was effective for targeting SARS-COV-2 sequences, two reporters expressing were created based on synthesized fragments of SARS-COV-2 fused to GFP (FIG. 2B: SARS-COV-2-F1 and SARS-COV-2-F2; and Table 2 (SEQ ID NO: 12251 and 12252) for SARS-COV-2 fragment sequences) for designing and testing effectiveness of the crRNAs. SARS-COV-2 infects mainly respiratory tract cells in patients; therefore, human lung epithelial A549 cells were chosen as the model cell line. A stable A549 cell line were created to express Cas13d through lentiviral transduction, followed by sorting for the mCherry marker that was co-expressed with Cas13d (FIG. 2C). The crRNAs were expressed from a U6 promoter.

TABLE 2
Synthesized SARS-COV-2 fragment sequences

(A) SARS-CoV-2-F1 reporter, SEQ ID NO: 12251
cttcagtcagctgatgcacaatcgtttttaaacgggtttgcggtgtaagtgcagcccgtcttacaccgtgcggcacaggcactagtac
tgatgtcgtatacagggcttttgacatctacaatgataaagtagctggttttgctaaattcctaaaaactaattgttgtcgcttccaagaaa
aggacgaagatgacaatttaattgattcttactttgtagttaagagacacactttctctaactaccaacatgaagaaacaatttataattta
cttaaggattgtccagctgttgctaaacatgacttctttaagtttagaatagacggtgacatggtaccacatatatcacgtcaacgtctta
ctaaatacacaatggcagacctcgtctatgctttaaggcattttgatgaaggtaattgtgacacattaaaagaaatacttgtcacataca
attgttgtgatgatgattatttcaataaaaaggactggtatgattttgtagaaaacccagatatattacgcgtatacgccaacttaggtga
acgtgtacgccaagctttgttaaaaacagtacaattctgtgatgccatgcgaaatgctggtattgttggtgtactgacattagataatca
agatctcaatggtaactggtatgatttcggtgatttcatacaaaccacgccaggtagtggagttcctgttgtagattcttattattcattgtt
aatgcctatattaaccttgaccagggctttaactgcagagtcacatgttgacactgacttaacaaagccttacattaagtgggatttgtt
aaaatatgacttcacggaagagaggttaaaactctttgaccgttattttaaatattgggatcagacataccacccaaattgtgttaactgt
ttggatgacagatgcattctgcattgtgcaaactttaatgttttattctctacagtgttcccacctacaagttttggaccactagtgagaaa
aatatttgttgatggtgttccatttgtagtttcaactggataccacttcagagagctaggtgttgtacataatcaggatgtaaacttacata
gctctagacttagttttaaggaattacttgtgtatgctgctgaccctgctatgcacgctgcttctggtaatctattactagataaacgcact
acgtgcttttcagtagctgcacttactaacaatgttgcttttcaaactgtcaaacccggtaattttaacaaagacttctatgactttgctgtg
tctaagggtttctttaaggaaggaagttctgttgaattaaaacacttcttctttgctcaggatggtaatgctgctatcagcgattatgacta
ctatcgttataatctaccaacaatgtgtgatatcagacaactactatttgtagttgaagttgttgataagtactttgattgttacgatggtgg
ctgtattaatgctaaccaagtcatcgtcaacaacctagacaaatcagctggttttccatttaataaatggggtaaggctagactttattat
gattcaatgagttatgaggatcaagatgcacttttcgcatatacaaaacgtaatgtcatccctactataactcaaatgaatcttaagtatg
ccattagtgcaaagaatagagctcgcaccgtagctggtgtctctatctgtagtactatgaccaatagacagtttcatcaaaaattattga
aatcaatagccgccactagaggagctactgtagtaattggaacaagcaaattctatggiggttggcacaacatgttaaaaactgtttat
agtgatgtagaaaaccctcaccttatgggttgggattatcctaaatgtgatagagccatgcctaacatgcttagaattatggcctcactt
gttcttgctcgcaaacatacaacgtgttgtagcttgtcacaccgtttctatagattagctaatgagtgtgctcaagtattgagtgaa
(B) SARS-CoV-2-F2 reporter (lower case = RRP2, upper case = N), SEQ ID NO: 12252
atggtcatgtgtggcggttcactatatgttaaaccaggtggaacctcatcaggagatgccacaactgcttatgctaatagtgtttttaac
atttgtcaagctgtcacggccaatgttaatgcacttttatctactgatggtaacaaaattgccgataagtatgtccgcaatttacaacaca
gactttatgagtgtctctatagaaatagagatgttgacacagactttgtgaatgagttttacgcatatttgcgtaaacatttctcaatgatg
atactctctgacgatgctgttgtgtgtttcaatagcacttatgcatctcaaggtctagtggctagcataaagaactttaagtcagttctttat
tatcaaaacaatgtttttatgtctgaagcaaaatgttggactgagactgaccttactaaaggacctcatgaattttgctctcaacatacaa
tgctagttaaacagggtgatgattatgtgtaccttccttacccagatccatcaagaatcctaggggccggctgttttgtagatgatatcg
taaaaacagatggtacacttatgattgaacggttcgtgtctttagctatagatgcttacccacttactaaacatcctaatcaggagtatgc
tgatgtctttcatttgtacttacaatacataagaaagctacatgatgagttaacaggacacatgttagacatgtattctgttatgcttactaa
tgataacacttcaaggtattgggaacctgagttttatgTCTGATAATGGACCCCAAAATCAGCGAAATG
CACCCCGCATTACGTTTGGTGGACCCTCAGATTCAACTGGCAGTAACCAGAAT
GGAGAACGCAGTGGGGCGCGATCAAAACAACGTCGGCCCCAAGGTTTACCCA
ATAATACTGCGTCTTGGTTCACCGCTCTCACTCAACATGGCAAGGAAGACCTTA
AATTCCCTCGAGGACAAGGCGTTCCAATTAACACCAATAGCAGTCCAGATGAC
CAAATTGGCTACTACCGAAGAGCTACCAGACGAATTCGTGGTGGTGACGGTAA
AATGAAAGATCTCAGTCCAAGATGGTATTTCTACTACCTAGGAACTGGGCCAG
AAGCTGGACTTCCCTATGGTGCTAACAAAGACGGCATCATATGGGTTGCAACT
GAGGGAGCCTTGAATACACCAAAAGATCACATTGGCACCCGCAATCCTGCTAA
CAATGCTGCAATCGTGCTACAACTTCCTCAAGGAACAACATTGCCAAAAGGCT
TCTACGCAGAAGGGAGCAGAGGCGGCAGTCAAGCCTCTTCTCGTTCCTCATCA
CGTAGTCGCAACAGTTCAAGAAATTCAACTCCAGGCAGCAGTAGGGGAACTTO
TCCTGCTAGAATGGCTGGCAATGGCGGTGATGCTGCTCTTGCTTTGCTGCTGCT
TGACAGATTGAACCAGCTTGAGAGCAAAATGTCTGGTAAAGGCCAACAACAAC
AAGGCCAAACTGTCACTAAGAAATCTGCTGCTGAGGCTTCTAAGAAGCCTCGG
CAAAAACGTACTGCCACTAAAGCATACAATGTAACACAAGCTTTCGGCAGACG
TGGTCCAGAACAAACCCAAGGAAATTTTGGGGACCAGGAACTAATCAGACAA
GGAACTGATTACAAACATTGGCCGCAAATTGCACAATTTGCCCCCAGCGCTTC
AGCGTTCTTCGGAATGTCGCGCATTGGCATGGAAGTCACACCTTCGGGAACGT
GGTTGACCTACACAGGTGCCATCAAATTGGATGACAAAGATCCAAATTTCAAA
GATCAAGTCATTTTGCTGAATAAGCATATTGACGCATACAAAACATTCCCACC
AACAGAGCCTAAAAAGGACAAAAAGAAGAAGGCTGATGAAACTCAAGCCITA
CCGCAGAGACAGAAGAAACAGCAAACTGTGACTCTTCTTCCTGCTGCAGATTT
GGATGATTTCTCCAAACAATTGCAACAATCCATGAGCAGTGCTGACTCAACTC
AGGCC

Regions of the SARS-COV-2 fragments were investigated to determine which were more susceptible to Cas13d-mediated targeting and cleavage. To do this, the SARS-COV-2 reporters were introduced into Cas13d A549 cells that had been transduced with pools of four crRNAs tiling adjacent SARS-COV-2 sequences targeting either RdRP or N (FIG. 2D). Pools of crRNAs were chosen to be evaluated to prevent live virus from escaping inhibition by mutating a single crRNA target site. A total of ten groups were tested for a combination of any one of SEQ ID NO: 12001-12040, 12101-12140, and 12201-12240. Twenty-four hours after SARS-COV-2 reporter transfection, flow cytometry analysis was performed to examine levels of GFP protein expression on single cell level and collected RNA from cells to perform quantitative real-time PCR (qRT-PCR) to examine mRNA transcript abundance.

### Example 5. SARS-CoV-2 Reporter Challenge Experiments

On day 1, cells were seeded at 60k cells per well of 12-well plate. On day 2, cells were transduced with pooled crRNAs. On day 3, cells were switched to fresh medium with puromycin at 2 μg/mL. 24 hours after puromycin selection, cells with crRNAs were challenged with SARS-COV-2 plasmids or SARS-COV-2 lentivirus. 24 hours after transfection or 48 hours after transduction, cells were tested by Flow cytometry and qRT-PCR.

Alternatively on day 0, 30,000 wild type (wt) A549 cells and 30,000 A549 cells stably expressing CasRx (hereafter referred to as CasRx A549) were plated in 24-well plates. On day 1, CasRx A549 cells were transfected with two pool of five guides targeting the two SARS-COV-2 reporters or a non-targeting guide along with the respective SARS-COV-2 reporters in an equimolar ratio using Mirus LT1 reagent (50 μL Opti-MEM reduced serum media, 0.136 μg guide pool, 0.364 μg SARS-COV-2 reporter, 1 μL Mirus LT1 per well). Wt cells were transfected with either the guide pools or reporter plasmids as compensation controls. Knockdown of GFP was measured on day 3 by flow cytometry.

For transduction of SARS-COV-2 reporter plasmid, on day 0, LentiX HEK cells were seeded to 1:6 from a 100 mm dish in a 150 mm dish. The following day, HEK cells were transfected with 27.18 μg of SARS-COV-2 reporter, 23.76 μg of dR8.91 and 2.97 μg of pMD2.G in 4.5 mL of Opti-MEM reduced serum media with 150 μL of Mirus TransIT-LT1 reagent. 30,000 CasRx A549 and wt A549 cells were also seeded in 24-well plates. On day 2, A549 cells were transfected with either pools of targeting guides or a non-targeting guide (50 μL Opti-MEM reduced serum media, 0.5 μg guide pool, 1 μL Mirus LT1 per well). On day 3, lentiviruses were harvested from the supernatant with a sterile syringe and filtered through a 0.22-μm polyvinylidene fluoride filter and precipitated with lentivirus precipitation solution and resuspended in 7 mL DMEM media. The media on the cells was replaced with either fresh media for the compensation controls or 0.5 mL virus-containing media at the following dilutions for the experimental conditions: undiluted, ½ dilution, ⅓ dilution, and ¼ dilution. On day 4, the media on all the cells was replaced with 0.5 mL fresh DMEM. On day 5, cells were measured by flow cytometry and assessed for GFP knockdown.

Real-time qRT-PCR was performed to determine the mRNA expression change of coronavirus fragment. For each sample, total RNA was isolated by using the RNeasy Plus Mini Kit, followed by cDNA synthesis using the iScript cDNA Synthesis Kit. Quantitative PCR was performed using the PrimePCR assay with the SYBR Green Master Mix, and run on a Biorad CFX384 real-time system, according to manufacturers' instructions. Cq values were used to quantify mRNA expression. The relative expression of the coronavirus fragments was normalized to a GAPDH internal control. Primers were ordered from Integrated DNA Technologies (IDT).

Microscopy for SARS-COV-2 experiments was performed on An inverted confocal microscope equipped with an Andor iXon Ultra-897 EM-CCD camera and 405 nm, 488 nm. 561 nm and 642 nm lasers, using the 20×PLAN APO air objective (NA=0.75). Image were taken using NIS Elements version 4.60 software. Microscopy for IAV experiments was performed on a Keyence BZ-X810 microscope equipped with a GFP and Texas Red filter was used to obtain images with a 20× objective lens.

For flow cytometry experiments, cells were washed with 1×PBS, treated with 1×Trypsin EDTA, and fixed in 1.6% PFA (final) for 10 minutes. Cells were washed with FACS buffer (1×PBS, 0.3% BSA, 1 mM EDTA). BD Accuri C6 plus flow cytometer was used to collect raw data. Raw data was processed with Cytobank software.

For guide preparation, T7 promoter dsDNA templates were prepared by annealing and PCR extension an upper oligo carrying the T7 promoter and RfxCas13d crRNA repeat with the bottom oligos which have an overlap of 16 bp with the upper oligo and the anti-sense crRNA spacer sequence. The templates were in vitro transcribed with the TranscriptAid T7 High Yield Transcription Kit at 37° C. overnight. Guide RNAs were purified with Monarch RNA Cleanup Kit and frozen at −80° C. For the preparation of the two GFP-Cov ssRNA template, a T7 promoter was added to the 5′ end of the dsDNA templates by PCR amplification. The PCR templates were then used for in vitro transcription and the RNA templates were purified as the production of the guides.

Purified RfxCas13d protein and guide RNA were mixed at 1:1 molar ratio in RNA Cleavage Buffer (25 mM Tris pH 7.5, 15 mM Tris pH 7.0, 1 mM DTT, 6 mM MgCl2). The reaction was prepared on ice and incubated at 37° C. for 15 minutes prior to the addition of target at 1:2 molar ratio relative to RfxCas13d. The reaction was subsequently incubated at 37° C. for 45 minutes and quenched with 0.2 ul Proteinase K (10 mg/mL) at 65° C. for 15 minutes. The reaction was then denatured with 2×RNA loading buffer), at 85° C. for 10 minutes, and separated on a fresh-made 1% agarose gel. Gels were stained with SYBR safe and imaged via the Gel imaging system.

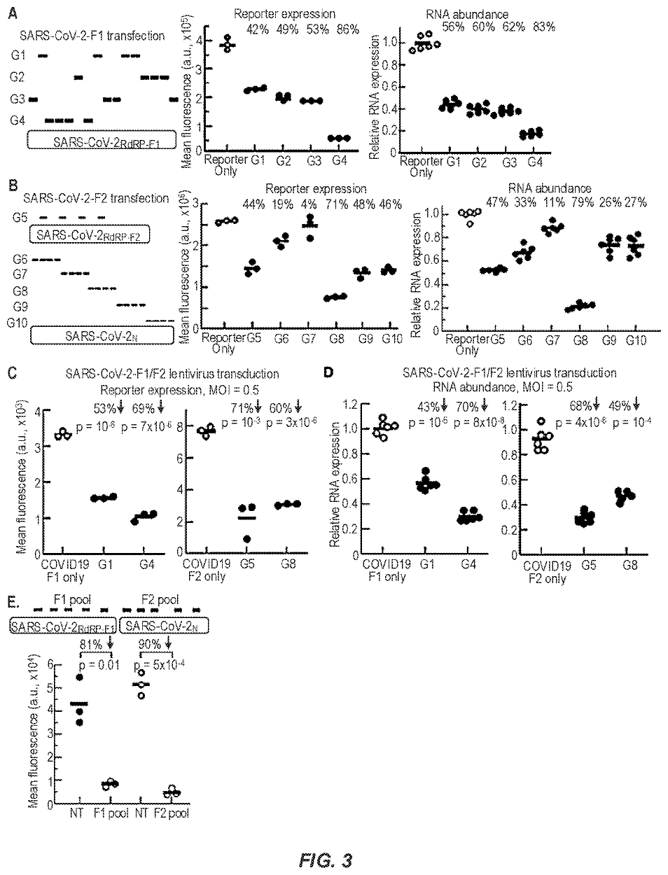

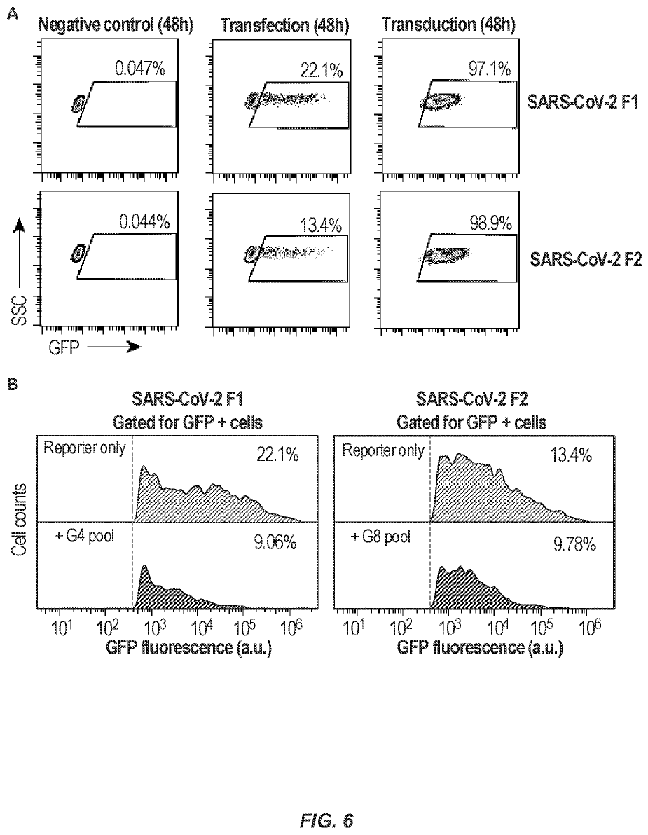

Compared to reporter only controls, most RdRP-targeting crRNA pools were able to repress reporter expression to some extent, and one pool targeting the middle of the SARS-COV-2-F1 RdRP region (Group 4; SARS-COV-2 genome coordinates 13,784-14149) was able to repress GFP by 86% (p=0.0003) (FIG. 3A, FIGS. 6A-B, and Table 3). One pool of N-targeting crRNAs (Group 8) was able to substantially repress SARS-COV-2-F2 GFP, causing a 71% decrease in expression (p=2×10⁻⁷, FIG. 3B, FIGS. 6A-B, and Table 3). The qRT-PCR analysis showed consistent results at the RNA level, with RdRP-targeting Group 4 inhibiting SARS-COV-2-F1 mRNA abundance by 83% (p=3×10⁻¹¹) and B-targeting Group 8 inhibiting SARS-COV-2-F2 mRNA abundance by 79% (p=2×10⁻¹²) (FIG. 3A, FIG. 3D, and Table 3). The variations in the ability of different crRNA pools to repress the SARS-COV-2 reporters may due to RNA secondary structure inherent in the SARS-COV-2 genome or differences in binding affinities for each crRNA sequence system.

TABLE 3
P values for crRNA screens in FIG. 3A and FIG. 3B

| | | |
|---|---|---|
| crRNA pools | Experiment | P value |

FIG. 3A

| | | |
|---|---|---|
| G1 | GFP expression | 5.41E−05 |
| G2 | GFP expression | 4.70E−05 |
| G3 | GFP expression | 1.66E−05 |
| G4 | GFP expression | 2.37E−06 |
| G1 | mRNA abundance | 4.88E−09 |
| G2 | mRNA abundance | 2.21E−09 |
| G3 | mRNA abundance | 8.13E−10 |
| G4 | mRNA abundance | 3.67E−11 |

FIG. 3B

| | | |
|---|---|---|
| G5 | GFP expression | 1.70E−04 |
| G6 | GFP expression | 3.40E−03 |
| G7 | GFP expression | 5.06E−01 |
| G8 | GFP expression | 2.37E−07 |
| G9 | GFP expression | 4.83E−05 |
| G10 | GFP expression | 8.66E−06 |
| G5 | mRNA abundance | 1.01E−10 |
| G6 | mRNA abundance | 4.47E−07 |
| G7 | mRNA abundance | 5.54E−04 |
| G8 | mRNA abundance | 1.52E−12 |
| G9 | mRNA abundance | 1.35E−05 |
| G10 | mRNA abundance | 3.51E−05 |

Next, the performance of RdRP-targeting adjacent crRNA pools was validated when SARS-COV-2-F1 was introduced via lentiviral transduction. This approach, in which the lentivirus-derived RNA serves as a PAC-MAN target prior to lentivirus integration into DNA, mimicked the crRNA targeting conditions that would apply for natural COVID-19 infection. A few selected crRNA pools, including the 3 best groups G4, G5, and G8 and one less efficient group G1, were tested with a lentiviral multiplicity of infection (MOI) of 0.5. The results revealed similar patterns to SARS-CoV-2 transfection experiments, with the best groups (G4, G5, G8) able to repress their reporters by 69%, 71%, and 60% (p=7×10⁻⁶, 10⁻³, 3×10⁻⁶ for G4, G5, G8, respectively, FIG. 3C). qRT-PCR analysis also showed consistent results, with G4, G5, and G8 repressing 70%, 68%, and 49% of RNA abundance (p=8×10⁻⁸, 4×10⁻⁶, 10⁻⁴ for G4, G5, G8 respectively, FIG. 3D).

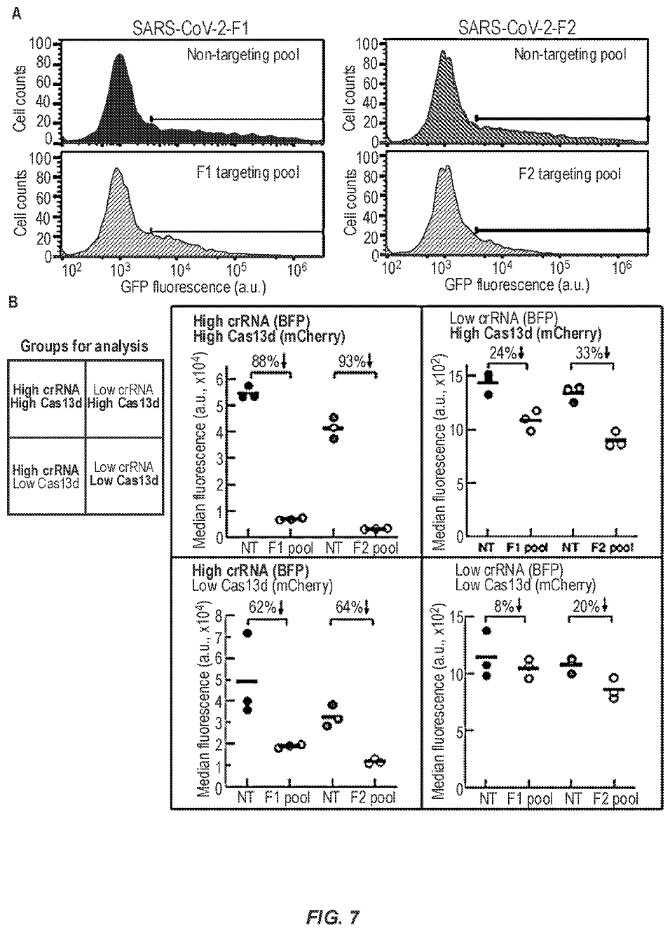

The effectiveness of Cas13d-mediated repression of SARS-COV-2 reporters was further validated by co-transfecting each reporter with a pool of 5 crRNAs tiled along the whole fragment, compared to a pool of 5 non-targeting control crRNAs (Table 1). It was found that the RdRP-targeting crRNAs were able to repress SARS-COV-2-F1 GFP by 81% (p=0.01), while B-targeting crRNAs were able to repress SARS-COV-2-F2 GFP by 90% (p=5×10⁻⁴, FIG. 3E and FIG. 7A). An analysis of the extent to which expression of Cas13d or crRNAs affected SARS-CoV-2 GFP reporter inhibition. The analysis revealed that SARS-CoV-2 inhibition was more sensitive to crRNA concentration in the samples, while lower Cas13d expression only moderately decreased inhibitory activity (FIG. 7B). Thus, maintaining a high level of crRNA concentration is likely a determining factor for more effective SARS-CoV-2 sequence targeting and cleavage. These data together demonstrated: 1) Cas13d PAC-MAN can be an effective system to target and degrade SARS-CoV-2 sequences in human cells and; 2) proper design of crRNAs are important for obtaining a high efficiency of SARS-CoV-2 inhibition.

### Example 6. Cas13d PAC-MAN May be a Pan-Coronavirus Inhibition Strategy

In the past two decades, multiple variants of coronavirus including SARS-COV-2, SARS and MERS emerged from animal reservoirs into humans, each time causing significant fatalities. It may be greatly beneficial to design a strategy that can target and prevent broad viral threats including those that currently reside in animals. As such, a minimal number of crRNAs that may target a majority of known coronaviruses found in both humans and animals was designed.

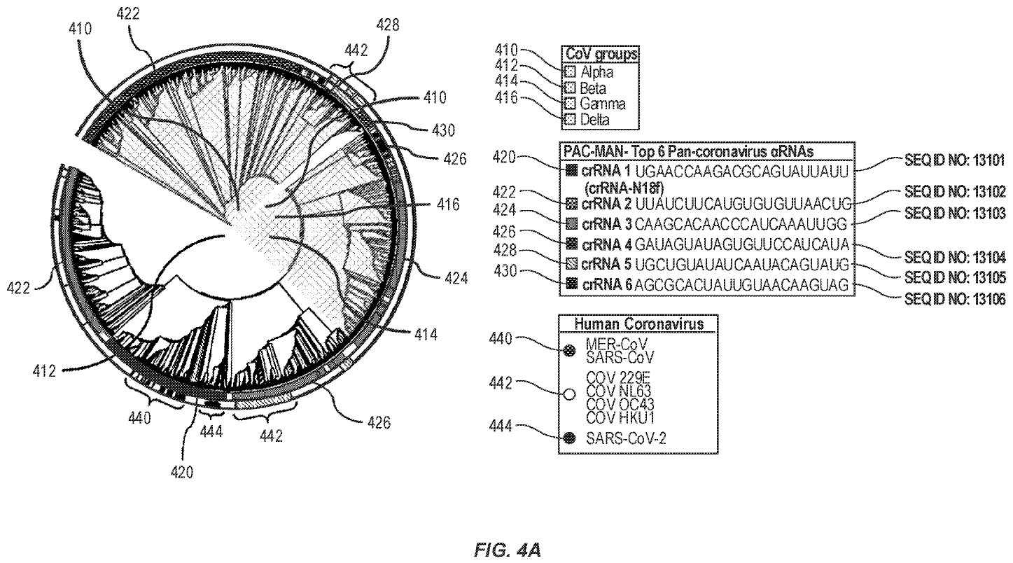

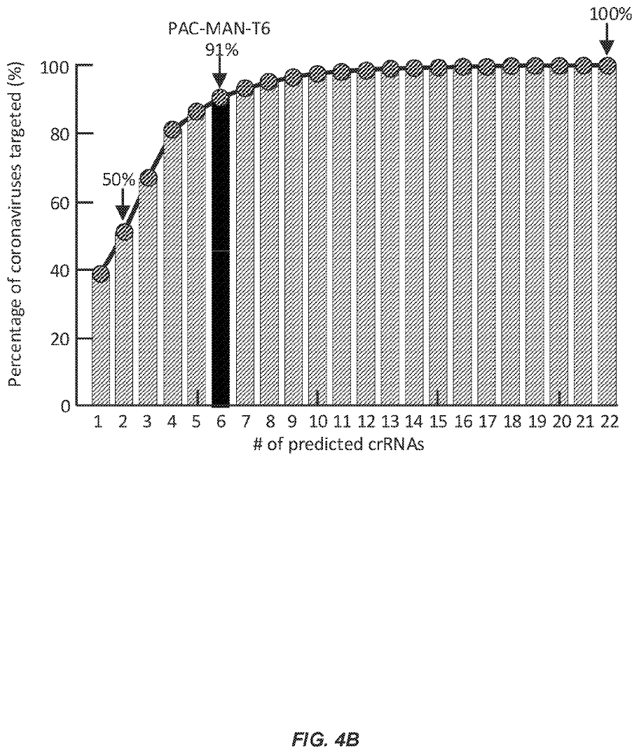

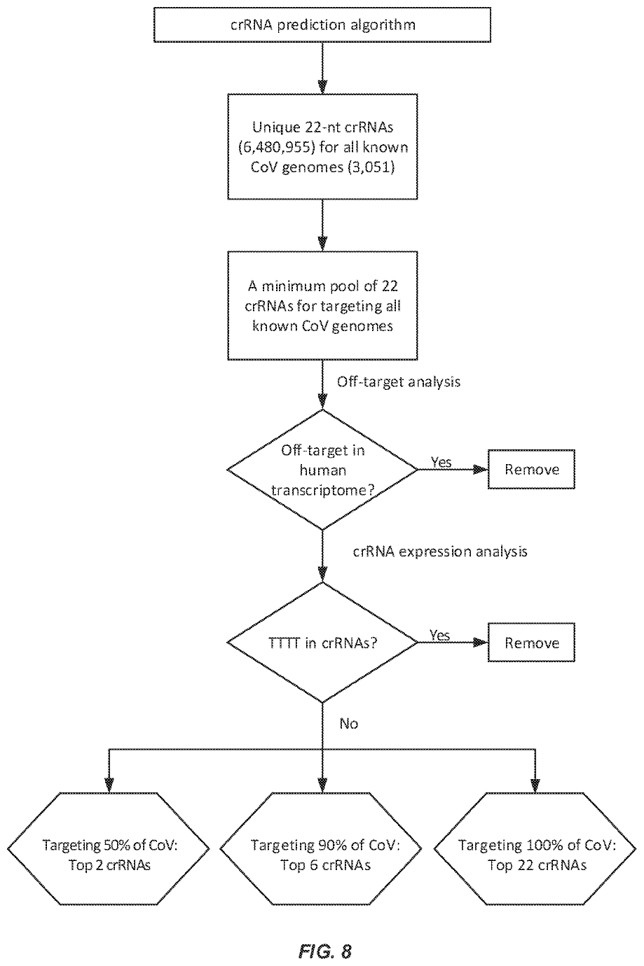

To do this, all known coronavirus genomes (3,150) was analyzed to identify all possible crRNAs that may target each of those genomes, ending up with approximately 6.48 million possible crRNAs (FIG. 4A and FIG. 8). The crRNAs were then narrowed down to define what was the lowest number of crRNAs that may target all known coronavirus genomes with zero mismatches. It was found that just two crRNAs may target ˜50% of coronavirus genomes including SARS-COV-2, SARS and MERS, 6 crRNAs were able to target ˜91% of coronavirus genomes, and 22 crRNAs may cover all known coronaviruses with no mismatches (FIG. 4B and Table 4). The ability to use a small number of crRNAs to possibly target broad strains of coronaviruses further highlighted the uniqueness of approach disclosed herein in contrast to traditional pharmaceutical or vaccination approaches.

Referring to FIG. 4A, about 3,150 coronavirus genomes are depicted in a pie chart 400. The coronavirus genomes are first grouped into the following four coronavirus (CoV) group: alphacoronavirus 410, betacoronavirus 412, deltacoronavirus 414, and gammacoronavirus 416. The inner region of the pie chart 400 indicates the respective CoV type of each genome. The pie chart 400 is surrounded by an inner ring to indicate which crRNAs of the top 6 pan-coronavirus crRNAs can bind (or target) each coronavirus genome. The top 6 pan-coronavirus crRNAs include: crRNA 420 (SEQ ID NO: 13101), crRNA 422 (SEQ ID NO: 13102), crRNA 424 (SEQ ID NO: 13103), crRNA 426 (SEQ ID NO: 13104), crRNA 428 (SEQ ID NO: 13105), and crRNA 430 (SEQ ID NO: 13106). Furthermore, the pie chart 400 is further surrounded by an outer ring to indicate the genomes that correspond to one of the three human coronaviruses: (1) MERS-CoV and SARS-CoV 440, (2) COV 229E, COV NL63, COV OC43, and COV HKU1 442, and (3) SARS-CoV-2 444.

TABLE 4
Sequences of predicted 22 crRNA targeting all coronaviruses with the
top 6 crRNA covering 91% of coronaviruses including all published
SARS-CoV-2 strains

| | | | | | Target viral |
|---|---|---|---|---|---|
| SEQ ID | Spacer DNA | SEQ ID | Spacer RNA | SEQ ID | genome RNA |
| NO | sequence | NO | sequence | NO | sequence |
| SEQ ID | TGAACCAAG | SEQ ID | UGAACCAAGA | SEQ ID | AAUAAUACUG |
| NO: | ACGCAGTATT | NO: | CGCAGUAUUA | NO: | CGUCUUGGUU |
| 13001 | ATT | 13101 | UU | 13201 | CA |
| SEQ ID | TTATCTTCAT | SEQ ID | UUAUCUUCAU | SEQ ID | ACUGGAAUUU |
| NO: | GTGTGTTAAC | NO: | GUGUGUUAAC | NO: | CACAUGGAAU |
| 13002 | TG | 13102 | UG | 13202 | AU |
| SEQ ID | CAAGCACAA | SEQ ID | CAAGCACAAC | SEQ ID | AGACCUGAAG |
| NO: | CCCATCAAAT | NO: | CCAUCAAAUU | NO: | GUUGCGUUGU |
| 13003 | TGG | 13103 | GG | 13203 | AA |
| SEQ ID | GATAGTATAG | SEQ ID | GAUAGUAUA | SEQ ID | AGGGUUGUGG |
| NO: | TGTTCCATCA | NO: | GUGUUCCAUC | NO: | UCUUUUUAAA |
| 13004 | TA | 13104 | AUA | 13204 | GA |
| SEQ ID | TGCTGTATAT | SEQ ID | UGCUGUAUAU | SEQ ID | AGUAUUAUUC |
| NO: | CAATACAGTA | NO: | CAAUACAGUA | NO: | AGGCAGUGCU |
| 13005 | TG | 13105 | UG | 13205 | UC |
| SEQ ID | AGCGCACTAT | SEQ ID | AGCGCACUAU | SEQ ID | CAAGGAUAGU |
| NO: | TGTAACAAGT | NO: | UGUAACAAGU | NO: | AUACCUGUGG |
| 13006 | AG | 13106 | AG | 13206 | UG |
| SEQ ID | ATATTCCATG | SEQ ID | AUAUUCCAUG | SEQ ID | CACAGAAUGC |
| NO: | TGAAATTCCA | NO: | UGAAAUUCCA | NO: | UGAGUCCCUG |
| 13007 | GT | 13107 | GU | 13207 | UC |
| SEQ ID | TTACAACGCA | SEQ ID | UUACAACGCA | SEQ ID | CACUGGAUGC |
| NO: | ACCTTCAGGT | NO: | ACCUUCAGGU | NO: | CAUUAUAGUU |
| 13008 | CT | 13108 | CU | 13208 | GA |
| SEQ ID | TCTTTAAAAA | SEQ ID | UCUUUAAAAA | SEQ ID | CAGGCUAUUA |
| NO: | GACCACAACC | NO: | GACCACAACC | NO: | ACUCUUCAUU |
| 13009 | CT | 13109 | CU | 13209 | GU |
| SEQ ID | GAAGCACTGC | SEQ ID | GAAGCACUGC | SEQ ID | CAGUUAACAC |
| NO: | CTGAATAATA | NO: | CUGAAUAAUA | NO: | ACAUGAAGAU |
| 13010 | CT | 13110 | CU | 13210 | AA |
| SEQ ID | CACCACAGGT | SEQ ID | CACCACAGGU | SEQ ID | CAUACUGUAU |
| NO: | ATACTATCCT | NO: | AUACUAUCCU | NO: | UGAUAUACAG |
| 13011 | TG | 13111 | UG | 13211 | CA |
| SEQ ID | GACAGGGAC | SEQ ID | GACAGGGACU | SEQ ID | CAUCCUUAGU |
| NO: | TCAGCATTCT | NO: | CAGCAUUCUG | NO: | GGCAAGUUUU |
| 13012 | GTG | 13112 | UG | 13212 | CU |
| SEQ ID | TCAACTATAA | SEQ ID | UCAACUAUAA | SEQ ID | CCAAUUUGAU |
| NO: | TGGCATCCAG | NO: | UGGCAUCCAG | NO: | GGGUUGUGCU |
| 13013 | TG | 13113 | UG | 13213 | UG |
| SEQ ID | ACAATGAAG | SEQ ID | ACAAUGAAGA | SEQ ID | CUACUUGUUJA |
| NO: | AGTTAATAGC | NO: | GUUAAUAGCC | NO: | CAAUAGUGCG |
| 13014 | CTG | 13114 | UG | 13214 | CU |
| SEQ ID | AGAAAACTTG | SEQ ID | AGAAAACUUG | SEQ ID | CUCACAUAAU |
| NO: | CCACTAAGGA | NO: | CCACUAAGGA | NO: | GUUCAGCCAU |
| 13015 | TG | 13115 | UG | 13215 | UG |
| SEQ ID | CAATGGCTGA | SEQ ID | CAAUGGCUGA | SEQ ID | GACAUUGCCA |
| NO: | ACATTATGTG | NO: | ACAUUAUGUG | NO: | CGUGUAUGCG |
| 13016 | AG | 13116 | AG | 13216 | UG |
| SEQ ID | CACGCATACA | SEQ ID | CACGCAUACA | SEQ ID | GAUGCUGUUA |
| NO: | CGTGGCAATG | NO: | CGUGGCAAUG | NO: | AUAAUGGUUC |
| 13017 | TC | 13117 | UC | 13217 | UC |
| SEQ ID | GAGAACCATT | SEQ ID | GAGAACCAUU | SEQ ID | UAGAUAAUCA |
| NO: | ATTAACAGCA | NO: | AUUAACAGCA | NO: | GGAUCUUAAU |
| 13018 | TC | 13118 | UC | 13218 | GG |
| SEQ ID | CCATTAAGAT | SEQ ID | CCAUUAAGAU | SEQ ID | UAUGAUGGAA |
| NO: | CCTGATTATC | NO: | CCUGAUUAUC | NO: | CACUAUACUA |
| 13019 | TA | 13119 | UA | 13219 | UC |
| SEQ ID | ATAGTAATGC | SEQ ID | AUAGUAAUGC | SEQ ID | UAUGCAUUGU |
| NO: | ATACAATGCA | NO: | AUACAAUGCA | NO: | AUGCAUUACU |
| 13020 | TA | 13120 | UA | 13220 | AU |
| SEQ ID | CGACCATAAC | SEQ ID | CGACCAUAAC | SEQ ID | UGGCUAUUGA |
| NO: | CATCAATAGC | NO: | CAUCAAUAGC | NO: | UGGUUAUGGU |
| 13021 | CA | 13121 | CA | 13221 | CG |
| SEQ ID | CAACATACTA | SEQ ID | CAACAUACUA | SEQ ID | UUUUAAUGAC |
| NO: | TTGTCATTAA | NO: | UUGUCAUUAA | NO: | AAUAGUAUGU |
| 13022 | AA | 13122 | AA | 13222 | UG |

As a map was provided to show how each of the top 6 crRNAs (PAC-MAN-T6) targeted various parts of the coronavirus phylogenetic tree (FIG. 4A, middle box denoted with PAC-MAN-Top 6 Pan-coronavirus crRNAs). Beta coronaviruses, the genus of SARS-COV-2, SARS, and MERS, were mostly covered by 3 crRNAs (crRNA 1-3), while the other genera were covered by these three plus the remaining 3 crRNAs. Altogether, the PAC-MAN-T6 can target all known human coronavirus with broad coverage against other animal coronaviruses. Table 5 illustrates the cumulative of percentage of SARS-COV-2 genomes being targeted with each additional targeting by the 22 crRNA targeting coronavirus as described herein.

TABLE 5
Percentage of Pan-coronavirus targeted with PAC-MAN crRNAs.

| | % of | |
|---|---|---|
| Number | Targeted | |
| of | CoV | |
| crRNAs | Genomes | crRNA list in the category |
| 1 | 12.28% | SEQ ID NO: 13101 |
| 2 | 51.09% | SEQ ID NO: 13101; SEQ ID NO: 13102 |
| 3 | 67.15% | SEQ ID NO: 13101; SEQ ID NO: 13102; SEQ ID NO: 13103 |
| 4 | 81.12% | SEQ ID NO: 13101; SEQ ID NO: 13102; SEQ ID NO: 13103; SEQ |
| | | ID NO: 13104 |
| 5 | 86.51% | SEQ ID NO: 13101; SEQ ID NO: 13102; SEQ ID NO: 13103; SEQ |
| | | ID NO: 13104; SEQ ID NO: 13105 |
| 6 | 90.61% | SEQ ID NO: 13101; SEQ ID NO: 13102; SEQ ID NO: 13103; SEQ |
| | | ID NO: 13104; SEQ ID NO: 13105; SEQ ID NO: 13106 |
| 7 | 93.15% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119 |
| 8 | 95.11% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117 |
| 9 | 96.41% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121 |
| 10 | 97.65% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108 |
| 11 | 98.16% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109 |
| 12 | 98.57% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115 |
| 13 | 98.95% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: 13107 |
| 14 | 99.17% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116 |
| 15 | 99.37% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113 |
| 16 | 99.52% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: 13112 |
| 17 | 99.65% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: |
| | | 13112; SEQ ID NO: 13122 |
| 18 | 99.75% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: |
| | | 13112; SEQ ID NO: 13122; SEQ ID NO: 13118 |
| 19 | 99.84% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: |
| | | 13112; SEQ ID NO: 13122; SEQ ID NO: 13118; SEQ ID NO: 13111 |
| 20 | 99.90% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: |
| | | 13112; SEQ ID NO: 13122; SEQ ID NO: 13118; SEQ ID NO: |
| | | 13111; SEQ ID NO: 13114 |
| 21 | 99.97% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: |
| | | 13112; SEQ ID NO: 13122; SEQ ID NO: 13118; SEQ ID NO: |
| | | 13111; SEQ ID NO: 13114; SEQ ID NO: 13120 |
| 22 | 100.00% | SEQ ID NO: 13101; SEQ ID NO: 13102; SEQ ID NO: 13103; SEQ |
| | | ID NO: 13104; SEQ ID NO: 13105; SEQ ID NO: 13106; SEQ ID |
| | | NO: 13119; SEQ ID NO: 13117; SEQ ID NO: 13121; SEQ ID NO: |
| | | 13108; SEQ ID NO: 13109; SEQ ID NO: 13115; SEQ ID NO: |
| | | 13107; SEQ ID NO: 13116; SEQ ID NO: 13113; SEQ ID NO: |
| | | 13112; SEQ ID NO: 13122; SEQ ID NO: 13118; SEQ ID NO: |
| | | 13111; SEQ ID NO: 13114; SEQ ID NO: 13120; SEQ ID NO: 13110 |

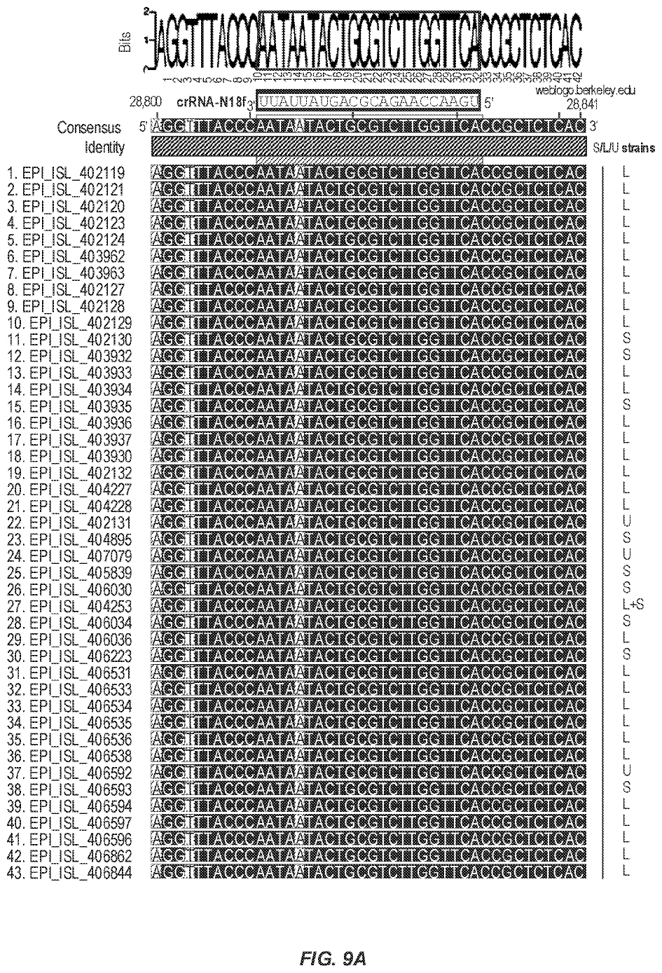

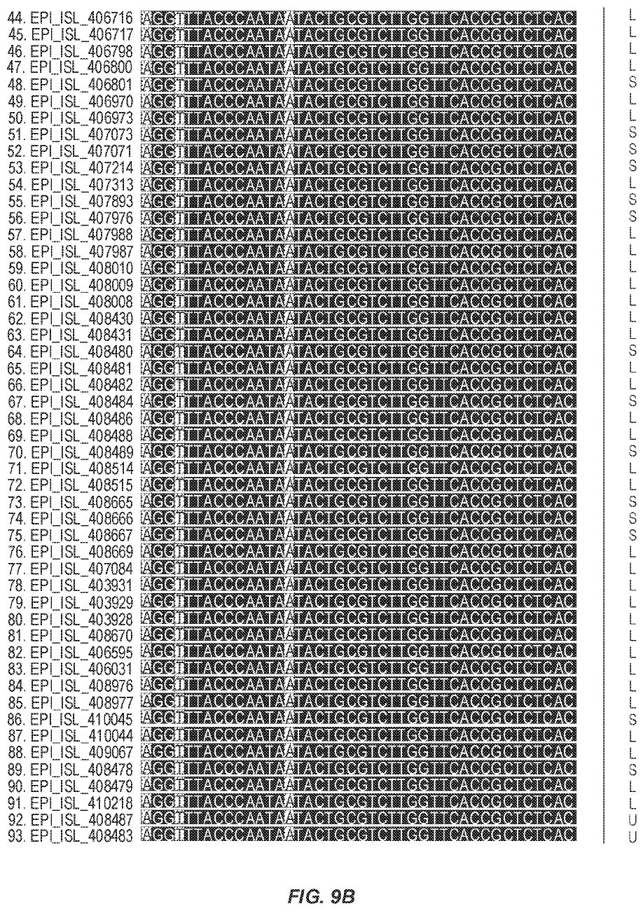

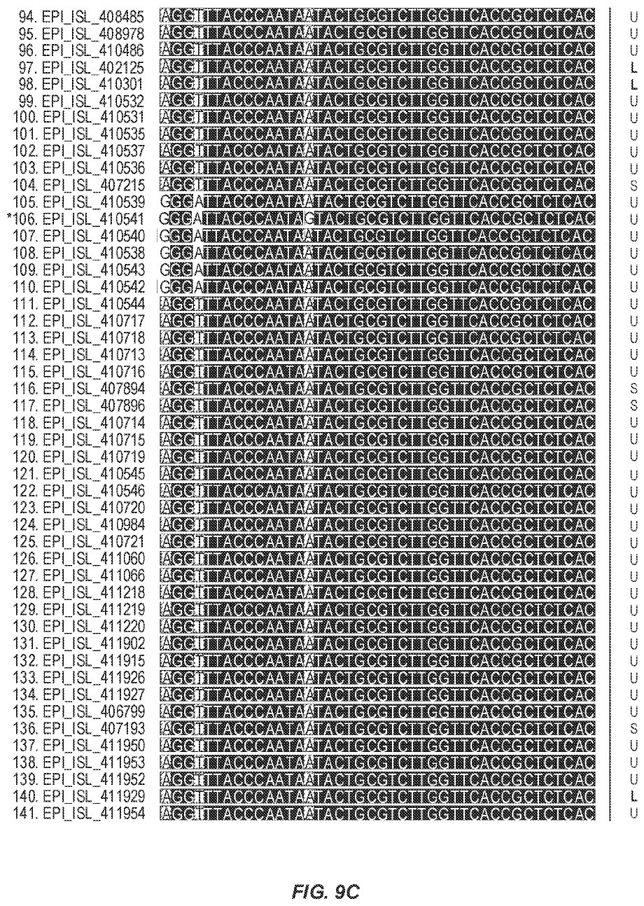

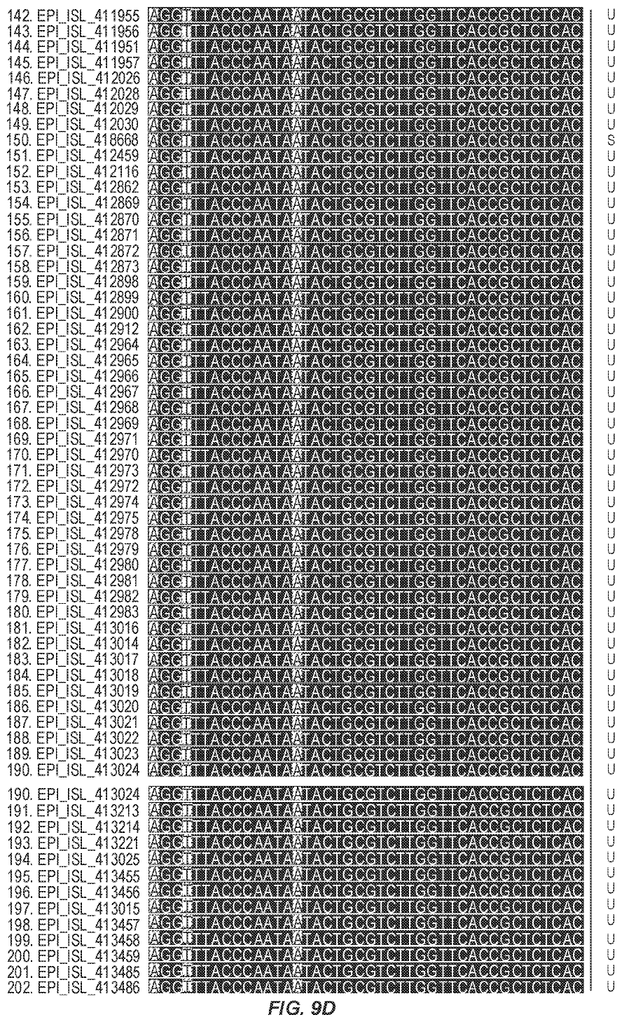

The minimal set of six crRNAs also included one crRNA (designated crRNA-N18f) that was validated (FIGS. 9A-9D). Analysis of 103 SARS-COV-2 genomes indicated that there are two major types (L and S) of SARS-COV-2 existing with different viral signature sequences. To verify whether crRNA-N18f may target all L and S strains, all recent sequenced SARS-COV-2 sequences were retrieved from Global Initiative on Sharing All Influenza Data (GISAID) for a set of 202 SARS-COV-2 genomes. Notably, crRNA_N18f targeted all 201 genomes with only one SARS-COV-2 sequence carried a single mismatch mutation. This may validate the approach described in the instant disclosure to target broad SARS-COV-2 strains and the need to use a pool of crRNAs to avoid mutational escapes.

### Example 7. Screening Guide RNAs to Target Human 229E Coronavirus Genome

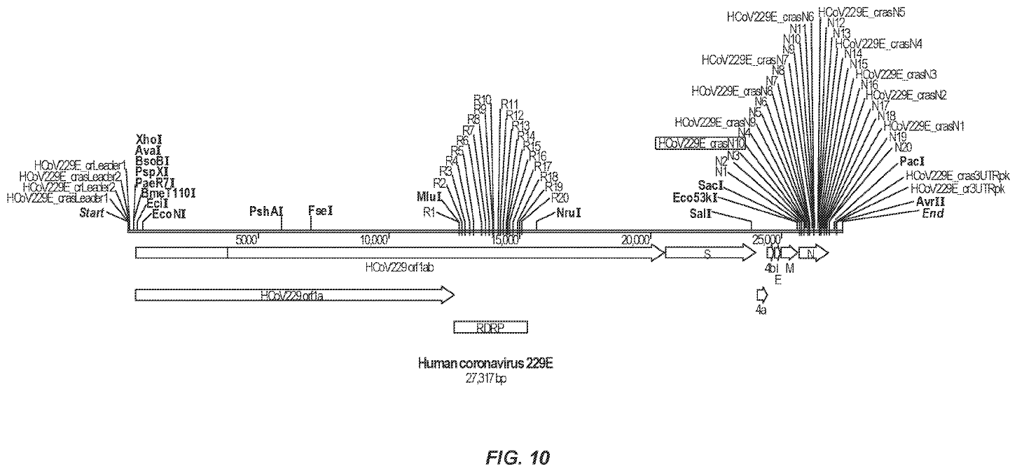

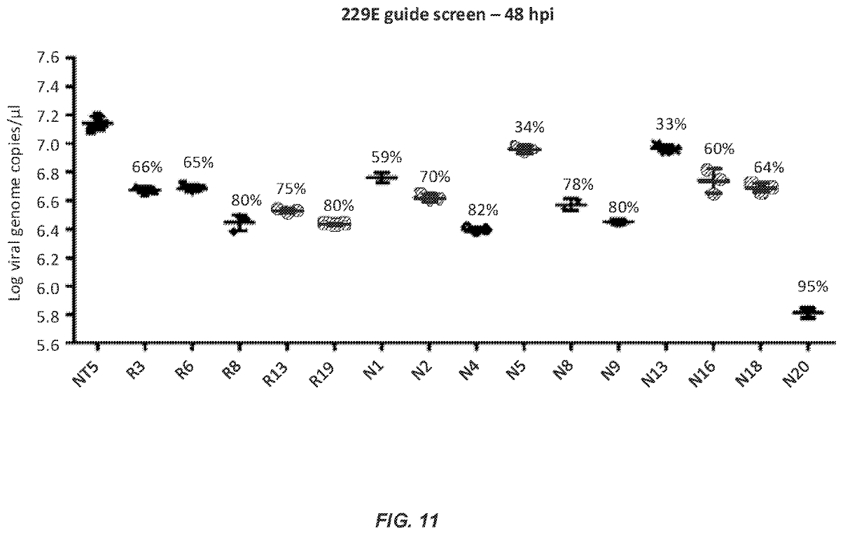

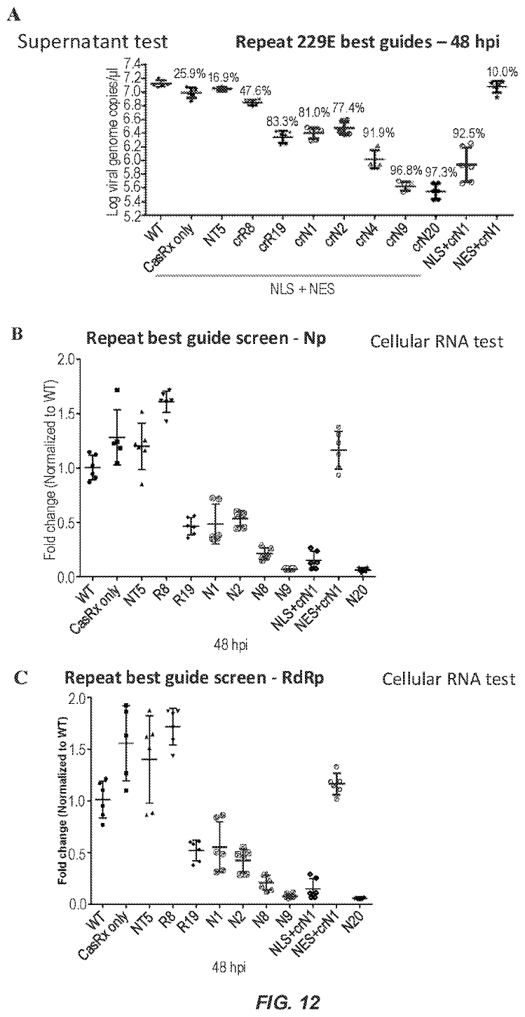

FIG. 10 illustrates crRNAs (guide RNA) design targeting the human 229E coronavirus genome. The crRNAs were designed to be complexed with any one of the Cas endonucleases described herein. For example, the crRNAs were designed to be complexed with Cas13d. 4 guides (crRNAs) targeted the 5′UTR region, which was also the leader sequence of all viral RNAs. 20 guides, R1-R20, targeted the RdRP, and 20 guides, N1-N20, targeted N gene. These two genes are the most conserved region of coronaviruses. Meanwhile, 10 guides, crasN1-N10, targeted the negative strand of N gene, and 2 guides target 3′UTR. 56 CasRx guides in total were designed to target the 229E genomic and subgenomic RNAs. RdRP and N gene are the most conserved region of 229E virus so that most guides, 20 for RdRP and 20 for N gene, were designed. Since all the viral RNAs, including genomic and subgenomic RNAs, are expressed carrying the same 5′UTR and 3′UTR sequence, several guides were designed to target these sequences. Besides, some guides targeting the negative stranded RNAs were also designed. FIG. 11 illustrates guide screening assay for determining the guide (40 crRNAs: SEQ ID NO: 13607-SEQ ID NO: 13646) with the best efficiency against 229E. Most guides inhibited more than 60% of the virus replication, and the best one, N20, may reach to 95% for targeting 229E in MRC-5 cells. The ones which helped cell survive better than control were selected. The viral titer in the supernatant were determined by RT-qPCR. Most guides may inhibit the virus replication to >60%, while the best one N20 may reach to 95%. FIG. 12 illustrates repeat testing of crRNAs targeting 229E. Virus titer in both the supernatant and cellular samples were determined by RT-qPCR. For quantification of cellular viral RNA, both N gene and RdRP gene were selected as target. Beta actin was the inner control. The CasRx with NLS or NES tag were used in 1:1 mixture or alone when testing the N1 guide. FIG. 12A illustrates crRNAs from the screen assay for repeat testing. While crR8 (SEQ ID NO: 13614), crR19 (SEQ ID NO: 13625), crN1 (SEQ ID NO: 13627), and crN2 (SEQ ID NO: 13628) inhibited about 80% of virus replication, crN4 (SEQ ID NO: 13630), crN9 (SEQ ID NO: 13635), and crN20 (SEQ ID NO: 13646) may reach to more than 90%. NLS CasRx were compared with NES version using the crN1 guide. The NLS version (92.5% inhibition) worked better than the NLS and NES mixture (81%). The NES version worked the worst (10%). The result from cellular RNA was in consistence with the supernatant. PAC-MAN may decrease the viral RNA in cellular and the released virus in supernatant. FIG. 12B illustrates repeat testing with cellular.

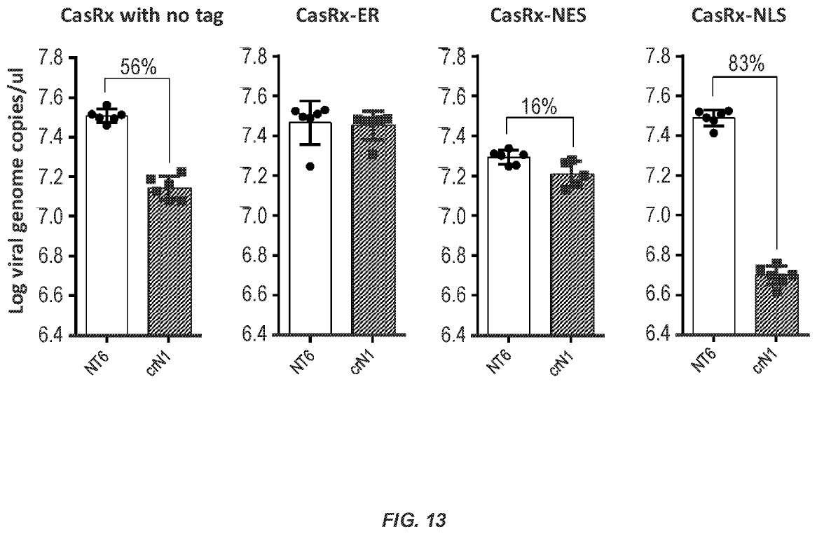

FIG. 13 illustrates testing of CasRx with no tags or tagged with nuclear localization signal (NLS), nuclear export signal (NES), or endoplasmic reticulum (ER) signal. Since the NLS Cas13d worked better than NES, Cas13d without a tag or was fused with ER location signal. From the result, Cas13d-NLS still worked the best, with a 83% inhibition effect, while Cas13d with no tag had 56% inhibition effect, Cas13d-ER had no effect, and Cas13d-NES had only 16% inhibition effect.

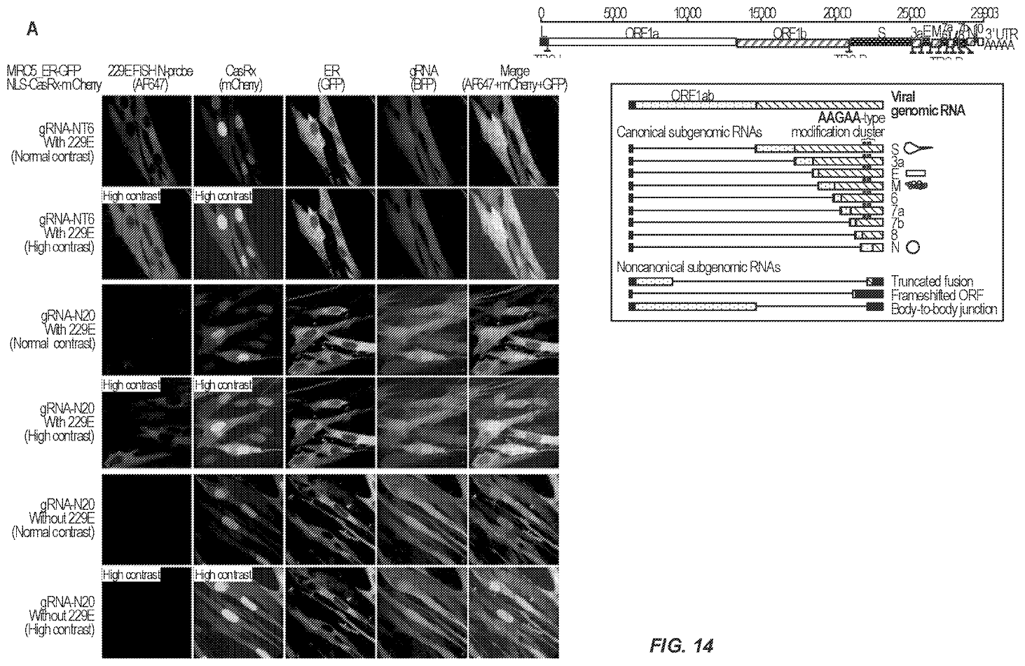

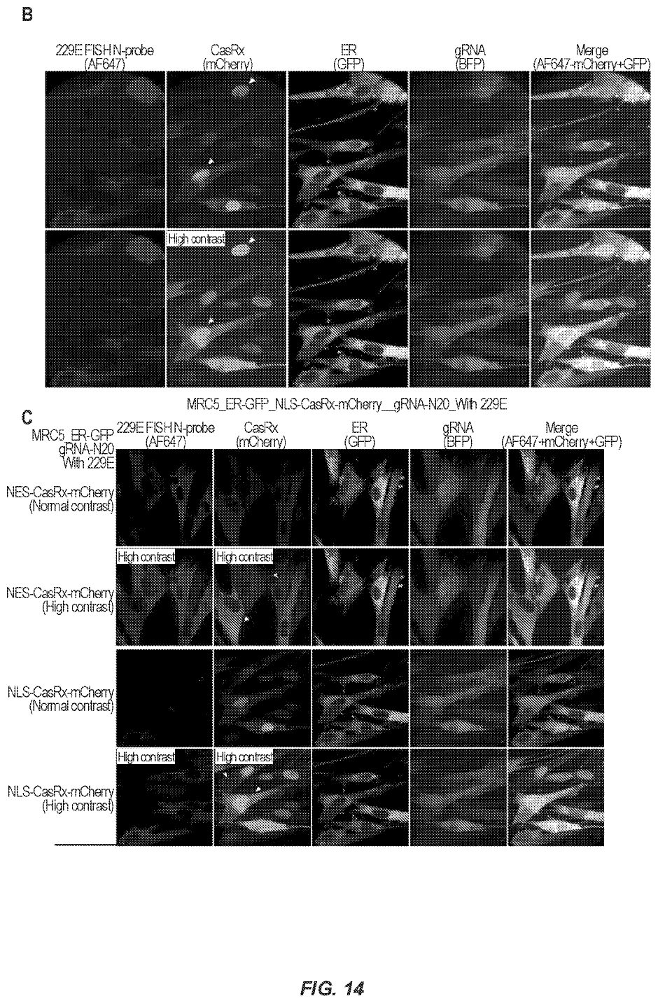

To examine the increased efficacy o of NLS-version of PAC-MAN, MRC5 cells were treated with NLS- or NES-version of PAC-MAN followed by infection of 0.1 MOI 229E. The cells were then stained with 229E RNA FISH N-probe and imaged by confocal. FIG. 14 illustrates how NLS-version of the PAC-MAN worked better than NES-version of the PAC-MAN. MRC5 cells were treated with NLS- or NES-version of PAC-MAN followed by infection of 0.1 MOI 229E. Then cells were stained with 229E RNA FISH N-probe and imaged by confocal. FIG. 14A illustrates RNA FISH probe set-1: targeting N gene (detecting all viral RNA). Same contrast adjustment was used for “Normal contrast” or “High contrast” lane of each group; thus, the intensity was comparable between each group. The signal of 229E FISH in N20 group was significantly decreased compared with non-target guide (NT6) control group, supporting the high efficiency of PAC-MAN. FIG. 14B illustrates the cell without detectable CasRx in cytosol (pointed by white arrow) had intensive signals of 229E FISH, while the cell with CasRx in cytosol (pointed by grey arrow) did not have detectable signals of 229E FISH, indicating that the cytosolic CasRx may be important for the high efficiency of PAC-MAN. FIG. 14C. The signals of 229E FISH were much more intensive in NES-CasRx group compared with that in NLS-CasRx group, though the signals of CasRx in cytosol of the two groups did not have significant difference as shown by the cells pointed by the white or grey arrows. The high efficiency of the NLS-version PAC-MAN compared with the NES-version PAC-MAN was due to the low efficient transport of gRNA to the cytosol and the disability of gRNA in cytosol. Both NES and NLS were expressed in cytosol to similar amount. Both of Cas13d (CasRx) proteins were distributed relatively even in cytosol, and there was also no difference on their localization with viral RNA. For the NLS high contrast figure, it was observed that when there's more Cas13d (CasRx) protein in cytosol, there was less viral RNA. In some cells, there was no Cas13d (CasRx) in cytosol. Accordingly, the Cas13d (CasRx) guide may not be efficiently exported from nuclear to cytosol, so NES version Cas13d (CasRx) may not work well. On the other hand, the NLS-version may enter the nucleus and then leaked to cytosol to function well at high expression.

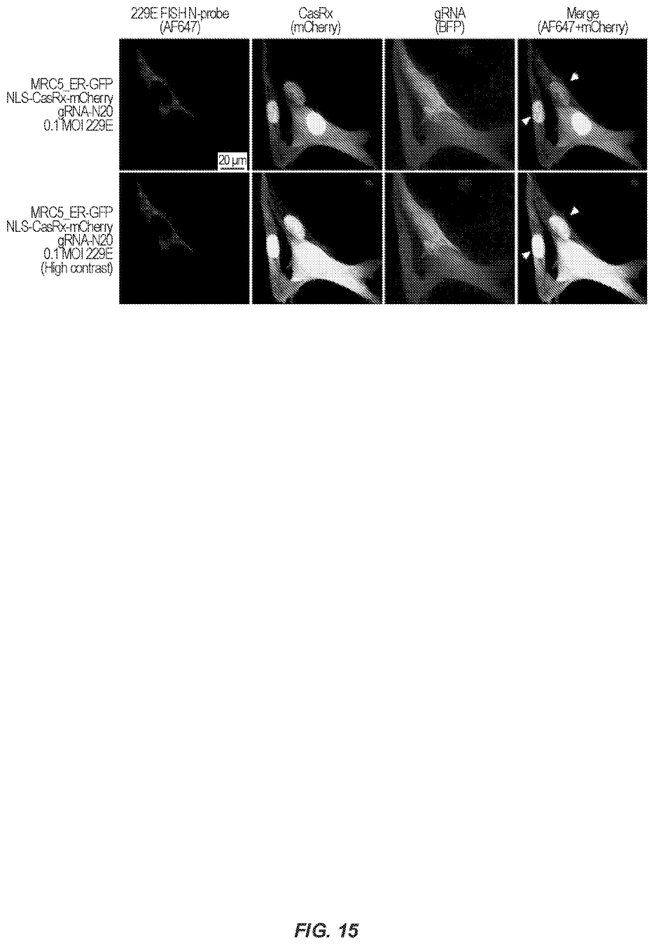

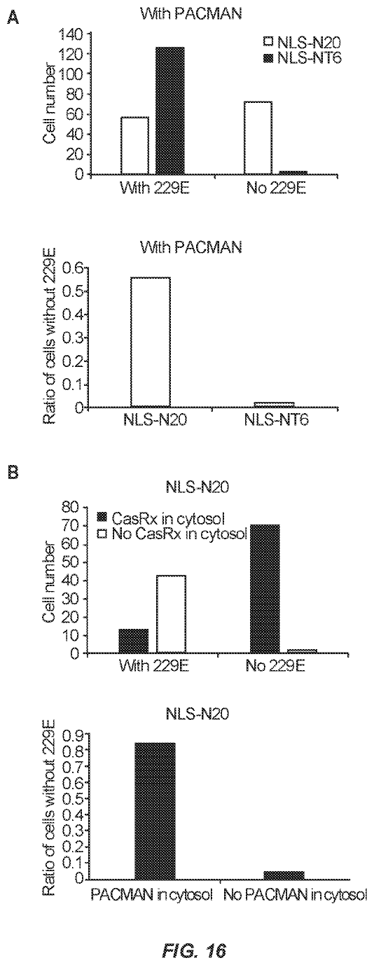

FIG. 15 illustrates another representative image of cells with/without cytosolic CasRx. High expression of Cas13d (CasRx) may leak some Cas13d to cytosol, these Cas13d-crRNA may repress virus replication. The merge picture showed that the cell with Cas13d cytoplasm expression delete the virus. Some cells may not delete virus, because of lack of Cas13d cytoplasm expression. FIG. 16 illustrates analysis of captured images of FIG. 14 and FIG. 15. FIG. 16A illustrates that for cells expressing NLS-CasRx and gRNA in N20 (SEQ ID NO: 13646) and NT6 groups, the number of cells with/without 229E FISH signal was counted. Then, the ratio of cells without 229E FISH signal was calculated. FIG. 16B illustrates that for cells with or without NLS-CasRx expressed in cytosol in N20 group, the number of cells with or without 229E FISH signal was counted. Then, the ratio of cells without 229E FISH signal was calculated. The expression level of CasRx in most cells with NLS-CasRx in cytosol was higher than that of cells without NLS-CasRx in cytosol.

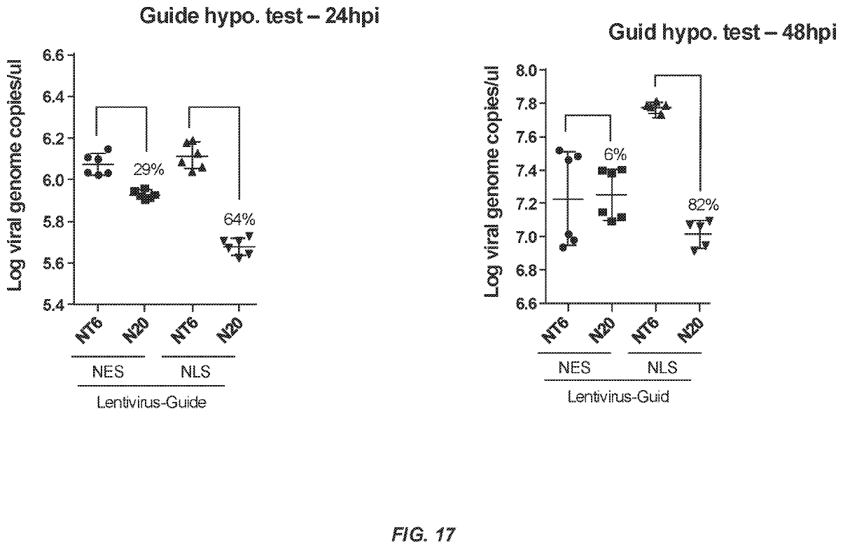

To further examine the location of the CasRx, the MRC-5 cells in group “lentivirus-guide” were co-transduced with NES- or NLS-CasRx and guide. 6 hour post transfection (6 hpi), the cells were infected with 229E at an MOI of 0.01. FIG. 17 illustrates that when using lentivirus to deliver the guide, the guide was transcribed in nucleus, the NLS-version of CasRx worked better than NES.-version of CasRx. These results suggested that the guide expressed by lentivirus were mostly kept in nucleus. Only the NLS-CasRx may enter the nucleus and bind the guide and a part of the RNP may be exported into cytosol and cut the viral RNA of coronavirus. While delivering guide RNA directly into cytosol, the NES-CasRx worked much better. FIG. 17 also illustrates that the lentivirus transduced gRNA stayed in nucleus, thus NLS-Cas13d may enter into the nucleus to complex with the gRNA and leached out of nucleus. In contrast, NES-Cas13d may not enter the nucleus, thus may not form a complex with the gRNA in the nucleus to the same extent, thus not as effective in reducing coronavirus replication. When using lentivirus to deliver the guide (crRNA), the guide were transcribed in nucleus, the NLS CasRx worked better than NES, which was 64% v.s. 29% of inhibition on virus replication at 24 hpi and 82% vs 6% at 48 hpi (FIG. 17). The NLS-CasRx may enter the nucleus and bind the guide and some of the RNP may be exported into cytosol and cut the viral RNA of coronavirus (FIGS. 14-16).

### Example 8. Cell Viability Measurement

To measure cell viability of the cells described herein, cells were seeded and transduced with CasR and the crRNAs described herein on day 0. On Day 2, the transduced cells were infected with 229E at an MOI of 0.01. 250 nM Cytotox Green Reagent was added in the media for measurement of viability. The cells were imaged for about 4 days using live-cell imaging system. Compared to non-target guide (NT6), all the tested 229E guides helped the cells surviving 229E infection. On day 3, more than 70% cells in the NT6 group (53% of the 75% PAC-MAN positive cells) were dead, while more than 90% cells in the crN1 group (86% of the 95% PAC-MAN positive cells) were alive.

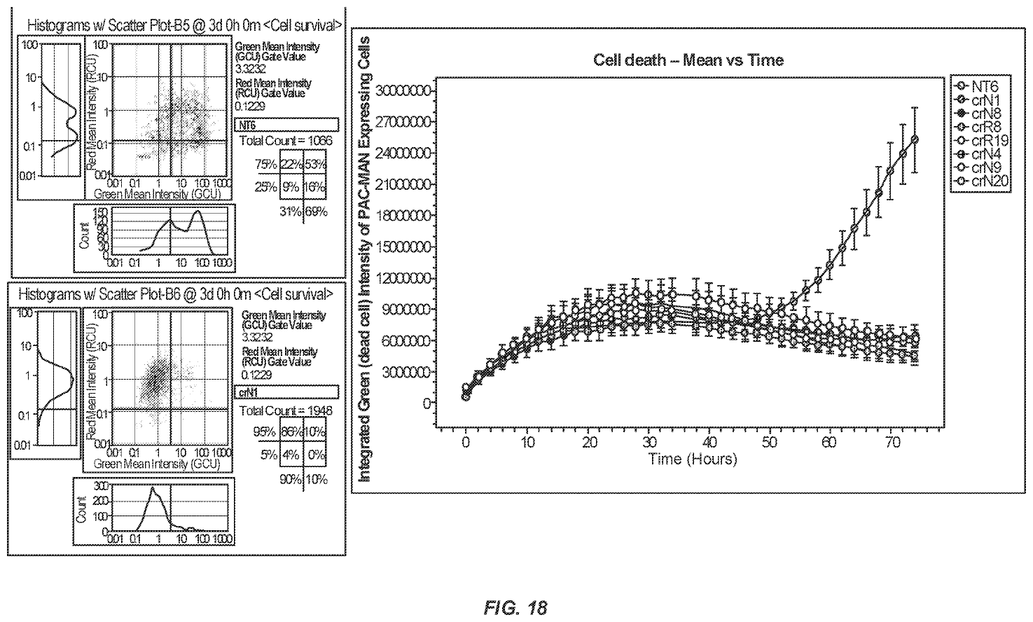

FIG. 18 illustrates how PAC-MAN helps cell surviving 229E infection. Compared to non-target guide (NT6), all the tested 229E guides, including crR8, crR19, crN1, crN4, crN8, crN9, and crN20, helped the cells survive 229E infection for at least 3 days. On day 3, while less than 30% cells in the NT6 group (22% of the 75% PAC-MAN positive cells) were alive, more than 90% cells in the crN1 group (86% of the 95% PAC-MAN positive cells) were alive.

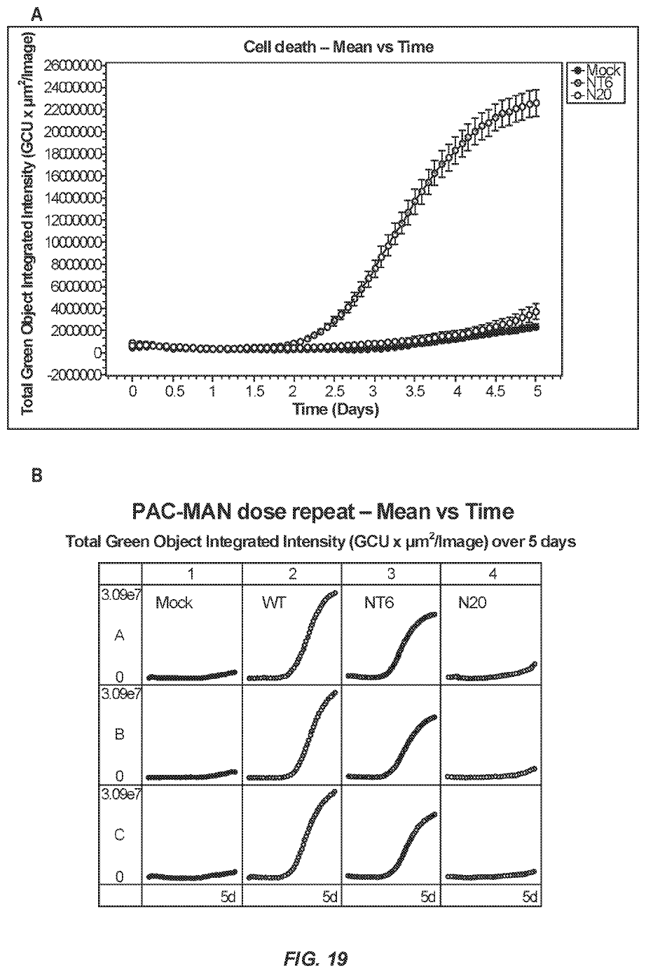

FIG. 19 illustrates repeat testing PAC-MAN with mock control. The total integrated intensity of the green dye (GCU×μm2/Image), which quantifies cell death, was plotted in both one curve (FIG. 19A) and microplate curve (FIG. 19B). FIG. 19A illustrates 229E targeting guide N20 protected the cells from death during a 5 day infection, while most cells transduced with a non-targeting guide (NT6) or mock control were dead. FIG. 19B illustrates integrated intensity of the cells transuded with NT6, N20, or mock control.

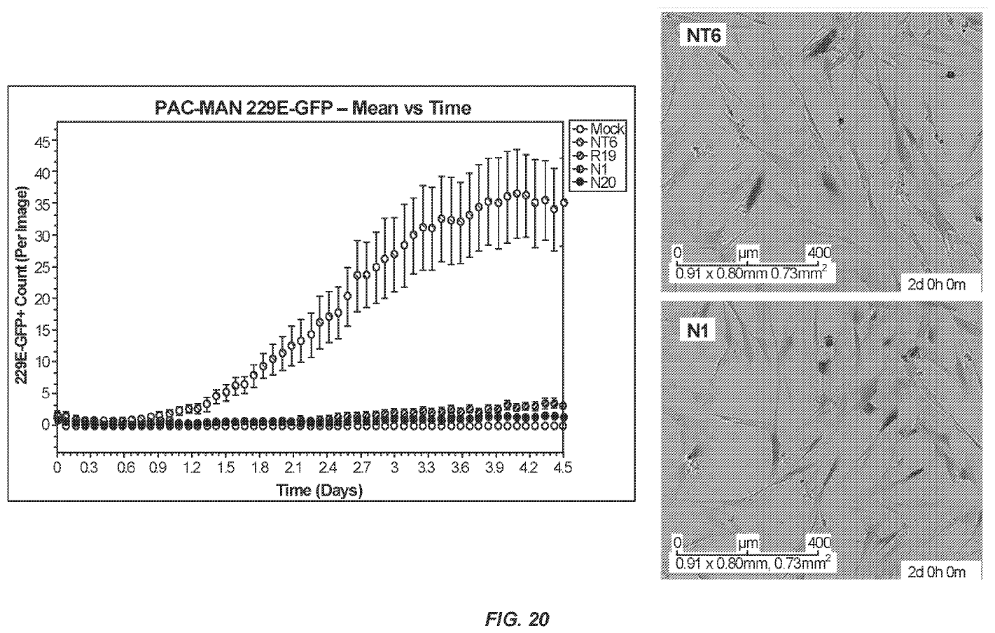

FIG. 20 illustrates testing with PAC-MAN with 229E fused with GFP (229E-GFP). The 229E-GFP virus was used to validate the effect of PAC-MAN. The virus may infect and replicate in the cells transduced with non-targeting guide with many GFP positive cells showed up, while not in the cells transduced with 229E targeting guides. The 229E targeting guide N20 protected the cells from death during a 5 day infection, while most cells transduced with a non-targeting guide NT6 were dead. The 229E-GFP virus was used to validate the effect of PAC-MAN. The virus infected and replicated in the cells transduced with non-targeting guide while not in the cells transduced with 229E targeting guides.

### Example 9. Different MOI and Virus Dosage Test

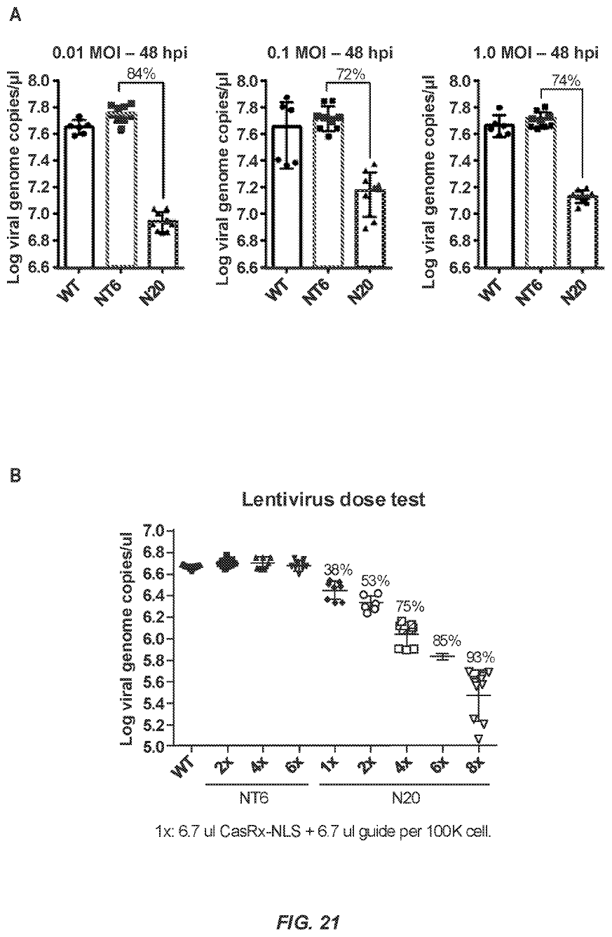

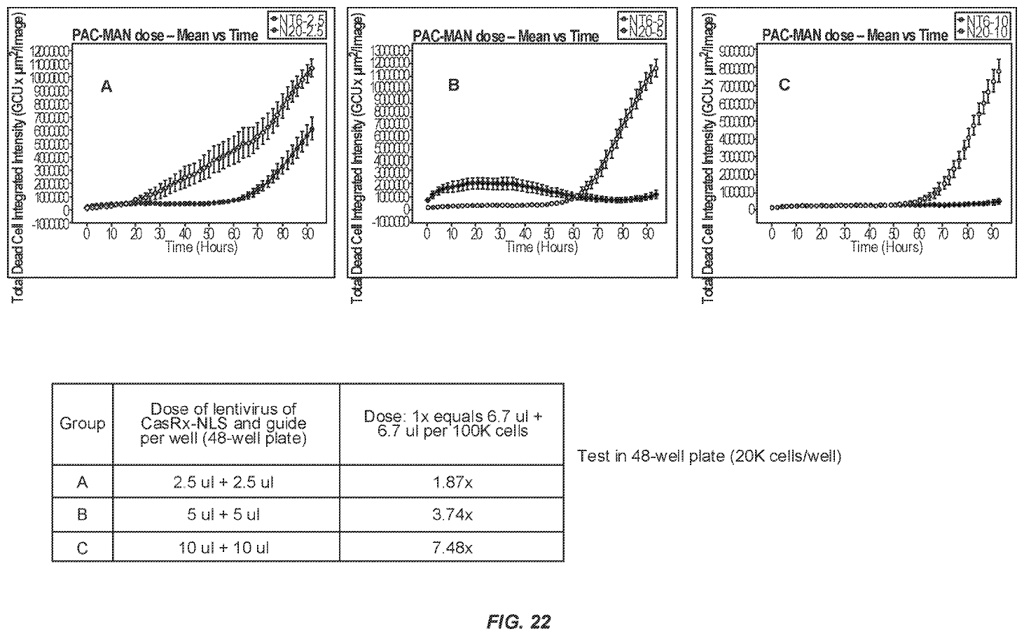

To test the PAC-MAN, cells were infected by 229E at different MOIs. FIG. 21 illustrates testing of PAC-MAN against 229E infection at different multiplicity of infections (MOIs). The 229E targeting guide N20 protected the cells from death, while most cells transduced with a non-targeting guide NT6 were dead. FIG. 21A illustrates that the PAC-MAN inhibited virus replication and protected cells from death even when infected at an MOI as high as 1.0, 100-fold higher than the frequently used 0.01 in the in vitro assays. FIG. 21B illustrates a lentiviral dose test. The inhibition effect of PAC-MAN on 229E virus showed a dose-dependent effect. The higher dose of the PAC-MAN components were used, the higher inhibition effect. The transduction of 100K cells with 6.7 ul of CasRx-NLS and 6.7 ul of guide lentivirus as 1×dose was defined. When using 8×dose, the inhibition was more than 90%. FIG. 22 illustrates PAC-MAN dose effect. The higher dose of PAC-MAN components were transduced, the higher number of cells survived the 229E infection and the longer the cells kept alive.

### Example 10. Synergistic Effect Testing for PAN-MAN and IFNα and Camostat

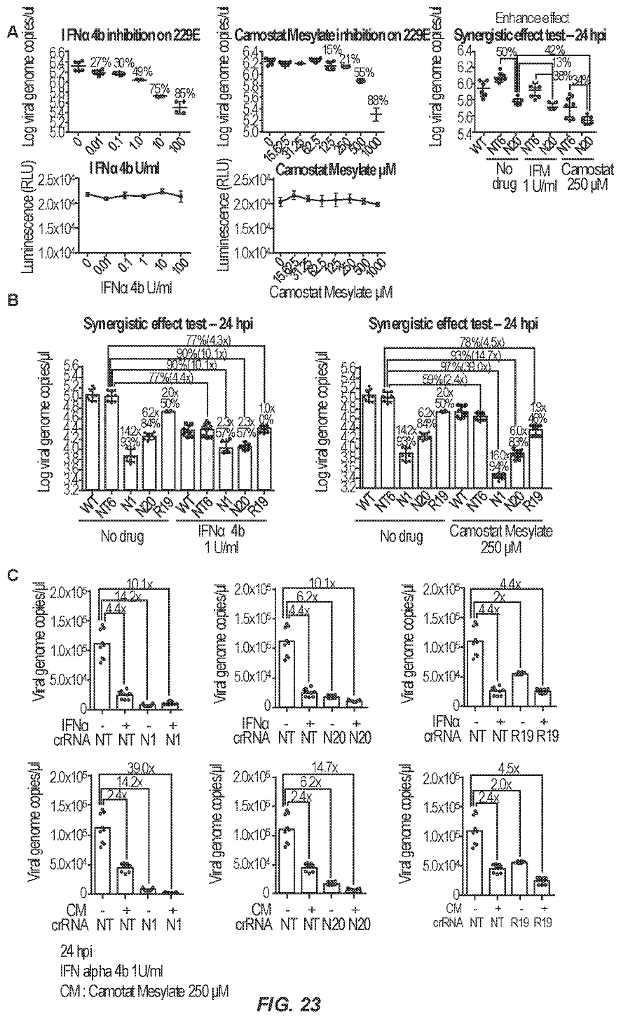

FIG. 23 illustrates synergistic effect of treating the infected cells with PAC-MAN and IFN alpha (IFNα) 4b and Camostat mesylate. FIG. 23A illustrates the cytotoxicity of the IFNα 4b and Camostat mesylate and their effect on inhibition of 229E virus. These two drug showed no obvious cytotoxicity at the tested concentrations. The EC50 of IFNα 4b was 1 U/ml. The EC50 of Camostat mesylate was about 500 μM. When combining these two drugs with PAC-MAN, an enhance effect was observed as the combination further decreased virus replication. FIG. 23B illustrates the synergistic effect at 24 hour post-infection (hpi). From this synergistic effect test, with no drug, the PAC-MAN with guide N1 inhibited 93% of the virus replication, N20 was 84%, and R19 was 50%. The combination of the PAC-MAN with IFNα 4b showed an enhanced effect on inhibition of the virus replication, except that N1 showed a minus effect, the inhibition effect decreased from 93% to 90%. It showed a very synergistic effect when the PAC-MAN was combined with Camostat mesylate. When combined with this inhibitor, PAC-MAN using guide N1 further inhibited the virus replication to 97% lower than the control (NT6 with no drug). While the PAC-MAN (N1) alone was 14.2 fold and the drug alone was 2.4 fold, the combination decreased the virus titer to 39 fold lower than the control. The guide N20 and R19 both showed a promising synergistic effect with the Camostat mesylate. Compared to the NT6 in “no drug” or “IFN” or “Camostat” group, the percentage of inhibition on virus titer was labelled above each bar, the fold change was also labelled. The inhibition effect between drug treated with no drug were labelled with lines above the bars.

The EC50 of Camostat mesylate was about 500 μM. When combining these two drugs with PAC-MAN, the additive effect from IFNα 4b and Camostat mesylate were observed (FIG. 23C). These combinations further decreased virus replication. The PAC-MAN using guide N20 decreased virus titer 6.2 fold (6.2×) lower than NT. IFNα 4b decreased virus titer by 4.4×. Their combination showed an additive effect, which was 10.1×(FIG. 23C top middle). While N1 and R19 showed a minus effect with IFNα 4b (FIG. 23C top left and right). It showed a synergistic effect when the PAC-MAN was combined with Camostat mesylate (FIG. 23C, bottom). While the PAC-MAN using guide N1 alone showed a 14.2 fold decrease of virus titer and the drug alone showed a 2.4 fold decrease, the combination decreased the virus titer to 39 fold lower than the control (FIG. 23C bottom left). The guide N20 and R19 both showed synergistic effect with the Camostat mesylate, too (FIG. 23C bottom middle and right).

### Example 11. Off-Target of PAC-MAN

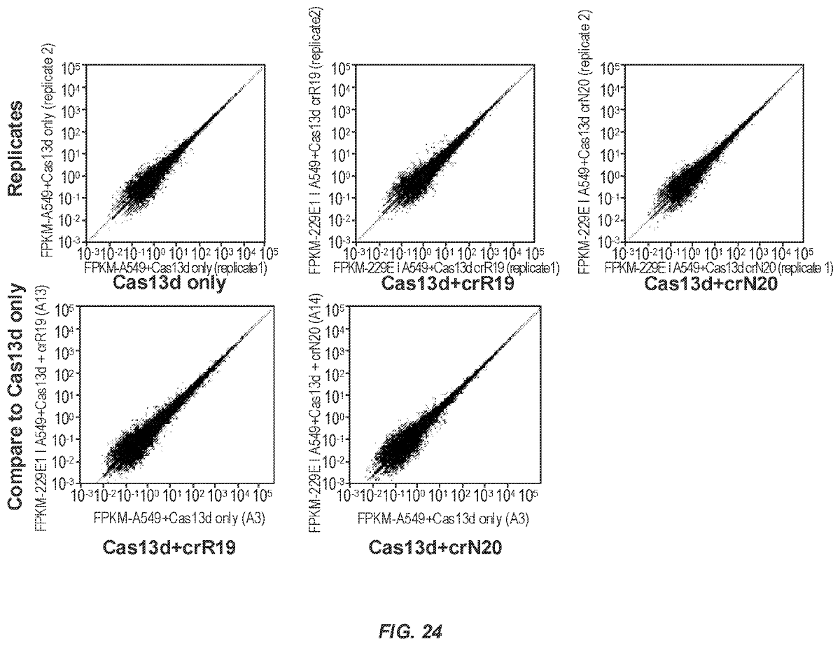

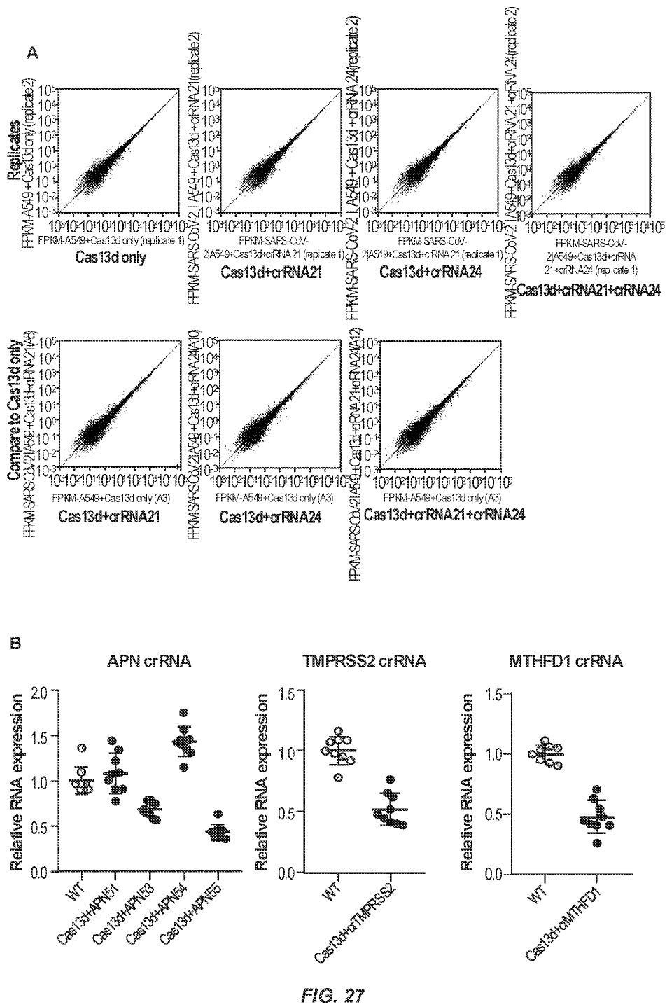

FIG. 24 illustrates RNAseq for determining the off-target effect of PAC-MAN targeting 229E. To analyze off-target effect of Cas13d, Cas13d, Cas13d plus 229E targeting crRNAs (FIG. 24), Cas13d plus SARS-CoV-2 targeting crRNAs (FIG. 27A) were transfected into A549 cells. After 72 hours, the cells were harvested for RNAseq to check the gene expression profile. Cells transfected with Cas13d, or Cas13d plus crRNAs had very similar gene expression pattern. These results suggested that there was no observable Cas13d off-target activities. The top graphs are replicates of every sample. The graphs show that the replicates are consistent. The bottom graphs show Cas13d+crRNA compared to Cas13d only sample, which doesn't show any off-target effect.

### Example 12. crRNA Screening of SARS-CoV-2

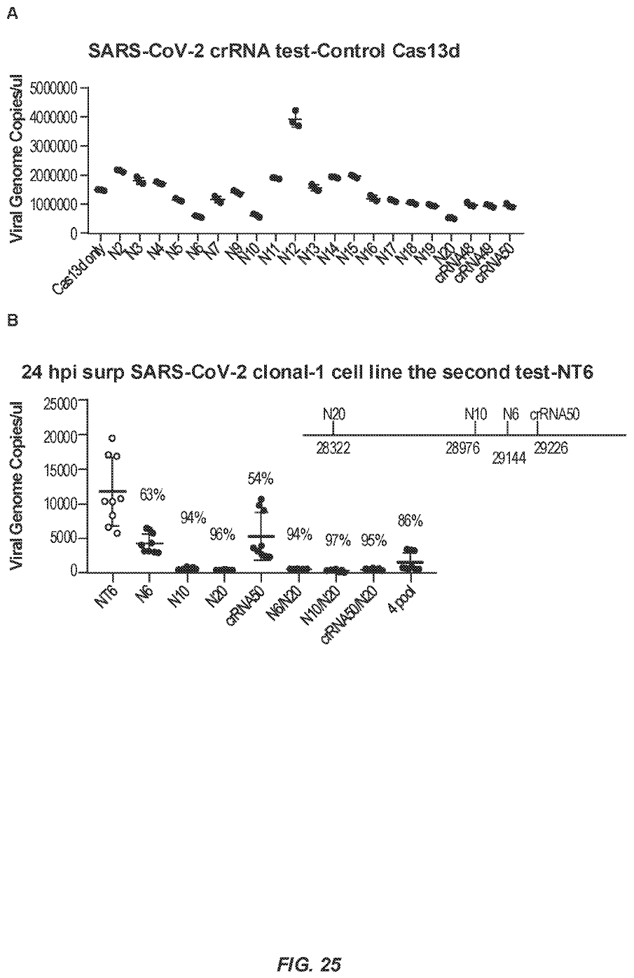

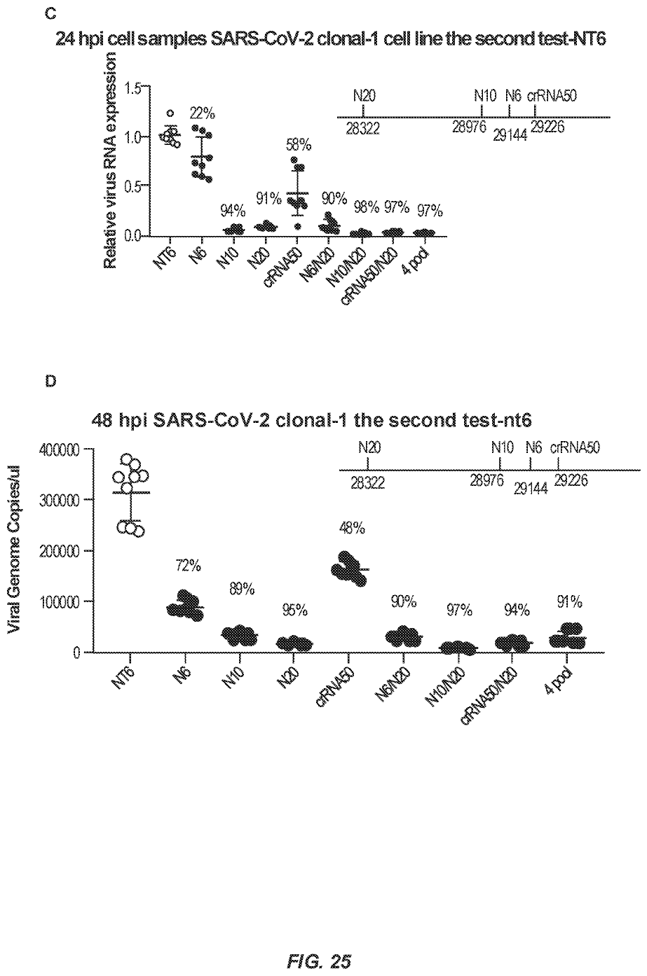

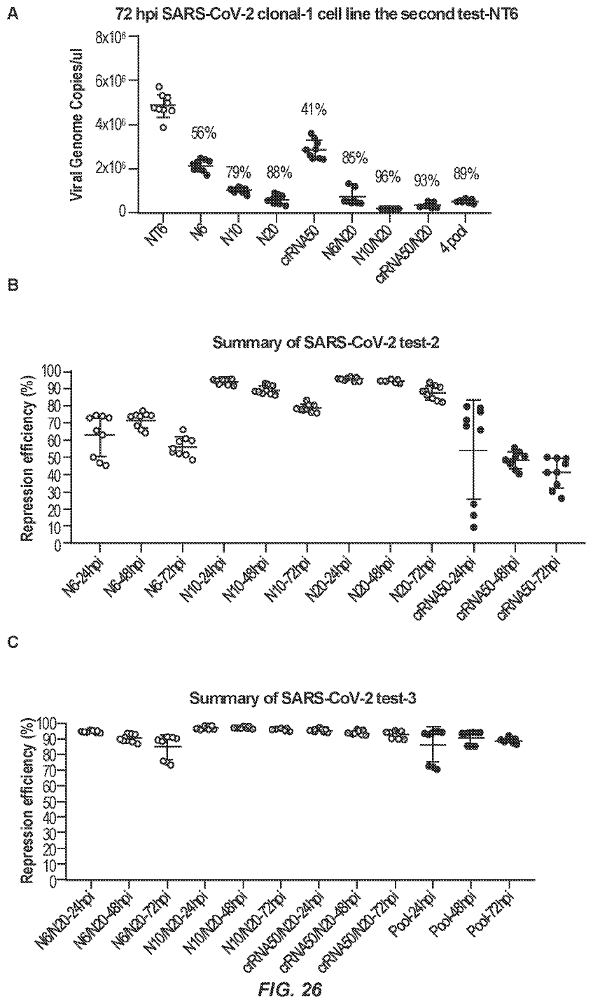

Cas13d expression-veroE6 stable cell line was used to test the protective effect of the crRNAs described herein. FIG. 25 illustrates screening of crRNAs targeting SARS-CoV-2. Using Cas13d expression-veroE6 cell line, crRNAs against SARS-CoV-2 were subjected to repeat testing. FIG. 25A illustrates supernatant test showing that many crRNAs may decrease viral RNA load. Supernatant test showed that crRNAs targeting SARS-CoV-2 decreased viral RNA load. The best crRNA was N20 with the repression efficiency of 96%. Combination of N20 and N10 may get 97% of repression efficiency. FIG. 25A also illustrates additional crRNAs (e.g., other than SEQ ID NOs: 13607-13646). For example, FIG. 25A also illustrates crRNA48-50 (SEQ ID NOs: 13647-13649) for screening purpose. FIG. 25B illustrates supernatant test at 24 hpi. Cellular viral RNA test showed similar results with supernatant results. FIG. 25C illustrates cell sample test at 24 hpi. FIG. 25D illustrates supernatant test at 48 hpi. FIG. 25D illustrates cell sample test at 48 hpi with Cas13d expression-veroE6 cell line. Supernatant test showed that crRNAs targeting SARS-CoV-2 may dramatically decrease viral RNA load. FIG. 26 illustrates screening of crRNAs targeting SARS-CoV-2 at 72 hpi. FIG. 26A illustrates supernatant test showing that crRNAs targeting SARS-CoV-2 may dramatically decrease viral RNA load. FIG. 26B and FIG. 26C illustrate summary of repeat testing of protecting cells from SARS-CoV-2 infection at 72 hpi. Under the same crRNA concentration, it was found that two crRNAs may protect cells from SARS-CoV-2 infection for a longer time. The repression efficiency of single crRNA gradually decreased. However, the combination of two crRNAs, they maintained the same repression efficiency. This indicated that multiple crRNAs were better than single crRNA for cell protection under the same concentration.

FIG. 27A illustrates SARS-CoV-2 off-target effect of PAC-MAN. RNAseq was performed to check the off-target effect of PACMAN (targeting SARS-CoV-2). The top graphs were two replicates of every sample. The bottom graphs showed Cas13d+crRNA compare to Cas13d only sample, which didn't show any off-target effect. FIG. 27B illustrates how the PAC-MAN with designed crRNAs efficiently knocked down endogenous genes: alanine aminopeptidase (APN, SEQ ID NOs: 13601-13064), transmembrane protease, serine 2 (TMPRSS2, SEQ ID NO: 13605), methylenetetrahydrofolate dehydrogenase 1 (MTHFD1, SEQ ID NO: 13606) and also helped blocking virus replication or propagation.

### Example 13. Design Prophylactic Antiviral CRISPR in huMAN Cells (PAC-MAN) as a Form of Genetic Vaccine to Target Influenza a Viruses

To inhibit RNA viruses in human cells, the CRISPR-Cas13d system derived from Ruminococcus flavefaciens XPD3002, a recently discovered RNA-guided RNA endonuclease, can be used. Cas13d uses CRISPR-associated RNAs (crRNAs) that contain designable 22 nucleotide (nt) spacer regions that can direct the Cas13d protein to specific RNA molecules for targeted RNA degradation. This provides a potential mechanism for targeting virus for specific viral RNA genome degradation and viral gene expression inhibition. Because of its small size (2.8 kb), high specificity, and strong catalytic activity, the Cas13d can target and destroy RNA viruses including all strains of the influenza viruses.

Described herein is a CRISPR strategy named Prophylactic Antiviral CRISPR in huMAN cells (PAC-MAN) developed as a form of genetic vaccine to target circulating influenza stain and potentially all sequenced of influenza viruses. A bioinformatic pipeline defined highly conserved regions across the influenza virus genomes and flagged these regions for genome degradation and viral gene inhibition by the use of CRISPR-dCas13d. A panel of designed crRNA pools was screened against influenza fragments for how well the crRNAs may target conserved viral regions in order to identify the most effective crRNAs. Such pools of crRNAs can then be part of a pan-influenza treatment regiments.

### Example 14. Cas13d PAC-MAN is Able to Inhibit IAV Infection in Human Lung Epithelial Cells

To create crRNAs targeting a broad range of IAV strains, all complete IAV genomes retrieved from the Influenza Research Database were aligned and searched for the most conserved regions. All complete, pre-aligned, complementary RNA (cRNA) segment sequences for IAV were downloaded from the Influenza Research database. All sequences were reverse-transcribed to viral RNA (vRNA) and filtered for the most-frequently occurring length for each segment. Each filtered segment was uploaded to Weblogo3 (http://weblogo.threeplusone.com/) to create weblogos for visualization. These segment ends are highly conserved for all 8 segments. The segment ends are attractive regions for targeting with Cas13d, because of the potential to inhibit a broad range of IAV strains and because of the previous evidence that interfering with the packaging of one segment encoded on the segment ends can decrease the packaging efficiency of other segments and overall virion packaging. Using sequence representation from ˜140 different strains of influenza, crRNAs were designed to robustly bind to as many different IAV strains as possible. The bioinformatics analysis resulted in 6 crRNAs targeting highly conserved genome regions for each of the 8 IAV segments, for a total of 48 crRNAs (SEQ ID NOs: 13301-13354, 13401-13448, or 13501-13548).

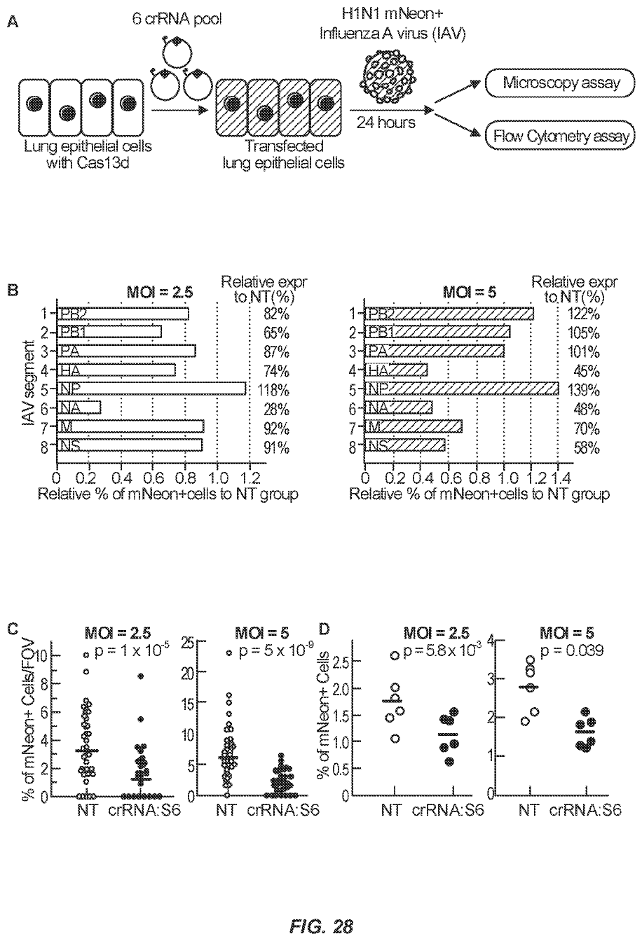

To test the efficiency of IAV-targeting crRNAs, a screen was performed with the crRNA pools in the stable Cas13d A549 cell line to determine which were most effective at inhibiting IAV infection (FIG. 28A). To do this, A549 cells were transfected with a pool of 6 crRNAs targeting a given IAV genome segment. Two days after crRNA transfection, the Cas13d A549 cells were challenged with PR8-mNeon, a strain of H1N1 IAV engineered to express the mNeonGreen gene, a fluorescent reporter protein (hereafter referred to as PR8-mNeon) at an MOI of 2.5 or 5.0. At 18 hours post-challenge, the cells were analyzed for IAV infection through microscopy and flow cytometry by examining the percentage of mNeon positive cells for each segment-targeting crRNA pool and the non-targeting crRNA pool.

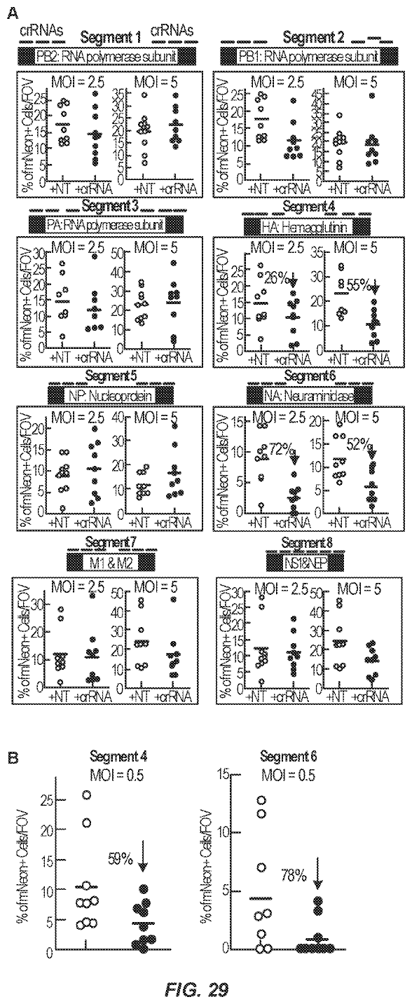

Out of all crRNAs, the crRNA pool targeting segment 6 (S6) delivered the most consistent and robust results across different MOIs (72% reduction for MOI=2.5 and 52% reduction for MOI=5: FIG. 28B and FIG. 29A). Additionally, the segment 4 (S4) pool also showed moderate but consistent inhibition. To verify Cas13d-mediated inhibitory effects on S4 and S6, a lower MOI of 0.5 was further tested, which showed better inhibition of IAV across samples (e.g., 79% reduction using crRNAs targeting S6; FIG. 29B). This confirmed consistent inhibitory activity of IAV by Cas13d when targeting these two segments. There is a possible trend of greater inhibition at lower viral titers, suggesting that Cas13d PAC-MAN may be a particularly effective strategy for prevention of new infections, which typically occurs from exposure to a low level of virus.

The S6-targeting crRNA pool was further characterized. S6 encodes the gene neuraminidase (NA), which releases budding virions from the host cell. Microscopy quantification was performed on mNeon+ cells and found that mNeon expression was reduced by 62% (p=10⁻⁵) and 73% (p=5×10⁻⁹) with an MOI of 2.5 and 5, respectively, with S6-targeting crRNAs (FIG. 29C), and this trend was corroborated by flow cytometry (FIG. 29D). Together, the data showed that Cas13d PAC-MAN was able to target highly conserved viral regions and robustly inhibit viral replication in human lung epithelial cells.

### Example 15. An Integrated Pipeline for Cas13 crRNA Analysis for Virus-Targeting Coverage, Efficiency, and Specificity

The COVID-19 pandemic caused by SARS-CoV-2 has led to significant mortality, highlighting the need for antivirals to combat RNA viruses. The RNA-guided RNA-targeting CRISPR-Cas13 system offers an antiviral strategy against SARS-CoV-2 sequences and live influenza A virus. In silico analysis of using PAC-MAN can be expanded to target notable pathogenic viral species and most human-infectious RNA viruses. An integrated pipeline described herein has been created to analyze crRNAs that can target broad virus strains with high predicted efficiency using a reported algorithm and transcriptome-wide specificity. Among 16 RNA virus families, analysis revealed that a group of 14 crRNAs (SEQ ID NOs: 13651-13664) were able to target 10 distinct families (24.407 strains; targeting SEQ ID NOs: 13671-13684) by targeting at least 90% of human-infectious viruses, as well as determinant numbers of crRNAs needed to cover a virus family. An online resource, COMBAT (COMputer-aided Broad-spectrum AnTiviral) was constructed, to disseminate the Cas13d crRNAs targeting RNA viruses. Validation of the crRNAs targeting SARS-CoV-2 sequences showed a high correlation between predicted and measured efficiency. This work provided useful analysis and a resource for using PAC-MAN to target broad pathogenic RNA viruses and facilitates CRISPR-based antiviral tool development.

Since December 2019, the world has been devastated by the COVID-19 pandemic, caused by the RNA virus, Severe Acute Respiratory Syndrome coronavirus 2 (SARS-CoV-2). Besides SARS-CoV-2, the outbreaks of many RNA viruses in recent years, including Zika virus, Ebola virus, Marburg virus, and MERS (Middle East Respiratory Syndrome) virus, have led the World Health Organization (WHO) to declare public health emergencies. While vaccines and therapeutic options against SARS-CoV-2 have advanced into clinical trials, most RNA viruses lack an approved therapy.

In search of potential antivirals, a CRISPR-Cas13d-based strategy, PAC-MAN (Prophylactic Antiviral CRISPR in huMAN cells) was developed to target and inhibit SARS-CoV-2 and IAV sequences. A few works also reported that CRISPR-Cas13a/b systems can be used to inhibit RNA viruses, including lymphocytic choriomeningitis virus (LCMV), vesicular stomatitis virus (VSV), human immunodeficiency virus type 1 (HIV-1), human respiratory syncytial virus (HRSV), Dengue virus, Turnip mosaic virus and tobacco turnip mosaic RNA virus (TuMV). The CRISPR-Cas13 systems (subtypes a, b, d) offer attractive prophylactic or therapeutic options due to several key features) CRISPR-Cas13 is highly programmable, which allows it to be rapidly adapted against new RNA viruses by designing the crRNA sequence) crRNAs can be designed to target highly conserved viral sequences to cover multiple strains and prevent viral mutational escape. One remaining question is whether the system can be expanded to target a broad-spectrum of RNA viruses.

Here, an in silico comprehensive analysis of using PAC-MAN was developed to target notable pathogenic viral species and all human-infectious RNA viruses in the Virus Pathogen Database and Analysis Resource (ViPR). An integrated crRNA analysis pipeline was first generated by incorporating multiple existing algorithms that compute the coverage, efficiency and specificity of crRNAs for targeting RNA viruses. Using this pipeline, crRNAs for targeting 16 RNA virus families were searched. 10 non-segmented RNA virus families were observed to be targeted by a small group of 14 crRNAs (SEQ ID NOs: 13651-13664) with more than 90% coverage of human-infectious viruses. A few families of viruses may be even targeted by 1 or 2 crRNAs for coverage of more than 90% of sequenced strains. In contrast, families of viruses with segmented RNA genomes required significantly more crRNAs for full targeting. An online resource, COMBAT (COMputer-aided Broad-spectrum AnTiviral), was built to disseminate Cas13d crRNAs targeting a broad spectrum of RNA viruses. A group of predicted crRNAs targeting the SARS-CoV-2 sequences was also experimentally tested and found a high correlation between predicted and validated efficiency. All computed crRNAs are publicly available at http://crispr-combat.stanford.edu/. This work provides a comprehensive analysis and useful resource for using PAC-MAN to target broad pathogenic RNA viruses and can facilitate CRISPR-based antiviral technology development.

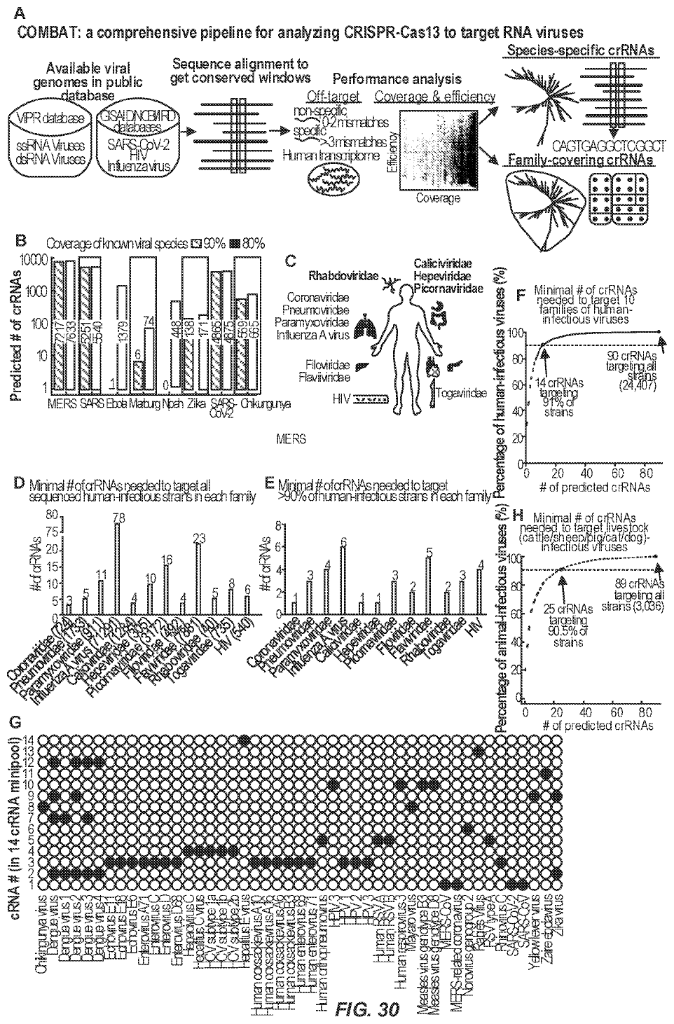

RNA virus genomes were retrieved from public databases, including 16 RNA virus families from the Virus Pathogen Database and Analysis Resource (ViPR), human immunodeficiency virus (HIV) from NCBI, and influenza A or B viruses (IAV or IBV) from the Influenza Research Database (IRD) (Table 7). This pipeline first analyzed conserved genome sequences (e.g., SEQ ID NO: 13740) within a designated group of viruses, which generated the primary regions for crRNA targeting (FIG. 30A). All possible crRNA candidates were generated with perfect matches to the conserved sequence. The pipeline eliminated crRNAs putative with off-target binding in the human transcriptome (<=2 mismatches), a threshold that was previously defined to distinguish specific vs. non-specific crRNAs. An algorithm that predicted crRNA efficiency score (range 0˜1, cut off >=0.5) was used and computed the coverage of virus strain targeting for every crRNA. The pipeline was used to analyze two types of crRNAs with different features. The first type is species-specific crRNAs, which were a comprehensive set of crRNAs that targeted conserved regions in closely related branches of viruses with predicted specificity and efficiency. The second type was family-covering crRNAs, which were minimal pools of crRNAs that collectively target all viruses in a family.

TABLE 7
Analyzed RNA virus families and species relating to FIG. 30

| Virus | | RNA | | All analyzed strains | Human-Host |
|---|---|---|---|---|---|
| Family/Featured | Data- | Virus | Segmented | (or segmented | analyzed |
| Virus Name | bases | Types | Genome? | genomes) | strains |
| arenaviridae | ViPR | −ssRNA | Yes | 1850 | 1127 |
| caliciviridae | ViPR | +ssRNA | NO | 2010 | 1284 |
| coronaviridae | ViPR | +ssRNA | NO | 3382 | 724 |
| filoviridae | ViPR | −ssRNA | NO | 871 | 492 |
| flaviviridae | ViPR | +ssRNA | NO | 14697 | 7881 |
| hantaviridae | ViPR | −ssRNA | Yes | 2865 | 270 |
| hepeviridae | ViPR | +ssRNA | NO | 643 | 305 |
| nairoviridae | ViPR | −ssRNA | Yes | 884 | 404 |
| paramyxoviridae | ViPR | −ssRNA | NO | 2614 | 911 |
| peribunyaviridae | ViPR | −ssRNA | Yes | 3489 | 449 |
| phenuiviridae | ViPR | −ssRNA | Yes | 3182 | 1832 |
| picomaviridae | ViPR | +ssRNA | NO | 5909 | 3172 |
| pneumoviridae | ViPR | −ssRNA | NO | 1840 | 1733 |
| reoviridae | ViPR | dsRNA | Yes | 44700 | 6605 |
| rhabdoviridae | ViPR | −ssRNA | NO | 2785 | 40 |
| togaviridae | ViPR | +ssRNA | NO | 1984 | 735 |
| HIV | NCBI | +ssRNA | NO | 540 | |
| Influenza_A_virus | IRD | −ssRNA | Yes | 71291 | |
| Influenza_B_virus | IRD | −ssRNA | Yes | 9193 | |

### Example 16. Targeting Pandemic/Epidemic-Causing Pathogenic RNA Virus Species Using PAC-MAN

crRNAs for each pandemic/epidemic-causing pathogenic RNA virus species per World Health Organization (WHO) were first identified, including SARS, SARS-CoV-2, MERS, Ebola virus, Marburg virus, Zika virus, Chikungunya virus, and Nipah virus. For most pathogenic virus species, a collection of crRNAs with predicted high specificity (>=3 mismatches) and efficiency (score>=0.5) was obtained, with each crRNA covering more than 90% of sequenced strains (FIG. 30B). One exception was Nipah virus, for which no crRNA candidate was able to cover 90% of sequenced strains. Nevertheless, lowering the requirement to 80% virus strain coverage showed a list of crRNAs that may target the Nipah virus.

### Example 17. Analysis of PAC-MAN for Targeting a Broad Spectrum of RNA Viruses

crRNA that may be used to target a broad spectrum of RNA virus families was next analyzed. 10 RNA virus families (calicividirae, coronaviridae, filoviridae, hepeviridae, paramyxoviridae, phenuiviridae, picomaviridae, pneumoviridae, rhabdoviridae, and togaviridae) from the ViPR database that infect various human organs and systems, including 6 positive-sense single-strand RNA (ssRNA) virus families, 4 negative-sense ssRNA virus families, and HIV and IAV species (FIG. 30C) were analyzed. For each family or HIV/IAV species, the smallest minipool of crRNAs that may collectively target every strain using a greedy approximation algorithm was determined.

10 RNA virus families and HIV may be targeted by a small set of crRNAs (3-23 crRNAs). One exception is IAV, which required 78 crRNAs for full coverage likely due to its high diversity and its segmented genome, which was split into 8 segments (FIG. 30D). Since many families contained rare strains with distinct genomes, if aiming to target at least 90% of viral strains, all families may be targeted by a very small number of crRNAs (1-6) (FIG. 30E).

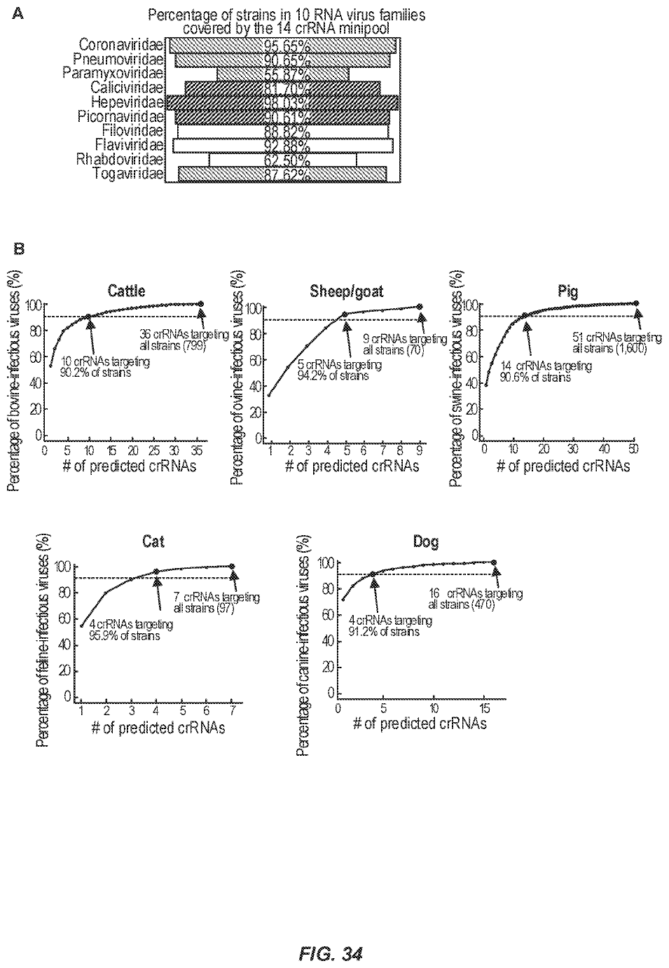

Importantly, a minipool of 14 crRNAs (SEQ ID NOs: 13651-13664) was able to target more than 90% of human-infectious viruses in the 10 RNA virus families (FIG. 30F and FIG. 34A). A total of 90 crRNAs may cover all strains (24,407) in the 10 families with zero mismatches. It was observed that this minipool also covered many common human-infectious viral species (FIG. 30G). This indicated that Cas13-mediated PAC-MAN may offer a potential broad-spectrum antiviral (BSA) strategy for broadly targeting human RNA viruses.

crRNAs that may target animal viruses were also analyzed. For this purpose, viruses were categorized according to their host, including common domestic livestock (cattle, sheep, pig, cat, and dog) that have close interaction with humans. For each category of virus, it was observed that a small set of crRNAs was able to target the majority of viruses (FIG. 30H and FIG. 34B). This analysis suggested that beyond human-infectious viruses, PAC-MAN may be used as a broad-spectrum antiviral strategy in animals, a field that was underdeveloped in biotechnology sector.

### Example 18. Determinants Required of crRNAs Targeting a Broad Spectrum of RNA Virus Families

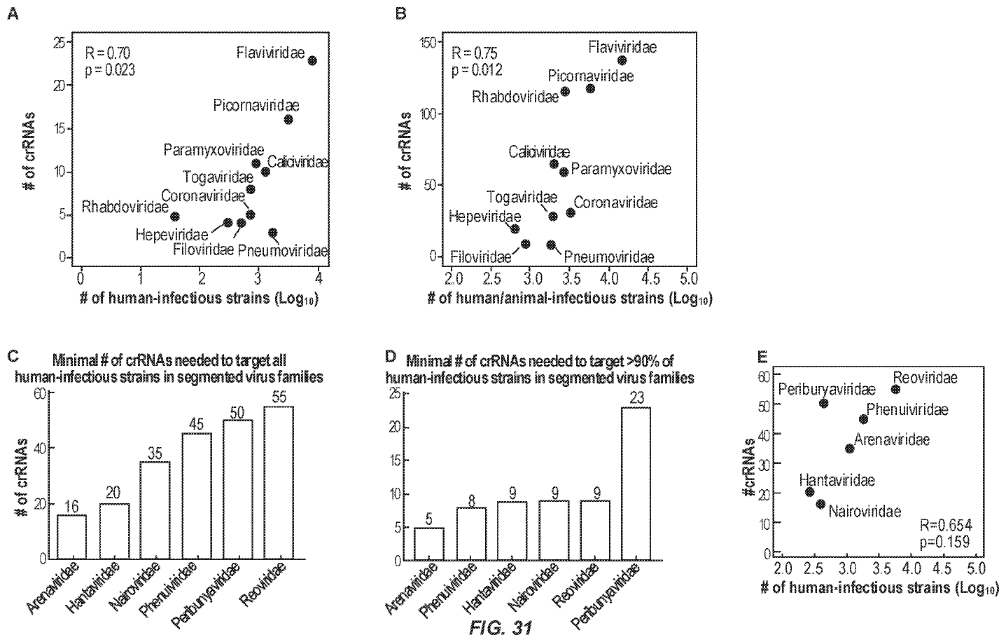

Whether families with more virus strains required more crRNAs were analyzed. For the 10 RNA virus families analyzed, it was observed that a linear relationship between strain number and the required crRNA number (R=0.70, p=0.023, FIG. 31A). When all human and animal viruses were included, a similar linear relationship (R=0.75. p=0.012, FIG. 31B) was observed. This suggested that the required crRNA numbers to cover an RNA virus family was strongly influenced by the species/strain number in that family.

The observation that IAV required more crRNAs for coverage compared to the other 10 families and HIV led to analysis of more RNA virus families with segmented genomes. To do this, 6 segmented RNA virus families were included, including 5 negative-sense ssRNA virus families and 1 double-stranded RNA (dsRNA) virus family. All analyzed segmented RNA virus families were observed to require significantly more crRNAs for targeting human-infectious species in that family (16˜55 crRNAs for full coverage and 5˜23 crRNAs for 90% coverage, FIG. 31C and FIG. 31D). This was due to the reassortment process, a shared feature of segmented RNA viruses that had the capacity to exchange genome segments during co-infection. Reassortment greatly increased the diversity of segmented viral genome sequences, making it more challenging to define very few crRNAs to broadly cover strains. Despite the nature of these segmented genomes, it was observed that the number of required crRNAs for coverage was strongly correlated with the number of strain in that family (R=0.654, p=0.158, FIG. 31E) Overall, this data suggests that PAC-MAN was most effective against non-segmented RNA virus families for broad-spectrum targeting.

### Example 19. COMBAT is an Online Resource to Use CRISPR-Cas13d for Broad-Spectrum Antiviral Targeting

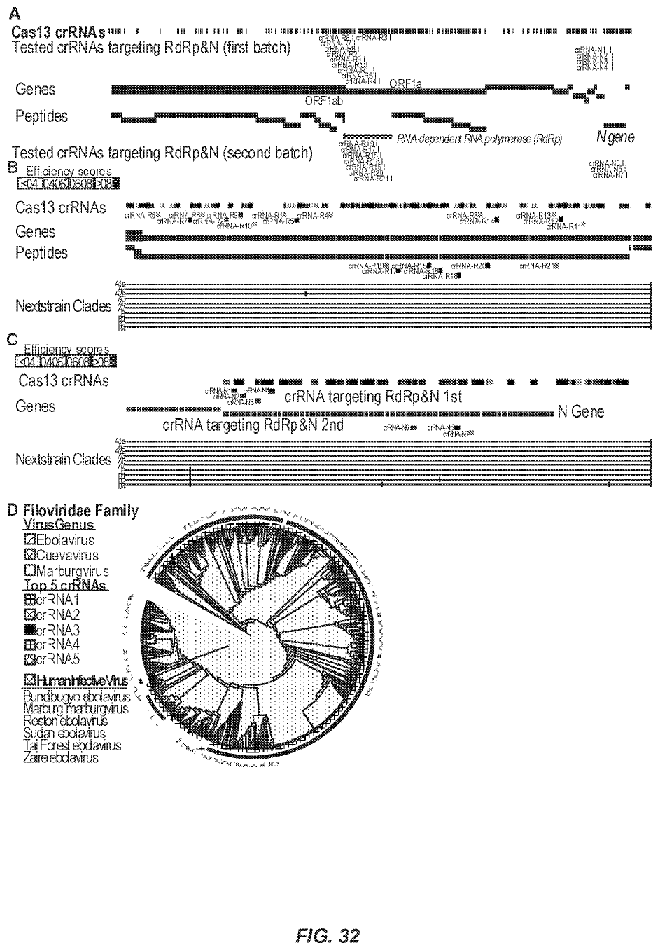

COMBAT (COMputer-aided Broad-spectrum AnTiviral), was built to facilitate the dissemination of analyzed Cas13d crRNAs for 16 RNA virus families, as well as for HIV, IAV and IBV. For species-specific crRNAs in each family, a library of crRNAs was defined with predicted high specificity (>=3 mismatches in the human transcriptome) and high efficiency (score >=0.5). An interactive tool was provided to facilitate the choice of crRNAs targeting specific genes on a virus. A graphical user interface (GUI) was also implemented for SARS-CoV-2, which integrated with the UCSC Genome Browser and created a visualization for the mapping of crRNAs to sequence position along the annotated viral genome for visualizing mapping of crRNAs to the annotated viral genome. An example is provided to show where predicted crRNAs target the SARS-CoV-2 genome (FIG. 32A, FIG. 32B, and FIG. 32C). For family-covering crRNAs, a phylogenetic tree was generated using the Interactive Tree Of Life (iTOL) tool to visualize the evolutionary relationship of the strains targeted by each crRNA. Each viral strain in the phylogenetic tree was annotated with the genus that the strain belonged to, whether the strain was a human host, and which (if any) of the first 5 crRNAs in the family-covering minipool targeted the strain. An example of this phylogenetic tree was provided to show family-covering crRNAs for the Filoviridae family including Ebola virus (FIG. 32D).

### Example 20. Validation of Predicted crRNAs for Targeting SARS-CoV-2 Sequences

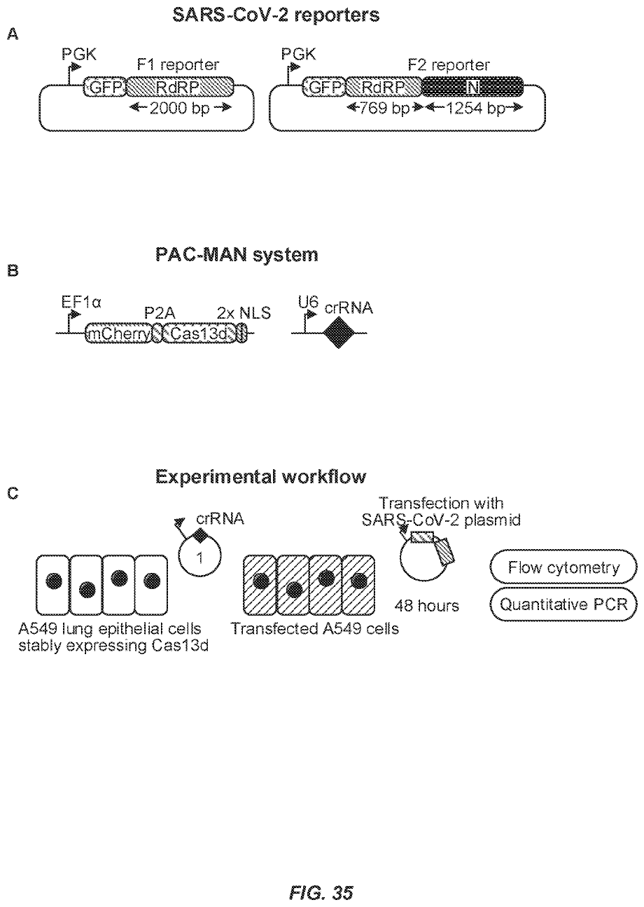

Whether the predicted species-specific crRNAs may enable effective Cas13d-mediated inhibition of the SARS-CoV-2 sequence was experimentally validated. SARS-CoV-2 reporters, F1 and F2, which encoded a GFP gene fused to SARS-CoV-2 sequences encoding RNA-dependent RNA polymerase (RdRP) was used, which maintained virus proliferation, or nucleocapside (N), which controlled viral packaging (FIG. 35A). The Cas13d and crRNA were expressed using two plasmids as reported before (FIG. 35B). To measure the inhibitory effects of crRNAs, individual crRNA plasmids and SARS-CoV-2 reporter plasmids were co-transfected into A549 lung epithelial cells that expressed Cas13d, and quantified GFP expression using flow cytometry and target RNA abundance using quantitative PCR (qPCR) 48 hours post transfection (FIG. 35C).

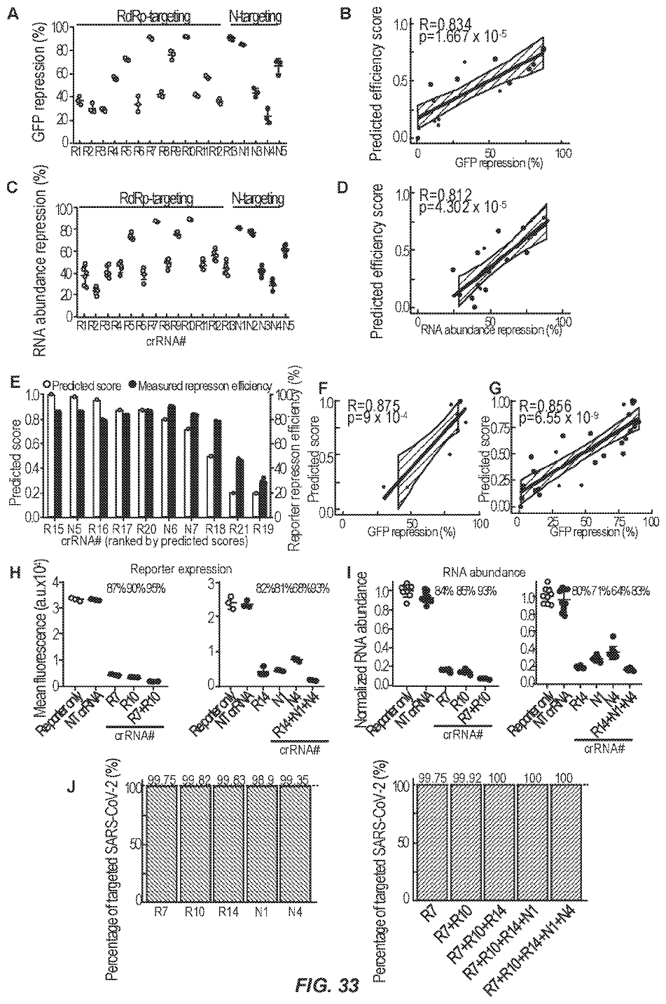

A list of crRNAs targeting RdRP or N without efficiency prediction was first (FIG. 32B, FIG. 32C, and Table 8). Varying repression effects on the SARS-CoV-2 reporters of individual crRNAs were observed using flow cytometry (FIG. 33A and Table 9). Interestingly, there was a statistically significant high correlation between percentage of GFP repression and the predicted efficiency scores using a recently reported algorithm (R=0.834, p=1.667×10⁻⁵: FIG. 33B). These results were consistent with the repressive effects on RNA abundance as measured by qPCR (R=0.812, p=4.302×10-5; FIG. 33C, FIG. 33D, and Table 10). These data together suggested the reported algorithm was able to faithfully predict efficient crRNAs for viral genome targeting, despite it being developed based on data from crRNAs targeting human transcripts.

To further validate the performance of COMBAT-predicted crRNAs, more predicted crRNAs targeting the SARS-CoV-2 sequence with varying efficiency scores were tested (FIG. 32B and FIG. 32C). Again, a strong correlation between the predicted efficiency and the measured reporter repression (R=0.875, p=0.0009; FIG. 33E and FIG. 33F) was observed. The combined two sets of crRNAs showed a correlation of 0.856 (p=6.55×10⁻⁹, FIG. 33G). A binary trend was observed: most crRNAs with an efficiency score higher than 0.5 showed efficient repression, whereas crRNAs with an efficiency score lower than 0.5 showed almost no repression. This justified the choice of 0.5 as the threshold to determine the efficient crRNAs. These independent experiments confirmed the efficacy of COMBAT using the reported efficiency-prediction algorithm, which also suggested COMBAT may be used to predict crRNAs that efficiently inhibited other RNA virus sequences.

TABLE 8
All sequences used in the study, including crRNAS tested for targeting SARS-
CoV-2 and non-targeting crRNAs, related to FIG. 32

| | | Position on | | Predicted | |
|---|---|---|---|---|---|
| | | SARS-CoV-2 | Target | Efficiency | SEQ ID |
| Name | Spacer DNA sequence | genome | gene | Score | NO: |
| R1 | tctgatcccaatatttaaaata | 14296-14317 | RdRP | 0.08 | 13701 |
| R2 | ggcgtacacgttcacctaagtt | 13969-13990 | RdRP | 0.33 | 13702 |
| R3 | cttgagcacactcattagctaa | 15403-15424 | RdRP | 0.19 | 13703 |
| R4 | tagcagggtcagcagcatacac | 14557-14578 | RdRP | 0.52 | 13704 |
| R5 | aaacattaaagtttgcacaatg | 14365-14368 | RdRP | 0.69 | 13705 |
| R6 | gacaacaattagtttttaggaa | 13582-13603 | RdRP | 0.00 | 13706 |
| R7 | atatatgtggtaccatgtcacc | 13762-13783 | RdRP | 0.78 | 13707 |
| R8 | attaccttcatcaaaatgcctt | 13833-13854 | RdRP | 0.32 | 13708 |
| R9 | cttgattatctaatgtcagtac | 14050-14071 | RdRP | 0.48 | 13709 |
| R10 | aagaatctacaacaggaactcc | 14128-14149 | RdRP | 0.76 | 13710 |
| R11 | atcataagtgtaccatctgtttt | 15985-16007 | RdRP | 0.15 | 13711 |
| R12 | agcaaaattcatgaggtccttt | 15859-15880 | RdRP | 0.67 | 13712 |
| R13 | aacattttgcttcagacataaa | 15817-15838 | RdRP | 0.16 | 13713 |
| R14 | cactattagcataagcagttgt | 15496-15517 | RdRP | 0.64 | 13714 |
| N1 | ttgaatctgagggtccaccaaa | 28322-28343 | N | 0.60 | 13715 |
| N2 | ccccactgcgttctccattctg | 28355-28376 | N | 0.47 | 13716 |
| N3 | tgaaccaagacgcagtattatt | 28412-28433 | N | 0.11 | 13717 |
| N4 | aacgccttgtcctcgagggaat | 28468-28489 | N | 0.42 | 13718 |
| R15 | tactacagatagagacaccagc | 15111-15133 | RdRP | 1.00 | 13719 |
| R16 | acatttaggataatcccaaccc | 15284-15306 | RdRP | 0.96 | 13720 |
| R17 | tattaaatggaaaaccagctga | 14940-14962 | RdRP | 0.87 | 13721 |
| R18 | acagtagctcctctagtggcgg | 15178-15200 | RdRP | 0.50 | 13722 |
| R19 | caccatcgtaacaatcaaagta | 14874-14896 | RdRP | 0.20 | 13723 |
| R20 | aacatatagtgaaccgccacac | 15443-15465 | RdRP | 1.00 | 13724 |
| R21 | ggtcagtctcagtccaacattt | 15831-15853 | RdRP | 0.20 | 13725 |
| N5 | aaattgtgcaatttgcggccaa | 29172-29194 | N | 0.98 | 13726 |
| N6 | gcagcagatttcttagtgacag | 29006-29028 | N | 0.80 | 13727 |
| N7 | tgtgacttccatgccaatgcgc | 29226-29248 | N | 0.72 | 13728 |
| crRNA-NC | gaactcgtatccgatgataaga | | | | 13729 |

TABLE 9
P values for crRNA screens, relating to FIG. 33

| | | |
|---|---|---|
| crRNAs | Experiment | P value |

FIG. 33A

| | | |
|---|---|---|
| R1 | SARS-CoV-2 reporter expression | 1.50E−04 |
| R2 | SARS-CoV-2 reporter expression | 5.48E−04 |
| R3 | SARS-CoV-2 reporter expression | 1.40E−04 |
| R4 | SARS-CoV-2 reporter expression | 1.09E−05 |
| R5 | SARS-CoV-2 reporter expression | 2.88E−06 |
| R6 | SARS-CoV-2 reporter expression | 1.36E−03 |
| R7 | SARS-CoV-2 reporter expression | 1.17E−06 |
| R8 | SARS-CoV-2 reporter expression | 5.27E−05 |
| R9 | SARS-CoV-2 reporter expression | 1.12E−05 |
| R10 | SARS-CoV-2 reporter expression | 9.06E−07 |
| R11 | SARS-CoV-2 reporter expression | 4.13E−07 |
| R12 | SARS-CoV-2 reporter expression | 5.50E−07 |
| R13 | SARS-CoV-2 reporter expression | 7.43E−06 |
| N1 | SARS-CoV-2 reporter expression | 6.06E−08 |
| N2 | SARS-CoV-2 reporter expression | 1.58E−08 |
| N3 | SARS-CoV-2 reporter expression | 3.81E−05 |
| N4 | SARS-CoV-2 reporter expression | 2.45E−03 |
| N5 | SARS-CoV-2 reporter expression | 6.37E−05 |

FIG. 33C

| | | |
|---|---|---|
| R1 | mRNA abundance | 1.88E−05 |
| R2 | mRNA abundance | 9.13E−05 |
| R3 | mRNA abundance | 1.62E−06 |
| R4 | mRNA abundance | 4.57E−07 |
| R5 | mRNA abundance | 2.19E−09 |
| R6 | mRNA abundance | 2.35E−06 |
| R7 | mRNA abundance | 2.98E−10 |
| R8 | mRNA abundance | 1.58E−07 |
| R9 | mRNA abundance | 1.43E−09 |
| R10 | mRNA abundance | 2.34E−10 |
| R11 | mRNA abundance | 4.80E−09 |
| R12 | mRNA abundance | 1.81E−09 |
| R13 | mRNA abundance | 4.71E−08 |
| N1 | mRNA abundance | 3.26E−12 |
| N2 | mRNA abundance | 7.41E−12 |
| N3 | mRNA abundance | 1.37E−08 |
| N4 | mRNA abundance | 9.81E−07 |
| N5 | mRNA abundance | 3.54E−10 |

FIG. 33E

| | | |
|---|---|---|
| R15 | SARS-CoV-2 reporter expression | 1.15E−06 |
| N5 | SARS-CoV-2 reporter expression | 7.80E−07 |
| R16 | SARS-CoV-2 reporter expression | 1.64E−06 |
| R17 | SARS-CoV-2 reporter expression | 1.29E−06 |
| R20 | SARS-CoV-2 reporter expression | 7.64E−07 |
| N6 | SARS-CoV-2 reporter expression | 6.51E−07 |
| N7 | SARS-CoV-2 reporter expression | 8.49E−07 |
| R18 | SARS-CoV-2 reporter expression | 1.87E−06 |
| R19 | SARS-CoV-2 reporter expression | 1.60E−04 |
| R21 | SARS-CoV-2 reporter expression | 1.12E−05 |

FIG. 33H (Left) and FIG. 33I (Left)

| | | |
|---|---|---|
| crRNA-NC | SARS-CoV-2 reporter expression | 7.35E−01 |
| R7 | SARS-CoV-2 reporter expression | 2.66E−07 |
| R10 | SARS-CoV-2 reporter expression | 1.62E−07 |
| R7 + R10 | SARS-CoV-2 reporter expression | 1.29E−07 |
| crRNA-NC | mRNA abundance | 8.31E−02 |
| R7 | mRNA abundance | 2.66E−17 |
| R10 | mRNA abundance | 3.13E−17 |
| R7 + R10 | mRNA abundance | 4.01E−18 |

FIG. 33H (Right) and FIG. 33I (Right)

| | | |
|---|---|---|
| crRNA-NC | SARS-CoV-2 reporter expression | 7.36E−01 |
| R14 | SARS-CoV-2 reporter expression | 1.00E−04 |
| N1 | SARS-CoV-2 reporter expression | 5.04E−05 |
| N4 | SARS-CoV-2 reporter expression | 1.21E−04 |
| R14 + N1 + N4 | SARS-CoV-2 reporter expression | 2.95E−05 |
| crRNA-NC | mRNA abundance | 3.60E−01 |
| R14 | mRNA abundance | 1.16E−14 |
| N1 | mRNA abundance | 1.74E−13 |
| N4 | mRNA abundance | 1.42E−11 |
| R14 + N1 + N4 | mRNA abundance | 6.46E−15 |

TABLE 10
Primers used in qRT-PCR. Related to FIG. 33

| | | SEQ ID NO: |
|---|---|---|
| SARS-COV-2-F1_F | aacgggtttgcggtgtaagt | 13731 |
| SARS-COV-2-F1_R | aatttagcaaaaccagctactttatcattgtag | 13732 |
| SARS-COV-2-F2_F | caggtggaacctcatcaggag | 13733 |
| SARS-COV-2-F2_R | gttaccatcagtagataaaagtgcattaacattg | 13734 |
| GAPDH_F | tgcaccaccaactgcttagc | 13735 |
| GAPDH R | ggcatggactgtggtcatgag | 13736 |

### Example 21. Optimized crRNAs Targeting SARS-CoV-2 Sequences Using COMBAT

Finally, a minipool of 5 highly efficient crRNAs (crRNA-MP5) targeting the SARS-CoV-2 genome was determined. Using SARS-CoV-2 reporters, it was confirmed that individual crRNAs may repress GFP up to 90% (FIG. 33H), with an 85% repression of RNA abundance (FIG. 33I). Pooling these crRNAs further enhanced repression up to 95% for GFP expression and 93% for RNA abundance. Sequences of 94,017 SARS-CoV-2 isolates from the Global Initiative on Sharing All Influenza Data (GISAID) database (as of 9/05/2020) were further retrieved. Each crRNA in crRNA-MP5 was able to target almost 99% of SARS-CoV-2 isolates, and the crRNA-MP5 together may target all known SARS-CoV-2 strains regardless of the variations in different SARS-CoV-2 clades as identified in Nextstrain (FIG. 33J). The optimized minipool of crRNA-MP5 provided a useful resource to be tested against live SARS-CoV-2 virus in vitro and in vivo in the future.

The rapid development of CRISPR-Cas13-based antivirals had raised the question of whether the system may be effectively used to broadly target many families of RNA viruses. Here, a comprehensive in silico analysis was performed of using CRISPR-Cas13 to target notable pathogenic viral species and 16 families of human- or animal-infectious RNA viruses in the ViPR database. The integrated crRNA analysis pipeline incorporated multiple existing algorithms that computed the coverage, efficiency, and specificity of crRNAs for RNA virus targeting. It was observed that 10 families of human-infectious viruses may be flexibly targeted by a minipool of 14 crRNAs (SEQ ID NOs: 13651-13664, Table 11) with >90% coverage. In some cases, the crRNAs can comprise nucleic acid sequences of SEQ ID NOs: 13671-13684 (Table 11). The strain number and diversity and segmented genomes was determined as potential determinant factors for the number of crRNAs required for coverage. It also observed that, similar to human-infectious viruses, PAC-MAN may also provide a broad-spectrum antiviral strategy for viruses that infected domestic livestock, thus expanding its potential applications.

TABLE 11
Exemplary crRNA sequences for targeting RNA virus

| | | SEQ ID | | SEQ ID |
|---|---|---|---|---|
| | 14 crRNAs Sequence | NO: | crRNA Target Region | NO: |
| Guide | ACAGCATTCTGTGAAT | 13651 | CTTATAATTCACAGAAT | 13671 |
| 1 | TATAAG | | GCTGT | |
| Guide | TCCCAGCGTCAATATG | 13652 | AAACAGCATATTGACGC | 13672 |
| 2 | CTGTTT | | TGGGA | |
| Guide | AAACACGGACACCCA | 13653 | ACTACTTTGGGTGTCCG | 13673 |
| 3 | AAGTAGT | | TGTTT | |
| Guide | ACGAGACCTCCCGGG | 13654 | CGAGTGCCCCGGGAGGT | 13674 |
| 4 | GCACTCG | | CTCGT | |
| Guide | ATGCCTATAACAAATG | 13655 | TCTGATCATTTGTTATA | 13675 |
| 5 | ATCAGA | | GGCAT | |
| Guide | GGGAGCCAGATTGCG | 13656 | GGGCGATCGCAATCTGG | 13676 |
| 6 | ATCGCCC | | CTCCC | |
| Guide | GAGAATCTCTTCGCCA | 13657 | CACAGTTGGCGAAGAG | 13677 |
| 7 | ACTGTG | | ATTCTC | |
| Guide | ACCTATTTAGGACCGC | 13658 | TGTACGGCGGTCCTAAA | 13678 |
| 8 | CGTACA | | TAGGT | |
| Guide | TTCCCATCATGTTGTA | 13659 | TGTGTGTACAACATGAT | 13679 |
| 9 | CACACA | | GGGAA | |
| Guide | TATAAAAAACTTAGGA | 13660 | CTTTGCTCCTAAGTTTTT | 13680 |
| 10 | GCAAAG | | TATA | |
| Guide | GATGAAGATTAAAAC | 13661 | GATGAAGGTTTTAATCT | 13681 |
| 11 | CTTCATC | | TCATC | |
| Guide | TCTCTCGCGTTTCAGC | 13662 | CAATATGCTGAAACGCG | 13682 |
| 12 | ATATTG | | AGAGA | |
| Guide | ATGTAACACCCCTACA | 13663 | ATCCATTGTAGGGGTGT | 13683 |
| 13 | ATGGAT | | TACAT | |
| Guide | GGGTTGGTTGGATGAA | 13664 | CCTATATTCATCCAACC | 13684 |
| 14 | TATAGG | | AACCC | |

### Example 22. Methods for Performing Experiments Described in Example 15-Example 22

A General Pipeline for crRNA Analysis

All RNA virus genomes were retrieved from public databases, including 16 RNA virus families from the Virus Pathogen Database and Analysis Resource (ViPR), human immunodeficiency virus (HIV) from NCBI, and influenza A or B viruses (IAV or IBV) from the Influenza Research Database (IRD) (Table 7).

The viral genomes were aligned by Multiple Alignment using Fast Fourier Transform (MAFFT) and a 22-nucleotide (nt) sliding window was passed over the alignment. In each window, if there was a 22-nt sequence that appears in >50% of genomes then that sequence was added to the crRNA set. In order to eliminate crRNAs with off-targets in the human genome, Bowtie was used to identify crRNAs that aligned to the human transcriptome with <=2 mismatches; these crRNAs as well as crRNAs with the poly-U (>=4U's) sequence that may prevent crRNA expression were removed from the guide set. All crRNAs were assigned a predicted efficiency score. Cas13 guide design method 1 was described in the paragraph below.

To assign each crRNA with a predicted efficiency score, a computational tool that generated Cas13 crRNAs and predicted efficiency scores given a single input sequence was used. First, for each viral group was chosen as a representative sequence, which was either a NCBI reference sequence if one existed in the viral group, otherwise a random sequence from the viral group. The Cas13 Guide Design Tool was used to generate crRNAs against this reference sequence. Because all crRNAs were 22 nt, in contrast to the longer crRNAs generated by the tool, each crRNA was assigned the average score of all output crRNAs of which it was a substring.

Species-specific crRNAs referred to those crRNAs that targeted conserved regions in a group of closely related viral genomes. The viruses in a group may be members of the same viral genus (e.g. betacoronaviruses), species (e.g. SARS-CoV-2 patient isolates), or may be members of the same viral family that shared a common host organism (e.g. all human-infecting coronaviruses).

The mini pool of family-covering crRNAs was determined through an iterative process. The crRNAs were ranked by the number of unique genomes they appear in and the crRNA that targeted most genomes was added to the list. The process was repeated, and the crRNAs were ranked by the number of unique genomes that were not already targeted. If multiple crRNAs were tied in a step, the crRNA with the fewest 3-mer repeats (e.g., “AAA” or “GGG”) was selected. The process ended when every virus strain in the family was targeted by at least one crRNA in the list. Note that the ordering of crRNAs in the list was significant; necessarily, the first crRNA in the list would target the largest number of virus strains.

### Format of Dataset in COMBAT Server

The downloadable file format of crRNAs is a CSV file, in which each crRNA was annotated with its genomic sequence, spacer DNA for synthesis, % GC content, and additional features. Family-covering crRNAs were annotated by the cumulative percentage of sequences in the virus family that may be targeted by this crRNA and preceding crRNAs in the list. The cumulative percentage of sequences targeted by the last crRNA in the list will be 100%. Users may click on coding sequences of interest to selectively download guides targeting those regions. A link to the iTOL website for each phylogenetic tree was included to enable users to interact with the tree.

### SARS-CoV-2 Targeting in COMBAT

A focused webpage was developed for users to access crRNAs targeting SARS-CoV-2 and the coronavirus family (FIG. 32A, FIG. 32B, and FIG. 32C; http://crispr-combat.stanford.edu/sarscov2). This webpage hosts: 1) species-specific crRNAs targeting conserved regions of SARS-CoV-2, 2) a coronavirus-covering crRNA minipool that collectively target all sequenced coronaviruses, and 3) custom crRNAs that can target both SARS-CoV-2 and another virus species, such as SARS-Cov or MERS-CoV. Because new sequences of SARS-CoV-2 patient isolates are frequently released, these crRNA sets are frequently updated. This page also highlights experimental validation data for SARS-CoV-2 crRNAs.

### Cell Culture

All cell lines were incubated at 37° C. and at 5% CO2. Human embryonic kidney (HEK293T) cells and A549 cells (ATCC CCL-185) were cultured in 10% fetal bovine serum (Thermo Fisher Scientific) in DMEM (Thermo Fisher Scientific).

Design and Cloning of RdRP- and N-Targeting crRNAs

The crRNA plasmids were cloned using standard restriction-ligation cloning. Forward and reverse oligos for each spacer (IDT) were annealed and inserted into the backbone using T4 DNA Ligase (NEB). Assembled oligos were either inserted into a pHR backbone, obtained and modified from Addgene (#121514 and #109054, respectively). Spacer sequences for all crRNAs can be found in Table 8.

### Design and Cloning of SARS-CoV-2 Reporters

To clone SARS-CoV-2 reporters, pHR-PGK-scFv GCN4-sfGFP (pSLQ1711) was digested with EcoR1 and Sbf1 and gel purified. The PGK promoter and sfGFP were PCR amplified from pSLQ1711 and gel purified. The SARS-CoV-2-F1 and SARS-CoV-2-F2 fragments were synthesized from Integrated DNA Technologies (IDT). At last, the PGK, sfGFP, and SARS-CoV-2-F1/F2 fragments were inserted into the linearized pSLQ1711 vector using In-Fusion cloning (Takara Bio).

### Lentiviral Packaging and Stable Cell Line Generation

On day 1, HEK293T cells were seeded into 100 mm dishes (Corning). On day 2, cells were ˜50-70% confluent at the time of transfection. For each dish, 6 μg of pHR vector containing the construct of interest, 4 μg of dR8.91 and 1 μg of pMD2.G (Addgene) were mixed in 1 mL of Opti-MEM reduced serum media (Gibco) with 30 μL of Mirus TransIT-LT1 reagent and incubated at room temperature for 30 minutes. The transfection complex solution was distributed evenly to HEK293T cultures dropwise. On day 5, lentiviruses are harvested from the supernatant with a sterile syringe and filtered through a 0.22-μm polyvinylidene fluoride filter (Millipore).

For A549 cells, lentivirus precipitation solution (Alstem) was added and precipitated as per the manufacturer's protocol. One well of a 6-well plate (Corning) of A549 cells at ˜50% confluency was transduced with the precipitated lentivirus. After 2-3 days of growth, the cell supernatant containing the virus was removed and the cells were expanded. Cells were then sorted for mCherry+ cells using a Sony SH800S cell sorter.

### SARS-CoV-2 Reporter Challenge Experiments

On day 1, A549 cells stably expressing Cas13d (hereafter referred to as Cas13d A549) were seeded at a density of 80,000 cells per well in a 12-well plate. On day 2, Cas13d A549 cells were transfected with crRNAs targeting the two SARS-CoV-2 reporters or a non-targeting crRNA along with the respective SARS-CoV-2 reporters in an equimolar ratio using Mirus LT1 reagent (100 μL Opti-MEM reduced serum media. 0.5 μg crRNA, 1 μg SARS-CoV-2 reporter, 4 μL Mirus LT1 per well). Wildtype cells were transfected with either the crRNA or SARS-CoV-2 reporter plasmids as compensation controls. GFP expression was measured on day 4 by flow cytometry on a Beckman-Coulter CytoFLEX S instrument. Data was analyzed using FlowJo v10. All p values are available in Table 9.

Quantitative RT-PCR (qRT-PCR)

Real-time qRT-PCR was performed to quantify RNA abundance. For each sample, total RNA was isolated by using the RNeasy Plus Mini Kit (Qiagen Cat #74134), followed by cDNA synthesis using the iScript cDNA Synthesis Kit (BioRad. Cat #1708890). qRT-PCR primers were ordered from Integrated DNA Technologies (IDT). Quantitative PCR was performed using the PrimePCR assay with the SYBR Green Master Mix (BioRad) and run on a Biorad CFX384 real-time system (C1000 Touch Thermal Cycler), according to manufacturers' instructions. Cq values were used to quantify RNA abundance. The relative abundance of the SARS-CoV-2 fragments was normalized to a GAPDH internal control. Primers used in qPCR are available in Table 10.

### Example 23. Targeting and Cleaving Viral Genome or Viral Transcript with Different Heterologous Endonuclease

Described herein are systems and methods for targeting and cleaving viral genomes or transcripts of viral genomes. In some cases, the viral genomes or viral transcripts are associated with any one of the strain of virus described herein. In some embodiments, the systems and methods can target and cleave a plurality of viral genomes or viral transcripts of plurality of strains of virus. In some embodiments, the systems and methods described herein utilize heterologous RNA polynucleotide to guide the heterologous endonuclease to the target viral genomes or viral transcripts. In some embodiments, the systems and methods described herein utilize heterologous endonuclease that targets and cleaves the viral genomes or transcripts of viral genomes independent of the heterologous RNA polynucleotide. In some embodiments, the systems and methods described herein utilize a plurality of different heterologous endonucleases (e.g., TALENs in conjunction with CRISPR/Cas). In some embodiments, the plurality of different heterologous endonuclease can be designed to target different viral genes of a virus (e.g., coronavirus) or different genes of different viruses (e.g., Coronaviridae or Pneumoviridae) and regulate (e.g., reduce) viral infection in a cell.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A system for reducing viral infection in a cell, comprising:
(1) a heterologous polypeptide comprising a gene regulating moiety; or
(2) one or more heterologous nucleic acid molecules,
wherein at least the gene regulating moiety or the one or more heterologous nucleic acid molecules are configured to specifically bind one or more target viral genes of a coronavirus in the cell, to reduce expression or activity of the one or more target viral genes of the coronavirus in the cell, and
wherein the one or more target viral genes encode (i) a viral RNA-dependent RNA polymerase (RdRP) or (ii) a viral nucleocapsid protein (N-protein).

2. The system of claim 1, wherein the one or more heterologous nucleic acid molecules comprises a plurality of different heterologous nucleic acid molecules that is collectively configured to bind (i) different portions of a same target viral gene of the coronavirus or (ii) different target viral genes of the coronavirus.

3. The system of claim 2, wherein the plurality of different heterologous nucleic acid molecules comprises at least 3, 4, 5, or 6 different heterologous nucleic acid molecules.

4. The system of any one of the preceding claims, wherein a heterologous nucleic acid molecule of the one or more heterologous nucleic acid molecules exhibits at most 3, 2, or 1 mismatch(es) when optimally aligned with a MERS-CoV genome or a SARS-CoV genome.

5. The system of any one of the preceding claims, wherein a heterologous nucleic acid molecule of the one or more heterologous nucleic acid molecules has at least about 80%, 85%, 90%, 95%, or 99% sequence identity or complementarity to a sequence selected from the group consisting of SEQ ID NOs: 4001-7203, 12101-12140, and 13101-13122.

6. The system of any one of the preceding claims, wherein the one or more target viral genes comprise a plurality of target viral genes encoding (i) the viral RdRP and (ii) the N-protein.

7. The system of any one of the preceding claims, wherein the system is sufficient to reduce expression or activity level of the RdRP in the cell by at least about 70% or 75%, as compared to a corresponding control.

8. The system of any one of the preceding claims, wherein the system is sufficient to reduce expression or activity level of the N-protein in the cell by at least about 70%, 75%, 80%, or 85%, as compared to a corresponding control.

9. The system of any one of the preceding claims, wherein the system is sufficient to reduce infection of at least about 30%, 35%, 40%, 50%, 60%, 70%, 80%, or 85% of a plurality of different strains of the coronavirus, as compared to a corresponding control.

10. The system of claim 9, wherein the plurality of different strains of the coronavirus comprises at least one strain from a member selected from the group consisting of alphacoronavirus, betacoronavirus, deltacoronavirus, and gammacoronavirus.

11. The system of claim 9, wherein the plurality of different strains of the coronavirus comprises at least one strain from each of two, three, or four members selected from the group consisting of alphacoronavirus, betacoronavirus, deltacoronavirus, and gammacoronavirus.

12. The system of any one of claims 9-11, wherein the plurality of different strains of the coronavirus comprises at least about 100, 500, 1000, 1500, 2000, or 2500 different strains.

13. The system of claim 1, wherein at least the gene regulating moiety is configured to specifically bind the one or more target viral genes of the coronavirus in the cell.

14. The system of claim 1, wherein at least the one or more heterologous nucleic acid molecules are configured to specifically bind the one or more target viral genes of the coronavirus in the cell.

15. The system of claim 1, wherein the gene regulating moiety and the one or more heterologous nucleic acid molecules are collectively configured to specifically bind the one or more target viral genes of the coronavirus in the cell.

16. The system of claim 15, wherein the gene regulating moiety is configured to form a complex with the one or more heterologous nucleic acid molecules, the complex being configured to bind the one or more target viral genes.

17. A system for reducing viral infection in a cell, comprising:
(1) a heterologous polypeptide comprising a gene regulating moiety; or
(2) one or more heterologous nucleic acid molecules,
wherein at least the gene regulating moiety or the one or more heterologous nucleic acid molecules are configured to specifically bind one or more non-coding regions of at least one target viral gene in the cell, to reduce expression or activity of the at least one target viral gene in the cell.

18. The system of claim 17, wherein the one or more heterologous nucleic acid molecules comprises a plurality of different heterologous nucleic acid molecules that is collectively configured to bind a plurality of different non-coding regions of the at least one target viral gene.

19. The system of claim 18, wherein the plurality of different non-coding regions is operatively coupled to a same target viral gene.

20. The system of claim 19, wherein a viral sequence encoding a viral protein is flanked by the plurality of different non-coding regions.

21. The system of claim 19 or 20, wherein the same target viral gene encodes a neuraminidase.

22. The system of claim 19 or 20, wherein the same target viral gene encodes a hemagglutinin.

23. The system of claim 18, wherein the plurality of different non-coding regions is operatively coupled to different target viral genes.

24. The system of claim 23, wherein (i) a first target viral gene of the different target viral genes encodes a neuraminidase and (ii) a second target viral gene of the different target viral genes encodes a viral protein selected from the group consisting of a polymerase, a hemagglutinin, a nucleoprotein, a matrix protein, a non-structural protein, and a nuclear export protein.

25. The system of claim 23, wherein (i) a first target viral gene of the different target viral genes encodes a hemagglutinin and (ii) a second target viral gene of the different target viral genes encodes a viral protein selected from the group consisting of a polymerase, a nucleoprotein, a matrix protein, a non-structural protein, and a nuclear export protein.

26. The system of any one of claims 18-25, wherein the plurality of different heterologous nucleic acid molecules comprises at least 3, 4, 5, or 6 different heterologous nucleic acid molecules.

27. The system of any one of claims 17-26, wherein at least one target viral gene is from an influenza virus.

28. The system of any one of claims 17-27, wherein one or more non-coding regions are conserved across a plurality of different influenza virus strains.

29. The system of claim 28, wherein the plurality of different influenza virus strains comprises at least one strain from a member selected from the group consisting of influenza A, influenza B, influenza C, and influenza D.

30. The system of claim 29, wherein the plurality of different influenza virus strains comprises subtypes of influenza A.

31. The system of claim 29, wherein the plurality of different influenza virus strains comprises at least one strain from each of two, three, or four members selected from the group consisting of influenza A, influenza B, influenza C, and influenza D.

32. The system of any one of claims 28-31, wherein the plurality of different influenza virus strains comprises at least about 10, 20, 50, 100, or 120 different influenza virus strains.

33. The system of any one of claims 17-32, wherein the one or more non-coding regions comprise a sequence having at least about 80%, 85%, 90%, 95%, or 99% sequence identity or complementarity to a sequence selected from the group consisting of SEQ ID NOs: 13501-13548.

34. The system of any one of claims 17-33, wherein the system is sufficient to reduce infection of an influenza virus in the cell by at least about 50%, 55%, 60%, 65%, or 70%, as compared to a corresponding control.

35. The system of claim 17, wherein at least the gene regulating moiety is configured to specifically bind the one or more non-coding regions of the at least one target viral gene.

36. The system of claim 17, wherein at least the one or more heterologous nucleic acid molecules are configured to specifically bind the one or more non-coding regions of the at least one target viral gene.

37. The system of claim 17, wherein the gene regulating moiety and the one or more heterologous nucleic acid molecules are collectively configured to specifically bind the one or more non-coding regions of the at least one target viral gene.

38. The system of claim 37, wherein the gene regulating moiety is configured to form a complex with the one or more heterologous nucleic acid molecules, the complex being configured to bind the one or more non-coding regions of the at least one target viral gene.

39. A system for reducing viral infection in a cell, comprising:
(1) a plurality of heterologous gene regulating moieties that comprises no more than 20 members: or
(2) a plurality of heterologous nucleic acid molecules that comprises no more than 20 members:
wherein the plurality of heterologous gene regulating moieties or the plurality of heterologous nucleic acid molecules is configured to specifically bind at least about 70% of a plurality of strains from a plurality of RNA virus families.

40. The system of claim 39, wherein the plurality of strains comprises at least about 1000, 5000, 10000, 15000, 20000, or 22000 different strains.

41. The system of claim 39 or 40, wherein the specific binding is to at least about 75%, 80%, 85%, or 90% of the plurality of strains from the plurality of RNA virus families.

42. The system of any one of claims 39-41, wherein the plurality of RNA virus families comprises two or members selected from the group consisting of Coronaviridae, Pneumoviridae, Paramyxoviridae, Caliciviridae, Hepeviridae, Picomaviridae, Filoviridae, Flaviviridae, Rhabdoviridae, Togaviridae, Orthomyxoviridae, and Retroviridae.

43. The system of any one of claims 39-42, wherein the plurality of RNA virus families comprises two or more members selected from the group consisting of Coronaviridae, Pneumoviridae, Paramyxoviridae, Caliciviridae, Hepeviridae, Picomaviridae, Filoviridae, Flaviviridae, Rhabdoviridae, and Togaviridae.

44. The system of any one of claims 39-43, wherein at least the plurality of heterologous gene regulating moieties is configured to specifically bind at least about 700% of the plurality of strains.

45. The system of any one of claims 39-43, wherein at least the plurality of heterologous nucleic acid molecules is configured to specifically bind at least about 70% of the plurality of strains.

46. The system of any one of claims 39-43, wherein at least the plurality of heterologous gene regulating moieties and the plurality of heterologous nucleic acid molecules are collectively configured to specifically bind at least about 70% of the plurality of strains.

47. The system of claim 46, wherein (i) a gene regulating moiety of the plurality of heterologous gene regulating moieties is configured to form a complex with (ii) a heterologous nucleic acid molecule of the plurality of plurality of heterologous nucleic acid molecules, the complex being configured to bind a target viral gene from the plurality of strains.

48. A system for reducing viral infection in a cell, comprising:
(1) a plurality of heterologous gene regulating moieties that comprises no more than 20 members; or
(2) a plurality of heterologous nucleic acid molecules that comprises no more than 20 members;
wherein the plurality of heterologous gene regulating moieties or the plurality of heterologous nucleic acid molecules is configured to specifically bind at least about 30% of a plurality of strains from each RNA virus family of a plurality of RNA virus families.

49. The system of claim 48, wherein the plurality of strains from said each RNA virus family comprises at least about 100, 200, 300, 400, 500, 1,000, 2,000, or more different strains.

50. The system of claim 48 or 49, wherein the specific binding is to at least about 35%, 40%, 45%, or 50% of the plurality of strains from said each RNA virus family.

51. The system of any one of claims 48-50, wherein the specific binding is to at least about 55%, 60%, 65%, 70%, 75%, or 80% of the plurality of strains of one or more RNA virus families selected from the group consisting of Coronaviridae, Pneumoviridae, Caliciviridae, Hepeviridae, Picomaviridae, Filoviridae, Flaviviridae, and Togaviridae.

52. The system of any one of claims 48-51, wherein the specific binding is to at least about 85% of the plurality of strains of one or more RNA virus families selected from the group consisting of Coronaviridae, Pneumoviridae, Hepeviridae, Picomaviridae, Filoviridae, Flaviviridae, and Togaviridae.

53. The system of any one of claims 48-52, wherein at least the plurality of heterologous gene regulating moieties is configured to specifically bind at least about 30% of the plurality of strains.

54. The system of any one of claims 48-52, wherein at least the plurality of heterologous nucleic acid molecules is configured to specifically bind at least about 30% of the plurality of strains.

55. The system of any one of claims 48-52, wherein at least the plurality of heterologous gene regulating moieties and the plurality of heterologous nucleic acid molecules are collectively configured to specifically bind at least about 30% of the plurality of strains.

56. The system of claim 55, wherein (i) a gene regulating moiety of the plurality of heterologous gene regulating moieties is configured to form a complex with (ii) a heterologous nucleic acid molecule of the plurality of plurality of heterologous nucleic acid molecules, the complex being configured to bind a target viral gene from the plurality of strains.

57. The system of any one of claims 39-56, wherein the plurality of RNA virus families is a plurality of human-infectious RNA virus families.

58. The system of claim 57, wherein the plurality of heterologous gene regulating moieties comprises between about 5 and about 20 different members, between about 8 and about 20 different members, between about 10 and about 18 different members, between about 12 and about 16 different members, or between about 13 and about 15 different members.

59. The system of claim 57, wherein the plurality of heterologous gene regulating moieties comprises about 14 different members.

60. The system of any one of claims 57-59, wherein a heterologous gene regulating moiety of the plurality of gene regulating moieties exhibits a specific binding to a target polynucleotide sequence, wherein the target polynucleotide sequence has at least about 50%, 60%, 70%, 80%, 85%, 90%, 95%, or 99% sequence identity or complementarity to a sequence selected from Table 11.

61. The system of any one of claims 39-56, wherein the plurality of heterologous nucleic acid molecules comprises between about 5 and about 20 different members, between about 8 and about 20 different members, between about 10 and about 18 different members, between about 12 and about 16 different members, or between about 13 and about 15 different members.

62. The system of claim 61, wherein the plurality of heterologous nucleic acid molecules comprises about 14 different members.

63. The system of claim 61 or 62, wherein a heterologous nucleic acid molecule of the plurality of heterologous nucleic acid molecules has at least about 50%, 60%, 70%, 80%, 85%, 90%, 95%, or 99% sequence identity or complementarity to a sequence selected from Table 11.

64. The system of any one of the preceding claims, wherein the heterologous nucleic acid molecule(s) has a length ranging from about 10 to about 30 nucleotides.

65. The system of any one of the preceding claims, wherein the heterologous nucleic acid molecule(s) is a heterologous guide RNA molecule.

66. The system of any one of the preceding claims, wherein the gene regulating moiety is a heterologous endonuclease.

67. The system of claim 66, wherein the heterologous endonuclease is an RNA-targeting endonuclease.

68. The system of any one of claims 66-67, wherein the heterologous endonuclease exhibits RNA cleavage activity.

69. The system of any one of claims 66-68, wherein the heterologous endonuclease comprises a CRISPR/Cas protein or a modification thereof.

70. The system of claim 69, wherein the CRISPR/Cas protein is Cas13.

71. The system of claim 70, wherein the CRISPR/Cas protein is Cas13d.

72. The system of any one of the preceding claims, wherein (i) the heterologous polypeptide or a gene encoding thereof or (ii) the heterologous nucleic acid molecule(s) or a gene encoding thereof is packaged in a delivery vehicle selected from the group consisting of a liposome, a nanoparticle, and a viral capsule.

73. A composition comprising (i) the system of any one of the preceding claims or (ii) one or more polynucleotide sequences encoding thereof.

74. A kit comprising the composition of claim 73.

75. A reaction mixture comprising (i) the system of any one of the preceding claims and (ii) a biological sample.

76. The reaction mixture of claim 75, wherein the biological sample is selected from the group consisting of urine, blood, cell, tissue, organ, organism, and a combination thereof.

77. A method of reducing viral infection in a population of cells, the method comprising contacting the population of cells with the system of any one of the preceding claims, wherein upon the contacting, viral infection in the population of cells is reduced.

78. The method of claim 77, wherein the contacting occurs in vivo, ex vivo, or in vitro.

79. The method of claim 77, further comprising expressing, in the population of cells, (i) the system or (ii) one or more polynucleotide sequences encoding thereof.

80. The method of claim 79, further comprising administering (i) the system or (ii) the one or more polynucleotide sequences to a subject in need thereof.

81. The method of claim 77, further comprising, subsequent to the contacting, administering one or more cells of the population of cells to a subject in need thereof.

82. A system for reducing viral infection in a cell, comprising:
(1) a first therapeutic agent configured to specifically bind a target viral gene, the first therapeutic agent comprising:
(i) a heterologous polypeptide comprising a gene regulating moiety; or
(ii) a heterologous nucleic acid molecule; and
(2) a second therapeutic agent that does not specifically bind a viral gene,
wherein a combination of the first and second therapeutic agents reduces viral infection in the cell.

83. The system of claim 82, wherein the combination effects enhanced reduction in the viral infection in the cell by at least about 10%, 20%, 30%, 40%, or 45% as compared to a corresponding control.

84. The system of claim 83, wherein the corresponding control comprises a cell lacking the first therapeutic agent or the second therapeutic agent.

85. The system of claim 83, wherein the corresponding control comprises a cell lacking the second therapeutic agent.

86. The system of any one of claims 82-85, wherein the (i) the first therapeutic agent or a gene encoding thereof and (ii) the second therapeutic agent are in a same composition or in different compositions.

87. The system of any one of claims 82-86, wherein a synergistic effect of the combination on the reduction of the viral replication in the cell is greater than an additive effect of the combination when performed separately.

88. The system of claim 87, wherein a fold decrease in the viral replication from the synergistic effect is greater than that from the additive effect by at least about 1-fold, 2-fold, 3-fold, 4-fold, 6-fold, 8-fold, or 10-fold.

89. The system of any one of claims 82-88, wherein the second therapeutic agent is an immune regulator.

90. The system of claim 89, wherein the immune regulator is a cytokine.

91. The system of claim 90, wherein the cytokine is interferon-gamma.

92. The system of any one of claims 82-88, wherein the second therapeutic agent is an antiviral agent.

93. The system of claim 92, wherein the antiviral agent is a small molecule.

94. The system of claim 92, wherein the antiviral agent is Camostat.

95. The system of any one of claims 82-88, wherein the second therapeutic agent is configured to specifically bind an target endogenous gene of the cell, the target endogenous gene encoding a protein involved in viral infection or replication.

96. The system of claim 95, wherein the protein is a transmembrane protease.

97. The system of claim 96, wherein the transmembrane protease is TMPRSS2.

98. The system of claim 95, wherein the protein is a MTHFD1.

99. The system of claim 95, wherein the protein is a peptidase.

100. The system of claim 95, wherein the protein is an aminopeptidase.

101. The system of claim 95, wherein the protein is ANPEP.

102. The system of any one of claims 95-101, wherein the second therapeutic agent comprises:
(i) an additional heterologous polypeptide comprising an additional gene regulating moiety; or
(ii) an additional heterologous nucleic acid molecule.

103. The system of claim 102, wherein the additional gene regulating moiety is configured to specifically bind the target endogenous gene.

104. The system of claim 102, wherein the additional heterologous nucleic acid molecule is configured to specifically bind the target endogenous gene.

105. The system of claim 102, wherein the additional gene regulating moiety is configured to form a complex with the additional heterologous nucleic acid molecule, the complex being configured to bind the target endogenous gene.

106. The system of claim 105, wherein the additional heterologous nucleic acid molecule is a guide nucleic acid molecule.

107. The system of any one of claims 82-106, wherein the gene regulating moiety is configured to specifically bind the target viral gene.

108. The system of any one of claims 82-106, wherein the heterologous nucleic acid molecule is configured to specifically bind the target viral gene.

109. The system of any one of claims 82-106, wherein the gene regulating moiety is configured to form a complex with the heterologous nucleic acid molecule, the complex being configured to bind the target viral gene.

110. The system of claim 109, wherein the heterologous nucleic acid molecule is a guide nucleic acid molecule.

111. The system of any one of claims 82-110, wherein the guide nucleic acid molecule has a length ranging from about 10 to about 30 nucleotides.

112. The system of any one of claims 82-111, wherein the gene regulating moiety is a heterologous endonuclease.

113. The system of claim 112, wherein the heterologous endonuclease is an RNA-targeting endonuclease.

114. The system of claim 112 or 113, wherein the heterologous endonuclease exhibits RNA cleavage activity.

115. The system of any one of claims 112-114, wherein the heterologous endonuclease comprises a CRISPR/Cas protein or a modification thereof.

116. The system of claim 115, wherein the CRISPR/Cas protein is Cas13.

117. The system of claim 115, wherein the CRISPR/Cas protein is Cas13d.

118. A composition comprising the system of any one of claims 82-117.

119. A kit comprising the composition of claim 118.

120. A reaction mixture comprising (i) the system of any one of claims 82-117 and (ii) a biological sample.

121. The reaction mixture of claim 120, wherein the biological sample is selected from the group consisting of urine, blood, cell, tissue, organ, organism, and a combination thereof.

122. A method for reducing viral infection in a cell, comprising:
(a) contacting the cell with a first therapeutic agent of the system of any one of claims 82-117, the first therapeutic agent comprising:
(i) a heterologous polypeptide comprising a gene regulating moiety; or
(ii) a heterologous nucleic acid molecule; and
(b) contacting the cell with a second therapeutic agent of the system of any one of claims 82-117, wherein a second therapeutic agent that does not specifically bind a viral gene, and wherein contacting the cell with the first and second therapeutic agents reduces viral infection in the cell.

123. The method of claim 122, wherein (a) is performed prior to, simultaneously with, or subsequent to (b).

124. The method of claim 122, wherein the contacting in (a) and (b) occurs in vivo, ex vivo, or in vitro.

125. The method of claim 122, further comprising administering (i) the system or (ii) one or more polynucleotide sequences encoding the system to a subject in need thereof, wherein the subject comprises the cell.

126. The method of claim 122, further comprising, subsequent to (b), administering the cell to a subject in need thereof.

127. The method of claim 130, wherein the first therapeutic agent and the second therapeutic agent are in a same composition or separate compositions.

128. A system for reducing viral infection in a cell, comprising:
(1) a heterologous polypeptide comprising a gene regulating moiety that is operatively coupled to a nuclear localization signal (NLS) (gene regulating moiety-NLS); and
(2) a heterologous polynucleotide sequence encoding a guide nucleic acid molecule,
wherein, upon expression of the guide nucleic acid molecule from the heterologous polynucleotide sequence in the cell, the gene regulating moiety-NLS forms a complex with the guide nucleic acid molecule, the complex being configured to bind a target viral gene to reduce the viral infection in the cell.

129. The system of claim 128, wherein the heterologous polynucleotide sequence is not integrated into a genome of the cell.

130. The system of claim 128 or 129, wherein the binding effects reduction of the viral infection in the cell by at least about 10%, 20%, 30%, 40%, or 50%, as compared to a corresponding control.

131. The system of claim 130, wherein the corresponding control comprises a cell that comprises (i) a gene regulating moiety in absence of the NLS and (ii) the heterologous polynucleotide sequence encoding the guide nucleic acid molecule.

132. The system of claim 131, wherein, in the corresponding control, the gene regulating moiety is operatively coupled to a nuclear export signal (NES).

133. The system of any one of claims 128-132, the viral infection is viral replication in the cell.

134. The system of any one of claims 128-133, wherein the guide nucleic acid molecule is a guide RNA molecule.

135. The system of any one of claims 128-134, wherein the guide nucleic acid molecule has a length ranging from about 10 to about 30 nucleotides.

136. The system of any one of claims 128-135, wherein the gene regulating moiety is a heterologous endonuclease.

137. The system of claim 136, wherein the heterologous endonuclease is an RNA-targeting endonuclease.

138. The system of claim 136 or 137, wherein the heterologous endonuclease exhibits RNA cleavage activity.

139. The system of any one of claims 136-138, wherein the heterologous endonuclease comprises a CRISPR/Cas protein or a modification thereof.

140. The system of claim 139, wherein the CRISPR/Cas protein is Cas13.

141. The system of claim 139, wherein the CRISPR/Cas protein is Cas13d.

142. The system of any one of claims 128-141, wherein heterologous polynucleotide sequence is part of a non-replicating viral vector.

143. A composition comprising the system of any one of claims 128-142.

144. A kit comprising the composition of claim 143.

145. A reaction mixture comprising (i) the system of any one of claims 128-142 and (ii) a biological sample.

146. The reaction mixture of claim 145, wherein the biological sample is selected from the group consisting of urine, blood, cell, tissue, organ, organism, and a combination thereof.

147. A method for reducing viral infection in a cell, comprising:
(a) contacting the cell with the system of any one of claims 128-142, the system comprising:
(1) a heterologous polypeptide comprising a gene regulating moiety that is operatively coupled to a nuclear localization signal (NLS) (gene regulating moiety-NLS); and
(2) a heterologous polynucleotide sequence encoding a guide nucleic acid molecule; and
(b) expressing the guide nucleic acid molecule in the cell from the heterologous polynucleotide sequence,
wherein, upon the expressing, the gene regulating moiety-NLS forms a complex with the guide nucleic acid molecule, and the complex binds a target viral gene to reduce the viral infection in the cell.

148. The method of claim 147, wherein the contacting occurs in vivo, ex vivo, or in vitro.

149. The method of claim 147, further comprising administering (i) the system or (ii) one or more polynucleotide sequences encoding the system to a subject in need thereof, wherein the subject comprises the cell.

150. The method of claim 147, further comprising, subsequent to (a) and (b), administering the cell to a subject in need thereof.

151. A nucleic acid composition comprising a RNA-binding guide RNA, wherein the RNA-binding guide RNA comprises a targeting sequence that has a length ranging between about 10 to about 30 nucleotides and is characterized by one or more members selected from the group consisting of:
(i) the targeting sequence exhibits at least about 80% sequence identity or complementarity to any one of SEQ ID NOs: 4001-7203, 12101-12140, and 13101-13122:
(ii) the targeting sequence is configured to bind a polynucleotide fragment within a coronavirus genome, wherein the polynucleotide fragment has a length of between about 10 and about 30 nucleotides and exhibits at least about 80% sequence identity or complementarity to a conserved region of the coronavirus genome that encodes (1) a RNA-dependent RNA polymerase (RdRP) (2) a nucleocapsid protein (N-protein); and
(iii) the targeting sequence comprises at most 3 mismatches when optimally aligned with a MERS-CoV genome or a SARS-CoV genome.

152. The nucleic acid composition of claim 151, wherein the targeting sequence exhibits at least about 80%, 85%, 90%, 95%, or 99% sequence identity or complementarity to any one of SEQ ID NOs: 4001-7203, 12101-12140, and 13101-13122.

153. The nucleic acid composition of claim 152, wherein the targeting sequence exhibits at least about 80%, 85%, 90%, 95%, or 99% sequence identity or complementarity to any one of SEQ ID NOs: 4001-7203.

154. The nucleic acid composition of claim 152, wherein the targeting sequence exhibits at least about 80%, 85%, 90%, 95%, or 99% sequence identity or complementarity to any one of SEQ ID NOs: 12101-12140.

155. The nucleic acid composition of claim 152, wherein the targeting sequence exhibits at least about 80%, 85%, 90%, 95%, or 99% sequence identity or complementarity to any one of SEQ ID NOs: 13101-13122.

156. The nucleic acid composition of claim 155, wherein the targeting sequence exhibits at least about 80%, 85%, 90%, 95%, or 99% sequence identity or complementarity to any one of SEQ ID NO: 13101-13106.

157. The nucleic acid composition of claim 151, wherein targeting sequence is configured to bind the polynucleotide fragment within the coronavirus genome, wherein the polynucleotide fragment has a length of between about 10 and about 30 nucleotides and exhibits at least about 80%, 85%, 90%, 95%, or 99% sequence identity or complementarity to the conserved region of the coronavirus genome that encodes (1) the RdRP or (2) the N-protein.

158. The nucleic acid composition of claim 151, wherein the targeting sequence comprises at most 3, 2, or 1 mismatch(es) when optimally aligned with the MERS-CoV genome or the SARS-CoV genome.

159. The nucleic acid composition of any one of the preceding claims, wherein a mismatch is an insertion or a deletion.

160. The nucleic acid composition of any one of the preceding claims, wherein the nucleic acid composition is characterized by two or more members selected from the group consisting of (i), (ii), and (iii).

161. The nucleic acid composition of any one of the preceding claims, wherein the nucleic acid composition is characterized by all of (i), (ii), and (iii).

162. The nucleic acid composition of any one of the preceding claims, comprising a plurality of different RNA-binding guide RNAs comprising the RNA-binding guide RNA, wherein each of the plurality of different RNA-binding guide RNAs is characterized by one or more members selected from the group consisting of (i), (ii), and (iii).

163. The nucleic acid composition of any one of the preceding claims, wherein each of the plurality of different RNA-binding guide RNAs is characterized by two or more members selected from the group consisting of (i), (ii), and (iii).

164. The nucleic acid composition of any one of the preceding claims, wherein each of the plurality of different RNA-binding guide RNAs is characterized by all of (i), (ii), and (iii).

165. The nucleic acid composition of any one of the preceding claims, wherein the plurality of different RNA-binding guide RNAs comprises at least 3, 4, 5, or 6 different RNA-binding guide RNAs.

166. A nucleic acid composition comprising a plurality of different RNA-binding guide RNAs, wherein no more than six members of the plurality collectively exhibits specific binding to at least about 50% of a plurality of different strains of coronaviruses selected from the group consisting of alphacoronavirus, betacoronavirus, deltacoronavirus, and gammacoronavirus.

167. A nucleic acid composition comprising a plurality of different RNA-binding guide RNAs, wherein no more than six members of the plurality collectively exhibits specific binding to at least about 50% of a plurality of different strains of coronaviruses selected from the group consisting of coronaviruses as identified by GenBank Access ID.

168. The nucleic acid composition of any one of claims 166-167, wherein the specific binding is to at least about 50%, 60%, 70%, 80%, 85%, 90%, 95%, or 99% of the plurality of different strains.

169. The nucleic acid composition of any one of claims 166-168, wherein the plurality of different strains of the coronavirus comprises at least about 100, 500, 1000, 1500, 2000, or 2500 different strains.

170. The nucleic acid composition of any one of claims 166-169, wherein the plurality of different strains of the coronavirus comprises at least one strain from each of two, three, or four members selected from the group consisting of alphacoronavirus, betacoronavirus, deltacoronavirus, and gammacoronavirus.

171. The nucleic acid composition of any one of claims 166-170, wherein the plurality of different RNA-binding guide RNAs is selected from the group consisting of the sequences in Table 4.

172. A nucleic acid composition comprising one or more RNA-binding guide RNAs, wherein a RNA-binding guide RNA of the one or more RNA-binding guide RNAs comprises a targeting sequence that has a length ranging between about 10 to about 30, and wherein the RNA-binding guide RNA has at least about 80% sequence identity or complementarity to any one of SEQ ID NOs: 13301-13348.

173. The nucleic acid composition of claim 172, wherein the RNA-binding guide RNA has at least about 85%, 90%, 95%, or 99% sequence identity or complementarity to any one of SEQ ID NOs: 13301-13348.

174. The nucleic acid composition of claim 172, wherein the RNA-binding guide RNA has at least about 80%, 85%, 90%, 95%, or 99% sequence identity or complementarity to any one of SEQ ID NOs: 13319-13324 and 13331-13336.

175. The nucleic acid composition of any one of claims 172-174, wherein the one or more RNA-binding guide RNAs comprise a plurality of different RNA-binding guide RNAs.

176. The nucleic acid composition of claim 175, wherein the plurality of different RNA-binding guide RNAs comprises between about two and about eight different RNA-binding guide RNAs.

177. The nucleic acid composition of claim 175, wherein the plurality of different RNA-binding guide RNAs comprises between about four and about eight different RNA-binding guide RNAs.

178. The nucleic acid composition of claim 175, wherein the plurality of different RNA-binding guide RNAs comprises between about five and about seven different RNA-binding guide RNAs.

179. The nucleic acid composition of claim 175, wherein the plurality of different RNA-binding guide RNAs comprises about six different RNA-binding guide RNAs.

180. The nucleic acid composition of any one of claims 175-179, wherein the plurality of different RNA-binding guide RNAs comprises (i) a first RNA-binding guide RNA having at least about 80%, 85%, 90%, 95%, or 99% sequence identity or complementarity to any one of SEQ ID NOs: 13319-13324 or (ii) a second RNA-binding guide RNA having at least about 80%, 85%, 90%, 95%, or 99% sequence identity or complementarity to any one of SEQ ID NOs: 13331-13336.

181. The nucleic acid composition of claim 180, wherein the plurality of different RNA-binding guide RNAs comprises the first RNA-binding guide RNA and the second RNA-binding guide RNA.

182. A nucleic acid composition comprising one or more RNA-binding guide RNAs, wherein a RNA-binding guide RNA of the one or more RNA-binding guide RNAs comprises a targeting sequence that has a length ranging between about 10 to about 30, and wherein the RNA-binding guide RNA has at least about 80% sequence identity or complementarity to a sequence selected from Table 11.

183. The nucleic acid composition of claim 182, wherein the RNA-binding guide RNA has at least about 85%, 90%, 95%, or 99% sequence identity or complementarity to a sequence selected from Table 11.

184. The nucleic acid composition of any one of claims 182-183, wherein the one or more RNA-binding guide RNAs comprise a plurality of different RNA-binding guide RNAs.

185. The nucleic acid composition of claim 184, wherein the plurality of different RNA-binding guide RNAs comprises between about 5 and about 20 different RNA-binding guide RNAs.

186. The nucleic acid composition of claim 184, wherein the plurality of different RNA-binding guide RNAs comprises between about 8 and about 20 different RNA-binding guide RNAs.

187. The nucleic acid composition of claim 184, wherein the plurality of different RNA-binding guide RNAs comprises between about 10 and about 18 different RNA-binding guide RNAs.

188. The nucleic acid composition of claim 184, wherein the plurality of different RNA-binding guide RNAs comprises between about 12 and about 16 different RNA-binding guide RNAs.

189. The nucleic acid composition of claim 184, wherein the plurality of different RNA-binding guide RNAs comprises about 14 different RNA-binding guide RNAs.
